SEQUENCE LISTING (3 sequences)

SEQ ID NO: 1 - protein, 297 aa, synthetic construct
GSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 2 - protein, 297 aa, synthetic construct
GSEIGTGFPFDPHYVEVLGERMHYVDVGPRDGTPVLFLHGEPTSSYVWRNIIPHVAPTHRCIAPDLIGMGKSDKPDLGYFFDDHVRFMDAFIEALGLEEVVLVIHEGGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGLRIPPAEAARLAKSLPNCKAVDIGPGLNLLQEDNPDLIGSEIARWLSTLEISG

SEQ ID NO: 3 - DNA, 40 bp, synthetic construct
  source 1..40: mol_type = other DNA
ggsggggsggggsggggsggggsggggsggggsggggsgg

(19) United States
(12) Patent Application Publication
(10) Pub. No.: US 2025/0213700 A1
(43) Pub. Date: Jul. 3, 2025

(54) COMPOUNDS, COMPOSITIONS, AND METHODS FOR CELL-SPECIFIC PHARMACOLOGY

(71) Applicants: Duke University, Durham, NC (US); Fox Chase Chemical Diversity Center, Inc., Doylestown, PA (US)

(72) Inventors: Michael Tadross, Durham, NC (US); Allen B. Reitz, Chalfont, PA (US); Mark McConnell, Doylestown, PA (US); Marie Loughran, Doylestown, PA (US)

(21) Appl. No.: 18/866,835

(22) PCT Filed: May 19, 2023
(86) PCT No.: PCT/US2023/023010

§ 371 Date: Nov. 18, 2024

Related U.S. Application Data
(60) Provisional application No. 63/343,598, filed on May 19, 2022.
(60) Provisional application No. 63/379,614, filed on Oct. 14, 2022.

(51) Int. Cl. A61K 47/55; A61K 49/00; G01N 33/533; G01N 33/573

(52) U.S. Cl. CPC A61K 47/55; A61K 49/0052; G01N 33/533; G01N 33/573; G01N 2333/914

(57) ABSTRACT

Disclosed herein are compounds that have improved cellular specificity. The compounds have linkers and other moieties that can both aid in cellular specificity and that can attach functional groups to a target cell. The compounds can be used, e.g., in methods of modulating, detecting, and labeling of proteins and cells. An example method includes the compound forming a covalent bond with a dehalogenase variant.

### CROSS-REFERENCE TO RELATED APPLICATIONS

This application claims priority to U.S. Provisional Patent Application No. 63/343,598 filed on May 19, 2022 and U.S. Provisional Patent Application No. 63/379,614 filed on Oct. 14, 2022, both of which are incorporated fully herein by reference.

### FEDERALLY SPONSORED RESEARCH

This invention was made with Government support under Federal Grant nos. RF1-MH117055-01 and DP2-MH119425-01 awarded by the National Institutes of Health (NIH). The Federal Government has certain rights to this invention.

### REFERENCE TO AN ELECTRONIC SEQUENCE LISTING

The contents of the electronic sequence listing (028193-0005-WO01.xml; Size: 4,294 bytes; and Date of Creation: May 19, 2023) is herein incorporated by reference in its entirety.

### TECHNICAL FIELD

The present disclosure relates to compounds and compositions designed for pharmacological cellular-specificity and their use in biomedical applications, such as modulating neurotransmission.

### INTRODUCTION

Pharmaceutical mechanisms of action are difficult to resolve within complex organ systems, despite mechanistic knowledge at the molecular scale. The challenge is more pronounced in the brain, where it remains unknown how canonical drug-receptor interactions are transformed, via brain cells, to alter mood, anxiety, addiction, psychosis, cognition, and motor coordination. Cell-specific pharmaceutical technologies may help to untangle this complexity by making it possible to deliver clinical drugs to one cell type at a time, observe ensuing behaviors and cellular dynamics, and reconstruct mechanisms from parts to the whole.

Drugs acutely restricted by tethering (DART) is a technology that can achieve cell-specific delivery of pharmaceuticals. The approach mirrors traditional pharmacology in its modulation of endogenous receptors, acute onset upon administration, and spatial restriction via local dosing. DART can transfigure local dosing to cellular dimensions. Cells of interest can be programmed to express the HaloTag Protein (HTP), providing them a unique ability to covalently capture and locally accumulate the HaloTag Ligand (HTL) along with any conjoined pharmaceutical cargo (Rx). Cell-specificity can thus be achieved in a simple manner, by boosting the local concentration of a drug, without the need to engineer a chemical switch into the drug to control its chemical potency.

### SUMMARY

In one aspect, disclosed are compounds of formula (I)

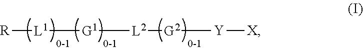

or a salt thereof, wherein: R is a functional group selected from the group consisting of a binding group, a detection label, a biologically active agent, a biorthogonal functional group, and a substrate; L¹, at each occurrence, is a linker; L² is a linker; G¹, at each occurrence, is a C₆₋₁₂arylene, a 5- to 12-membered heteroarylene, C₃₋₆cycloalkylene, or a 4- to 6-membered heterocyclylene, wherein G¹ is optionally substituted with 1-4 R^{1x}, wherein, at each occurrence, R^{1x} is independently selected from the group consisting of halogen, oxo, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, and —C₁₋₃alkylene-Q¹; R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a}; R^{1d}, at each occurrence, is independently C₁₋₆alkyl, C₁₋₆haloalkyl, G^{1a}, or —C₁₋₃alkylene-G^{1a}; G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂; Q¹, at each occurrence, is independently-OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂; G², at each occurrence, is a C₆₋₁₂arylene, a 5- to 12-membered heteroarylene, C₃₋₆cycloalkylene, or a 4- to 6-membered heterocyclylene, wherein G² is optionally substituted with 1-4 R^{2x}, wherein, at each occurrence, R^{2x} is independently selected from the group consisting of halogen, oxo, C₁₋₆alkyl, C₁₋₆haloalkyl, C₂₋₆alkenyl, —OR^{2a}, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, and —C₁₋₃alkylene-Q²; R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a}; R^{2d}, at each occurrence, is independently C₁₋₆alkyl, C₁₋₆haloalkyl, G^{2a}, or —C₁₋₃alkylene-G^{2a}; G^{2a}, at each occurrence, is independently a C₃₋₈cycloalkyl, a 4- to 12-membered heterocyclyl, a 6- to 12-membered aryl, or a 5- to 12-membered heteroaryl, wherein G^{2a} is optionally substituted with 1-5 substituents independently selected from the group consisting of halogen, oxo, C₁₋₄alkyl, —OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂; Q², at each occurrence, is independently-OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂; Y is alkylene, alkenylene, or alkynylene; and X is halogen, wherein at least one of G¹ and G² is present.

In another aspect, disclosed are compositions including a protein linked to a functional group by a linker of formula (II)

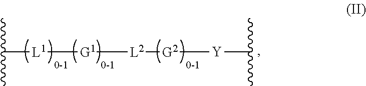

wherein: the functional group is selected from the group consisting of a binding group, a detection label, a biologically active agent, a biorthogonal functional group, and a substrate; L¹, G¹, L², G², and Y are defined as for compounds of formula (I); the protein comprises a mutant dehalogenase, and at least one of G¹ and G² is present.

In another aspect, disclosed are recombinant proteins including a mutant dehalogenase having at least 85% sequence identity to SEQ ID NO: 1, wherein the recombinant protein has three amino acid substitutions within a catalytic triad of the mutant dehalogenase and two amino acid substitutions within a tunnel entrance of the mutant dehalogenase.

In another aspect, disclosed are methods of modulating a cell, the method including: contacting a cell comprising a mutant dehalogenase on a surface of the cell with a compound as disclosed herein, wherein the mutant dehalogenase forms a bond with the compound, and wherein R binds to a receptor on a surface of the cell.

In another aspect, disclosed are methods of labeling a cell, the method including contacting a cell comprising a mutant dehalogenase located at a surface of the cell with a compound as disclosed herein, wherein the mutant dehalogenase forms a bond with the compound, thereby labeling the cell with R.

In another aspect, disclosed are methods of detecting or determining a presence or an amount of a mutant dehalogenase, the method including: contacting a mutant dehalogenase with a compound as disclosed herein, wherein the mutant dehalogenase forms a bond with the compound; and detecting or determining the presence or the amount of R, thereby detecting or determining the presence or the amount of the mutant dehalogenase.

In another aspect, disclosed are methods of modulating neurotransmission in a subject in need thereof, the method including administering to the subject an effective amount of a compound as disclosed herein, or a pharmaceutically acceptable salt thereof, optionally in combination with a pharmaceutically acceptable excipient.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows the development of HTL.2. Top Left: HTL₅₀ measured on ⁺HTP neurons; biotin-PEG₁₂-HTL applied (at a given dose) for 15 min. Streptavidin label quantifies captured biotin, dTomato estimates HTP expression. Bottom Left: capture dose-response, each symbol is mean±SEM estimated from >100 cells via regression of streptavidin vs dTomato. Right: Structural model (PDB 4KAJ) of HTP in complex with HTL.1 (top), HTL.2 (middle), and ^{dd}HTP mutations (bottom).

FIG. 2A-2I shows that the DART.2 platform can provide thousandfold cell specificity. (FIG. 2A) Modular chemistry. ‘Rx fragment’ (Rx-short spacer-alkyne) is small. ‘HTL module’ (azide-long linker-HTL) is larger due to the long PEG₃₆ linker. Final assembly is via alkyne-azide cycloaddition (e.g., gabazine.7^{alkyne}+azide^{DART.2}→gabazine.7^{DART.2}). (FIG. 2B and FIG. 2C) Wider Dosing Window: Comparison of YM90K^{DART.2} vs YM90^{KDART.1} in cultured-neuron assay of AMPA-dependent synaptic transmission, where the structure shown in FIG. 1A is Rx-PEG36-HTL. On control neurons (−HTP) both compounds have similar Rx₅₀. However, on HaloTag Protein-expressing neurons (+HTP), there is a pronounced difference in HTL₅₀. The result is that YM90^{KDART.2} has a 10-fold wider Dosing Window (DW=Rx₅₀/HTL₅₀). (FIG. 2D and FIG. 2E) Characterization in live mice expressing HTP in dSPNs of the left dorsal striatum. Alexa^{DART.2} co-infused with YM90^{KDART.2} ligand provides permanent marker of drug capture, tight correspondence between histology and behavioral potency, and 3-fold increase in behavioral potency in comparison to its predecessor. (FIGS. 2F-2I) Gabazine^{DART} in live mice for comparison.

FIG. 3A-3F show quantitative engagement of the DART.2 platform. (FIG. 3A) Mice with ⁺HTP or ^{dd}HTP in D1 cells of left dorsal striatum. Open field (1 hr) before and after Alexa488.1^{DART.2}+YM90K.1^{DART.2} (3 UM dye+30 UM drug, 1 μL in 10 min). Histology performed 24 hr later. Left, dTomato expression and Alexa488.1^{DART.2} capture from a ⁺HTP mouse. No dye capture detected in ^{dd}HTP mice. Right, open-field turning vs dye capture. Each symbol is one mouse. ^{dd}HTP mice exhibited little turning. ⁺HTP mice turned in proportion to dye capture. Dye capture can be used as a behavior-independent inclusion criterion (shading), allowing exclusion of 4 ⁺HTP mice in this dataset. (FIG. 3B) Older technology lacked tracer^{DART}. Here, an estimate of technical success with a non-DART dye (fluorogold) was attempted and a numerical algorithm dye-virus overlap was estimated. This did not provide a predictive histology marker of target engagement (right, correlation is absent when considering only the ⁺HTP mice). (FIG. 3C and FIG. 3D) Characterization in live mice expressing HTP in dSPNs of the left dorsal striatum. Alexa^{DART.2} co-infused with YM90^{KDART.2} ligand provided permanent marker of drug capture, tight correspondence between histology and behavioral potency, and 3-fold increase in behavioral potency in comparison to its predecessor. (FIG. 3E and FIG. 3F) YM90^{KDART.1} in live mice for comparison with above.

FIG. 4A-4F show the DART.2 platform can enable safe use of epileptogenic pharmaceuticals in awake behaving animals. (FIG. 4A) Validation of gabazine.1^{DART.2} in cerebellar slice. Top: voltage-clamp schematic. Bottom: tonic GABA_{A}R in granule cells expressing ⁺HTP or ^{dd}HTP. Application of gabazine.1^{DART.2} (100 nM) antagonizes the GABA_{A}R in ⁺HTP but not ^{dd}HTP cells. Subsequent application of traditional gabazine blocks all GABA_{A}R current. Data are mean±SEM, cells normalized to baseline. (FIG. 4B) In vivo delivery of gabazine.1^{DART.2} to cerebellar granule cells. Top right: schematic of experimental approach, GCaMP6f granule-cell activity obtained in the absence of sensory stimuli. Top left: example field of view, GCaMP6f signal averaged across trials. Bottom: example calcium traces (ΔF/F) in an awake ⁺HTP mouse before (baseline) and after (1 μL of 1 μM gabazine.1^{DART.2}) infusion. (FIG. 4C) In vivo, 1 μL of 1 μM gabazine.1^{DART.2} to ⁺HTP mice. Color indicates ΔF/F, each row is one cell before (left) and after (right) gabazine.1^{DART.2} infusion. Full dataset from 6 mice (n=1707 cells, p<0.0001, paired t-test, before vs after infusion). (FIG. 4D) In vivo, 1 μL of 1 μM gabazine.1^{DART.2} to ^{dd}HTP mice. Format as in panel C. Full dataset from 4 mice (n=1084 cells, p=0.76, paired t-test, before vs after infusion). (FIG. 4E) Validation of gabazine.7^{DART.2} in VTA slice. Top: evoked-IPSC configuration. Bottom: 300 nM gabazine.7^{DART.2} produced a nearly complete block of evoked IPSCs on ⁺HTP neurons (n=10), with no impact on ^{dd}HTP cells (n=8). Data are mean±SEM, cells normalized to baseline. Control experiments confirmed the GABA_{A}R specificity of the manipulation. (FIG. 4F) In vivo, 1.2 μL of 10 μM gabazine.7^{DART.2} infusion into the VTA increased locomotor speed in VTA_{DA} ⁺HTP mice (n=17) but not in ^{dd}HTP control mice (n=13). Data are mean±SEM, normalized to baseline speed of each animal. The difference in ⁺HTP vs ^{dd}HTP post-infusion speed demonstrated efficacy of the tethered, but not the ambient compound (p<0.01, unpaired t-test).

FIG. 5 shows quantitative target engagement and whole-brain delivery. Top left: Mice expressing pan-neuronal GCaMP8s with SOM-specific ⁺HTP. Window over V1. DART delivered via the contralateral ventricle. Bottom left: In-vivo and ex-vivo expression and dye capture. Right: Mice presented oriented visual gratings, 2P Ca²⁺ imaging obtained before and 5-6 hr after infusion of 0.3 nmole Alexa647.1^{DART.2}+3 nmole YM90K.1^{DART.2} (2 μL volume) into the contralateral ventricle. Data show mean±SEM of cells from 3 mice. Following the manipulation, responses in ⁺HTP SOM cells is reduced (p=0.01, n=26, paired t-test), and responses in ⁻HTP, putative pyramidal cells, were unchanged or increased (p=0.04, n=223, paired t-test).

FIG. 6A-6C show bidirectional manipulation of AMPAR and GABA_{A}R neurotransmission. (FIG. 6A) Viral strategy (top panel). Intravitreal AAV₇ₘ₈-DIO-HTP_{GPI}-2A-dTomao achieves retinal ganglion cell (RGC) expression in PV::cre mice. Experimental overview (second panel from the top to bottom panel). Top: whole retina; synaptic current is evoked (stim) and recorded (voltage-clamp of dTomato+ RGC), with ionic and pharmaceutical conditions chosen to isolate the AMPAR or GABA_{A}R. A 10 min baseline is followed by 15 min application of 300 nM Rx^{DART.2}+30 nM Alexa^{647.1DART.2}. After wash, tethered dye (bottom panel) is seen on ⁺HTP but not ^{dd}HTP cells. (FIG. 6B) CMPDA.2^{DART.2} (left). Electropositive spacer (PDB 3RNN). AMPAR evoked EPSC integral (total charge transfer) was boosted 3-fold on ⁺HTP but not ^{dd}HTP cells (mean±SEM, cells normalized to baseline). EPSC waveform before and after CMPDA.2^{DART.2}. AMPAR structural model (middle) (composite of PDB 5WEO, 3RNN, and 1FTL). Cylinder depicts cell membrane. CMPDA.2^{DART.2} binds within an inter-clamshell interface (2 per receptor). YM90K.1^{DART.2} binds the orthosteric glutamate binding site in the clamshell (4 per receptor). YM90K.1^{DART.2} (right). Structural model (PDB 1FTL). AMPAR evoked EPSC peak is blocked on ⁺HTP cells but not on ^{dd}HTP cells (mean±SEM, cells normalized to baseline). EPSC waveforms before and after YM90K.1^{DART.2}. (FIG. 6C) diazepam.1^{DART.2} (left). Structural model (PDB 6HUP). GABA_{A}R evoked IPSC integral (total charge transfer) is boosted 2-fold on ⁺HTP but not ^{dd}HTP cells (mean±SEM, cells normalized to baseline). IPSC waveform before and after diazepam.1^{DART.2}. GABA_{A}R (middle) structural model (composite of PDB 6HUP and 6HUK). Cylinder depicts cell membrane; diazepam.1^{DART.2} binds the α/δ interface (1 per receptor). gabazine.1^{DART.2} binds the orthosteric GABA-binding site in the α/β interface (2 per receptor). gabazine.1^{DART.2} (right). Structural model (PDB 6HUK). Bottom left: GABA_{A}R evoked IPSC peak is blocked on ⁺HTP cells but not on ^{dd}HTP cells (mean±SEM, cells normalized to baseline). Bottom right: IPSC waveforms before and after gabazine.1^{DART.2}.

### DETAILED DESCRIPTION

DART promises genetic control over pharmaceuticals. The technology can achieve cellular specificity by instructing cells to express the HaloTag Protein (HTP), which can capture and locally accumulate a drug linked to the HaloTag Ligand (HTL). The approach offers design simplicity as it boosts the local abundance of a drug, without the need for a chemical switch to mask and locally unmask the drug. Nevertheless, because pharmaceutical dose-response curves are graded, the strategy only approximates an on/off switch. Here, the present application discloses the DART.2 platform, comprising at least three advances. The first is an improvement in cellular specificity by improving the HTL, which can enable rapid accumulation of drug molecules to ˜1,000-times the ambient concentration within minutes. Second, the establishment of new standards of rigor with control reagents and fluorescent tracers for quantitative target engagement. Third, the extension of this approach to positive allosteric modulators, demonstrating compatibility with this clinically significant class. New DART.2 pharmaceuticals are shown, enabling antagonism or positive-allosteric modulation of excitatory (AMPAR) or inhibitory (GABAAR) synapses. Reagents were tested in the mouse basal ganglia, cerebellum, retina, and visual cortex. Collectively, it was found that DART.2 can enable cell-specific pharmacology over large brain volumes, dosing from a distance, and safe delivery of even epileptogenic drugs. The present disclosure also provides a distribution platform for viral and chemical reagents, including click-chemistry modules for extension to diverse pharmaceuticals.

### 1. Definitions

Unless otherwise defined, all technical and scientific terms used herein have the same meaning as commonly understood by one of ordinary skill in the art. In case of conflict, the present document, including definitions, will control. The materials, methods, and examples disclosed herein are illustrative only and not intended to be limiting. Methods and materials similar or equivalent to those described herein can be used in practice or testing of the disclosed invention. All publications, patent applications, patents and other references mentioned herein are incorporated by reference in their entirety.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are contemplated, and for the range 1.5-2, the numbers 1.5, 1.6, 1.7, 1.8, 1.9, and 2 are contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

The term “alkoxy,” as used herein, refers to a group-O-alkyl. Representative examples of alkoxy include, but are not limited to, methoxy, ethoxy, propoxy, 2-propoxy, butoxy and tert-butoxy.

The term “alkyl,” as used herein, refers to a straight or branched, saturated hydrocarbon chain containing from 1 to 30 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and n-dodecyl. The alkyl group may be substituted or unsubstituted.

The term “alkenyl” as used herein, means a straight or branched hydrocarbon chain containing from 2 to 30 carbon atoms with at least one carbon-carbon double bond. The alkenyl group may be substituted or unsubstituted.

The term “alkynyl,” as used herein, refers to straight or branched hydrocarbon chain containing from 2 to 30 carbon atoms with at least one carbon-carbon triple bond. The alkynyl group may be substituted or unsubstituted.

The term “alkylene,” as used herein, refers to a divalent alkyl group, examples of which include, but are not limited to, —CH₂CH₂—, —CH₂CH₂CH₂—, —CH₂CH₂CH₂CH₂—, and —CH₂CH₂CH₂CH₂CH₂—. An alkylene group may be optionally substituted with one or more substituents.

The term “alkenylene,” as used herein, refers to a divalent alkenyl group, examples of which include, but are not limited to —CH═CH—, —CH═CH—CH₂—, —CH═CH—CH₂—CH₂— and —CH₂—CH═CH—CH₂—. An alkenylene group may be optionally substituted with one or more substituents.

The term “alkynylene” refers to a divalent alkynyl group, examples of which include, but are not limited to —C≡C—, —C≡C—CH₂—, —C≡C—CH₂—CH₂— and —CH₂—C≡C—CH₂—. An alkynylene group may be optionally substituted with one or more substituents.

The term “aryl,” as used herein, refers to a phenyl or a phenyl appended to the parent molecular moiety and fused to a cycloalkane group (e.g., the aryl may be indan-4-yl), fused to a 6-membered arene group (i.e., the aryl is naphthyl), or fused to a non-aromatic heterocycle (e.g., the aryl may be benzo[d][1,3]dioxol-5-yl). The term “phenyl” is used when referring to a substituent and the term 6-membered arene is used when referring to a fused ring. The 6-membered arene is monocyclic (e.g., benzene or benzo). The aryl may be monocyclic (phenyl) or bicyclic (e.g., a 9- to 12-membered fused bicyclic system).

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

The term “arylene” refers to a divalent group derived from an aromatic ring or ring system. For example, a “6-membered 1,4-arylene” is a divalent group derived from a monocyclic 6-membered aromatic ring having points of attachment or substitution on two carbon ring atoms in 1,4 relation to each other, i.e., para substitution, and a “6-membered 1,3-arylene” is a divalent group derived from a monocyclic 6-membered aromatic ring and having points of attachment or substitution on two carbon ring atoms in 1,3 relation to each other, i.e., meta substitution. For purposes of illustration, an example of a 6-membered 1,4-arylene is

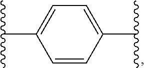 [SMILES: *c1ccc(*)cc1]

and an example of a 6-membered 1,3-arylene is

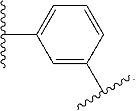 [SMILES: *c1cccc(*)c1]

The term “carbocyclyl” means a “cycloalkyl” or a “cycloalkenyl.” The term “carbocycle” means a “cycloalkane” or a “cycloalkene.” The term “carbocyclyl” refers to a “carbocycle” when present as a substituent.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. A control may be a subject or cell without a compound or genetic construct as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

The term “cycloalkyl” or “cycloalkane,” as used herein, refers to a saturated ring system containing all carbon atoms as ring members and zero double bonds. The term “cycloalkyl” is used herein to refer to a cycloalkane when present as a substituent. A cycloalkyl may be a monocyclic cycloalkyl (e.g., cyclopropyl), a fused bicyclic cycloalkyl (e.g., decahydronaphthalenyl), or a bridged cycloalkyl in which two non-adjacent atoms of a ring are linked by an alkylene bridge of 1, 2, 3, or 4 carbon atoms (e.g., bicyclo[2.2.1]heptanyl). Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, and bicyclo[1.1.1]pentanyl.

The terms cycloalkylene and heterocyclylene refer to divalent groups derived from a cycloalkane and a heterocycle, respectively. For purposes of illustration, examples of cycloalkylene and heterocyclylene include, respectively,

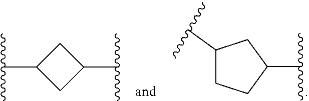 [SMILES: *C1CC(*)C1 ; *C1CCC(*)C1]

Cycloalkylene and heterocyclylene include a geminal divalent groups such as 1,1-C₃₋₆cycloalkylene

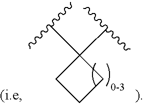

A further example is 1,1-cyclopropylene

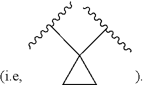 [SMILES: *C1(*)CC1]

The term “effective dosage” or “therapeutic dosage” or “therapeutically effective amount” or “effective amount,” as used herein, refers to an amount sufficient to effect beneficial or desirable biological and/or clinical results, to modulate a biological process, and/or treat a disease or one or more of its symptoms and/or to prevent or reduce the risk of the occurrence or reoccurrence of the disease or disorder or symptom(s) thereof. A therapeutically effective amount is also one in which any toxic or detrimental effects of substance are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount. In reference to neurotransmission an effective or therapeutically effective amount can include an amount sufficient to, among other things, affect a behavioral response (e.g., locomotion of a subject).

“Genetic construct” as used herein refers to the DNA or RNA molecules that comprise a polynucleotide that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operable linked to a coding sequence that encodes a protein such that when present in the cell of the individual, the coding sequence will be expressed. For example, a genetic construct may encode a HaloTag protein that may be expressed in a cell of interest.

The term “halogen” or “halo,” as used herein, means Cl, Br, I, or F.

The term “haloalkyl,” as used herein, means an alkyl group, as defined herein, in which one, two, three, four, five, six, seven or eight hydrogen atoms are replaced by a halogen.

The term “heterologous” as used herein refers to nucleic acid comprising two or more subsequences that are not found in the same relationship to each other in nature. For instance, a nucleic acid that is recombinantly produced typically has two or more sequences from unrelated genes synthetically arranged to make a new functional nucleic acid, for example, a promoter from one source and a coding region from another source. The two nucleic acids are thus heterologous to each other in this context. When added to a cell, the recombinant nucleic acids would also be heterologous to the endogenous genes of the cell. Thus, in a chromosome, a heterologous nucleic acid would include a non-native (non-naturally occurring) nucleic acid that has integrated into the chromosome, or a non-native (non-naturally occurring) extrachromosomal nucleic acid. Similarly, a heterologous protein indicates that the protein comprises two or more sequences or subsequences that are not found in the same relationship to each other in nature (for example, a “fusion protein,” where the two subsequences are encoded by a single nucleic acid sequence).

The term “heteroalkyl,” as used herein, means an alkyl group, as defined herein, in which one or more of the carbon atoms has been replaced by a heteroatom selected from S, O, P and N. Representative examples of heteroalkyls include, but are not limited to, alkyl ethers, secondary and tertiary alkyl amines, amides, and alkyl sulfides.

The term “heteroalkylene” as used herein, means an alkylene group, as defined herein, in which one or more of the carbon atoms has been replaced by a heteroatom selected from S, Si, O, P and N. Examples include, but are not limited to, —CH₂OCH₂—, —CH₂NHCH₂—, polyethylene glycol groups (e.g., —(CH₂CH₂O)ₙ—), polyethyleneimine groups (e.g., —(CH₂CH₂NH)ₙ—), and the like. A heteroalkylene group may be optionally substituted with one or more substituents.

The term “heteroaryl,” as used herein, refers to an aromatic monocyclic heteroatom-containing ring (monocyclic heteroaryl) or a bicyclic ring system containing at least one monocyclic heteroaromatic ring (bicyclic heteroaryl). The term “heteroaryl” is used herein to refer to a heteroarene when present as a substituent. The monocyclic heteroaryl are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered aromatic monocyclic rings have three double bonds. The bicyclic heteroaryl is an 8- to 12-membered ring system and includes a fused bicyclic heteroaromatic ring system (i.e., 10π electron system) such as a monocyclic heteroaryl ring fused to a 6-membered arene (e.g., quinolin-4-yl, indol-1-yl), a monocyclic heteroaryl ring fused to a monocyclic heteroarene (e.g., naphthyridinyl), and a phenyl fused to a monocyclic heteroarene (e.g., quinolin-5-yl, indol-4-yl). A bicyclic heteroaryl/heteroarene group includes a 9-membered fused bicyclic heteroaromatic ring system having four double bonds and at least one heteroatom contributing a lone electron pair to a fully aromatic 10π electron system, such as ring systems with a nitrogen atom at the ring junction (e.g., imidazopyridine) or a benzoxadiazolyl. A bicyclic heteroaryl also includes a fused bicyclic ring system composed of one heteroaromatic ring and one non-aromatic ring such as a monocyclic heteroaryl ring fused to a monocyclic carbocyclic ring (e.g., 6,7-dihydro-5H-cyclopenta[b]pyridinyl), or a monocyclic heteroaryl ring fused to a monocyclic heterocycle (e.g., 2,3-dihydrofuro[3,2-b]pyridinyl). The bicyclic heteroaryl is attached to the parent molecular moiety at an aromatic ring atom. Other representative examples of heteroaryl include, but are not limited to, indolyl (e.g., indol-1-yl, indol-2-yl, indol-4-yl), pyridinyl (including pyridin-2-yl, pyridin-3-yl, pyridin-4-yl), pyrimidinyl, pyrazinyl, pyridazinyl, pyrazolyl (e.g., pyrazol-4-yl), pyrrolyl, benzopyrazolyl, 1,2,3-triazolyl (e.g., triazol-4-yl), 1,3,4-thiadiazolyl, 1,2,4-thiadiazolyl, 1,3,4-oxadiazolyl, 1,2,4-oxadiazolyl, imidazolyl, thiazolyl (e.g., thiazol-4-yl), isothiazolyl, thienyl, benzimidazolyl (e.g., benzimidazol-5-yl), benzothiazolyl, benzoxazolyl, benzoxadiazolyl, benzothienyl, benzofuranyl, isobenzofuranyl, furanyl, oxazolyl, isoxazolyl, purinyl, isoindolyl, quinoxalinyl, indazolyl (e.g., indazol-4-yl, indazol-5-yl), quinazolinyl, 1,2,4-triazinyl, 1,3,5-triazinyl, isoquinolinyl, quinolinyl, imidazo[1,2-a]pyridinyl (e.g., imidazo[1,2-a]pyridin-6-yl), naphthyridinyl, pyridoimidazolyl, thiazolo[5,4-b]pyridin-2-yl, and thiazolo[5,4-d]pyrimidin-2-yl.

The term “heterocycle” or “heterocyclic,” as used herein, means a monocyclic heterocycle, a bicyclic heterocycle, or a tricyclic heterocycle. The term “heterocyclyl” is used herein to refer to a heterocycle when present as a substituent. The monocyclic heterocycle is a three-, four-, five-, six-, seven-, or eight-membered ring containing at least one heteroatom independently selected from the group consisting of O, N, and S. The three- or four-membered ring contains zero or one double bond, and one heteroatom selected from the group consisting of O, N, and S. The five-membered ring contains zero or one double bond and one, two or three heteroatoms selected from the group consisting of O, N and S. The six-membered ring contains zero, one or two double bonds and one, two, or three heteroatoms selected from the group consisting of O, N, and S. The seven- and eight-membered rings contains zero, one, two, or three double bonds and one, two, or three heteroatoms selected from the group consisting of O, N, and S. Representative examples of monocyclic heterocyclyls include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, 2-oxo-3-piperidinyl, 2-oxoazepan-3-yl, oxadiazolinyl, oxadiazolidinyl, oxazolinyl, oxazolidinyl, oxetanyl, oxepanyl, oxocanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a 6-membered arene, or a monocyclic heterocycle fused to a monocyclic cycloalkane, or a monocyclic heterocycle fused to a monocyclic cycloalkene, or a monocyclic heterocycle fused to a monocyclic heterocycle, or a monocyclic heterocycle fused to a monocyclic heteroarene, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. The bicyclic heterocyclyl is attached to the parent molecular moiety at a non-aromatic ring atom (e.g., indolin-1-yl). Representative examples of bicyclic heterocyclyls include, but are not limited to, chroman-4-yl, 2,3-dihydrobenzofuran-2-yl, 2,3-dihydrobenzothien-2-yl, 1,2,3,4-tetrahydroisoquinolin-2-yl, 2-azaspiro[3.3]heptan-2-yl, 2-oxa-6-azaspiro[3.3]heptan-6-yl, azabicyclo[2.2.1]heptyl (including 2-azabicyclo[2.2.1]hept-2-yl), azabicyclo[3.1.0]hexanyl (including 3-azabicyclo[3.1.0]hexan-3-yl), 2,3-dihydro-1H-indol-1-yl, isoindolin-2-yl, octahydrocyclopenta[c]pyrrolyl, octahydropyrrolopyridinyl, tetrahydroisoquinolinyl, 7-oxabicyclo[2.2.1]heptanyl, hexahydro-2H-cyclopenta[b]furanyl, 2-oxaspiro[3.3]heptanyl, and 3-oxaspiro[5.5]undecanyl. Tricyclic heterocycles are exemplified by a bicyclic heterocycle fused to a 6-membered arene, or a bicyclic heterocycle fused to a monocyclic cycloalkane, or a bicyclic heterocycle fused to a monocyclic cycloalkene, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.13,7]decane), and oxa-adamantane (2-oxatricyclo[3.3.1.13,7]decane). The monocyclic, bicyclic, and tricyclic heterocyclyls are connected to the parent molecular moiety at a non-aromatic ring atom.

The term “heteroarylene” refers to a divalent group derived from a heteroaromatic ring or ring system. For example, a “6-membered 1,4-heteroarylene containing 1-2 nitrogen atoms” is a divalent group derived from a monocyclic 6-membered heteroaromatic ring having 1-2 nitrogen atoms and having points of attachment or substitution on two carbon ring atoms in 1,4 relation to each other, i.e., para substitution. For purposes of illustration, examples of a 6-membered 1,4-heteroarylene containing 1-2 nitrogen atoms are

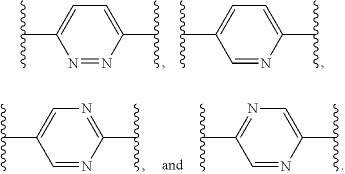

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

The term “linker” “linked” or “linking” refers to a chemical moiety that attaches two moieties together, such as G¹ to a functional group (R) as described herein. The linking can be via covalent bonds, ionic bonds, hydrophobic interactions, hydrogen bonds, van der Waals forces (e.g., London dispersion forces), dipole-dipole interactions, and the like. The linking can be a direct linkage between the two moieties being linked, or indirectly, such as via a linker.

A “protein” or “polypeptide” is a linked sequence of 50 or more amino acids linked by peptide bonds. A peptide is a linked sequence of 2 to 50 amino acids linked by peptide bonds. The polypeptide can be natural, synthetic, or a modification or combination of natural and synthetic. Peptides and polypeptides include proteins such as binding proteins and receptors. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. These structures are commonly known as domains, for example, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Promoter” as used herein means a synthetic or naturally derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

The term “small molecule,” as used herein, refers to inorganic or organic compounds having a molecular weight of less than 3,000 Daltons.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamsters, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, or an infant. The subject may be male. The subject may be female. In some embodiments, the subject has a specific genetic marker. The subject may be undergoing other forms of treatment.

“Substantially identical” means that a first and second amino acid sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, or 1100 amino acids. This can also be referred to as X % sequence identity, where a first and second amino acid sequence are at least X % identical over a region of amino acids as listed above.

The term “substituted” refers to a group that may be further substituted with one or more non-hydrogen substituent groups. Substituent groups include, but are not limited to, halogen, ═O (oxo), ═S (thioxo), cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

“Vector” as used herein means a nucleic acid sequence containing an origin of replication. A vector may be a viral vector, bacteriophage, bacterial artificial chromosome, or yeast artificial chromosome. A vector may be a DNA or RNA vector. A vector may be a self-replicating extrachromosomal vector, and preferably, is a DNA plasmid. For example, the vector may encode a protein that binds a ligand as described herein such as a HaloTag protein.

Terms such as “alkyl,” “cycloalkyl,” “alkylene,” etc. may be preceded by a designation indicating the number of atoms present in the group in a particular instance (e.g., “C₁₋₄alkyl,” “C₃₋₆cycloalkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

### 2. Compounds of Formula (I)

Provided herein are compounds with improved cellular specificity. The compounds can be of formula (I)

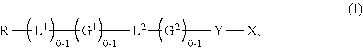

or a salt thereof, wherein: R is a functional group selected from the group consisting of a binding group, a detection label, a biologically active agent, a biorthogonal functional group, and a substrate; L¹, at each occurrence, is a linker; L² is a linker; G¹, at each occurrence, is a C₆-12arylene, a 5- to 12-membered heteroarylene, C₃₋₆cycloalkylene, or a 4- to 6-membered heterocyclylene, wherein G¹ is optionally substituted with 1-4 R^{1x}, wherein, at each occurrence, R^{1x} is independently selected from the group consisting of halogen, oxo, C₁₋₆alkyl, C₁₋₈haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, and —C₁₋₃alkylene-Q¹; R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a}; R^{1d}, at each occurrence, is independently C₁₋₆alkyl, C₁₋₆haloalkyl, G^{1a}, or —C₁₋₃alkylene-G^{1a}; G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂; Q¹, at each occurrence, is independently-OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂; G², at each occurrence, is a C₆₋₁₂arylene, a 5- to 12-membered heteroarylene, C₃₋₆cycloalkylene, or a 4- to 6-membered heterocyclylene, wherein G² is optionally substituted with 1-4 R^{2x}, wherein, at each occurrence, R^{2x} is independently selected from the group consisting of halogen, oxo, C₁₋₆alkyl, C₁₋₆haloalkyl, C₂₋₆alkenyl, —OR^{2a}, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, and —C₁₋₃alkylene-Q²; R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a}; R^{2d}, at each occurrence, is independently C₁₋₆alkyl, C₁₋₆haloalkyl, G^{2a}, or —C₁₋₃alkylene-G^{2a}; G^{2a}, at each occurrence, is independently a C₃₋₈cycloalkyl, a 4- to 12-membered heterocyclyl, a 6- to 12-membered aryl, or a 5- to 12-membered heteroaryl, wherein G^{2a} is optionally substituted with 1-5 substituents independently selected from the group consisting of halogen, oxo, C₁₋₄alkyl, —OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂; Q², at each occurrence, is independently —OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂; Y is alkylene, alkenylene, or alkynylene; and X is halogen, wherein at least one of G¹ and G² is present.

An example compound of formula (I) includes:

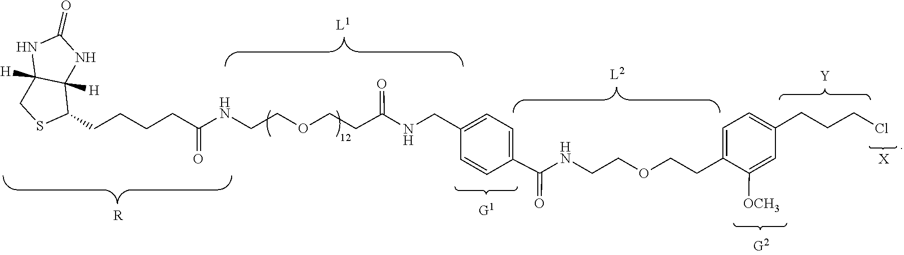

Compounds of formula (I) have a reactive group that can be recognized by a mutant protein (e.g., mutant dehalogenase), which can form a covalent bond thereto. The reactive group can include —Y—X of the compounds of formula (I). In some embodiments, Y is C₁₋₁₀alkylene, C₁₋₁₀alkenylene, or C₁₋₁₀alkynylene. In some embodiments, Y is C₁₋₄alkylene, C₁₋₄alkenylene, or C₁₋₄alkynylene. In some embodiments, Y is C₂₋₄alkylene, C₂₋₄alkenylene, or C₂₋₄alkynylene. In some embodiments, X is F, Cl, Br, or I. In some embodiments, X is Cl, Br, or I.

The cyclic groups of the compounds of formula (I) can improve bond formation of the compounds to a mutant dehalogenase by improving its positioning within the binding pocket. The cyclic groups can include G¹ and G². As discussed above, at least one of G¹ and G² is present in the compound. In some embodiments, only G¹ is present, only G² is present, or both are present in the compound. In some embodiments, both G¹ and G² are present. G¹ and G² can be attached to different moieties in varying positions (e.g., meta, para, or ortho). For example, G² can be attached to L² and Y at meta or para positions. In addition, G¹ and G² can be varying distances from each other by linearly connected atoms. For example, G¹ can be separated from G² by 2 to 20 linearly connected atoms, such as 3 to 18 linearly connected atoms, 4 to 16 linearly connected atoms, 5 to 15 linearly connected atoms, 4 to 10 linearly connected atoms, or 5 to 8 linearly connected atoms.

In some embodiments, G¹ is optionally substituted with 1-2 R^{1x}, wherein, at each occurrence, R^{1x} is selected from the group consisting of halogen, C₁₋₆haloalkyl, —OR^{1a}, —NR^{1a}R^{2b}, —SR^{1a}, —NR^{1a}C(O)R^{1c}, —C(O)OR^{1a}, —C(O)NR^{1a}R^{1b}, and —C(O)R^{1c}; and R^{1a}, R^{1b}, and R^{1c}, at each occurrence, are each independently hydrogen, C₁₋₆alkyl, or C₁₋₆haloalkyl.

In some embodiments, G¹ is optionally substituted with 1 R^{1x}, wherein R^{1x} is selected from the group consisting of halogen, C₁₋₆haloalkyl, —OR^{1a}, —C(O)OR^{1a}, —C(O)NR^{1a}R^{1b}, and —C(O)R^{1c}; and R^{1a}, R^{1b}, and R^{1c}, at each occurrence, are each independently hydrogen, C₁₋₆alkyl, or C₁₋₆haloalkyl.

In some embodiments, R^{1x} is —OR^{1a} or —C(O)OR^{1a}; and R^{1a}, at each occurrence, is independently hydrogen or C₁₋₄alkyl.

In some embodiments, G² is optionally substituted with 1-2 R^{2x}, wherein, at each occurrence, R^{2x} is selected from the group consisting of halogen, C₁₋₆haloalkyl, —OR^{2a}, —NR^{2a}R^{2b}, —SR^{2a}, —NR^{2a}C(O)R^{2c}, —C(O)OR^{2a}, —C(O)NR^{2a}R^{2b}, —C(O)R^{2c}, —SO₂R^{2d}, and —SO₂NR^{2a}R^{2b}; and R^{2a}, R^{2b}, R^{2c}, and R^{2d}, at each occurrence, are each independently hydrogen, C₁₋₆alkyl, or C₁₋₆haloalkyl.

In some embodiments, G² is substituted with 1-2 R^{2x}, wherein, at each occurrence, R^{2x} is selected from the group consisting of halogen, C₁₋₆haloalkyl, —OR^{2a}, —NR^{2a}R^{2b}, —SR^{2a}, —NR^{2a}C(O)R^{2c}, —C(O)OR^{2a}, —C(O)NR^{2a}R^{2b}, —C(O)R^{2c}, —SO₂R^{2d}, and —SO₂NR^{2a}R^{2b}; and R^{2a}, R^{2b}, R^{2c}, and R^{2d}, at each occurrence, are each independently hydrogen, C₁₋₆alkyl, or C₁₋₆haloalkyl.

In some embodiments, R^{2x} is —OR^{2a}; and R^{2a} is methyl.

In some embodiments, G¹ is C₆₋₈arylene, C₄₋₆cycloalkylene, the 4- to 6-membered heterocyclylene, or the 5- to 12-membered heteroarylene, where the ring system of the 5- to 12-membered heteroarylene at G¹ is a 5- to 8-membered heteroarylene.

In some embodiments, G¹ is C₆₋₈arylene, C₄₋₆cycloalkylene, the 4- to 6-membered heterocyclylene, or the 5- to 12-membered heteroarylene, where the ring system of the 5- to 12-membered heteroarylene at G¹ is a 5- to 8-membered heteroarylene, wherein G¹ is optionally substituted with 1 R^{1x}, wherein R^{1x} is selected from the group consisting of —OR^{1a} or —C(O)OR^{1a}; and R^{1a}, at each occurrence, is independently hydrogen or C₁₋₄alkyl; G² is C₆₋₈arylene, C₄₋₆cycloalkylene, the 4- to 6-membered heterocyclylene, or the 5- to 12-membered heteroarylene, where the ring system of the 5- to 12-membered heteroarylene at G² is a 5- to 8-membered heteroarylene, wherein G² is substituted with 1-2 R^{2x}, wherein, at each occurrence, R^{2x} is independently selected from the group consisting of halogen, C₁₋₆haloalkyl, —OR^{2a}, —NR^{2a}R^{2b}, —SR^{2a}, —NR^{2a}C(O)R^{2c}, —C(O)OR^{2a}, —C(O)NR^{2a}R^{2b}, —C(O)R^{2c}, —SO₂R^{2d}, and —SO₂NR^{2a}R^{2b}; and R^{2a}, R^{2b}, R^{2c}, and R^{2d} at each occurrence, are each independently hydrogen, C₁₋₆alkyl, or C₁₋₆haloalkyl.

In some embodiments, G¹ is C₆₋₈arylene, C₄₋₆cycloalkylene, the 4- to 6-membered heterocyclylene, or the 5- to 12-membered heteroarylene, where the ring system of the 5- to 12-membered heteroarylene at G¹ is a 5- to 8-membered heteroarylene; G² is C₆₋₈arylene, C₄-6cycloalkylene, the 4- to 6-membered heterocyclylene, or the 5- to 12-membered heteroarylene, where the ring system of the 5- to 12-membered heteroarylene at G² is a 5- to 8-membered heteroarylene, wherein G² is substituted with 1-2 R^{2x}, wherein, at each occurrence, R^{2x} is independently selected from the group consisting of halogen, C₁₋₆haloalkyl, —OR^{2a}, —NR^{2a}R^{2b}, —SR^{2a}, —NR^{2a}C(O)R^{2c}, —C(O)OR^{2a}, —C(O)NR^{2a}R^{2b}, —C(O)R^{2c}, —SO₂R^{2d}, and —SO₂NR^{2a}R^{2b}; and R^{2a}, R^{2b}, R^{2c}, and R^{2d}, at each occurrence, are each independently hydrogen, C₁₋₆alkyl, or C₁₋₆haloalkyl.

In some embodiments, G¹ is C₆₋₈arylene, the 5- to 8-membered heteroarylene, C₄-6cycloalkylene, or the 4- to 6-membered heterocyclylene, wherein G¹ is optionally substituted with 1 R^{1x}, wherein R^{1x} is selected from the group consisting of —OR^{1a} or —C(O)OR^{1a}; and R^{1a} at each occurrence, is independently hydrogen or C₁₋₄alkyl; G² is C₆₋₈arylene, the 5- to 8-membered heteroarylene, C₄₋₆cycloalkylene, or the 4- to 6-membered heterocyclylene, wherein G² is substituted with 1 R^{2x}, wherein R^{2x} is selected from the group consisting of halogen and —OR^{2a}; and R^{2a} is hydrogen or C₁₋₄alkyl.

In some embodiments, G¹ is C₆₋₈arylene, the 5- to 8-membered heteroarylene, C₄₋₆cycloalkylene, or the 4- to 6-membered heterocyclylene; G² is C₆₋₈arylene, the 5- to 8-membered heteroarylene, C₄₋₆cycloalkylene, or the 4- to 6-membered heterocyclylene, wherein G² is substituted with 1 R^{2x}, wherein R^{2x} is selected from the group consisting of halogen and —OR^{2a}; and R^{2a} is hydrogen or C₁₋₄alkyl.

Linkers of the compounds of formula (I) can also aid in improving the bond formation of the compounds to a mutant dehalogenase by improving its positioning within the binding pocket. Linkers can include L¹ and L². L¹ can be any suitable linker that can attach (e.g., covalently bond) different moieties of the compounds of formula (I) (e.g., G¹ to R). In addition, in some embodiments, L¹ is not present, e.g., where G¹ is attached directly to R. L² can be any suitable linker that can attach different moieties of the compounds of formula (I) (e.g., G¹ to G²). L² can be of varying length. For example, L² can be 2 to 50 linearly connected atoms, such as 2 to 30 linearly connected atoms, 2 to 20 linearly connected atoms, 2 to 15 linearly connected atoms, 4 to 15 linearly connected atoms, 4 to 10 linearly connected atoms, 5 to 9 linearly connected atoms, 7 to 50 linearly connected atoms, 7 to 30 linearly connected atoms, 7 to 15 linearly connected atoms, or 6 to 8 linearly connected atoms. In some embodiments, L² is greater than or equal to 7 linearly connected atoms.

In some embodiments, L¹ is alkylene, heteroalkylene, heteroalkylene-C(O)NR^{a}-heteroalkylene, heteroalkylene-C(O)NR^{a}-alkylene, heteroalkylene-C(O)O-heteroalkylene, heteroalkylene-OC(O)NR^{a}-heteroalkylene, heteroalkylene-C(O)O-heteroalkylene-C(O)-alkylene, heteroalkylene-C(O)NR^{a}-heteroalkylene-C(O)-alkylene, or heteroalkylene-OC(O)NR^{a}-heteroalkylene-C(O)-alkylene; L² is —C(O)NR^{a}-heteroalkylene, —C(O)NR^{a}-alkylene-NR^{a}C(O)—, or —C(O)NR^{a}-alkylene-C(O)NR^{a}-alkylene; and R^{a}, at each occurrence, is independently hydrogen or alkyl.

In some embodiments, L¹ is

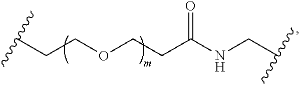

wherein m is 1 to 6,000. In some embodiments, m is 1 to 5,000, 1 to 1,000, 1 to 500, or 1 to 100. In some embodiments, m is greater than 1, greater than 2, greater than 3, greater than 4, greater than 5, greater than 10, greater than 15, greater than 20, or greater than 30. In some embodiments, m is less than 6,000, less than 5,000, less than 4,000, less than 3,000, or less than 2,000. The foregoing description regarding m can be applied to other instances herein where m is used to describe L¹. The foregoing description regarding m can also be applied to n, e.g., in formulas (I-a) and (I-b).

In some embodiments, L² is —C(O)NR^{a}—C₂₋₁₀heteroalkylene, —C(O)NR^{a}—C₁₋₁₀alkylene-NR^{a}C(O)—, or —C(O)NR^{a}—C₁₋₁₀alkylene-C(O)NR^{a}—C₁₋₆alkylene; and R^{a} is hydrogen or C₁₋₃alkyl.

In some embodiments, L² is —C(O)NR^{a}—C₄₋₈heteroalkylene, —C(O)NR^{a}—C₁₋₄alkylene-NR^{a}C(O)—, or —C(O)NR^{a}—C₁₋₄alkylene-C(O)NR^{a}—C₁₋₄alkylene; and R^{a} is hydrogen.

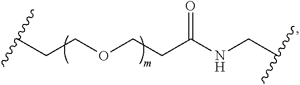

In some embodiments, L¹ is wherein m is 1 to 6,000; L² is —C(O)NR^{a}—C₂₋₁₀heteroalkylene, —C(O)NR^{a}—C₁₋₁₀alkylene-NR^{a}C(O)—, or —C(O)NR^{a}—C₁₋₁₀alkylene-C(O)NR^{a}—C₁₋₆alkylene; and R^{a} is hydrogen or C₁₋₃alkyl.

In some embodiments, the compound has formula (I-a)

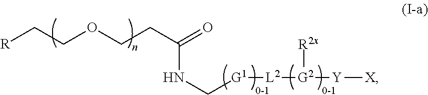

or a salt thereof, wherein: R is a functional group selected from the group consisting of a binding group, a detection label, a biologically active agent, a biorthogonal functional group, and a substrate; L² is

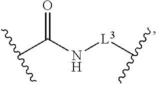

wherein: L³ is heteroalkylene, alkylene-NR^{a}C(O)—, or alkylene-C(O)NR^{a}-alkylene; R^{a}, at each occurrence, is hydrogen or C₁₋₄alkyl; G¹, at each occurrence, is C₆₋₈arylene, C₄₋₆cycloalkylene, the 4- to 6-membered heterocyclylene, or the 5- to 12-membered heteroarylene, where the ring system of the 5- to 12-membered heteroarylene at G¹ is a 5- to 8-membered heteroarylene, wherein G¹ is optionally substituted with 1-2 R^{1x}, wherein, at each occurrence, R^{1x} is selected from the group consisting of halogen, C₁₋₆haloalkyl, —OR^{1a}, —NR^{1a}R^{2b}, —SR^{1a}, —NR^{1a}C(O)R^{1c}, —C(O)OR^{1a}, —C(O)NR^{1a}R^{1b}, and —C(O)R^{1c}; and R^{1a}, R^{1b}, and R^{1c} at each occurrence, are each independently hydrogen, C₁₋₆alkyl, or C₁₋₆haloalkyl; G², at each occurrence, is C₆₋₈arylene, C₄₋₆cycloalkylene, the 4- to 6-membered heterocyclylene, or the 5- to 12-membered heteroarylene, where the ring system of the 5- to 12-membered heteroarylene at G² is a 5- to 8-membered heteroarylene, R^{2x} is selected from the group consisting of halogen, C₁₋₆haloalkyl, —OR^{2a}, —NR^{2a}R^{2b}, —SR^{2a}, —NR^{2a}C(O)R^{2c}, —C(O)OR^{2a}, —C(O)NR^{2a}R^{2b}, —C(O)R^{2c}, —SO₂R^{2d}, and —SO₂NR^{2a}R^{2b}; R^{2a}, R^{2b}, R^{2c}, and R^{2d}, at each occurrence, are each independently hydrogen, C₁₋₆alkyl, or C₁₋₆haloalkyl; Y is alkylene, alkenylene, or alkynylene; X is halogen; and n is 1 to 6,000, wherein at least one of G¹ and G² is present.

In some embodiments, the compound has formula (I-a), or a salt thereof, wherein: G¹ is optionally substituted with 1 R^{1x}, wherein R^{1x} is selected from the group consisting of halogen, C₁₋₆haloalkyl, —OR^{1a}, —C(O)OR^{1a}, —C(O)NR^{1a}R^{1b}, and —C(O)R^{1c}; and R^{1a}, R^{1b}, and R^{1c}, at each occurrence, are each independently hydrogen, C₁₋₆alkyl, or C₁₋₆haloalkyl.

In some embodiments, the compound has formula (I-a), or a salt thereof, wherein: R^{1x} is —C(O)OR^{1a}; and R^{1a} is hydrogen or C₁₋₃alkyl.

In some embodiments, the compound has formula (I-a), or a salt thereof, wherein: R^{2x} is selected from the group consisting of halogen, C₁₋₆haloalkyl, —OR^{2a}, —NR^{2a}R^{2b}, —SR^{2a}, —NR^{2a}C(O)R^{2c}, —C(O)OR^{2a}, —C(O)NR^{2a}R^{2b}, and —C(O)R^{2c}; and R^{2a}, R^{2b}, and R^{2c}, at each occurrence, are each independently hydrogen, C₁₋₆alkyl, or C₁₋₆haloalkyl.

In some embodiments, the compound has formula (I-a), or a salt thereof, wherein: R^{2x} is —OR^{2a}; and R^{2a} is methyl.

In some embodiments, the compound has formula (I-a), or a salt thereof, wherein: G¹, at each occurrence, is C₆₋₈arylene, or C₄₋₆cycloalkylene; G², at each occurrence, is C₆₋₈arylene, or C₄₋₆cycloalkylene; R^{2x} is selected from the group consisting of halogen, C₁₋₆haloalkyl, —OR^{2a}, —NR^{2a}R^{2b}, —NR^{2a}C(O)R^{2c}, —C(O)OR^{2a}, —C(O)NR^{2a}R^{2b}, and —C(O)R^{2c}; and R^{2a}, R^{2b}, and R^{2c}, at each occurrence, are each independently hydrogen, C₁₋₅alkyl, or C₁₋₅haloalkyl.

In some embodiments, the compound has formula (I-a), or a salt thereof, wherein: Y is C₁₋₄alkylene, C₁₋₄alkenylene, or C₁₋₄alkynylene.

In some embodiments, the compound has formula (I-a), or a salt thereof, wherein: L³ is C₂₋₁₀heteroalkylene, C₁₋₁₀alkylene-NR^{a}C(O)—, or C₁₋₁₀alkylene-C(O)NR^{a}—C₁₋₆alkylene. In some embodiments, the compound has formula (I-b)

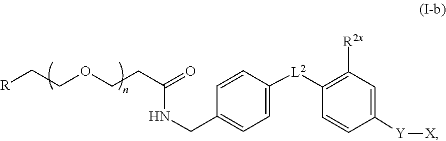

or a salt thereof, wherein: R is a functional group selected from the group consisting of a binding group, a detection label, a biologically active agent, a biorthogonal functional group, and a substrate; L² is

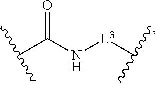

wherein: L³ is heteroalkylene, alkylene-NR^{a}C(O)—, or alkylene-C(O)NR^{a}-alkylene; R^{a}, at each occurrence, is hydrogen or C₁₋₄alkyl; Y is alkylene, alkenylene, or alkynylene; X is halogen; R^{2x} is C₁₋₆haloalkyl, —OR^{2a}, —NR^{2a}R^{2b}, —SR^{2a}, —NR^{2a}C(O)R^{2c}, —C(O) OR^{2a}, —C(O)NR^{2a}R^{2b}, —C(O)R^{2c}, —SO₂R^{2d}, or —SO₂NR^{2a}R^{2b}; R^{2a}, R^{2b}, R^{2c}, and R^{2d} at each occurrence, are each independently hydrogen, C₁₋₆alkyl, or C₁₋₆haloalkyl; and n is 1 to 6,000.

In some embodiments, the compound has formula (I-b), or a salt thereof, wherein: L³ is C₂₋₁₀heteroalkylene, C₁₋₁₀alkylene-NR^{a}C(O)—, or C₁₋₁₀alkylene-C(O)NR^{a}—C₁₋₆alkylene; Y is C₁₋₁₀alkylene, C₁₋₁₀alkenylene, or C₁₋₁₀alkynylene; R^{2x} is halogen or —OR^{2a}; R^{2a} is hydrogen or C₁₋₄alkyl; and n is 1 to 1,000.

In some embodiments, the compound has formula (I-b), or a salt thereof, wherein: L³ is C₅₋₈heteroalkylene, wherein 1 carbon atom of the heteroalkylene is replaced by O.

It is noted that for compounds disclosed herein, options for different moieties are read from left to right in how they are orientated with adjacent moieties. For example, in embodiments where L² is —C(O)NR^{a}-heteroalkylene and G¹ and G² are both present, the carbonyl of L² is bonded to G¹ and the heteroalkylene of L² is bonded to G². Similarly, in embodiments where L¹ is

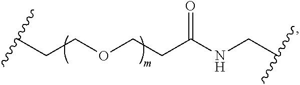

and G¹ is present, the PEG side of L¹ is bonded to R and the amide side of L¹ is bonded to G¹.

Functional groups (e.g., R) useful for the compounds of formula (I) are molecules that can be useful in the isolation, purification, detection, localization, immobilization, modulation, etc. of a protein, a cell, a tissue, or the like. A functional group can be capable of being covalently linked to the compounds of formula (I) through L¹ G¹, or L² and, as part of the compound, retains the desired property (e.g. activity, binding, etc.) as a functional group which is not linked to a compound. Functional groups thus can have one or more properties that can facilitate detection, isolation, immobilization, modulation, etc. of stable complexes formed between a compound having the functional group and a mutant protein (e.g., dehalogenase) as well as proteins adjacent to the mutant dehalogenase (e.g., receptors). A functional group may have more than one functional property, such as being capable of detection and of being bound to another molecule.

The functional group can be a binding group. The binding group is one that is capable of binding another molecule, and in some instances specifically binding another molecule. As used herein, the term “specifically binds” is generally meant that a molecule binds to a target molecule when it binds to that target molecule more readily than it would bind to a random, unrelated target. Examples of binding agents include, but are not limited to, a peptide, a protein, a carbohydrate, a lipid, a small molecule, a nucleic acid, or a combination thereof. In some embodiments, the binding agent is biotin, an antibody, a nanobody, a single chain variable fragment, or an aptamer.

The functional group can be a detection label. The detection label allows the detection of the compounds of formula (I), a protein, a cell, a tissue, or the like to be detected and/or visualized directly or indirectly. The detectable label can include any composition detectable by spectroscopic, photochemical, biochemical, immunochemical, electrical, optical, or chemical means. Example detectable labels include, but are not limited to, fluorescent nanoparticles (e.g., quantum dots (Qdots)), metal nanoparticles, (e.g., gold nanoparticles) fluorescent dyes (e.g., Alexa 647, Alexa 488, fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, ³²P, ⁹⁹Tc, ²⁰³Pb, ⁶⁷Ga, ⁶⁸Ga, ⁷²As, ¹¹¹In, ^{113m}In, ⁹⁷Ru, ⁶²Cu, ⁵²Fe, ^{52m}Mln, ⁵¹Cr, ¹⁸⁶Re, ¹⁸⁸Re, ⁷⁷As, ⁹⁰Y, ⁶⁷Cu, ¹⁶⁹Er, ¹²¹Sn, ¹²⁷Te, ¹⁴²Pr, ¹⁴³Pr, ¹⁹⁸Au, ¹⁹⁹Au, ¹⁶¹Tb, ¹⁰⁹Pd, ¹⁶⁵Dy, ¹⁴⁹Pm, ¹⁵¹Pm, ¹⁵³Sm, ¹⁵⁷Gd, ¹⁵⁹Gd, ¹⁶⁶Ho, ¹⁷²Tm, ¹⁶⁹Yb, ¹⁷⁵Yb, ¹⁷⁷Lu, ¹⁰⁵R h, ¹¹¹Ag, and the like), enzymes (e.g., horse radish peroxidase, alkaline phosphatase and others commonly used in an ELISA), various colorimetric labels, magnetic or paramagnetic labels (e.g., magnetic and/or paramagnetic nanoparticles), spin labels, radio-opaque labels, and the like. In some embodiments, the detection label includes a chromophore, a fluorophore, a radiolabel, a polynucleotide, a small molecule, an enzyme, a nanoparticle, a microparticle, a quantum dot, or an upconverter. In some embodiments, the detection label includes a fluorophore. In some embodiments, the detection label includes Alexa 647, Alexa 488, TMR, or Oregon Green®.

The functional group can be a biologically active agent. As used herein, the term “biologically active agent” refers to a substance that can act on a cell, virus, tissue, organ, organism, or the like, to create a change in the functioning of the cell, virus, tissue, organ, or organism. Examples of a biologically active agent include, but are not limited to, biologics, small molecule drugs, lipids, proteins, peptides, and nucleic acids. A biologically active agent can be capable of treating and/or ameliorating a condition or disease or one or more symptoms thereof, and/or modulating biological activity (e.g., neurotransmission) in a subject. Biologically active agents of the present disclosure also include prodrug forms and pharmaceutically acceptable salt forms of the agent. The biologically active agent can be a class of drugs. For example, the biologically active agent can be an agonist, an antagonist, an allosteric modulator, an inverse agonist, a partial agonist, or a biased agonist. In some embodiments, the biologically active agent includes gabazine, gabazine variants (which include those described for compounds of formula (III)), diazepam, flumazenil, CMPDA, YM90K, atropine, naloxone, oxymorphone, THC rimonabant, PPHT, NAPS, or haloperidol, or a pharmaceutically acceptable salt thereof. In some embodiments, the biologically active agent includes gabazine, diazepam, flumazenil, CMPDA, YM90K, atropine, naloxone, oxymorphone, THC rimonabant, PPHT, NAPS, or haloperidol, or a pharmaceutically acceptable salt thereof.

The functional group can be a biorthogonal functional group. As used herein, “biorthogonal chemistry” refers to a chemical reaction that can occur inside of living systems without interfering with native biochemical processes. Bioorthogonal reactions proceed in high yield under physiological conditions and result in covalent bonds between reactants that are otherwise stable in these settings. Bioorthogonal reactions are reactions of materials with each other, wherein each material has limited or substantially no reactivity with functional groups found in vivo. For example, the efficient reaction between an azide and a terminal alkyne, i.e., the most widely studied example of “click” chemistry, is known as a useful example of a bioorthogonal reaction. Click chemistry refers to a group of reactions that generate substances quickly and reliably and provide high product yields. In some embodiments, the biorthogonal functional group includes an azide, an alkyne, a maleimide, an iodoacetamide, a thiol, a disulfide, a NHS ester, a tetrazine, a trans-cyclooctene, a ketone/aldehyde, a hydrazine, a hydrazide, or a thioacid. In some embodiments, the biorthogonal functional group includes an azide or an alkyne.

The functional group can be a substrate. For example, the substrate can be a solid support (e.g., microsphere, membrane, polymeric plate, beads (e.g., glass, magnetic polymeric, etc.), glass slides, and the like), or a mutant protein (e.g., mutant dehalogenase) bound to a solid support that may be used to generate protein arrays, cell arrays, vesicle/organelle arrays, gene arrays, and/or cell membrane arrays. In some embodiments, the substrate includes a particle, an electrical conducting support, or a magnetic bead.

In some embodiments, the compound of formula (I), formula (I-a), or formula (I-b) is selected from the group consisting of:

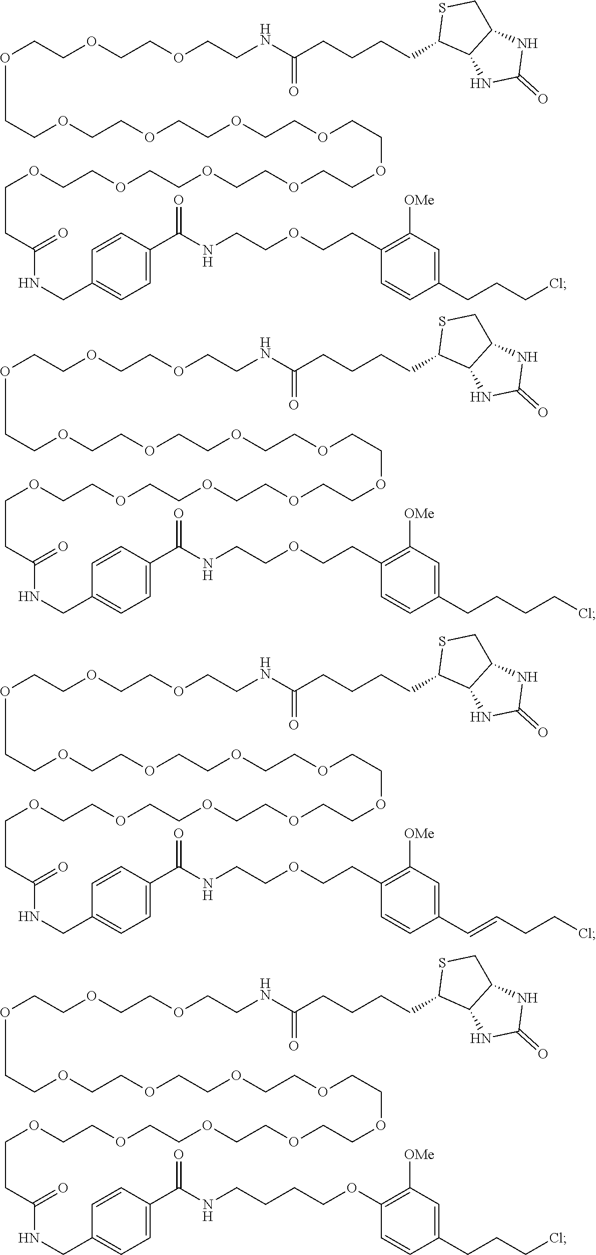

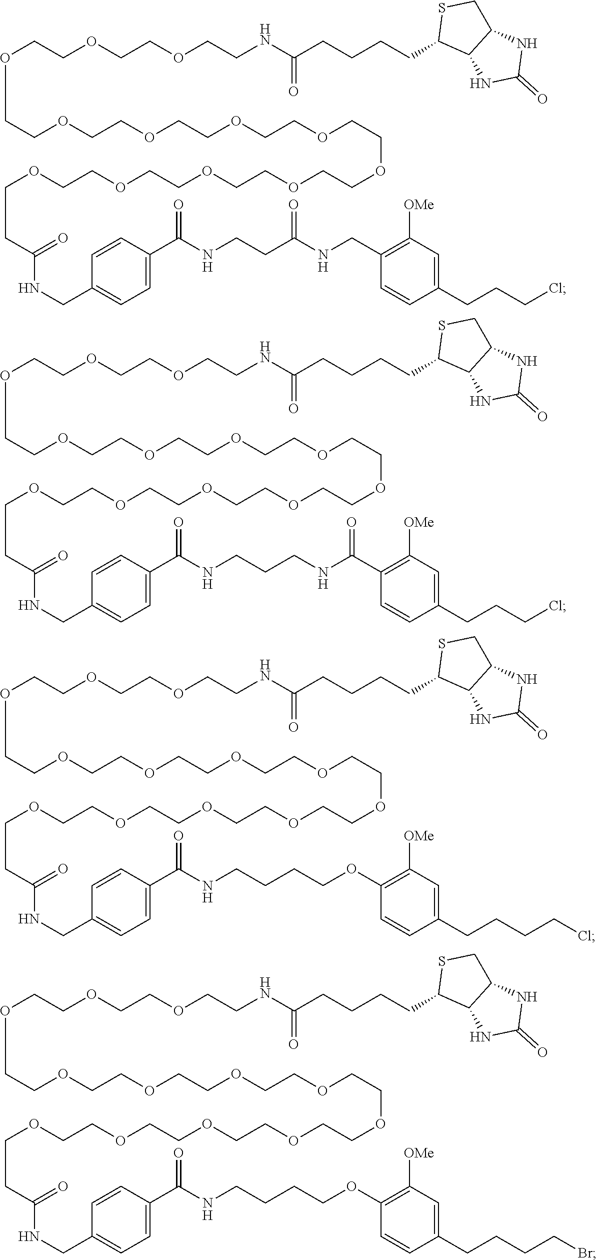

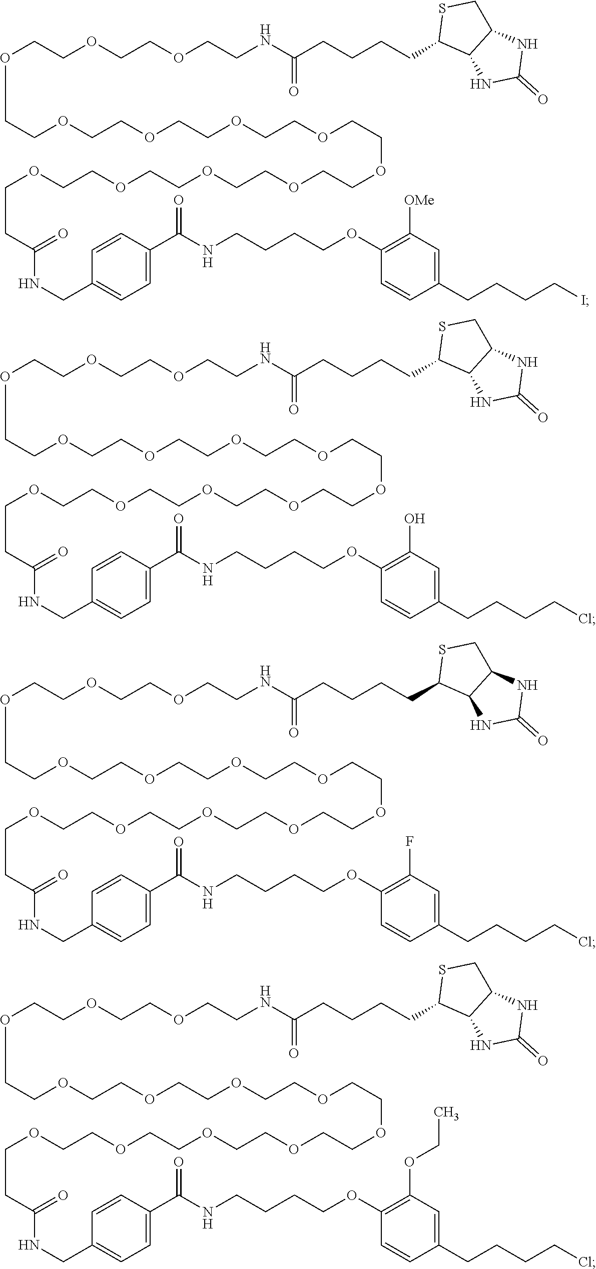

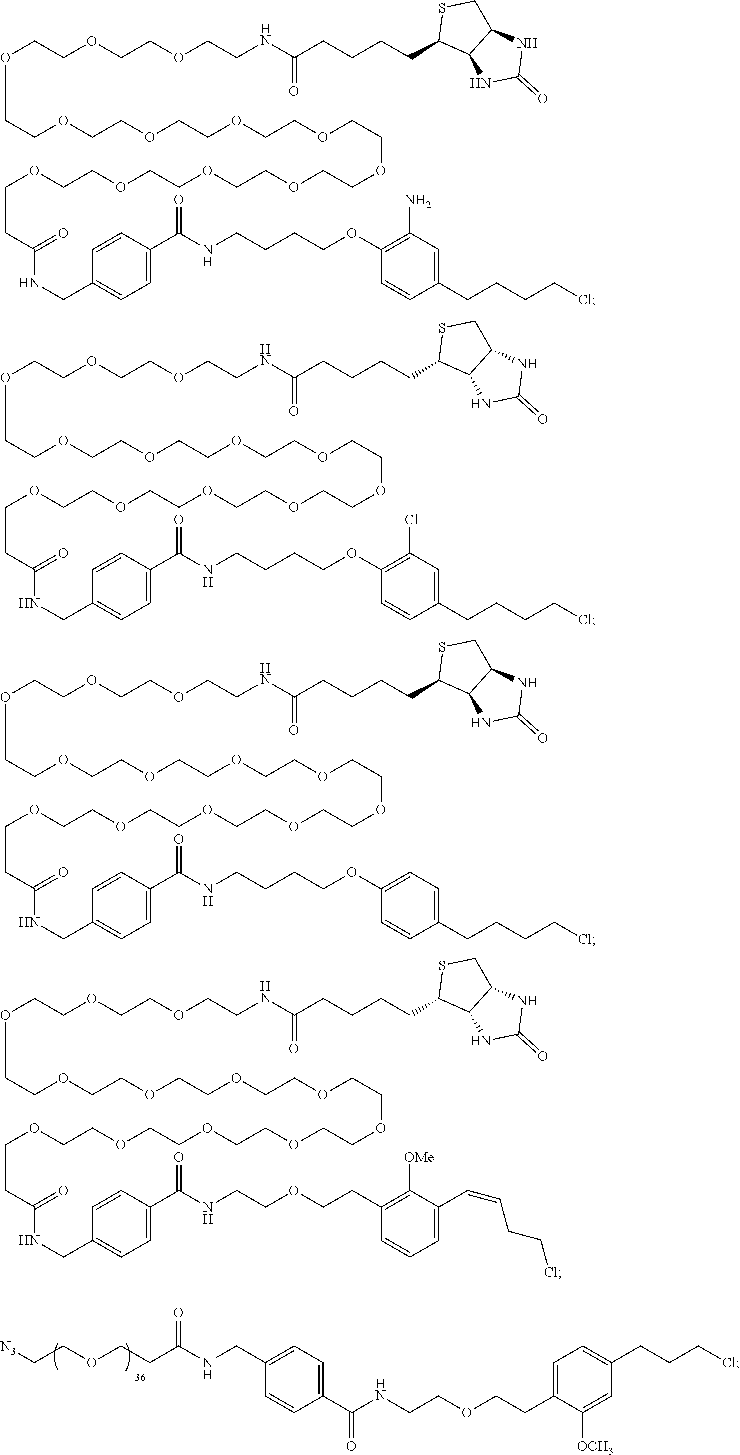

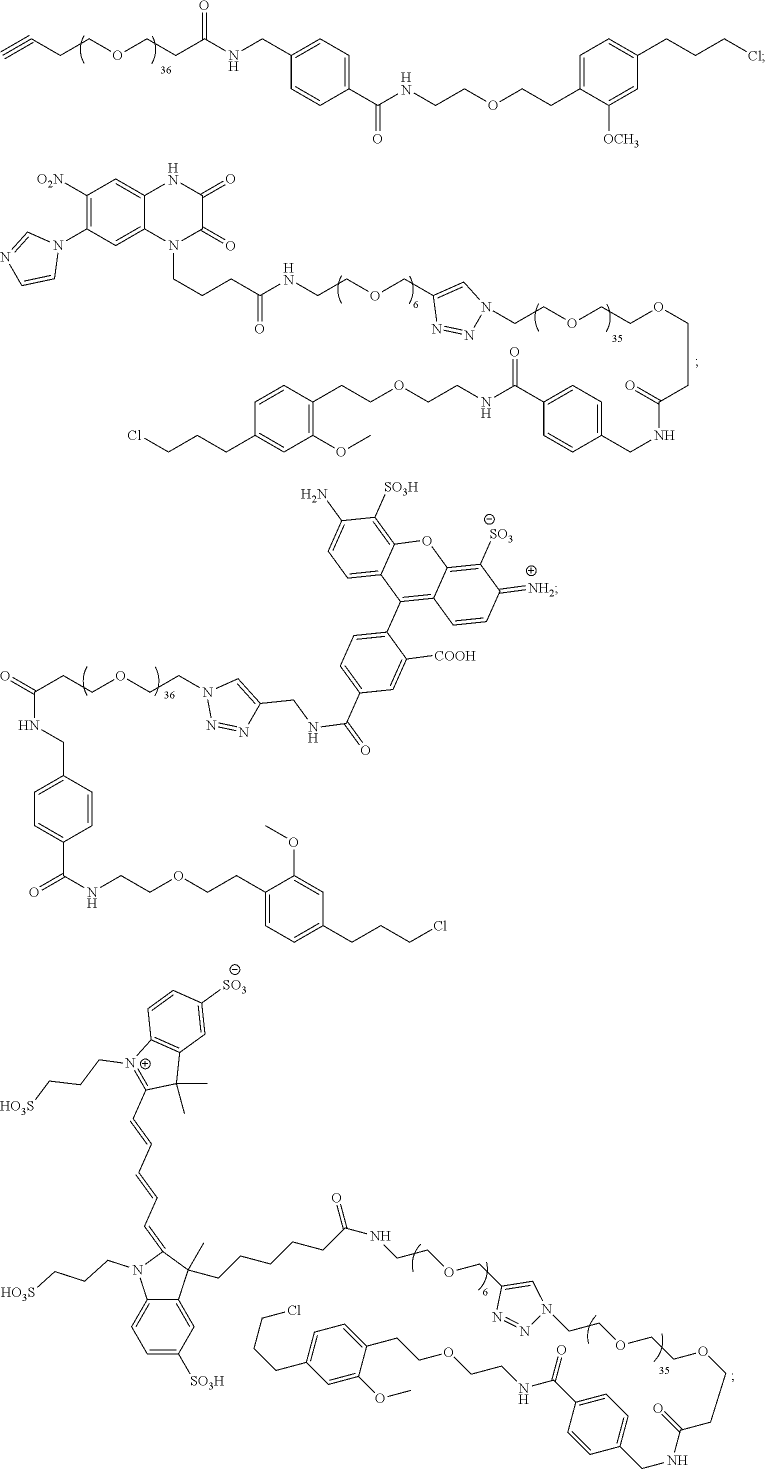

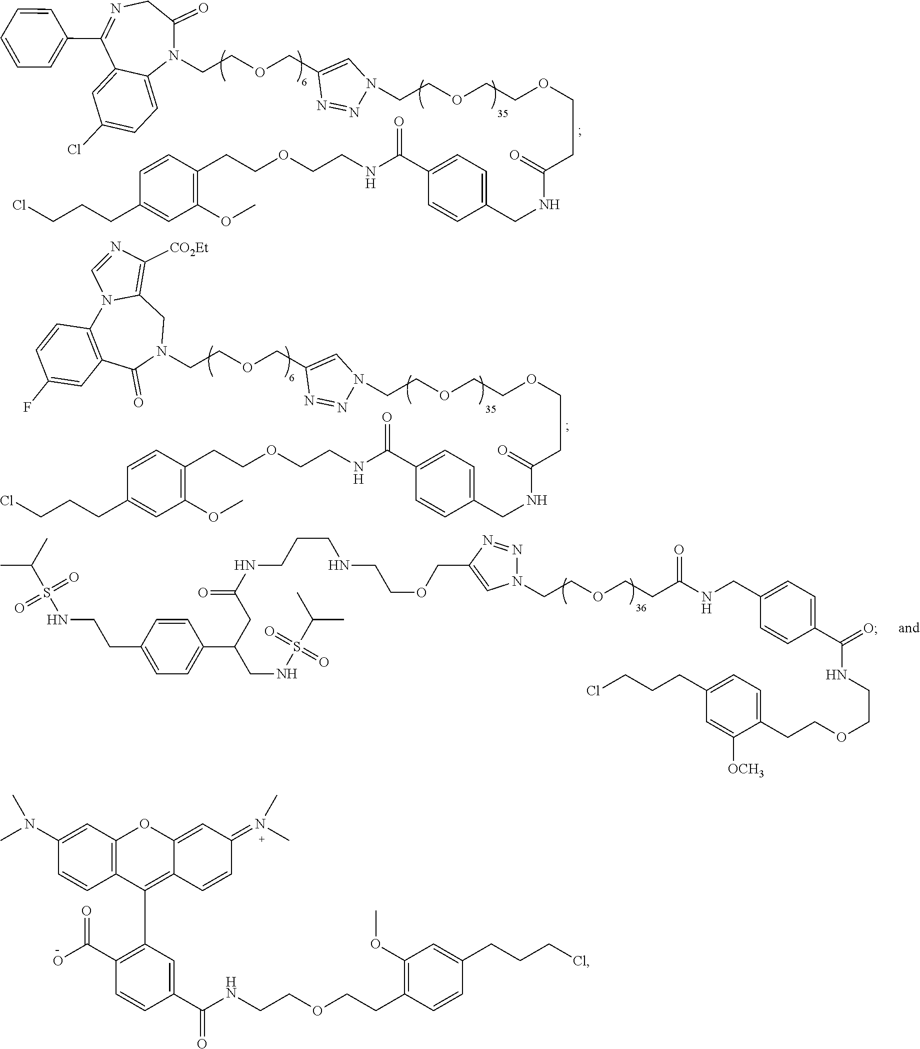

or a salt thereof.

In some embodiments, the compound of formula (I), formula (I-a), or formula (I-b) is selected from the group consisting of:

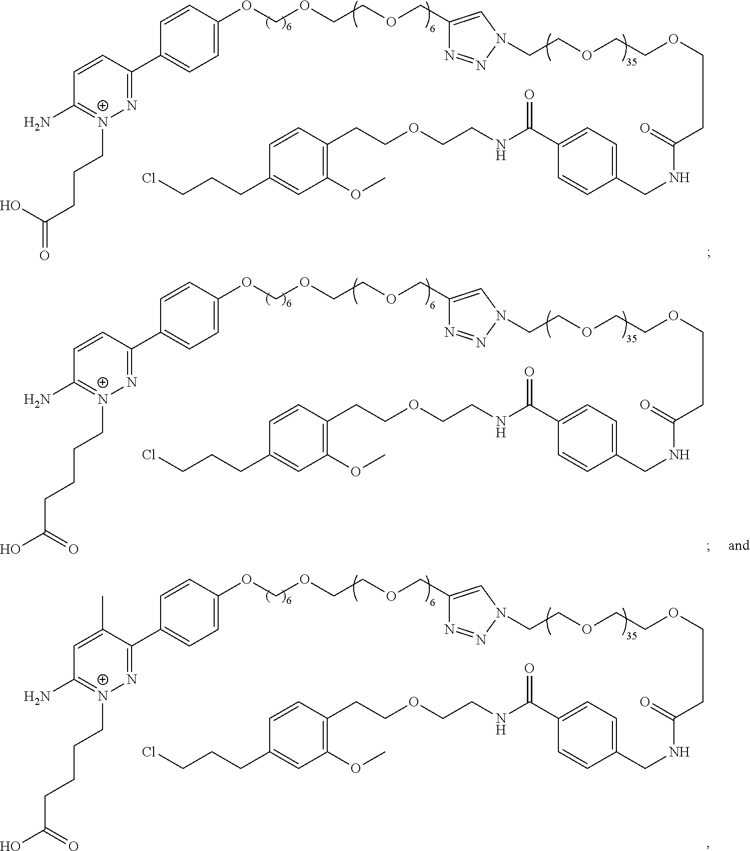

or a salt thereof.

The disclosed compounds may exist as salts, such as pharmaceutically acceptable salts. The term “pharmaceutically acceptable salt” refers to salts or zwitterions of the compounds which are water or oil-soluble or dispersible, suitable for administration to a subject (e.g., treatment of disorders) without undue toxicity, irritation, and allergic response, commensurate with a reasonable benefit/risk ratio and effective for their intended use. The salts may be prepared during the final isolation and purification of the compounds or separately by reacting an amino group of the compounds with a suitable acid. For example, a compound may be dissolved in a suitable solvent and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, trichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides and iodides such as methyl, ethyl, propyl, isopropyl, butyl, lauryl, myristyl, stearyl and the like.

Basic addition salts may be prepared during the final isolation and purification of the disclosed compounds by reaction of a carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### A. General Synthesis

Compounds of formula (I) or any of its subformulas, may be synthesized as shown in the following schemes. In the following general schemes G¹, G², L³, Y, X, R, and m are as defined herein.

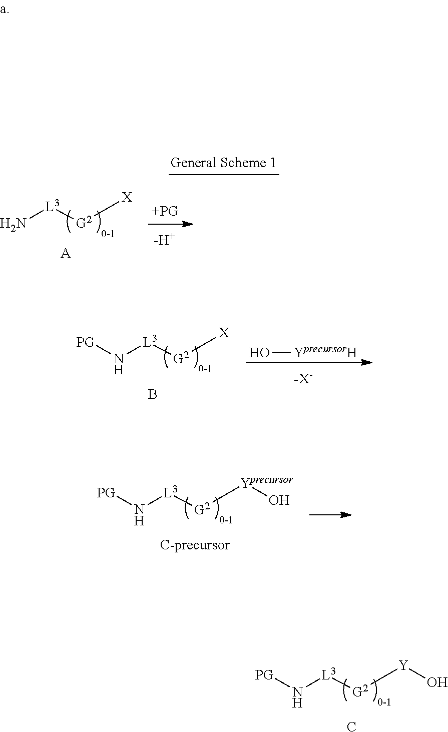

General Scheme 1 illustrates a synthetic route to provide intermediate compounds of formula C. As shown in General Scheme 1, first, halo (X)-substituted amines of formula A may be protected with a suitable amine protecting group (“PG,” e.g., Boc) to form the corresponding protected amines of formula B. Intermediate amine B may then be reacted with HO—Y^{precursor}H under suitable reaction conditions to form a precursor compound C-precursor which, under suitable conditions, may be subsequently transformed into a compound of formula C.

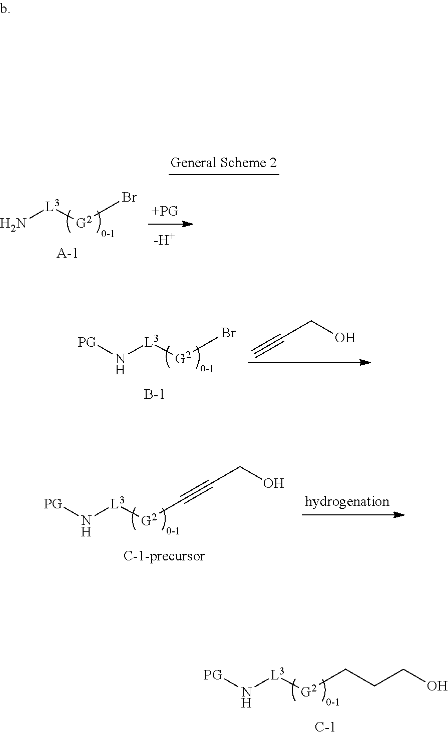

For example, as shown in General Scheme 2, protected Br-substituted amines of formula B-1 may be reacted with prop-2-yn-1-ol C-1-precursor under suitable conditions (e.g., Sonogashira coupling conditions), followed by hydrogenation under suitable hydrogenation conditions (e.g., Pd/C) to provide propan-1-ol compounds of formula C-1.

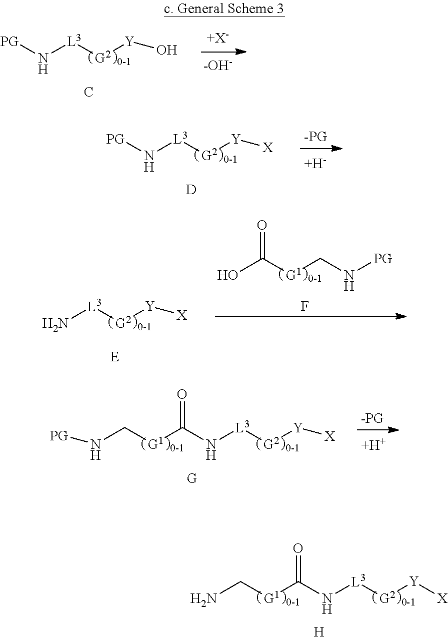

As shown in General Scheme 3, amine-protected, alcohol-substituted compounds of formula C may be halogenated under suitable halogenation reaction conditions to provide amine-protected, halo (X)-substituted compounds of formula D, which may be subsequently deprotected under suitable deprotection conditions (e.g., acid deprotection conditions) to provide intermediate amine compounds of formula E. Next, the intermediate amine of formula E may be coupled with a protected amine-substituted carboxylic acid of formula F under suitable coupling conditions, e.g., a base and a peptide coupling agent (e.g., HATU, HBTU, PyBOP etc.), to provide an amide compound of formula G, which may be subsequently deprotected under suitable deprotection conditions (e.g., acidic deprotection conditions) to provide compounds of formula H.

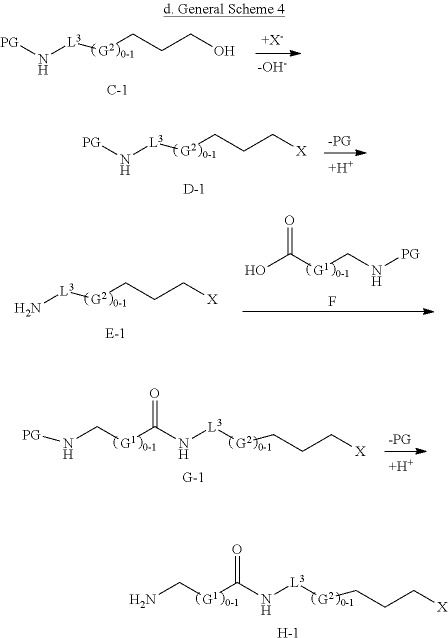

For example, General Scheme 4 shows the synthetic route of General Scheme 3, beginning with halogenation of the propan-1-ol intermediate C-1 under suitable halogenation conditions to provide the corresponding 1-halopropyl intermediate D-1, which may be carried forward as described above for General Scheme 3, to provide 1-halopropyl compounds of formula H-1.

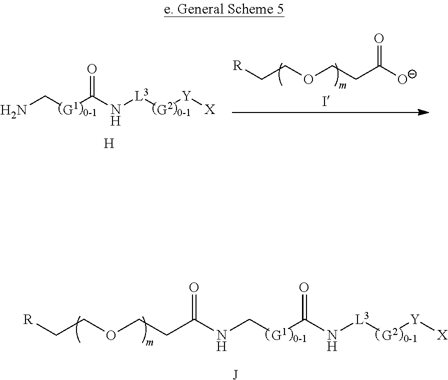

As shown in General Scheme 5, compounds of formula H may be coupled with a carboxylate of formula I′ under suitable reaction conditions to provide compounds of formula J.

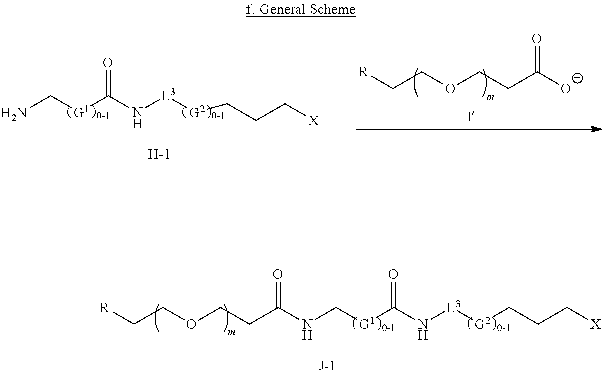

General Scheme 6 shows the synthetic route of General Scheme 5, 1-halopropyl compounds of formula H-1 may be coupled with a carboxylate of formula I′ under suitable conditions to provide compounds of formula J-1.

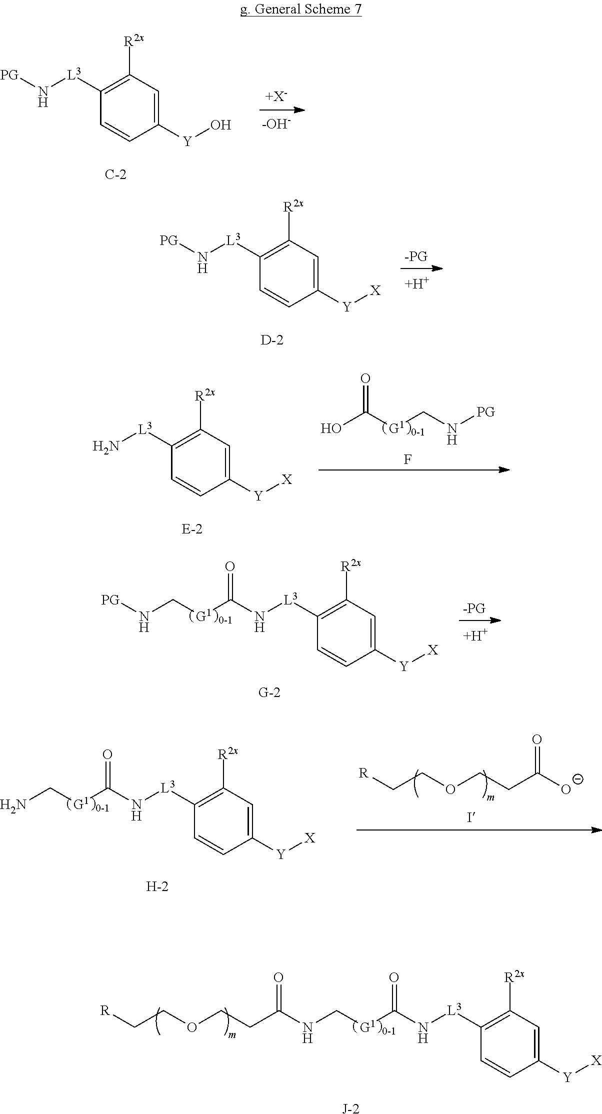

General Scheme 7 shows the preparation of example compounds of formula J-2 according to the general syntheses outlined in General Schemes 3-6 above.

The compounds and intermediates may be isolated and purified by methods well-known to those skilled in the art of organic synthesis. Examples of conventional methods for isolating and purifying compounds can include, but are not limited to, chromatography on solid supports such as silica gel, alumina, or silica derivatized with alkylsilane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry”, 5^{th} edition (1989), by Furniss, Hannaford, Smith, and Tatchell, pub. Longman Scientific & Technical, Essex CM20 2JE, England.

A disclosed compound may have at least one basic nitrogen whereby the compound can be treated with an acid to form a desired salt. For example, a compound may be reacted with an acid at or above room temperature to provide the desired salt, which is deposited, and collected by filtration after cooling. Examples of acids suitable for the reaction include, but are not limited to tartaric acid, lactic acid, succinic acid, as well as mandelic, atrolactic, methanesulfonic, ethanesulfonic, toluenesulfonic, naphthalenesulfonic, benzenesulfonic, carbonic, fumaric, maleic, gluconic, acetic, propionic, salicylic, hydrochloric, hydrobromic, phosphoric, sulfuric, citric, hydroxybutyric, camphorsulfonic, malic, phenylacetic, aspartic, or glutamic acid, and the like.

Optimum reaction conditions and reaction times for each individual step can vary depending on the reactants employed and substituents present in the reactants used. Specific procedures are provided in the Examples section. Reactions can be worked up in the conventional manner, e.g., by eliminating the solvent from the residue and further purified according to methodologies generally known in the art such as, but not limited to, crystallization, distillation, extraction, trituration, and chromatography. Unless otherwise described, the starting materials and reagents are either commercially available or can be prepared by one skilled in the art from commercially available materials using methods described in the chemical literature. Starting materials, if not commercially available, can be prepared by procedures selected from standard organic chemical techniques, techniques that are analogous to the synthesis of known, structurally similar compounds, or techniques that are analogous to the above-described schemes or the procedures described in the synthetic examples section.

Routine experimentations, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the invention. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006), which is incorporated herein by reference in its entirety. Synthesis of the compounds of the invention can be accomplished by methods analogous to those described in the synthetic schemes described hereinabove and in specific examples.

When an optically active form of a disclosed compound is required, it can be obtained by carrying out one of the procedures described herein using an optically active starting material (prepared, for example, by asymmetric induction of a suitable reaction step), or by resolution of a mixture of the stereoisomers of the compound or intermediates using a standard procedure (such as chromatographic separation, recrystallization, or enzymatic resolution).

Similarly, when a pure geometric isomer of a compound is required, it can be obtained by carrying out one of the above procedures using a pure geometric isomer as a starting material, or by resolution of a mixture of the geometric isomers of the compound or intermediates using a standard procedure such as chromatographic separation.

It can be appreciated that the synthetic schemes and specific examples as described are illustrative and are not to be read as limiting the scope of the invention as it is defined in the appended claims. All alternatives, modifications, and equivalents of the synthetic methods and specific examples are included within the scope of the claims.

### 3. Mutant Dehalogenases

Also provided herein are recombinant proteins that include a mutant dehalogenase. A mutant protein, hydrolase and/or dehalogenase, as described in more detail, for example, in U.S. Pat. Nos. 7,238,842; 7,425,436; 7,429,472; 7,867,726; each of which is herein incorporated by reference in their entireties, includes at least one amino acid substitution relative to a corresponding wild-type protein, hydrolase or dehalogenase. Mutant dehalogenases typically can form a stable (e.g., covalent) bond with compounds of formula (I). In some embodiments, the mutant dehalogenase has an amino acid sequence of SEQ ID NO:1. However, the present disclosure also provides mutant dehalogenases that include one or more amino acid substitutions which render the mutant dehalogenase incapable of forming a stable (e.g., covalent) bond with compounds of formula (I). In some embodiments, the mutant dehalogenase that does not form a stable bond with the disclosed compounds has an amino acid sequence of SEQ ID NO:2.

The recombinant protein can include more than one amino acid substitution, which is located in different regions of the mutant dehalogenase. For example, the recombinant protein can have three amino acid substitutions within a catalytic triad of the mutant dehalogenase, two amino acid substitutions within a tunnel entrance (e.g., where the compounds of formula (I) enter the protein) of the mutant dehalogenase, or a combination thereof. In some embodiments, the recombinant protein has amino acid substitutions at residues 41, 106, 107, 245, and 246 of a mutant dehalogenase having SEQ ID NO:1. In some embodiments, the amino acid substitutions are N41E, D106E, W107G, V245L, and L246R of a mutant dehalogenase having SEQ ID NO:1.

In some embodiments, the recombinant protein includes a mutant dehalogenase having at least 85% sequence identity to SEQ ID NO:1, at least 90% sequence identity to SEQ ID NO: 1, at least 95% sequence identity to SEQ ID NO: 1, at least 96% sequence identity to SEQ ID NO:1, at least 97% sequence identity to SEQ ID NO: 1, at least 98% sequence identity to SEQ ID NO:1, or at least 99% sequence identity to SEQ ID NO:1.

In some embodiments, the mutant dehalogenase is Rhodococcus dehalogenase having the amino acid sequence of SEQ ID NO:2.

### 4. Protein Compositions

Further provided herein are compositions that include a protein linked to a functional group by a linker of formula (II)

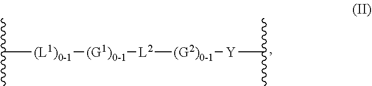

wherein: L¹, G¹, L², G², and Y are defined as described above for formula (I), along with all the varying combinations described above for formula (I). The protein can include a mutant dehalogenase, and the functional group can be selected from the group consisting of a binding group, a detection label, a biologically active agent, a biorthogonal functional group, and a substrate. The mutant dehalogenase and the functional group (e.g., R) are defined as described above.

### 5. Compounds of Formula (III)

Further provided herein are variants of gabazine. Gabazine includes of a 3-amino, 6-phenoxy-pyridazine ring, with GABA embedded in the 2-position (2 butyric acid). Disclosed variants of gabazine can include compounds of formula (III)

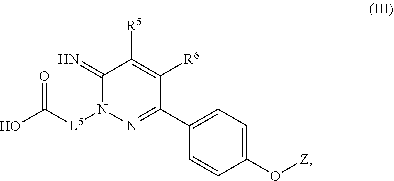

or a salt thereof, wherein: R⁵ is hydrogen, halogen, C₁₋₆alkyl, C₁₋₆haloalkyl, C₂₋₆alkenyl, or C₂₋₆alkynyl; R⁶ is halogen, C₁₋₆alkyl, C₁₋₆haloalkyl, C₂₋₆alkenyl, or C₂₋₆alkynyl; L⁵ is alkylene, alkenylene, or alkynylene; Z is hydrogen, C₁₋₆alkyl, L²⁰-N₃, or L²⁰-alkynyl; and L²⁰, at each occurrence, is a linker.

In some embodiments, the compound has formula (III), or a salt thereof, wherein: R⁵ is hydrogen, halogen, or C₁₋₄alkyl; R⁶ is halogen, C₁₋₄alkyl, or C₁₋₄haloalkyl; L⁵ is C₁₋₁₀alkylene, C₁₋₁₀alkenylene, or C₁₋₁₀alkynylene; and Z is hydrogen, C₁₋₁₀alkyl, C₁₋₁₀₀alkylene-N3, C₁₋₁₀₀heteroalkylene-N₃, C₁₋₁₀₀alkylene-alkynyl, or C₁₋₁₀₀heteroalkylene-alkynyl.

In some embodiments, the compound has formula (III), or a salt thereof, wherein: R⁵ is hydrogen; R⁶ is C₁₋₂alkyl; and L⁵ is C₁₋₅alkylene, C₁₋₅alkenylene, or C₁₋₅alkynylene.

In some embodiments, the compound has formula (III), or a salt thereof, wherein: L⁵ is C₄₋₁₀alkylene, C₄₋₁₀alkenylene, or C₄₋₁₀alkynylene.

### 6. Genetic Constructs

The compounds of formula (I) may bind to a protein (e.g., mutant dehalogenase) expressed on the surface of a cell. The protein expressed on a surface of a cell may be encoded by or comprised within a genetic construct, which is referred to as a “genetically encoded protein” herein. The genetically encoded protein may be one or more of HaloTag protein (e.g., SEQ ID NO:1). The genetic construct, such as a plasmid or expression vector, may include a nucleic acid that encodes a protein. In certain embodiments, a genetic construct may encode one genetically encoded protein, and optionally a marker protein. In some embodiments, a genetic construct may encode two genetically encoded proteins, i.e., a first genetically encoded protein and a second genetically encoded protein, and optionally a marker protein. In some embodiments, a first genetic construct may encode one genetically encoded protein, i.e., a first genetically encoded protein, and optionally a marker protein, and a second genetic construct may encode one genetically encoded protein, i.e., a second genetically encoded protein, and optionally a marker protein. In some embodiments, a first genetic construct may encode one genetically encoded protein, i.e., a first genetically encoded protein, and a second genetic construct may encode a marker protein. The marker protein may be one or more of lacZ (b-galactosidase), xyIE (catechol 2,3-dioxygenase), lux (bacterial luciferase), luc (insect luciferase), phoA (alkaline phosphatase), gusA and gurA (beta-glucuronidase), GFP (green fluorescent protein), mCherry, dTomato, EGFP (Enhanced green fluorescent protein), DsRed (Discosoma sp. red fluorescent protein), Hygro (hygromycin), bla (beta-lactamase) and other antibiotic resistance markers, and the like.

Genetic constructs may include polynucleotides such as vectors and plasmids. The genetic construct may be a linear minichromosome including centromere, telomeres, or plasmids or cosmids. The vector may be an expression vector or system to produce protein by routine techniques and readily available starting materials including Sambrook et al., Molecular Cloning and Laboratory Manual, Second Ed., Cold Spring Harbor (1989), which is incorporated fully by reference herein in its entirety. The construct may be recombinant. The genetic construct may be part of a genome of a recombinant viral vector, including recombinant lentivirus, recombinant rabies virus, recombinant adenovirus, and recombinant adenovirus associated virus. The genetic construct may include regulatory elements for gene expression of the coding sequences of the nucleic acid. The regulatory elements may be a promoter, an enhancer, an initiation codon, a stop codon, or a polyadenylation signal.

The genetic construct may include a heterologous nucleic acid encoding the genetically encoded protein and may further include an initiation codon, which may be upstream of the genetically encoded protein coding sequence, and a stop codon, which may be downstream of the genetically encoded protein coding sequence. The initiation and termination codon may be in frame with the genetically encoded protein coding sequence. The vector may also include a promoter that is operably linked to the genetically encoded protein coding sequence. The promoter may be a constitutive promoter, an inducible promoter, a repressible promoter, or a regulatable promoter. The promoter may be a ubiquitous promoter. The promoter may be a tissue-specific promoter. The tissue specific promoter may be a neuronal subtype-specific promoter. The tissue specific promoter may be a cardiomyocyte-specific promoter. The genetically encoded protein may be under the light-inducible or chemically inducible control to enable the dynamic control of expression of the genetically encoded protein in space and time. The promoter operably linked to the genetically encoded protein coding sequence may be a promoter any promoter known in the art. Examples of promoters include, but are not limited to, glial fibrillary acidic protein (GFAP), Tet-On, Tet-Off, simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, a Rous sarcoma virus (RSV) promoter, a CMV early enhancer/chicken β actin (sCAG) promoter, a human cytomegalovirus (hCMV) promoter, a mouse phosphoglycerate kinase (mPGK) promoter, or a human synapsin (hSYN) promoter.

The vector may also include a polyadenylation signal, which may be downstream of the genetically encoded protein coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal.

Coding sequences in the genetic construct may be optim/zed for stability and high levels of expression.

The genetic construct may also include an enhancer upstream of the genetically encoded protein coding sequence. The enhancer may be necessary for DNA expression. The enhancer may be any enhancer commonly used in the art. Examples of enhancers include, but are not limited to, human actin, human myosin, human hemoglobin, human muscle creatine, or a viral enhancer such as one from CMV, HA, RSV, or EBV. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered.

The genetic construct may be useful for transfecting cells with nucleic acid encoding the genetically encoded protein, where the transformed host cell may be cultured and maintained under conditions wherein expression of the genetically encoded protein takes place. The genetic construct may be transformed or transduced into a cell. The genetic construct may be formulated into any suitable type of delivery vehicle including, for example, a viral vector, lentiviral expression, electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a genetically encoded protein as detailed herein. Suitable cell types are detailed herein. In some embodiments, the cell is a stem cell. The stem cell may be a human stem cell. In some embodiments, the cell is an embryonic stem cell. The stem cell may be a human pluripotent stem cell (iPSCs). Further provided are stem cell-derived neurons, such as neurons derived from iPSCs transformed or transduced with a DNA targeting system or component thereof as detailed herein

### A. Viral Vectors

A genetic construct may be a viral vector. Further provided herein is a viral delivery system. Viral delivery systems may include, for example, lentivirus, retrovirus, adenovirus, mRNA electroporation, or nanoparticles. In some embodiments, the vector may be a modified lentiviral vector. In some embodiments, the viral vector may be an adeno-associated virus (AAV) vector. The AAV vector is a small virus belonging to the genus Dependovirus of the Parvoviridae family that infects humans and some other primate species.

AAV vectors may be used to deliver the genetically encoded protein using various construct configurations. In some embodiments, the AAV vector may be a modified AAV vector. The modified AAV vector may have enhanced neuronal, cardiac muscle, and/or skeletal muscle tissue tropism. The modified AAV vector may be capable of delivering and expressing the genetically encoded protein in the cell of a mammal. The modified AAV vector may be based on one or more of several capsid types, including AAV1, AAV2, AAV5, AAV6, AAV8, and AAV9. The modified AAV vector may be based on an AAV pseudotype with alternative AAV capsids. For example, the AAV vector may be based on an AAV1, AAV6, or AAV7 pseudotype with alternative neuron-tropic AAV capsids, such as AAVrh10, AAV7m8, AAV2retro. In another example, the AAV vector may be based on an AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery.

### 7. Pharmaceutical Compositions

Further provided herein are pharmaceutical compositions including the above-described compounds. The compounds as detailed herein may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation may be used. Generally, additives for isotonicity may include sodium chloride, dextrose, mannitol, sorbitol, and lactose. In some cases, isotonic solutions such as phosphate buffered saline may be used. Stabilizers may include gelatin and albumin. A vasoconstriction agent may be added to the formulation.

The pharmaceutical composition may further include a pharmaceutically acceptable excipient. The term “pharmaceutically acceptable excipient,” as used herein, means a non-toxic, inert solid, semi-solid or liquid filler, diluent, encapsulating material or formulation auxiliary of any type. The pharmaceutically acceptable excipient may be functional molecules as vehicles, adjuvants, carriers, or diluents. Pharmaceutically acceptable excipients include, for example, diluents, lubricants, binders, disintegrants, colorants, flavors, sweeteners, antioxidants, preservatives, glidants, solvents, suspending agents, wetting agents, surfactants, emollients, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analogs including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents. The transfection facilitating agent may be a polyanion, polycation, including poly-L-glutamate (LGS), or lipid. The transfection facilitating agent may be poly-L-glutamate.

The above description of pharmaceutical compositions for the disclosed compounds is also applicable to genetically encoded proteins (e.g., viral vectors encoding mutant dehalogenases).

In some embodiments, the pharmaceutical composition includes a compound as disclosed herein, or a pharmaceutically acceptable salt thereof, and a pharmaceutically acceptable excipient.

### 8. Administration

The compounds as detailed herein may be administered or delivered to a cell, to a subject, or to a cell in a subject. The compounds or proteins as described herein, or the pharmaceutical compositions thereof, may be administered or delivered to a cell, to a subject, or to a cell in a subject. Such compositions can be administered separately or together. Such compositions may be administered in dosages and by techniques well known to those skilled in the medical arts taking into consideration such factors as the age, sex, weight, and condition of the particular subject, and the route of administration.

The compounds or proteins as detailed herein, or the pharmaceutical compositions thereof, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular, or combinations thereof. The compounds or proteins as detailed herein may be delivered to a subject by several technologies including stereotactic injection, robotic implantation (e.g., Neuralink, Neuralink Corporation, San Francisco, CA), and the like.

The genetic constructs as described herein, or the pharmaceutical compositions comprising the same, may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. Any of the compositions may be injected into the brain or other component of the central nervous system. Any of the compositions may be injected into the skeletal muscle or cardiac muscle. For veterinary use, the compounds as detailed herein, and the genetic constructs as described herein, or the pharmaceutical compositions comprising the same, may be administered as a suitably acceptable formulation in accordance with normal veterinary practice. The veterinarian may readily determine the dosing regimen and route of administration that is most appropriate for a particular animal. The compounds as detailed herein, and the genetic constructs as described herein, or the pharmaceutical compositions comprising the same, may be administered by traditional syringes, needleless injection devices, “microprojectile bombardment gone guns,” or other physical methods such as electroporation (“EP”), “hydrodynamic method”, intravenous injection, retro-orbital injection, or ultrasound.

Upon delivery of the presently disclosed compounds or genetic constructs as described herein, or the pharmaceutical compositions including the same, into the cells of a subject, the cells may express the genetically encoded protein on the surface of the cells. Upon delivery of the presently disclosed compounds of compositions thereof, into a subject, the compounds including a ligand for the genetically encoded protein may bind to the cells expressing the genetically encoded protein.

### A. Cell Types

Any of the delivery methods and/or routes of administration detailed herein can be utilized with a number of different cell types, for example, those cell types currently under investigation for neurotransmission. These cell types include, but are not limited to, neurons, muscle cells (e.g., skeletal, cardiac, and smooth), endocrine cells (e.g., insulin-releasing pancreatic β cells), keratinocytes, glia, cell lines expressing voltage-gated ion channels, immune cells, and CAR-T cells.

### 9. Methods

In comparison to other drug tethering technologies (e.g., germline knock-in), DART.2 offers non-overlapping features, by accepting the speed and isotype specificity of traditional drugs, and focusing on providing cell specificity. To simplify use, knock-in animals are not needed; instead a virus can be used to select the cells of interest, which can leverage the growing list of technologies to provide cell-specific AAV expression, with cell type defined by promoters, enhancers, axonal anatomy, neural activity, or combinations thereof. Once a given viral expression approach is established, DART.2 can deliver virtually any drug to the chosen cells, all in the same animal. To further simplify use, photo-activation is not required, enabling interrogation of large brain volumes, without complexities of light penetration, or concerns about photo-switch performance over a wide range of tethered-drug concentrations. Accordingly, the disclosed compounds of formula (I) and mutant proteins (e.g., dehalogenases) are useful to isolate, detect, identify, image, display, or localize molecules of interest, label cells, including live cell imaging, modulate cells, or label proteins in vitro and/or in vivo.

For the methods disclosed below, numerous types of cells can be used and is not generally limited. Example cells include, but are not limited to, a neuron, a muscle cell, an endocrine cell, a keratinocyte, a glial cell, a cell line expressing voltage-gated ion channels, an immune cell, or a CAR-T cell. Furthermore, while the methods can be done in vitro, they can also be done in vivo. For example, the cell can be in a subject.

Provided herein are methods of modulating a cell. The method can include contacting a cell including a mutant dehalogenase on a surface of the cell with a disclosed compound or combination of disclosed compounds, wherein the mutant dehalogenase forms a bond with the compound, and wherein R binds to a receptor on a surface of the cell. The receptor that R binds to is not generally limited and aspects of the compounds can be modulated to effectively access different receptors by, e.g., altering L¹, G¹, L², G², or a combination thereof. Example receptors include, but are not limited to, an ionotropic receptor (e.g., a GAB_{A}A receptor, an AMPA receptor, etc.) a metabotropic receptor (e.g., GPCR), a voltage-gated ion channel (e.g., NaV voltage-gated channel, KV voltage-gated channel, CaV voltage-gated channel, etc.), or a tyrosine kinase receptor (e.g., TrkB).

Also provided herein are methods of labeling a cell. The method can include contacting a cell including a mutant dehalogenase located at a surface of the cell with a disclosed compound or combination of disclosed compounds, wherein the mutant dehalogenase forms a bond with the compound, thereby labeling the cell with R.

Also provided herein are methods of detecting or determining a presence or an amount of a mutant dehalogenase. The method can include contacting a mutant dehalogenase with a disclosed compound or combination of disclosed compounds, wherein the mutant dehalogenase forms a bond with the compound. The method can further include detecting or determining the presence or the amount of R, thereby detecting or determining the presence or the amount of the mutant dehalogenase. In some embodiments, the mutant dehalogenase is located at a surface of a cell.

Further provided herein are methods of modulating neurotransmission, e.g., in a subject in need thereof. The method can include administering to the subject an effective amount of a disclosed compound or combination of disclosed compounds, or a pharmaceutically acceptable salt thereof, optionally in combination with a pharmaceutically acceptable excipient, wherein R is a biologically active agent. R could also potentially be other molecules, such as a binding group—as longs as it can modulate or alter neurotransmission in a subject.

Modulating neurotransmission of the subject can take on many different forms. For example, modulating neurotransmission in the subject can affect locomotion, mood, anxiety, addiction, attention, psychosis, inflammation, or a combination thereof of the subject. In some embodiments, modulating neurotransmission in the subject affects locomotion of the subject.

The compounds of the present disclosure can afford cell specific and localized delivery of functional groups tethered to the compound (e.g., R). In some embodiments, the compound, or a pharmaceutically acceptable salt thereof, is localized to a specific organ or tissue of the subject. In some embodiments, the compound, or a pharmaceutically acceptable salt thereof, is localized to a specific region of the subject's brain.

As mentioned elsewhere, the compounds disclosed herein can be administered in variety of ways depending on the cell, tissue, or subject receiving the administration. In some embodiments, the compound, or a pharmaceutically acceptable salt thereof, is administered to the subject through the cerebrospinal fluid of the subject.

The description of the compounds, proteins (and genetic constructs thereof), or compositions thereof above can also be applied to methods disclosed herein.

### 10. Kits

Also provided herein are kits, which may be used to carry out the disclosed methods. The kits may include one or more of the compounds, proteins, genetic constructs, and/or compositions as described above.

The kits also may include instructions for using the components included in the kits. Instructions included in kits may be affixed to packaging material or may be included as a package insert. While the instructions are typically written on printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

In some embodiments, the kit includes a compound, genetic construct, or both as disclosed herein; and one or more packages, receptacles, labels, or instructions for use.

The description of the compounds, proteins (and genetic constructs thereof), or compositions thereof above can also be applied to kits disclosed herein.

The disclosed invention has multiple aspects, illustrated by the following non-limiting examples.

### 11. Examples

### Materials and Methods for the Examples

All experiments involving animals were approved by the Duke Institutional Animal Care and Use Committee, an AAALAC accredited program registered with both the USDA Public Health Service and the NIH Office of Animal Welfare Assurance.

Genetic Construct Design: Genetic elements were concatenated via PCR, restriction digest, ligation, and sequence verification, as follows: aavCAG-DIO-+HTPGPI-2A-dTomato-WPRE. Coding Seq: SSnlg-+HTP-(GGSGG)₈-ThylGPI-2A-dTomato. SSnlg, the signal peptide (residues 1-49) of mouse neuroligin-1; +HTP, mammalian codon-optimized variant of the HaloTag Protein (Los et al., HaloTag: A Novel Protein Labeling Technology for Cell Imaging and Protein Analysis, ACS Chem. Biol. 2008, 3, 6, 373-382, which is incorporated by reference herein in its entirety); (GGSGG)₈, linker with 8 repeats of gly-gly-ser-gly-gly; ThylGPI from Addgene_163696. Marker of Expression: 2A-dTomato, P2A ribosomal skip sequence and dTomato. Backbone: aavCAG-DIO-WPRE vector is from Addgene_100842. aavCAG-DIO-ddHTPGPI-2A-dTomato-WPRE. Coding Seq: SSnlg-ddHTP-(GGSGG)₈-ThylGPI-2A-dTomato. ddHTP is the HTP with N41E, D106E, W107G, V245L, and L246R mutations. Other elements as above. aavCAG-DIO-+HTPGPI-IRES-dTomato-W3SL. Coding Seq: SSnlg-+HTP-(GGSGG)₈-ThylGPI-IRES-dTomato. IRES is the internal ribosomal entry site. W3SL is from Addgene_61463. Other elements as above. aavSYN-ChR2-HA-dsfGFP-WPRE. Coding Seq: ChR2(H134R)-HA-dsfGFP. ChR2(H134R) is the optogenetic channelrhodopsin2; dsfGFP is superfolder GFP with T65G and Y66G mutations to eliminate fluorescence. Backbone: aavSYN-WPRE vector is from Addgene_100843. aavCAG-+HTP NLG (432-648)ERXL-2A-dTomato-WPRE. Coding Seq: SSnlg-HA-+HTP-NLG (432-648)-ERXL. HA, the hemagglutinin epitope tag; NLG (432-648), the esterase-truncated 71-residue extracellular domain, the 19-residue predicted trans-membrane domain, and the 127-residue C terminus of mouse neuroligin-1; ERXL, the peptide sequence KSRITSEGEYIPLDQIDINVGGSGFCYENEV, a fusion of the trafficking and ER export signals from Kir2.1. Other elements as above. aavCAG-ddHTP NLG (432-648) ERXL-2A-dTomato-WPRE. Coding Seq: SSnlg-HA-ddHTP-NLG (432-648)-ERXL. Elements all as above

Electrophysiology. Preparation of Brain slices: Mice (8-10 weeks) were injected AAV10 HaloTag™-dTomato to striatum or ventral tegmental area (VTA). After 3-5 weeks, mice were deeply anesthetized with isoflurane and euthanized by decapitation. Coronal brain slices (300 mm) containing striatum or VTA were prepared by standard methods using a Vibratome (Leica, VT 1200S), in ice-cold high sucrose cutting solution containing (in mM) 220 sucrose, 3 KCl, 1.25 NaH₂PO₄, 25 NaHCO₃, 12 MgSO4.7H₂O, 10 glucose, and 0.2 CaCl₂) bubbled with 95% O₂ and 5% CO₂. The slices were placed into modified artificial cerebrospinal fluid (aCSF) containing (in mM): 120 NaCl, 3.3 KCl, 1.23 NaH₂PO₄, 1 MgSO₄, 2 CaCl₂), 25 NaHCO₃, and 10 glucose at pH 7.3, previously saturated with 95% O₂ and 5% CO₂. For recovery, the slices were incubated at 33° C. for 40-60 min in the bubbled ACSF solution and then allowed to cool to room temperature (22-24° C.) until the recordings were initiated.

Preparation of Retina: Mice were intravitreal injected AAV10 HaloTag™-dTomato to eyes. After 8-12 weeks, the mice killed by cervical dislocation. Both eyes were removed and dissected under a stereo-microscope. Retinas were isolated from the eyecups and placed in oxygenated (95% O₂/5% CO₂) cold-Ames medium (Sigma-Aldrich), supplemented with 21 mM NaHCO₃. After the vitreous body was removed, a retina was cut into 3 pieces, leaving each retina piece attached to a filter paper with 2 mm hole by retina ganglion-cell-side-down. The pieces of retina were stored in bubbled Ames solution at room temperature, then transferred to recording chamber with retina ganglion-cell-side-up for recording.

Whole-cell patch-clamp recording: The brain slices or retinas were perfused with bubbled aCSF at 29-30° C. with a 2 ml/min flow rate. Recordings were made by whole cell patch recording techniques using a Multiclamp 700B amplifier (Molecular Devices, Axon Instruments Inc., Union City, CA). The signals were filtered at 10 KHz and acquired using Digitate 1440A and pClamp 10.7 (Molecular Devices). The recording pipettes (4 MΩ-6 MΩ) were filled with internal solutions. For GAB_{A}A receptor-mediated evoked inhibitory postsynaptic currents (sIPSCs) recording, the pipettes filled with Cesium Chloride-based internal solution (in mM) 135 CsCl, 2 MgCl₂, 0.5 EGTA, 10 HEPES, 4 Mg-ATP, 0.5 Na-GTP, 10 Na₂-phosphocreatine, and QX314 (lidocaine N-ethyl bromide), pH adjusted to 7.3 with CsOH (290 mosm). For AMPA-receptor mediated evoked excitatory postsynaptic currents (eEPSCs) recording, the pipette solution contained (in mM) 130 CsMeS, 1 MgCl₂, 0.5 EGTA, 10 HEPES, 4 Mg-ATP, 0.5 Na-GTP, 10 Na₂-phosphocreatine, and 4 QX314, pH adjusted to 7.3 with CsOH (290 mosm). For experiment of brain slices and retinas, the eIPSCs or eEPSCs were elicited by electrical stimuli at 0.067 Hz in 0.3-ms duration and 150-300-ρA (60-70% maximum responses) stimulation intensity at a holding potential of −70 mV. The stimulating electrode was placed within 60-100 μm from the recording neuron under voltage-clamp mode. GABA-receptor mediated eIPSCs were isolated in the presence of DNQX (20 mM AMPAR antagonist) and AP-V (50 mM, NMDA antagonist) in the bath solution. For AMPA-receptor mediated eEPSCs, added picrotoxin (50 mM, GABAAR antagonist) and AP-V (50 mM) to the bath solution. For current-clamp recording, the pipette solution contained (in mM) 130 K-gluconate, 5 KCl, 2 MgCl₂, 0.2 EGTA, 10 HEPES, 4 Mg-ATP, 0.5 Na-GTP, and 10 phosphocreatine, pH adjusted to 7.3 with KOH (290 mosM). The liquid junction potential was estimated to be 15.9 mV for the normal aCSF solution and was not corrected. The recordings with stable access and holding currents for at least 5 min were used. Series resistance was monitored using 5-10 mV hyperpolarizing step throughout the experiment, and cells were discarded if series resistance changed more than ˜15% during the experiment. The stored data signals were processed using Clampfit 10.7 (Axon Instruments). All averaged data are presented as mean±SEM and n represents the number of cells tested per condition. Statistical significance was determined using Student's t or one-way or two-way ANOVA tests. eIPSCs or eEPSCs decay time constants were obtained by fitting a single-exponential function, I(t)=I exp(−t/T)+ISS, where I(t) is the amplitude of the current at time t and ISS is the steady-state current, I is instantaneous current subtracted from Iss, and t is the time constant of decay.

Cultured neuronal assays: Preparation of hippocampal cultures: Mixed glial and neuronal cultures were prepared from the hippocampus of postnatal day 0 to 1 Sprague-Dawley rat pups. Separate pools of dissociated cells were transfected via electroporation (LONZA, V4SP-3096) with plasmid constructs, and depending on assay directives, either plated individually or mixed with a different pool of electroporated cells after a 10 min recovery period. Cells were cultured on coverslips (Deckglaser) pre-treated with high molecular weight poly-D-lysine (Sigma, P7405; 0.05 mg/mL) in 24-well plates and maintained in NbActiv4 (BrainBits, Nb4-500) at 37 C, 5% CO₂. Media was changed by half at DIV3 or 4 and then weekly thereafter; experiments were performed between DIV16 to DIV18.

For any given neuronal assay, coverslips were first washed briefly in a resting BASE solution containing (in mM): 4 KCl, 2 CaCl₂), 2 MgCl₂, 150 NaCl, 10 HEPES, 10 glucose, pH 7.4, before transfer to a glass-bottom, 24-well imaging plate (Cellvis, P24-1.5H—N). Live cell imaging was performed on an Olympus IX83 inverted fluorescent microscope (10× objective, NA 0.) with LED illumination for GFP (Excitation=485 nm), TR (Ex=560 nm), Cy5/Alexa647 (Ex=640 nm) and Cy7/AlexaFluor750 (Cy7=740 nm) depending on the specific assay directives.

AMPAR: Dissociated neurons were electroporated with constructs and cultured for 2 weeks. The assay was performed in the same BASE solution as described above, but with the addition of synaptic blockers CPP (NMDAR antagonist; Tocris) and gabazine (GABAR antagonist; Tocris) to isolate AMPAR activity (=AMPAR-BASE). After a brief rinse in AMPAR-BASE, coverslips were transferred to a glass-bottom imaging plate containing 500 μL of AMPAR-BASE, and a single region of interest (ROI) was defined for each coverslip using the dTomato fluorescent marker signal, indicating either +HTP cells (expressing the active HaloTag protein) or ddHTP cells (expressing the double dead, non-active version). Post-synaptic action potentials (APs) were visualized via a change in the GCamp6s signal, recorded after optogenetic stimulation of pre-synaptic cells containing ChR2. An imaging cycle consisted of a 4 min delay (to allow for consistent Rx/Rx-DART incubation), then six rounds of an image of TR, 5 ms then 20 ms (to allow for cell alignment between multiple imaging cycles on a given coverslip) followed by x pulses of GFP, 50 ms to elucidate APs.

Dosing was obtained via a DRUG plate: a 24-well plate initially containing 500 μL of AMPAR-BASE for each coverslip represented in the assay; in other words, one DRUG plate well (DPW) corresponds to one imaging plate well (IPW)/coverslip. In the DPWs, increasing concentrations of a drug (Rx) or drug-DART (Rx-DART) are added over sequential imaging cycles to create a dose response curve for a given coverslip. Slowly, the 500 uL volume of DPW is added to the 500 uL in the IPW, the 2 volumes are mixed via pipetting (mix=add 500 uL from DPW to corresponding IPW withdraw 500 uL from IPW add back to same IPW), and then 500 ul from the IPW is removed and returned back to the corresponding DPW. In this way, each coverslip in the imaging plate is exposed to a calculated level of Rx/Rx-DART prior to the next imaging cycle while maintaining a set volume.

First, a baseline imaging recording was obtained in the original 500 uL of AMPAR-BASE in the IPW, then a round of AMPAR-BASE from the DPW was added and removed as described above, followed by an imaging cycle, to verify and normalize the neuronal baseline recording after any disturbance from the solution pipetting technique. Each coverslip in the assay was assigned to one of three “DoseGroups”, which allowed for the addition of small volumes of highly concentrated Rx/Rx-DARTs to obtain more dynamic dosing for a given coverslip and maintain live assays within a set, allowable time-frame (typically 1.45-2 hr duration). Three rounds of increasing Rx/Rx-DART concentrations were applied for each DoseGroup, each drug add-mix-remove application followed by its corresponding imaging cycle. Finally, a WASH was performed on all wells, which consisted of six rounds of add-mix-remove of 500 uL of AMPAR-BASE to the IPWs, using fresh AMPAR-BASE for each round to, in theory, exponentially dilute any Rx/Rx-DART remaining in the DPW to residue levels and effectively washing out any non-covalently captured drug.

Using custom MatLab codes, images across cycles from a given coverslip were aligned, individual cell bodies were identified (with authenticity confirmed by human proof-reading), and differentials in GFP fluorescence were calculated and normalized. Data plots of GFP intensity were generated as a function of dose to demonstrate the effect of the Rx/Rx-DART on neuronal activity.

Gabazine: Dissociated neurons were electroporated with constructs and cultured for 2 weeks. The assay was performed in the same BASE solution as described above, but with the addition of synaptic blockers CPP (NMDAR antagonist; Tocris) and NBQX (AMPAR antagonist; Abcam, ab120046) to isolate GABAR activity, and a small amount of GABA (GABAR agonist; Sigma) to ensure GABAR activity (=GABAR-BASE). The imaging assay was performed in a similar fashion as described above for the AMPAR assay, with respect to the creation of a DRUG plate and three DoseGroups, but no final washout was performed. Instead, a high dose of regular gabazine was used to completely silence any cells that may not have had realized full antagonism by the end of the assay. Imaging alignment, proof-reading and analysis were performed in a similar fashion to the AMPAR assay using MatLab.

Diazepam/Flumazenil: Dissociated neurons were electroporated with constructs and cultured for 2 weeks. The assay was performed in a modified GABAR-BASE, with an increased KCl concentration to enhance neuronal excitability and a lowered GABA level for a baseline. Again, a DRUG plate and three DoseGroups were used as described above for dosing, but no final WASH. For this assay, a high dose of regular diazepam was used to evoke complete modulation for cells that may not have had realized full positive allosteric conformation, followed by a high dose of gabazine to demonstrate that the activity revealed during the assay was due to GABAR interrogation. Imaging alignment, proof-reading and analysis were performed in a similar fashion to the GABAR assay using MatLab.

Virus characterization: Dissociated neurons were either plated without modification, for use with non-DIOflexed viruses, or were electroporated with the aav.syn-iCre construct from AddGene (find 5773A origins and ref AG contributor), for use with DIOflexed viruses, and cultured as described above. At DIV 3-4 along with the first media change, neurons were infected with a serial dilution of virus made in pre-acclimated NbActiv4, then maintained for ˜2 weeks. Coverslips were rinsed briefly in BASE solution containing 1% BSA (Sigma A7904), incubated with either 1 uM Alx647.1-DART.2 for 15 min at RT (diluted in BASE+BSA) or with 10 nM Alx647.1-DART.2 for 1 hr at 37 C (diluted in conditioned media+BSA), then rinsed two to three times. Coverslips were transferred to a glass-bottom imaging plate containing BASE+BSA. Each coverslip was imaged in its entirety by combining several 10× images (TR at 10 ms; Cy5 at 300 ms) together using the Olympus CellSens software stitching algorithm. Where possible, a serotype-specific virus with a previously determined functional titer based on the fluorescent dTomato marker was used as a reference for new lots or viruses with the same serotype. If not available, other markers including the +HTP capture of the Alx647.1-DART.2 were used to estimate functional titers. The median pixel intensity at a fix scale was recorded for a given fluorescent channel, and the values for the coverslips used in the serial dilution of a virus were plotted using Excel, against the reference virus, as a function of dose to determine the functional titer.

biotin-HTL: Dissociated neurons were electroporated with either the +HTP or ddHTP construct and cultured as described above. A dilution series for both the biotin-PEG12-HTL.1 and biotin-PEG12-HTL.2 compounds was made in BASE solution containing 1% BSA (Sigma A7904), representing 12 final concentrations (in uM) between 10 and 0.001. After a brief rinse in BASE+BSA to acclimate the live neurons to room temperature and avoid temperature effects to the binding kinetics, a single coverslip was incubated in one of the twelve dilutions for 15 min, washed twice in BASE+BSA, then incubated in a 1:500 dilution of Streptavidin-AlexaFluor488 (Invitrogen) for 15 min. The neurons were rinsed four times then transferred to a glass-bottom imaging plate containing BASE+BSA. Each coverslip was imaged in its entirety by combining several 10× images (TR at 10 ms; GFP at 100 ms) together using the Olympus CellSens software stitching algorithm. A dose-response curve was created from the individual coverslips using MatLab.

Mice: For behavioral experiments, mice strains included Drd1A-Cre (GENSAT EY262). For slice experiments, Drd1A-Cre were used. Mice were singly housed post-surgery, in a 12-hour reverse light cycle, with food and water provided ad libitum. Recombinant adeno-associated viral (rAAV) vectors.

Behavioral assays: Assays were performed at least 3 weeks after viral injection to allow for recombinant protein expression. Mice were acclimated to head-fixation for 3 consecutive days prior to behavioral experiments. Reagents were infused into the brain via the cranial cannula. Immediately following infusion, mice were placed into open-field chambers (27 cm×27 cm) in the dark, and behavior was recorded using infrared video.

Histology. Mice were deeply anesthetized with isoflurane and fixed by transcardial perfusion of 5 mL PBS then 50 mL ice-cold 4% paraformaldehyde (PFA) in 0.1M PB, PH 7.4. Brains were excised from the skull, further post-fixed in 50 mL of 4% PFA at 4 C overnight then washed with PBS. Brains were embedded in 5% agarose and sliced along the coronal axis at 50 uM (Leica, VT1200S). Sections were mounted onto glass slides (VWR 48311-703) and coverslipped with Vectashield mounting medium (Vector Labs, H-1400 or H-1800). Fluorescent images (DAPI, GFP, TRITC, Cy5) were collected at 10× magnification with either an Olympus IX83 inverted microscope or an Olympus VS200 slide scanner. Images were analyzed for pixel intensity using MATLAB.

General Chemical Synthesis Information: All chemical reagents were obtained from commercial suppliers and used without further purification unless otherwise stated. Anhydrous solvents were purchased from Sigma-Aldrich, and dried over 3 Å molecular sieves when necessary. Normal-phase flash column chromatography was performed using Biotage KP-Sil 50 μm silica gel columns and ACS grade solvents on a Biotage Isolera flash purification system or Teledyne Combiflash using Silicycle silica gel columns. Reverse phase preparative HPLC was performed with the following conditions: Phenomenex Gemini-NX C18, 110 Å, 150×21.2 mm; 5 μm. Eluting with a gradient of acetonitrile:water specified for each DART with 0.1% formic acid over 25 min, then 1 min at 100% acetonitrile. For Alexa647.1^{DART.2} (R11), 0.1% TFA rather than 0.1% formic acid was used and the flow rate was 20 mL/min. For Alexa488.1^{DART.2} (R10), the following prep HPLC conditions were used: Phenomenex Kinetex C18 100 Å, 50×30 mm; 5 μm, and a gradient of 15 to 80% acetonitrile in water (0.1% FA) at 50 mL/min for 12 min. Analytical thin layer chromatography (TLC) was performed on EM Reagent 0.25 mm silica gel 60 F254 plates and visualized by UV light. Proton (1H), and carbon (13C) NMR spectra were recorded on a 500 MHz Bruker Avance III with direct cryoprobe spectrometer. Chemical shifts were reported in ppm (0) and were referenced using residual nondeuterated solvent as an internal standard (CDCl₃ at 7.24 ppm for 1H-NMR and 77.0 for 13C-NMR. CD₃OD at 3.33 ppm for 1H-NMR and 47.6 for 13C-NMR. DMSO-de at 2.52 ppm for 1H-NMR and 39.9 ppm for 13C-NMR). Proton coupling constants are expressed in hertz (Hz). The following abbreviations were used to denote spin multiplicity for proton NMR: s=singlet, d=doublet, t=triplet, q=quartet, m=multiplet, br.s=broad singlet, dd=doublet of doublets, dt=doublet of triplets, quin=quintet, tt=triplet of triplets. Low resolution liquid chromatography/mass spectrometry (LCMS) was performed on a Waters Acquity-H UPLC/MS system with a 2.1 mm×50 mm, 1.7 μm, reversed phase BEH C18 column and LCMS grade solvents. A gradient elution from 95% water +0.1% formic acid/5% acetonitrile +0.1% formic acid to 95% acetonitrile +0.1% formic acid/5% water +0.1% formic acid over 2 min plus a further minute continuing this mixture at a flow rate of 0.85 mL/min was used as the eluent (for Alexa647.1^{DART.2} (R11), 0.1% TFA rather than 0.1% FA was used). Total ion current traces were obtained for electrospray positive and negative ionization (ESI+/ESI−). High-resolution mass spectra were obtained using an Agilent 6210 LC-TOF spectrometer in the positive ion mode using electrospray ionization with an Agilent G1312A HPLC pump and an Agilent G1367B autoinjector at the Integrated Molecular Structure Education and Research Center (IMSERC). All microwave-assisted reactions were carried out in a Biotage® initiator. All IUPAC compound names were generated using Chemdraw Version 19.1.1.21 or Instant J Chem.

### Example 1

### Synthesis of reagent biotin-PEG₁₂-HTL.1 (R1)

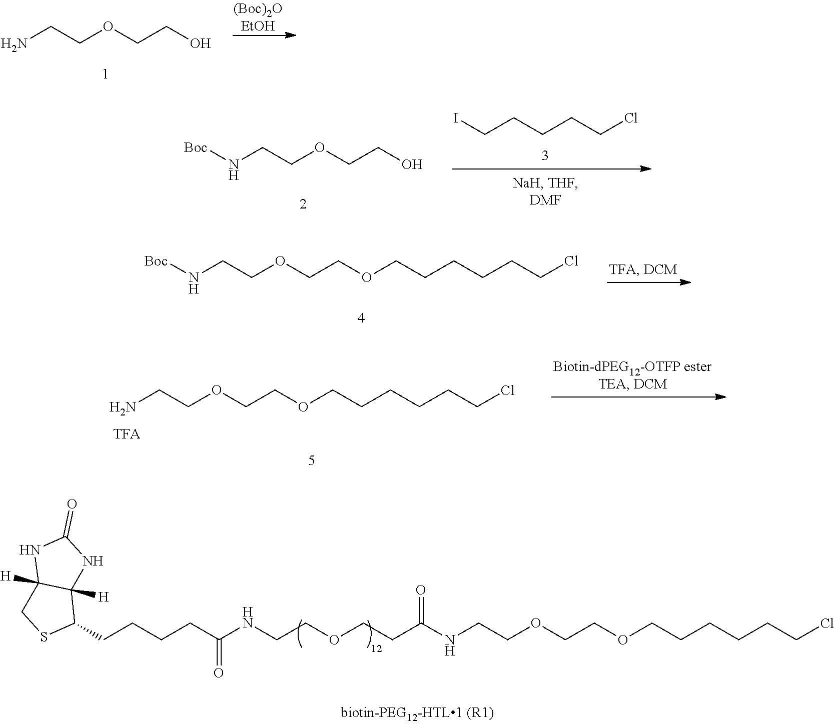

### Step 1: tert-butyl (2-(2-hydroxyethoxy)ethyl) carbamate, 2

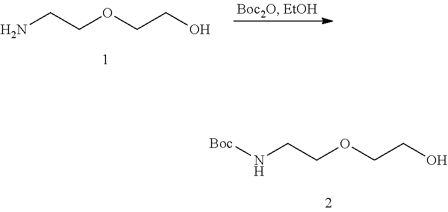 [SMILES: NCCOCCO ; CC(C)(C)OC(=O)NCCOCCO]

To a solution of 2-(2-aminoethoxy) ethan-1-ol (3.2 g, 30.4 mmol) in EtOH (10 mL) was added di-tert-butyl dicarbonate (6.64 g, 30.4 mmol) and reaction mixture was maintained at room temperature for 24 h. After the complete consumption of starting material (LC-MS), the volatiles were removed in vacuo, and the residue was concentrated. Purification by flash chromatography (24 g silica cartridge, 0-10% MeOH/CH₂Cl₂) afforded the title compound in 5.8 g, 92% yield. (ES-LCMS) m/z 228 (M+Na)⁺.

### Step 2: tert-butyl (2-(2-((6-chlorohexyl)oxy) ethoxy)ethyl) carbamate, 4

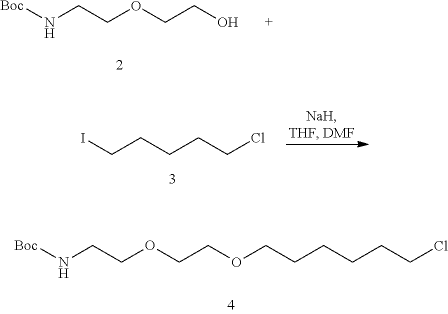 [SMILES: CC(C)(C)OC(=O)NCCOCCO ; ClCCCCCI ; CC(C)(C)OC(=O)NCCOCCOCCCCCCCl]

In a flame-dried 250 mL round-bottom flak under nitrogen was placed sodium hydride (60% dispersion in mineral oil, 0.76 g, 19.0 mmol) followed by dry THF (30 mL). The mixture was cooled in an ice bath to 0° C. and a solution of tert-butyl (2-(2-hydroxyethoxy)ethyl) carbamate (3 g, 14.6 mmol) in dry THF (5 mL) was slowly added. After the reaction was stirred for one hour at 0° C., a solution of 1-chloro-5-iodopentane (5.41 g, 21.9 mmol) in dry THF (5 mL) was slowly added. The reaction was kept at 0° C. overnight then poured into water and the product extracted with ethyl acetate (3×). The combined organic extracts were filtered to remove dark brown precipitates then washed with brine, dried over Na₂SO₄, filtered and concentrated in vacuo. The crude material was purified by flash chromatography (40 g silica column, 0-100% EtOAc/hexane gradient) to obtain the product as a light yellow oil (3.1 g, 65% yield). (ES-LCMS) m/z 343 (M+Na)⁺.

### Step 3:2-(2-((6-chlorohexyl)oxy) ethoxy) ethan-1-amine TFA salt, 5

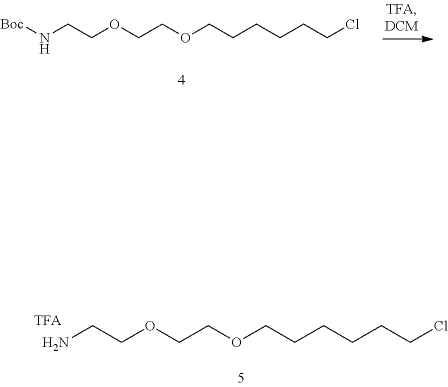 [SMILES: CC(C)(C)OC(=O)NCCOCCOCCCCCCCl ; NCCOCCOCCCCCCCl]

To a solution of tert-butyl (2-(2-((6-chlorohexyl)oxy) ethoxy)ethyl) carbamate (3 g, 9.28 mmol) in dichloromethane (20.0 mL) added trifluoroacetic acid (5 mL) and maintained at room temperature for 3.0 h. The reaction was concentrated under vacuum and triturated with two 5 mL portions of ether and ether decanted away and residual ether of was concentrated to yield a dark yellow product 3.11 g, 104% yield. ¹H NMR (CHLOROFORM-d, 300 MHz): δ=11.01-11.63 (m, 1H), 3.71-3.80 (m, J=10.0 Hz, 2H), 3.65-3.71 (m, 2H), 3.63 (br. s., 2H), 3.43-3.57 (m, 4H), 3.16-3.31 (m, 2H), 1.76 (s, 2H), 1.59 (s, 2H), 1.25-1.50 ppm (m, 4H); (ES-LCMS) m/z 224 (M+Na)⁺.

Step 4: N-(2-(2-((6-chlorohexyl)oxy) ethoxy)ethyl)-1-(5-((3aS,4S,6aR)-2-oxohexahydro-1H-thieno[3,4-d]imidazol-4-yl) pentanamido)-3, 6, 9, 12, 15, 18, 21, 24, 27, 30, 33,36-dodecaoxanonatriacontan-39-amide, R1

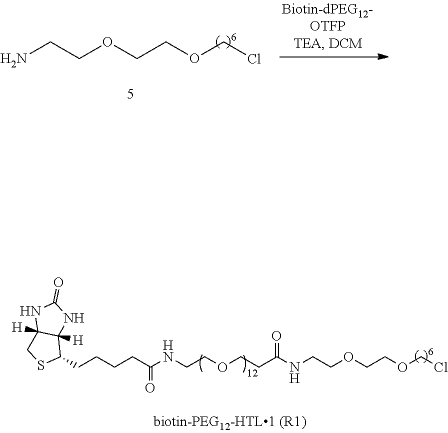

DIPEA (125 μL, 0.717 mmol) was added to a solution of 2-(2-((6-chlorohexyl)oxy) ethoxy) ethan-1-amine TFA salt (5) (20 mg, 0.089 mmol) and biotin-dPEG12-OTFP ester (71 mg, 0.071 mmol, Quanta Biodesign) in DCM (1 mL). After two hours the solvent was removed in vacuo and the residue was purified by Gilson prep HPLC (Sunfire Prep C18 OBD, 10 μm, 30×150 mm column, 15-75% ACN/H₂O (both containing 0.1% TFA) gradient). The product fractions were combined and concentrated to remove the acetonitrile before freeze drying overnight to obtain a colorless semi-solid product (20 mg, 21% yield). ES-LCMS: m/z=1049.5 (M+H)⁺; ¹H NMR (METHANOL-d₄, 300 MHz): δ=4.45-4.53 (m, 1H), 4.26-4.35 (m, 1H), 3.45-3.76 (m, 58H), 3.33-3.41 (m, 5H), 3.21 (dd, J=8.8, 4.7 Hz, 1H), 2.87-2.98 (m, 1H), 2.66-2.76 (m, 1H), 2.45 (s, 2H), 2.22 (s, 2H), 1.32-1.84 ppm (m, 14H); HRMS (ESI+): m/z calcd for C₄₇H₉₀ClN₄O₁₇S: 1049.5711 [M+H]⁺; found: 1049.5704, [M+H]⁺.

### Example 2

### Synthesis of reagent biotin-PEG₁₂-HTL.2 (R2)

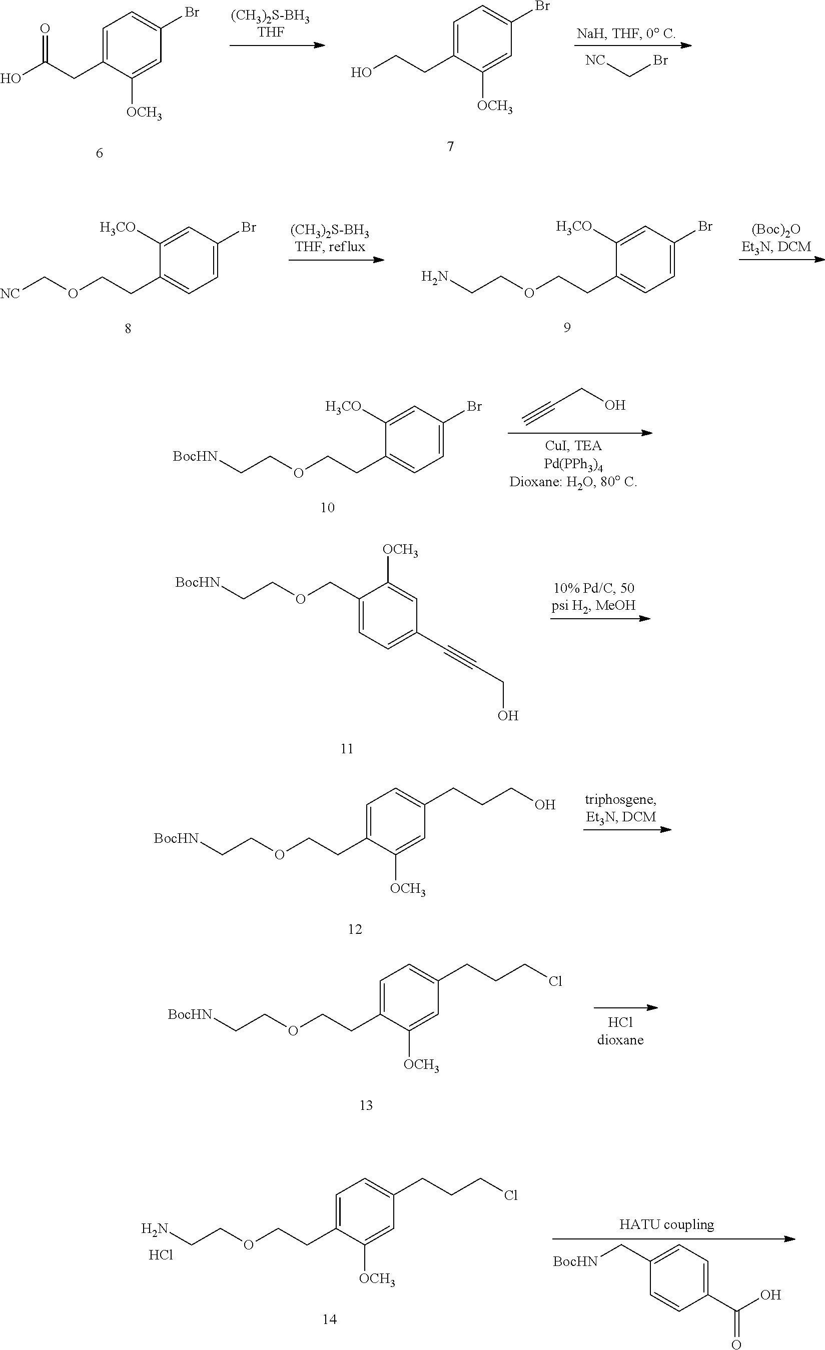

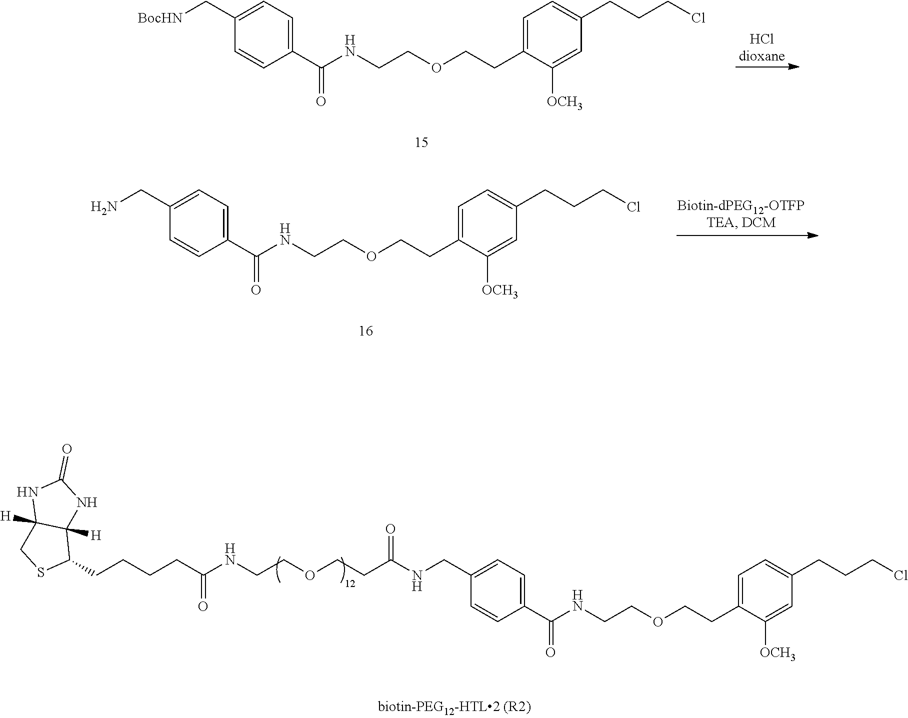

### Step 1:2-(4-bromo-2-methoxyphenyl) ethan-1-ol, 7

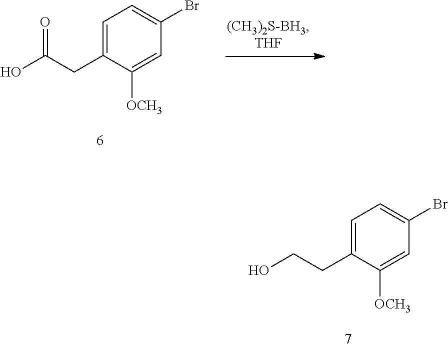 [SMILES: COc1cc(Br)ccc1CC(=O)O ; COc1cc(Br)ccc1CCO]

To a solution of 2-(4-bromo-2-methoxyphenyl) acetic acid (10.0 g, 41.0 mmol, CombiBlocks) in THF (100 mL) at 0° C. was added dropwise borane dimethylsulfide (21.1 mL, 246.0 mmol), keeping the temperature below 5° C. during the addition. The reaction was allowed to stir overnight as the ice bath warmed to ambient temperature. The reaction was poured into dry methanol (100 mL), stirred for 5 minutes and then the solvent was removed in vacuo. The crude product was obtained as a yellowish brown oil (13.45 g, 98% yield). ¹H NMR (METHANOL-d₄, 300 MHz): δ=6.94-7.09 (m, J=8.8 Hz, 3H), 3.78 (s, 3H), 3.67 (t, J=7.0 Hz, 2H), 2.77 ppm (t, J=7.0 Hz, 2H); ES-LCMS: m/z 232 (M+H)⁺.

### Step 2: [2-(4-Bromo-2-methoxy-phenyl)-ethoxy]-acetonitrile, 8

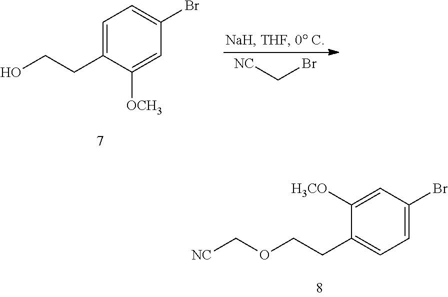

In a flame-dried 100 mL round-bottom flask under nitrogen was placed sodium hydride (60% dispersion in mineral oil, 9.35 g, 70.1 mmol) followed by dry THF (90 mL). The mixture was cooled in an ice bath to 0° C. and a solution of 2-(4-bromo-2-methoxy-phenyl)-ethanol (13.45 g, 58.4 mmol) in dry THF (50 mL) was slowly added. After the reaction was stirred for one hour at 0° C., a solution of bromoacetonitrile (4.89 mL, 70.1 mmol) in dry THF (50 mL) was slowly added. The reaction was kept at 0° C. overnight then poured into water and the product extracted with ethyl acetate (3×). The combined organic extracts were filtered to remove dark brown precipitates then washed with brine, dried over Na₂SO₄, filtered and concentrated in vacuo. The crude material was purified by flash chromatography (40 g silica column, 0-2% MeOH/DCM gradient) to obtain the product as a colorless oil (8.57 g, 54% yield). ¹H NMR (CHLOROFORM-d, 300 MHz): δ=7.03 (br. s., 3H), 4.23 (br. s., 2H), 3.67-3.91 (m, 5H), 2.89 ppm (d, J=5.9 Hz, 2H); ES-LCMS: m/z 271.5 (M+H)⁺.

### Step 3:2-[2-(4-Bromo-2-methoxy-phenyl)-ethoxy]-ethylamine, 9

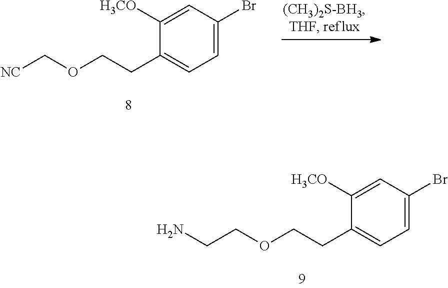 [SMILES: COc1cc(Br)ccc1CCOCC#N ; COc1cc(Br)ccc1CCOCCN]

A 2M solution of borane dimethyl sulfide complex in THF (47.8 mL, 95.5 mmol) was added to a solution of [2-(4-bromo-2-methoxy-phenyl)-ethoxy]-acetonitrile (8.57 g, 31.8 mmol) in THF (50 mL) and the mixture was heated at 70° C. for 3 hours. The reaction as cooled and then quenched with methanol (50 mL). The reaction was concentrated in vacuo and then concentrated twice more from methanol. The crude oil was place on under high vacuum overnight to afford compound 9 (5.2 g, 60% yield). ES-LCMS: m/z 274.5, 276.4 (M, M+2H)⁺.

Alternatively, Steps 2 and 3 can be done as follows:

### tert-butyl N-{2-[2-(4-bromo-2-methoxyphenyl) ethoxy]ethyl}carbamate (8)

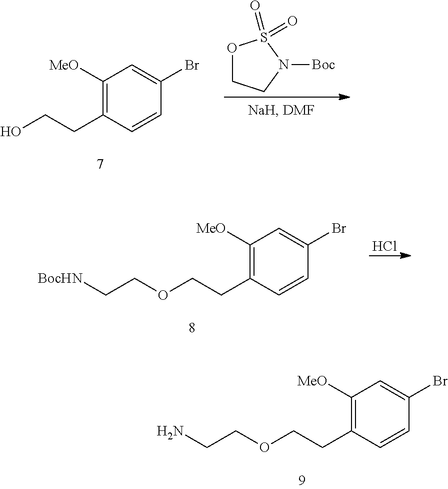

To a stirred, ice-cold solution of 2-(4-bromo-2-methoxyphenyl) ethan-1-ol (7) that had been chromatographed on silica gel (3.28 g, 14.2 mmol) in dry DMF (70 mL) was added 60% NaH in oil (1.14 g, 28.3 mmol). The mixture was stirred in the ice bath for 1.5 h and tert-butyl 2,2-dioxo-1,2/6,3-oxathiazolidine-3-carboxylate (3.17 g, 14.2 mmol) was added. The ice bath was allowed to melt and the mixture was stirred at rt for 3 d. The mixture was poured into ice-cold aq NH₄Cl (250 mL) and extracted with EtOAc (3×125 mL). The combined EtOAc layer was washed with brine (50 mL), dried over Na₂SO₄ and concentrated to leave an oil (8.13 g). Chromatography on a 120 g silica cartridge, eluted with a 0-40% EtOAc in hexanes gradient, afforded unreacted 7 (510 mg, 15%) and 8 (2.88 g, 54%). ¹H NMR (400 MHZ, CDCl₃) Shift=7.03-6.99 (m, 2H), 6.96 (s, 1H), 3.80 (s, 3H), 3.58 (t, J=6.8 Hz, 2H), 3.47 (t, J=5.3 Hz, 2H), 3.30-3.23 (m, 2H), 2.82 (t, J=6.8 Hz, 2H), 1.44 (s, 9H). LC-MS t_{R}=5.84 min, m/z 396 (M+Na⁺), 274 (M−100+H⁺), 213.

### 2-[2-(4-bromo-2-methoxyphenyl) ethoxy]ethan-1-amine (9)

To a stirred solution of 8 from several runs of the previous step (4.55 g, 12.2 mmol) in CH2Cl2 (25 mL) was added 4 M HCl in dioxane (12.5 mL, 50 mmol). The mixture was stirred at rt for 3 h and concentrated to leave the HCl salt of 9 (3.19 g, 84%) as a white powder. LC-MS tR=3.67 min, m/z 274 (M+H+), 213.

### Step 4: tert-butyl (2-(4-bromo-2-methoxyphenethoxy)ethyl) carbamate, 10

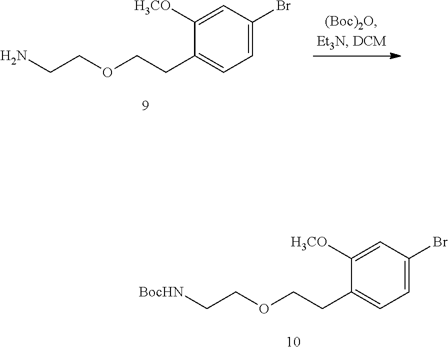 [SMILES: COc1cc(Br)ccc1CCOCCN ; COc1cc(Br)ccc1CCOCCNC(=O)OC(C)(C)C]

To a solution of 2-(4-bromo-2-methoxyphenethoxy) ethan-1-amine (8.6 g, 31.3 mmol) in DCM (50 mL) was added di-tert-butyl dicarbonate (8.9 g, 40.8 mmol) and the reaction mixture was maintained at 45° C. for 24 h. After the complete consumption of starting material (LC-MS), the volatiles were removed in vacuo, and the residue was dissolved in dichloromethane (30 mL). The solution was washed successively with 1% HCl (60 mL), brine (2×30 mL), and deionized water (30 mL), then dried over MgSO₄, filtered, and concentrated. Purification by flash chromatography (40 g silica cartridge, 0-100% EtOAc/Hexanes) afforded the title compound in 9 0 g, 77% yield. ¹H NMR (CHLOROFORM-0,300 MHZ): d=6.92-7.12 (m, 3H), 4.70-4.93 (m, 1H), 3.73-3.93 (m, 3H), 3.54-3.66 (m, 2H), 3.43-3.53 (m, 2H), 3.21-3.33 (m, 2H), 2.76-2.90 (m, 2H), 1.44 ppm (br. s., 9H); (ESI) m/z 397, 399 (M+Na, M+Na+2)⁺.

### Step 5: tert-butyl (2-((4-(3-hydroxyprop-1-yn-1-yl)-2-methoxybenzyl)oxy)ethyl) carbamate, 11

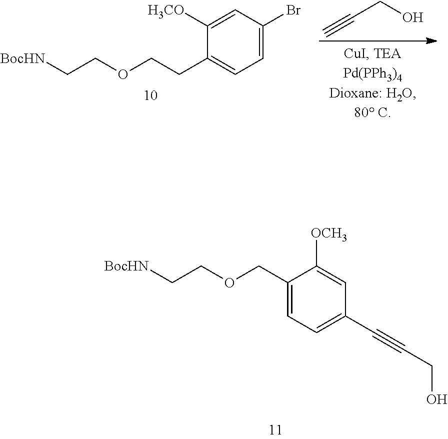

To mixture of compounds tert-butyl (2-(4-bromo-2-methoxyphenethoxy)ethyl) carbamate (7.0 g, 19.0 mmol), K₂CO₃ (5.25 g, 38.0 mmol), copper (I) iodide (0.36 g, 1.90 mmol), Pd(PPh)₄ (1.06 g, 0.95 mmol), and dioxane/H₂O (100 mL/10 mL) were added to a round bottom flask. The reaction mixture was placed under vacuum and then vented with nitrogen (3 times). The prop-2-yl-1-ol (13.3 g, 190.0 mmol) was added and the reaction heated to 75° C. overnight. Reaction monitored by LC-MS until complete. The solvent was removed under reduced pressure and crude residue purified by flash chromatography (40 g silica cartridge, 0-70% EtOAc/Hexanes) to afford the title compound in 2.16 g 33% yield. ¹H NMR (CHLOROFORM-d, 300 MHz): δ=7.03-7.11 (m, 1H), 6.93-7.01 (m, 1H), 6.86-6.92 (m, 1H), 4.77-5.01 (m, 1H), 4.46 (br. s., 2H), 4.24-4.33 (m, 1H), 3.78 (s, 3H), 3.59 (s, 2H), 3.46 (br. s., 2H), 3.20-3.31 (m, 2H), 2.85 (s, 2H), 1.43 ppm (s, 9H); (ESI) m/z 372 (M+Na)⁺.

### Step 6: tert-butyl (2-(4-(3-hydroxypropyl)-2-methoxyphenethoxy)ethyl) carbamate, 12

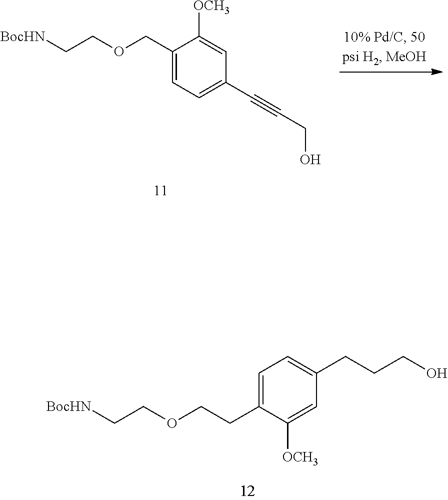 [SMILES: COc1cc(C#CCO)ccc1COCCNC(=O)OC(C)(C)C ; COc1cc(CCCO)ccc1CCOCCNC(=O)OC(C)(C)C]

tert-Butyl (2-((4-(3-hydroxyprop-1-yn-1-yl)-2-methoxybenzyl)oxy)ethyl) carbamate (1.8 g, 5.15 mmol) in EtOH (20 mL) was hydrogenated using 5% Pt/C (˜100 mg) in a small Parr vessel (250 mL) with hydrogen (55 psi) overnight. The reaction was filtered through celite while under nitrogen and the filtrate was then concentrated in vacuo. The crude product was purified by flash chromatography (24 g silica, 0-100% EtOAc/hexane gradient) to obtain product 12 (1.11 g, 61% yield). ¹H NMR (CHLOROFORM-d, 300 MHz): δ=6.99-7.15 (m, 1H), 6.69 (m, 2H), 4.91 (br. s., 1H), 3.80 (s, 3H), 3.56-3.72 (m, J=6.2, 6.2 Hz, 4H), 3.49 (t, J=5.0 Hz, 2H), 3.27 (d, J=5.3 Hz, 2H), 2.85 (t, J=7.3 Hz, 2H), 2.61-2.74 (m, J=7.6, 7.6 Hz, 1H), 1.81-1.95 (m, 2H), 1.44 ppm (s, 9H); ES-LCMS: m/z 377 (M+Na)⁺.

### Step 7: tert-butyl (2-(4-(3-chloropropyl)-2-methoxyphenethoxy)ethyl) carbamate, 13

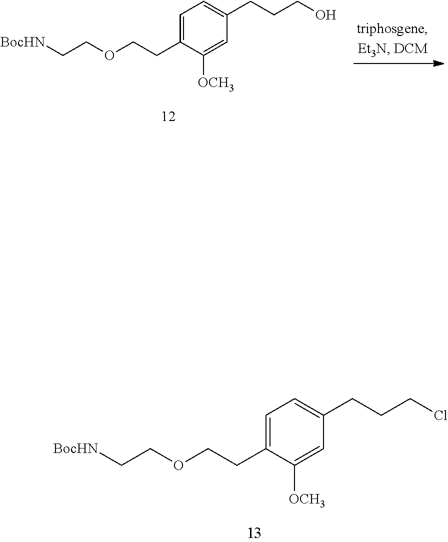 [SMILES: COc1cc(CCCO)ccc1CCOCCNC(=O)OC(C)(C)C ; COc1cc(CCCCl)ccc1CCOCCNC(=O)OC(C)(C)C]

To tert-butyl (2-(4-(3-hydroxypropyl)-2-methoxyphenethoxy)ethyl) carbamate (1.11 g, 3.14 mol) and triethylamine (1.09 mL, 7.85 mmol) in DCM (20 mL) was added triphosgene (0.46 g, 1.57 mmol) and the reaction stirred at room temperature for 1 h. Aqueous sodium bicarbonate was added and the water layer extracted twice with dichloromethane. The organic extracts were combined, dried (Na₂SO₄), and concentrated under reduced pressure to provide crude product. Purification by flash chromatography (24 g silica cartridge, 0-10% MeOH/CH₂Cl₂) afforded the title compound (1.05 g, 89% yield). ¹H NMR (CHLOROFORM-d, 300 MHz): δ=7.07 (d, J=7.6 Hz, 1H), 6.65-6.77 (m, 2H), 4.85 (br. s., 1H), 3.82 (s, 3H), 3.44-3.66 (m, 6H), 3.28 (q, J=4.9 Hz, 2H), 2.86 (t, J=7.0 Hz, 2H), 2.75 (t, J=7.3 Hz, 2H), 2.01-2.14 (m, 2H), 1.45 ppm (s, 9H); (ES-LCMS) m/z 395 (M+Na)⁺.

### Step 8:2-(4-(3-chloropropyl)-2-methoxyphenethoxy) ethan-1-amine, 14

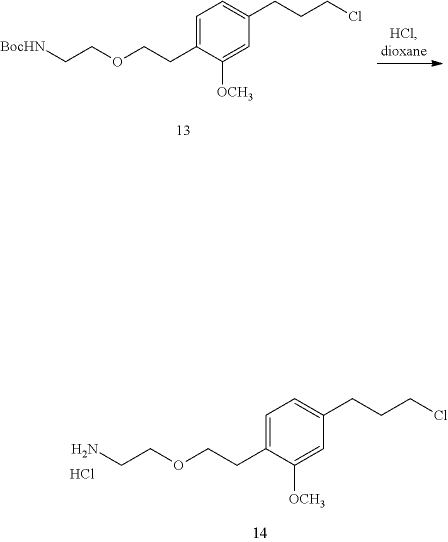 [SMILES: COc1cc(CCCCl)ccc1CCOCCNC(=O)OC(C)(C)C ; COc1cc(CCCCl)ccc1CCOCCN ; Cl]

To a solution of tert-butyl (2-(4-(3-chloropropyl)-2-methoxyphenethoxy)ethyl) carbamate (203 mg, 0.55 mmol) in dichloromethane (2.0 mL) was added 4N HCl in dioxane (0.5 mL) and maintained at room temperature for 3.0 h. The reaction was concentrated under vacuum and triturated with two portions of ether (5 mL) and the ether decanted off to yield product (160.2 mg, 99% yield). ¹H NMR (CHLOROFORM-d, 300 MHz): δ=8.29 (br. s., 2H), 7.07 (d, J=7.6 Hz, 1H), 6.62-6.78 (m, 2H), 3.80 (s, 3H), 3.62-3.76 (m, J=5.0, 5.0 Hz, 4H), 3.52 (t, J=6.4 Hz, 2H), 3.20 (br. s., 2H), 2.88 (t, J=7.0 Hz, 2H), 2.73 (t, J=7.3 Hz, 2H), 1.92-2.20 ppm (m, 2H); (ES-LCMS) m/z 272 (M+H)⁺.

### Step 9: tert-butyl (4-((2-(4-(3-chloropropyl)-2-methoxyphenethoxy)ethyl)carbamoyl)benzyl)carbamate, 15

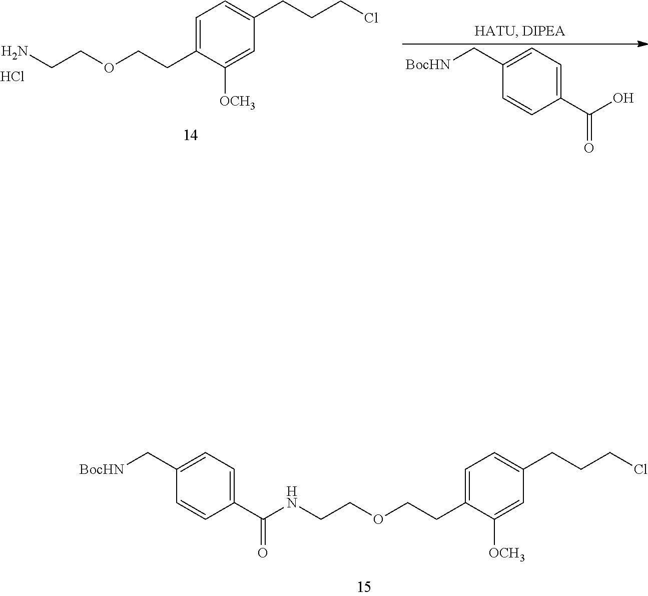 [SMILES: COc1cc(CCCCl)ccc1CCOCCN ; Cl ; CC(C)(C)OC(=O)NCc1ccc(C(=O)O)cc1 ; COc1cc(CCCCl)ccc1CCOCCNC(=O)c1ccc(CNC(=O)OC(C)(C)C)cc1]

2-(4-(3-Chloropropyl)-2-methoxyphenethoxy) ethan-1-amine (276 mg, 1.01 mmol), 4-Boc-aminomethyl)benzoic acid (305.9 mg, 1.21 mmol), HATU (964.5 mg, 2.53 mmol), and DIPEA (708 μL, 4.05 mmol) were combined in DMF (5 mL). The mixture was stirred for 4 hours then poured into water and the product extracted with ethyl acetate (3×). The combined organic extracts were washed with brine then dried over Na₂SO₄, filtered and concentrated in vacuo. The crude material was purified by flash chromatography (24 g silica, 15-70% EtOAc/hexane gradient) to obtain the product (339.8 mg, 66% yield). ¹H NMR (CHLOROFORM-d, 300 MHz): δ=7.68 (d, J=7.6 Hz, 2H), 7.34 (d, J=8.2 Hz, 2H), 7.08 (d, J=7.6 Hz, 1H), 6.67 (br. s., 2H), 6.44 (br. s., 1H), 4.86-5.00 (br. s, 1H), 4.22-4.47 (m, 2H), 3.80 (s, 3H), 3.47-3.71 (m, 8H), 2.61-2.98 (m, J=6.7, 6.7 Hz, 4H), 2.05 (t, J=6.7 Hz, 2H), 1.47 ppm (s, 9H); (ES-LCMS) m/z 506 (M+H)⁺.

### Step 10:4-(aminomethyl)-N-(2-(4-(3-chloropropyl)-2-methoxyphenethoxy)ethyl)benzamide, 16

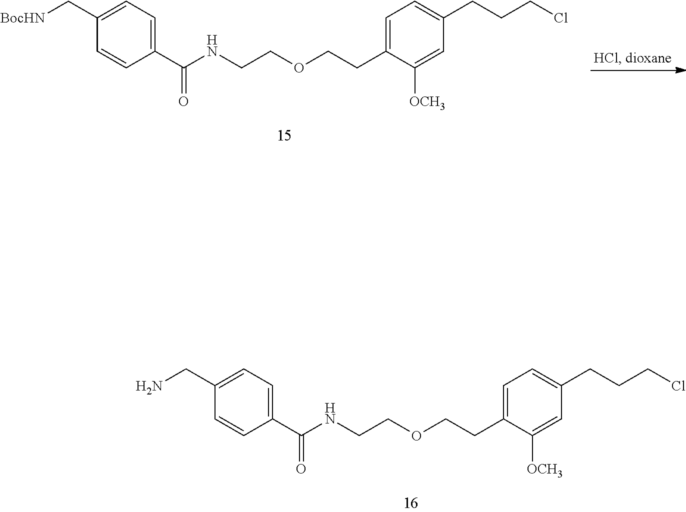 [SMILES: COc1cc(CCCCl)ccc1CCOCCNC(=O)c1ccc(CNC(=O)OC(C)(C)C)cc1 ; COc1cc(CCCCl)ccc1CCOCCNC(=O)c1ccc(CN)cc1]

To a solution of tert-butyl (2-(4-(3-chloropropyl)-2-methoxyphenethoxy)ethyl) carbamate (203 mg, 0.55 mmol) in dichloromethane (2.0 mL) was added 4N HCl (0.5 mL) in dioxane and the reaction maintained at room temperature for 3.0 h. The reaction was then concentrated under vacuum and triturated with two portions of ether (5 ml) that were subsequently decanted off to yield product (160.2 mg, 99% yield). ¹H NMR (CHLOROFORM-d, 300 MHz): δ=8.41 (br. s., 2H), 7.52 (br. s., 2H), 7.31 (br. s., 2H), 6.96-7.04 (br. s., 1H), 6.59-6.70 (m, 2H), 3.99 (s., 2H), 3.70-3.78 (s, 3H), 3.39-3.67 (m, 8H), 2.83 (m, 2H), 2.57-2.74 (m, 2H), 1.90-2.09 ppm (m, J=5.3 Hz, 2H); (ES-LCMS) m/z 406 (M+H)⁺.

### Step 11: Synthesis of Reagent Biotine-PEG₁₂-HTL.2 (R2)

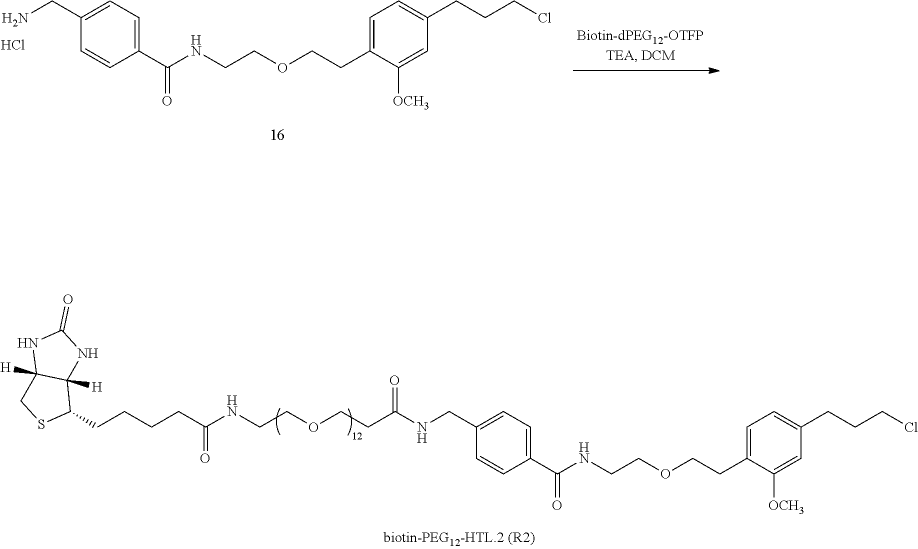

To a solution of solution of 2-(2-((6-chlorohexyl)oxy) ethoxy) ethan-1-amine HCl salt (16) (20 mg, 0.089 mmol) and biotin-dPEG12-OTFP (71 mg, 0.071 mmol, Quanta Biodesign) in DCM (1 mL) was added DIPEA (69 μL, 0.395 mmol) and the reaction stirred at room temperature overnight. The solvent was then removed in vacuo and the residue was purified by Gilson prep HPLC (Sunfire Prep C18 OBD, 10 μm, 30×150 mm column, 15-75% ACN/H₂O gradient (both solvents containing 0.1% TFA). The product fractions were combined and concentrated to remove acetonitrile before freeze drying overnight to obtain a colorless semi-solid product (20 mg, 21% yield). ¹H NMR (METHANOL-d₄, 300 MHz): δ=7.77 (d, J=8.2 Hz, 2H), 7.40 (d, J=8.2 Hz, 2H), 7.05 (d, J=7.6 Hz, 1H), 6.75 (s, 1H), 6.64 (d, J=7.6 Hz, 1H), 4.42-4.51 (m, 3H), 4.29 (dd, J=7.9, 4.4 Hz, 1H), 3.73-3.81 (m, 4H), 3.56-3.68 (m, 48H), 3.47-3.56 (m, 6H), 3.36 (d, J=5.3 Hz, 2H), 3.15-3.24 (m, J=8.5, 5.0 Hz, 1H), 2.79-2.96 (m, J=12.9, 4.7 Hz, 3H), 2.66-2.75 (m, J=2.3 Hz, 3H), 2.52 (t, J=6.2 Hz, 2H), 2.21 (t, J=7.3 Hz, 2H), 1.96-2.08 (m, 2H), 1.53-1.80 (m, J=2.3 Hz, 4H), 1.44 ppm (q, J=7.0 Hz, 2H); (ES-LCMS) m/z 1231.63 (M+H)⁺; HRMS (ESI): m/z calcd for C₅₉H₉₇ClN₅O₁₈S: 1230.6236 [M+H]⁺; found: 1230.6232, [M+H]⁺.

### Example 3

### Synthesis of reagent azido-PEG₃₆-HTL.1 (azido DART.1, R3)

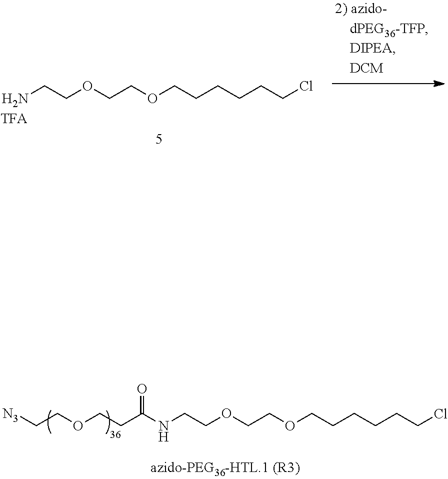

DIPEA (125 μL, 0.717 mmol) was added to a solution of 2-(2-((6-chlorohexyl)oxy) ethoxy) ethan-1-amine TFA salt (5) (20.5 mg, 0.109 mmol) and biotin-dPEG36-OTFP (202 mg, 0.109 mmol, Quanta Biodesign) in DCM (2 mL). After two hours the solvent was removed in vacuo and the residue was purified by Gilson prep HPLC (Sunfire Prep C18 OBD, 10 μm, 30×150 mm column, 15-80% MeCN/H₂O gradient (both solvents 0.1% TFA). The product fractions were combined and concentrated to remove the acetonitrile before freeze drying overnight to obtain a colorless semi-solid for the product (54 mg, 23% yield). ES-LCMS: m/z=1929.3 (M+Na)⁺; ¹H NMR (METHANOL-d₄, 300 MHz): δ=3.45-3.88 (m, 158H), 3.33-3.42 (m, 6H), 2.39-2.50 (m, 2H), 1.71-1.84 (m, 2H), 1.54-1.66 (m, 2H), 1.32-1.53 ppm (m, 4H); HRMS (ESI): m/z calcd for C₈₅H₁₆₉ClN₄NaO₃₉: 1928.0937 [M+Na]⁺; found: 1928.0944, [M+Na]⁺.

### Example 4

### Synthesis of reagent azido-PEG₃₆-HTL.2 (^{azido}DART.2, R4)

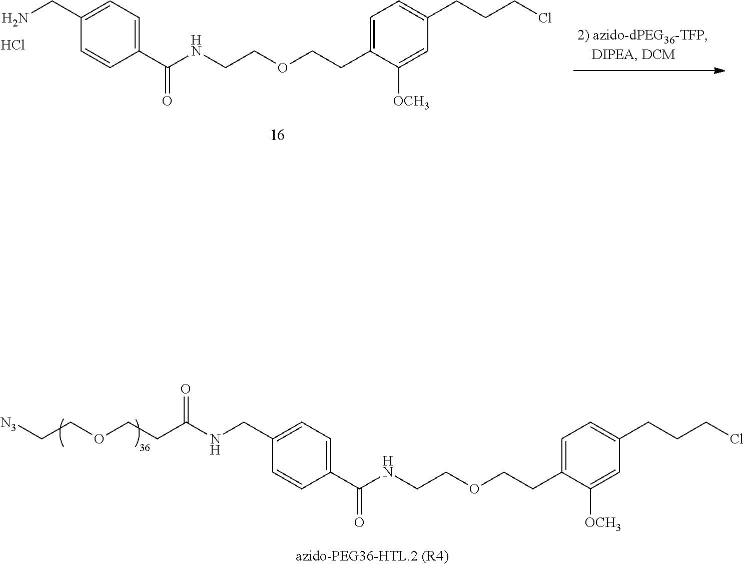

DIPEA (125 μL, 0.717 mmol) was added to a solution of 2-(2-((6-chlorohexyl)oxy) ethoxy) ethan-1-amine TFA salt (5) (20.5 mg, 0.109 mmol) and biotin-dPEG36-OTFP ester (202 mg, 0.109 mmol, Quanta Biodesign) in DCM (2 mL). After two hours the solvent was removed in vacuo and the residue was purified by Gilson prep HPLC (Sunfire Prep C18 OBD, 10 μm, 30×150 mm column, 15-80% ACN/H₂O gradient (both 0.1% TFA). The product fractions were combined and concentrated to remove the acetonitrile before freeze drying overnight to obtain a colorless semi-solid for the product (54 mg, 24%). ¹H NMR (METHANOL-d₄, 300 MHz): δ=7.77 (d, J=8.2 Hz, 2H), 7.40 (d, J=8.2 Hz, 2H), 7.04 (s, 1H), 6.75 (s, 1H), 6.59-6.69 (m, 1H), 4.43-4.51 (m, 2H), 3.78-3.80 (m, 3H), 3.53-3.71 (m, 150H), 3.34-3.41 (m, 4H), 2.80-2.88 (m, 2H), 2.66-2.75 (m, 2H), 2.47-2.55 (m, 2H), 2.02 ppm (s, 2H); (ES-LCMS) m/z 1053.74 ((M+Na)/2)⁺; HRMS (ESI): m/z calcd for C₉₇H₁₇₇ClN₅O₄₀: 2087.1645 [M+H]⁺; found: 2087.1652, [M+H]⁺.

### Example 5

### Experimental for YM90K.1^{DART.1} (R5)

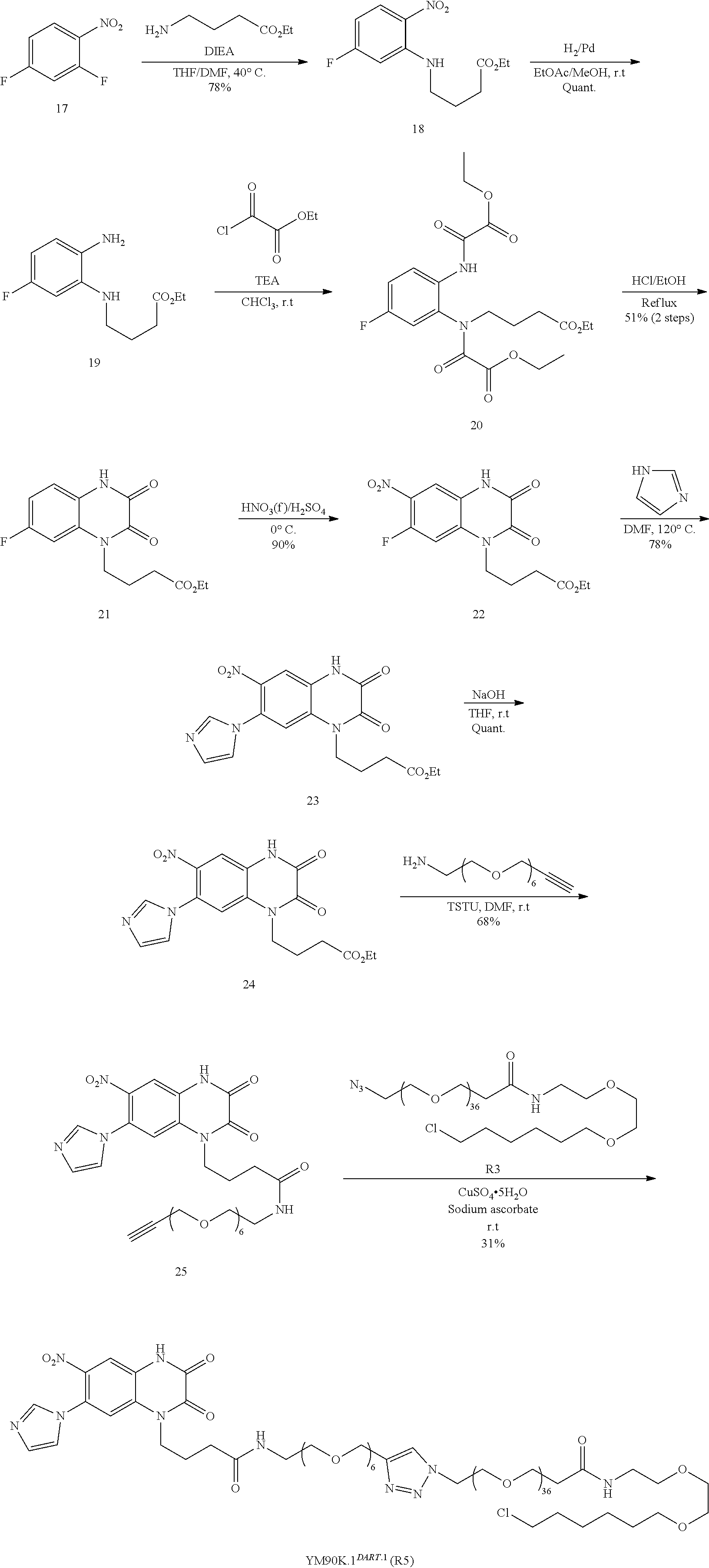

### Step 1: Synthesis of ethyl 4-((5-fluoro-2-nitrophenyl)amino) butanoate, 18

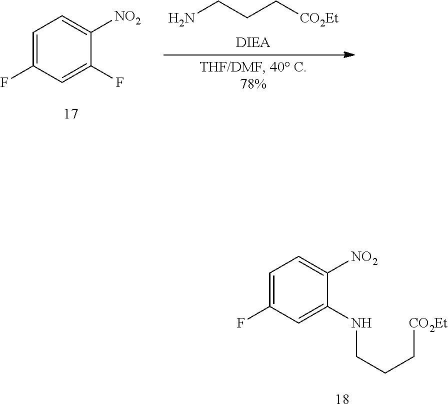 [SMILES: O=[N+]([O-])c1ccc(F)cc1F ; CCOC(=O)CCCN ; CCOC(=O)CCCNc1cc(F)ccc1[N+](=O)[O-]]

To a vial was added 2,4-difluoro-1-nitrobenzene, 17 (1.993 g, 1.374 mL, 12.53 mmol) and THF (10 mL)/DMF (5 mL) after which ethyl-4-aminobutanoate·HCl (2.000 g, 11.93 mmol) and triethylamine (1.449 g, 2.0 mL, 14.32 mmol) were added. The reaction was stirred at 40° C. overnight after which the mixture was concentrated, taken up in DCM (˜200 mL), washed with water (2×20 mL), brine (20 mL), filtered through an isolute phase separator, and concentrated to a yellow oil which was purified by silica gel chromatography eluting with 0 to 50% ethyl acetate in hexanes to give ethyl-4-((5-fluoro-2-nitrophenyl)amino) butanoate, 18 (2.5 g, 78%) as a yellow/orange oil. ¹H NMR (500 MHz, Chloroform-d) δ 8.19 (dd, J=9.5, 6.1 Hz, 2H), 6.52 (dd, J=11.5, 2.6 Hz, 1H), 6.35 (ddd, J=9.7, 7.2, 2.5 Hz, 1H), 4.14 (q, J=7.2 Hz, 2H), 3.32 (q, J=6.6 Hz, 2H), 2.44 (t, J=7.0 Hz, 2H), 2.02 (p, J=7.1 Hz, 2H), 1.30-1.20 (m, 3H). (ES-LCMS) m/z 271.2 (M+H)⁺.

### Step 2: Synthesis of ethyl 4-((2-amino-5-fluorophenyl)amino) butanoate, 19

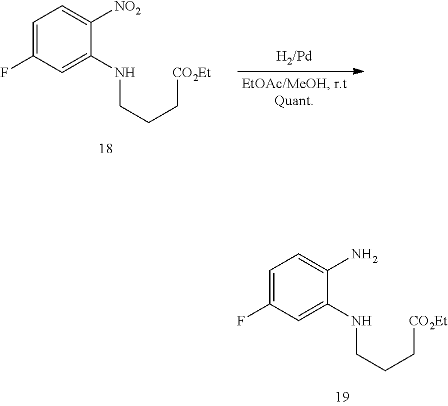 [SMILES: CCOC(=O)CCCNc1cc(F)ccc1[N+](=O)[O-] ; CCOC(=O)CCCNc1cc(F)ccc1N]

To a flask containing palladium on carbon (0.30 g, 0.28 mmol) was added (under a stream of nitrogen) a solution of ethyl 4-((5-fluoro-2-nitrophenyl)amino) butanoate, 18 (2.5 g, 9.3 mmol) in methanol (30 mL)/ethyl acetate (30 mL). The reaction was stirred under an atmosphere of hydrogen overnight after which it was filtered through celite and the filtrate concentrated to give ethyl 4-((2-amino-5-fluorophenyl)amino) butanoate, 19 (2.3 g, quant.) as a purple oil. ¹H NMR (500 MHZ, Chloroform-d) δ 6.59 (dd, J=8.3, 5.6 Hz, 1H), 6.36-6.23 (m, 2H), 4.12 (q, J=7.1 Hz, 2H), 3.12 (t, J=6.9 Hz, 2H), 3.07 (s, 2H), 2.44 (t, J=7.1 Hz, 2H), 1.98 (p, J=7.0 Hz, 2H), 1.24 (t, J=7.1 Hz, 4H). (ES-LCMS) m/z 241.3 (M+H)⁺.

### Steps 3/4: Synthesis of ethyl 4-(7-fluoro-2,3-dioxo-3,4-dihydroquinoxalin-1 (2H)-yl) butanoate, 21

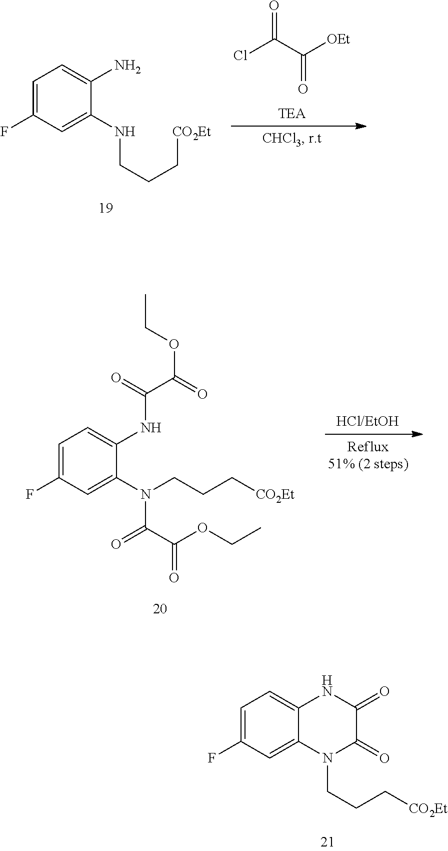 [SMILES: CCOC(=O)CCCNc1cc(F)ccc1N ; CCOC(=O)C(=O)Cl ; CCOC(=O)CCCN(C(=O)C(=O)OCC)c1cc(F)ccc1NC(=O)C(=O)OCC ; CCOC(=O)CCCn1c(=O)c(=O)[nH]c2ccc(F)cc21]

A solution of ethyl 4-((2-amino-5-fluorophenyl)amino) butanoate, 19 (2.3 g, 9.6 mmol) and triethylamine (1.9 g, 2.7 mL, 19 mmol) in chloroform (40 mL) was cooled in an ice/water bath and placed under nitrogen. Ethyl 2-chloro-2-oxoacetate (2.6 g, 2.1 mL, 19 mmol) in chloroform (5 mL) was added dropwise. The ice bath was removed upon dropwise addition and the reaction was allowed to stir at room temperature for 1.5 h after LC/MS indicated the desired intermediate, 20. The reaction was diluted with chloroform (15 mL) and transferred into a separatory funnel. The organic layer was washed with water (15 mL), saturated aqueous NaHCO₃ solution (2×15 mL), saturated NH₄Cl solution (2×15 mL), brine (15 mL), and filtered through an isolute phase separator. The filtrate was concentrated to a black/purple oil which was taken up in ethanol (30 mL). Concentrated HCl (0.5 mL) was added and the reaction was heated to 100° C. for 1 h after which LC/MS indicated starting material. Additional concentrated HCl (0.5 mL) was added and heating was continued for another 1.5 after the reaction was allowed to cool to room temperature and solids precipitated out of solution. The mixture was filtered and the collected solids washed with ethanol to give ethyl 4-(7-fluoro-2,3-dioxo-3,4-dihydroquinoxalin-1 (2H)-yl) butanoate, 21 (1.45 g, 51%) as a tan solid. ¹H NMR (500 MHZ, DMSO-d₆) δ 12.04 (s, 1H), 7.43 (dd, J=11.1, 2.6 Hz, 1H), 7.19 (dd, J=8.8, 5.5 Hz, 1H), 7.06 (td, J=8.5, 2.5 Hz, 1H), 4.17-3.98 (m, 4H), 2.47 (t, J=7.2 Hz, 2H), 1.87 (p, J=7.3 Hz, 2H), 1.20 (t, J=7.1 Hz, 3H). (ES-LCMS) m/z 295.2 (M+H)⁺.

### Step 5: Synthesis of ethyl 4-(7-fluoro-6-nitro-2,3-dioxo-3,4-dihydroquinoxalin-1 (2H)-yl) butanoate, 22

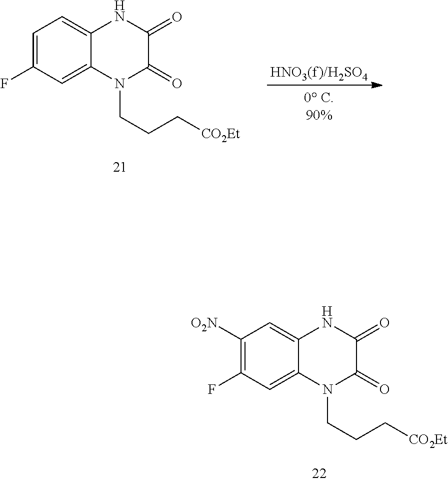 [SMILES: CCOC(=O)CCCn1c(=O)c(=O)[nH]c2ccc(F)cc21 ; CCOC(=O)CCCn1c(=O)c(=O)[nH]c2cc([N+](=O)[O-])c(F)cc21]

A round bottom flask containing ethyl 4-(7-fluoro-2,3-dioxo-3,4-dihydroquinoxalin-1 (2H)-yl) butanoate, 21 (1.45 g, 4.93 mmol) was immersed in an ice/water bath and concentrated sulfuric acid (15 mL) was added. The mixture was stirred and nitric acid (0.34 g, 0.24 mL, 4.93 mmol) (red, fuming) was added dropwise. The resulting reaction mixture was stirred for 30 min after which it was poured into a mixture of ice and water, and yellow solids precipitated out. The suspension was thoroughly stirred then filtered to give ethyl 4-(7-fluoro-6-nitro-2,3-dioxo-3,4-dihydroquinoxalin-1 (2H)-yl) butanoate, 22 (1.51 g, 90.3%) as a light yellow solid. ¹H NMR (500 MHZ, DMSO-d₆) δ 12.25 (s, 1H), 7.92 (d, J=7.1 Hz, 1H), 7.73 (d, J=13.4 Hz, 1H), 4.19-3.99 (m, 4H), 2.49 (t, J=7.2 Hz, 2H), 1.87 (p, J=7.3 Hz, 2H), 1.20 (t, J=7.1 Hz, 3H). (ES-LCMS) m/z 340.2 (M+H)⁺.

### Step 6: Synthesis of ethyl 4-(7-(1H-imidazol-1-yl)-6-nitro-2,3-dioxo-3,4-dihydroquinoxalin-1 (2H)-yl) butanoate, 23

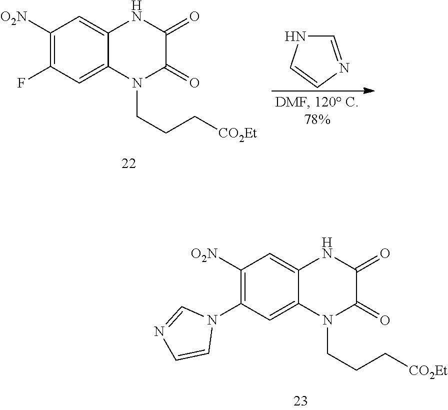 [SMILES: CCOC(=O)CCCn1c(=O)c(=O)[nH]c2cc([N+](=O)[O-])c(F)cc21 ; c1c[nH]cn1 ; CCOC(=O)CCCn1c(=O)c(=O)[nH]c2cc([N+](=O)[O-])c(-n3ccnc3)cc21]

To a vial containing ethyl 4-(7-fluoro-6-nitro-2,3-dioxo-3,4-dihydroquinoxalin-1 (2H)-yl) butanoate, 22 (1.5 g, 4.4 mmol) was added DMF (10 mL) after which imidazole (0.63 g, 9.3 mmol) was added and the reaction was heated overnight at 80° C. after which LC/MS still indicated starting material. Additional imidazole (100 mg, 0.7 mmol) was added and the temperature was increased to 100° C. for 2 h after which LC/MS still starting material. Additional imidazole (100 mg, 0.7 mmol) was added and heating was continued at 100° C. for 2 h then 120° C. for 2 h after which the reaction was finally complete. The reaction mixture was poured into a mixture of ice/water and stirred vigorously then filtered. The collected solid was isolated to give ethyl 4-(7-(1H-imidazol-1-yl)-6-nitro-2,3-dioxo-3,4-dihydroquinoxalin-1 (2H)-yl) butanoate, 23 (1.34 g, 78%) as an orange solid. ¹H NMR (500 MHZ, DMSO-d₆) δ 12.57 (s, 1H), 9.45 (s, 1H), 8.17 (s, 1H), 8.05 (d, J=1.7 Hz, 1H), 8.00 (s, 1H), 7.89 (d, J=1.7 Hz, 1H), 4.13 (t, J=7.5 Hz, 2H), 2.40 (t, J=7.3 Hz, 2H), 1.87 (p, J=7.4 Hz, 2H). (ES-LCMS) m/z 388.3 (M+H)⁺.

### Step 7:4-(7-(1H-imidazol-1-yl)-6-nitro-2,3-dioxo-3,4-dihydroquinoxalin-1 (2H)-yl) butanoic acid, 24

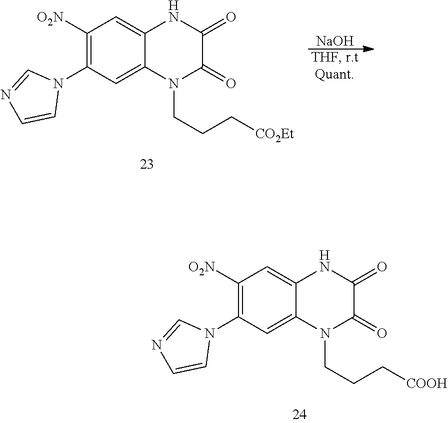 [SMILES: CCOC(=O)CCCn1c(=O)c(=O)[nH]c2cc([N+](=O)[O-])c(-n3ccnc3)cc21 ; "NaOH" ; "THF, r.tQuant." ; O=C(O)CCCn1c(=O)c(=O)[nH]c2cc([N+](=O)[O-])c(-n3ccnc3)cc21]

To a vial containing ethyl 4-(7-(1H-imidazol-1-yl)-6-nitro-2,3-dioxo-3,4-dihydroquinoxalin-1 (2H)-yl) butanoate, 23 (1 g, 3 mmol) was added NaOH (1 M in water, 8 mL, 4 mmol) and THF (2 mL) and the reaction was stirred at room temperature for 4 h. THF was removed under a stream of nitrogen and to the resulting solution was added 2 M HCl (˜ 6 mL) [pH˜ 2] and solids precipitated. The mixture was vigorously stirred then filtered to give 4-(7-(1H-imidazol-1-yl)-6-nitro-2,3-dioxo-3,4-dihydroquinoxalin-1 (2H)-yl) butanoic acid, 24 (1.12 g, quant.) as a yellow solid. ¹H NMR (500 MHZ, DMSO-d₆) δ 12.57 (s, 1H), 9.45 (s, 1H), 8.17 (s, 1H), 8.05 (d, J=1.7 Hz, 1H), 8.00 (s, 1H), 7.89 (d, J=1.7 Hz, 1H), 4.13 (t, J=7.5 Hz, 2H), 2.40 (t, J=7.3 Hz, 2H), 1.87 (p, J=7.4 Hz, 2H). (ES-LCMS) m/z 360.3 (M+H)⁺.

### Step 8: Synthesis of 4-(7-(1H-imidazol-1-yl)-6-nitro-2,3-dioxo-3,4-dihydroquinoxalin-1 (2H)-yl)-N-(3, 6, 9, 12, 15, 18-hexaoxahenicos-20-yn-1-yl) butanamide, 25

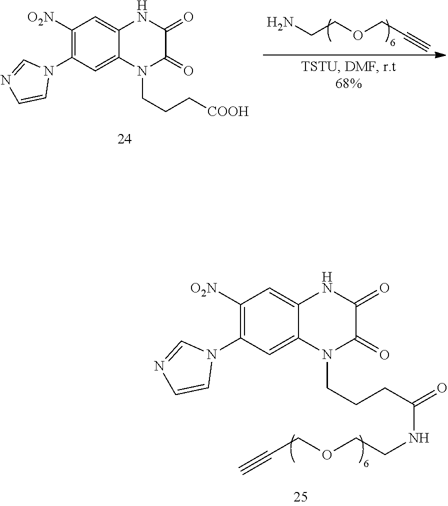

To 4-(7-(1H-imidazol-1-yl)-6-nitro-2,3-dioxo-3,4-dihydroquinoxalin-1 (2H)-yl) butanoic acid, 24 (100 mg, 278 μmol) was added DMF (1.5 mL), DIPEA (71.9 mg, 97 μL. 557 μmol) and 1-(bis(dimethylamino)(tetrafluoro-15-boraneyl)methoxy)pyrrolidine-2,5-dione (TSTU) (83.8 mg, 278 μmol). The reaction was stirred for 15 min after 3, 6, 9, 12, 15, 18-hexaoxahenicos-20-yn-1-amine (88.9 mg, 278 μmol) was added and the reaction was stirred at room temperature for 1 h after which it was directly purified by reverse phase prep HPLC eluting from 5 to 50% acetonitrile in water (0.1% FA and conditions as described in the general chemical synthesis information) to give 4-(7-(1H-imidazol-1-yl)-6-nitro-2,3-dioxo-3,4-dihydroquinoxalin-1 (2H)-yl)-N-(3, 6, 9, 12, 15, 18-hexaoxahenicos-20-yn-1-yl) butanamide, 25 (125 mg, 68%) as a yellow oil. ¹H NMR (500 MHZ, CDCl₃) δ 8.08 (s, 1H), 7.78 (s, 1H), 7.71 (s, 1H), 7.19 (s, 1H), 7.15 (s, 1H), 6.66 (t, J=5.5 Hz, 1H), 4.27 (t, J=7.5 Hz, 2H), 4.13 (d, J=2.4 Hz, 2H), 3.69-3.55 (m, 20H), 3.51 (t, J=5.1 Hz, 2H), 3.38 (q, J=5.2 Hz, 2H), 2.49-2.26 (m, 3H), 2.11 (q, J=7.0 Hz, 2H). (ES-LCMS) m/z 661.4 (M+H)⁺.

### Step 9: Synthesis of YM90K.1^{DART.1} (R5)

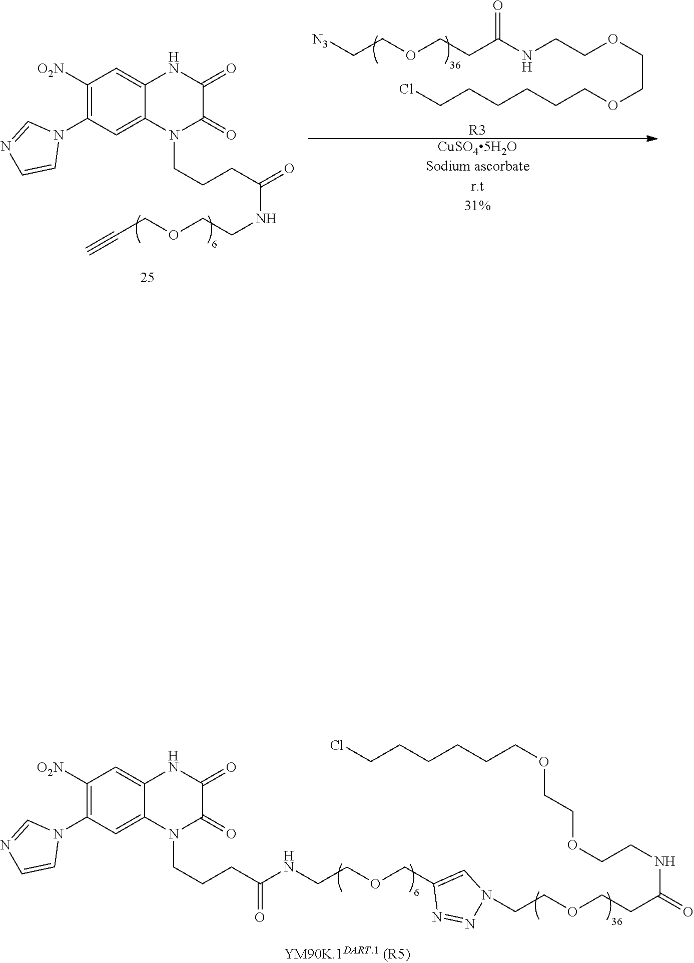

To 4-(7-(1H-imidazol-1-yl)-6-nitro-2,3-dioxo-3,4-dihydroquinoxalin-1 (2H)-yl)-N-(3, 6, 9, 12, 15, 18-hexaoxahenicos-20-yn-1-yl) butanamide, 25 (5 mg, 7.6 μmol) was added EtOH (0.4 mL)/t-BuOH (0.2 mL)/Water (0.2 mL) after which azido-PEG36-HTL1.0, R3 (14 mg, 7.6 μmol) was added. Finally, Cu(II) sulfate pentahydrate (0.1 mg, 0.38 μmol) and sodium ascorbate (0.14 mg, 0.76 μmol) were added and the reaction mixture was stirred overnight at room temperature. The mixture was directly purified by reverse phase prep HPLC eluting from 5 to 70% acetonitrile in water (0.1% FA, 220 nm collection wavelength and other parameters as described in the general chemical synthesis section) and lyophilized to give 1-(4-(24-(7-(1H-imidazol-1-yl)-6-nitro-2,3-dioxo-3,4-dihydroquinoxalin-1 (2H)-yl)-21-oxo-2, 5, 8, 11, 14, 17-hexaoxa-20-azatetracosyl)-1H-1,2,3-triazol-1-yl)-N-(2-(2-((6-chlorohexyl)oxy) ethoxy)ethyl)-3, 6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108-hexatriacontaoxaundecahectan-111-amide, YM90K.1^{DART.1}, R5 (6 mg, 31%). ¹H NMR (500 MHZ, CDCl₃) δ 8.11 (s, 1H), 7.79-7.62 (m, 2H), 7.20 (s, 1H), 7.14 (s, 1H), 6.64 (s, 1H), 6.59 (s, 1H), 4.62 (s, 2H), 4.50 (q, J=5.2 Hz, 2H), 4.27 (t, J=7.4 Hz, 1H), 3.84 (t, J=5.1 Hz, 2H), 3.77-3.34 (m, 178H), 2.45 (t, J=6.1 Hz, 2H), 2.32 (t, J=6.5 Hz, 1H), 2.11-2.00 (m, 2H), 1.75 (p, J=6.9 Hz, 2H), 1.58 (p, J=6.8 Hz, 2H), 1.43 (q, J=8.3 Hz, 2H), 1.38-1.29 (m, 2H). HRMS (ESI+): m/z calcd for C₁₁₅H₂₁₁ClN₁₀O₅₀: 1284.1994 [M+2H]²⁺; found: 1284.1989, [M+2H]²⁺.

### Example 6

### Experimental for YM90K.1^{DART.2} (R6)

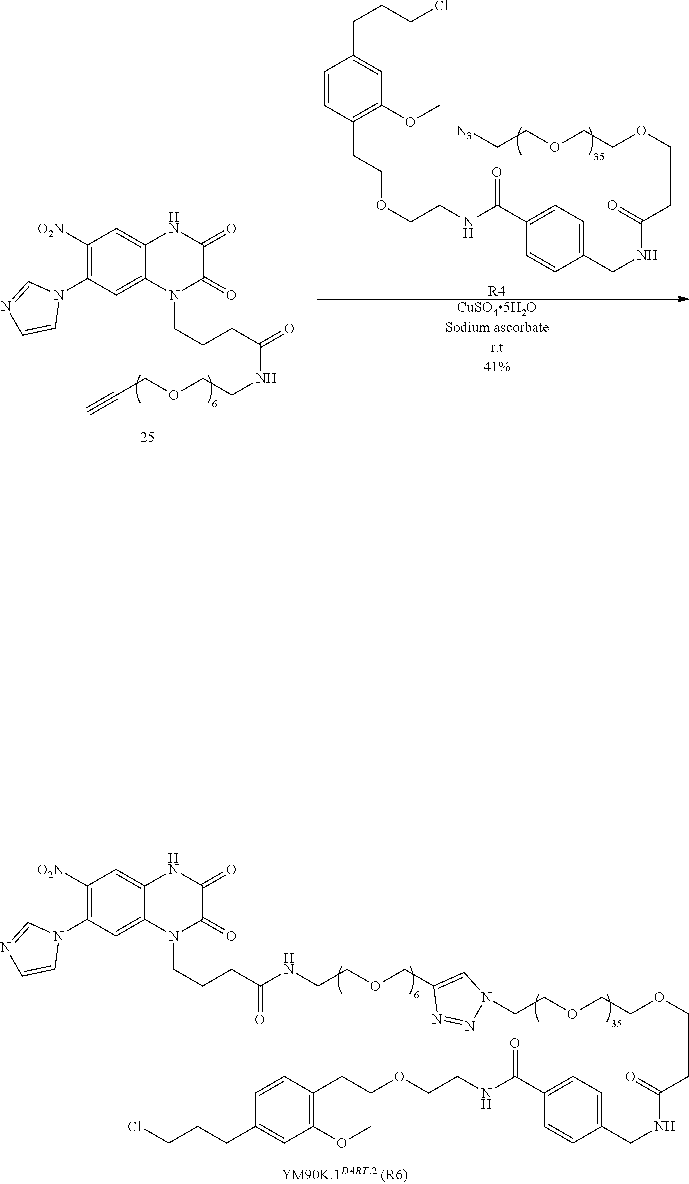

To 4-(7-(1H-imidazol-1-yl)-6-nitro-2,3-dioxo-3,4-dihydroquinoxalin-1 (2H)-yl)-N-(3, 6, 9, 12, 15, 18-hexaoxahenicos-20-yn-1-yl) butanamide, 25 (10 mg, 15 μmol) was added EtOH (0.4 mL)/t-BuOH (0.2 mL)/Water (0.2 mL) after which azido-PEG36-HTL2.0, R4 (32 mg, 15 μmol) was added. Finally, Cu(II) sulfate pentahydrate (0.19 mg, 0.76 μmol) and sodium ascorbate (0.27 mg, 1.5 μmol) were added and the reaction mixture was stirred overnight at room temperature. The mixture was directly purified by reverse phase prep HPLC eluting from 5 to 70% acetonitrile in water (0.1% FA, 220 nm collection wavelength and other parameters as described in the general chemical synthesis section) and lyophilized to give 4-(113-(4-(24-(7-(1H-imidazol-1-yl)-6-nitro-2,3-dioxo-3,4-dihydroquinoxalin-1 (2H)-yl)-21-oxo-2, 5, 8, 11, 14, 17-hexaoxa-20-azatetracosyl)-1H-1,2,3-triazol-1-yl)-3-oxo-6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111-hexatriacontaoxa-2-azatridecahectyl)-N-(2-(4-(3-chloropropyl)-2-methoxyphenethoxy)ethyl)benzamide, YM90K.1^{DART.2}, R6 (17 mg, 41%). ¹H NMR (500 MHZ, CDCl₃) δ 8.15-8.12 (m, 2H), 7.90 (br.s, 1H), 7.73 (s, 2H), 7.66 (d, J=8.1 Hz, 2H), 7.32 (d, J=8.0 Hz, 2H), 7.25-7.12 (m, 2H), 7.04 (d, J=7.8 Hz, 1H), 6.79 (d, J=7.0 Hz, 1H), 6.65 (d, J=6.3 Hz, 2H), 6.54 (d, J=5.4 Hz, 1H), 4.61 (s, 2H), 4.51-4.42 (m, 4H), 4.24 (t, J=7.5 Hz, 2H), 3.83 (t, J=5.0 Hz, 2H), 3.77 (s, 3H), 3.75 (t, J=5.7 Hz, 3H), 3.68-3.47 (m, 163H), 3.36 (q, J=5.2 Hz, 2H), 2.85 (t, J=7.0 Hz, 2H), 2.70 (t, J=7.4 Hz, 2H), 2.53 (t, J=5.6 Hz, 2H), 2.33 (d, J=6.4 Hz, 2H), 2.06-2.00 (m, 4H). HRMS (ESI+): m/z calcd for C₁₂₇H₂₁₈ClN₁₁O₅₁: 1374.7257 [M+2H]²⁺; found: 1374.7217, [M+2H]²⁺.

### Example 7

### Experimental for Gabazine.1^{DART.2} (R7)

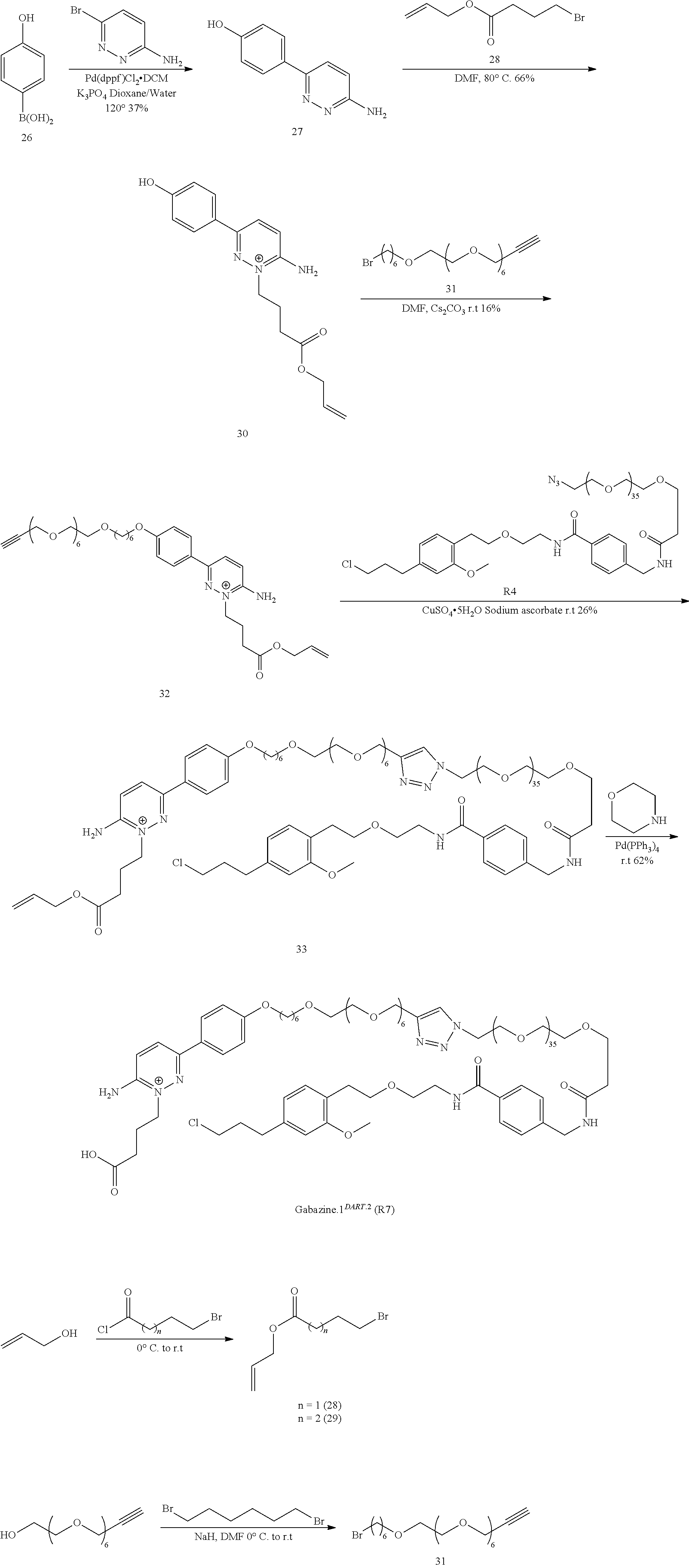

### Synthesis of allyl 4-bromobutanoate, 28

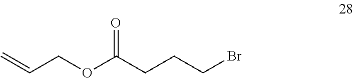

To cooled (ice/water bath) mixture of allyl alcohol (1.9 g, 2.2 mL, 6 equiv., 32 mmol) under nitrogen was added 4-bromobutanoyl chloride (1.0 g, 0.62 mL, 1 equiv., 5.0 mmol) dropwise. The reaction was allowed to stir at 0° C. for 2 h followed by room temperature for 3 h after TLC (1:1 EtOAc:Hex, iodine/silica stain) indicated conversion. The reaction mixture was directly concentrated to give allyl 4-bromobutanoate, 28 (1.035 g, 4.998 mmol, 93%) as a brown oil. ¹H NMR (500 MHZ, CDCl₃) δ 5.90 (ddt, J=16.7, 11.1, 5.6 Hz, 1H), 5.27 (dd, J=38.1, 13.8 Hz, 2H), 4.59 (d, J=5.8 Hz, 2H), 3.46 (t, J=6.3 Hz, 2H), 2.54 (t, J=7.1 Hz, 2H), 2.18 (p, J=6.7 Hz, 2H). (ES-LCMS) m/z 209.2 (M+2H)⁺.

### Synthesis of allyl 5-bromopentanoate, 29

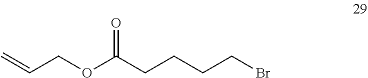

To a cooled (ice/water bath) mixture of allyl alcohol (1.7 g, 2.0 mL, 6 equiv., 30 mmol) under nitrogen was added 5-bromopentanoyl chloride (1.0 g, 0.62 mL, 1 equiv., 5.0 mmol) dropwise. The reaction was allowed to stir at 0° C. for 2 h followed by room temperature for 3 h after TLC (1:1 EtOAc:Hex, iodine/silica stain) indicated conversion. The reaction mixture was directly concentrated to give allyl 5-bromopentanoate, 29 (0.77 g, 3.48 mmol, 69%) as a brown oil. ¹H NMR (500 MHZ, CDCl₃) δ 5.94-5.86 (m, 1H), 5.30 (d, J=17.1 Hz, 1H), 5.22 (d, J=10.4 Hz, 1H), 4.57 (d, J=5.6 Hz, 2H), 3.40 (t, J=6.5 Hz, 2H), 2.37 (t, J=7.2 Hz, 2H), 1.89 (q, J=7.0 Hz, 2H), 1.79 (q, J=7.3 Hz, 2H). (ES-LCMS) m/z 223.1 (M+2H)⁺.

### Synthesis of 28-bromo-4, 7, 10, 13, 16, 19,22-heptaoxaoctacos-1-yne, 31

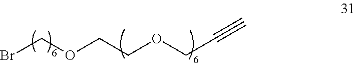

To 3, 6, 9, 12, 15, 18-hexaoxahenicos-20-yn-1-ol (1.00 g, 1 equiv., 3.12 mmol) in DMF (12 mL) in an ice/water bath and under nitrogen was added NaH (137 mg, 60% Wt, 1.1 equiv., 3.43 mmol) and the mixture was stirred for 20 min after which 1,6-dibromohexane (1.22 g, 768 μL, 1.6 equiv., 4.99 mmol) was added quickly. After 2 h of stirring as the ice bath expired, the reaction was quenched with saturated aqueous ammonium chloride solution and diluted with ethyl acetate and water. The aqueous layer was extracted once with ethyl acetate. Combined organics were washed with water, 10% LiCl (aqueous) solution, brine, filtered through an isolute phase separator, and concentrated to a residue which was purified by silica gel chromatography eluting with 0 to 15% MeOH in DCM to give 28-bromo-4, 7, 10, 13, 16, 19,22-heptaoxaoctacos-1-yne, 31 (865 mg, 1.79 mmol, 57.3%) as a light yellow oil. ¹H NMR (500 MHz, CDCl₃) δ 4.18 (d, J=2.4 Hz, 2H), 3.69-3.59 (m, 22H), 3.55 (dd, J=5.9, 3.8 Hz, 2H), 3.43 (t, J=6.6 Hz, 2H), 3.38 (t, J=6.8 Hz, 2H), 2.41 (t, J=2.4 Hz, 1H), 1.86-1.80 (m, 2H), 1.60-1.54 (m, 2H), 1.46-1.39 (m, 2H), 1.37-1.30 (m, 2H). (ES-LCMS) m/z 485.3 (M+2H)⁺.

### Step 1: Synthesis of 4-(6-aminopyridazin-3-yl) phenol, 27

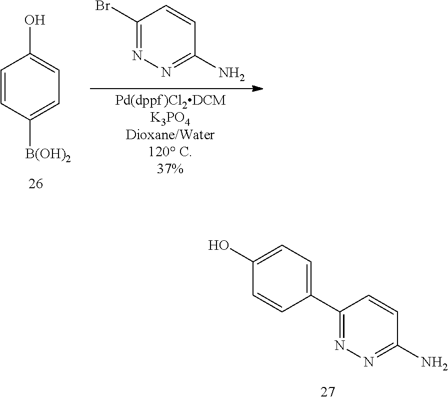 [SMILES: OB(O)c1ccc(O)cc1 ; Nc1ccc(Br)nn1 ; Nc1ccc(-c2ccc(O)cc2)nn1]

To (4-hydroxyphenyl) boronic acid, 26 (2 g, 14.5 mmol) was added K₃PO₄ (6.16 g, 29 mmol), Pd(dppf)Cl2·DCM (0.71 g, 0.87 mmol), 6-bromopyridazin-3-amine (2.47 g, 14.2 mmol) and dioxane (44 mL)/water (28 mL), and the reaction was heated in the microwave at 120° C. for 10 min (note: separated into 4 portions which were each heated separately at 120° C. for 10 min). The combined reaction mixture was diluted with ethyl acetate (50 mL), filtered through a pad of silica, and the contents transferred to a separatory funnel. The organic layer was washed with water (20 mL), brine (20 mL), and concentrated to a black solid which was triturated from dichloromethane. This solid was further purified by silica gel chromatography eluting with 0 to 35% methanol in dichloromethane (product eluted in a wide band) to give 4-(6-aminopyridazin-3-yl) phenol, 27 (1 g, 5.34 mmol, 36.8% yield) as a dark, brown solid. ¹H NMR (500 MHZ, CD₃OD) δ 7.73 (dd, J=9.0, 2.5 Hz, 3H), 7.00 (d, J=9.3 Hz, 1H), 6.92-6.84 (m, 2H). (ES-LCMS) m/z 188.1 (M+H)⁺.

### Step 2: Synthesis of allyl 4-(3-(4-hydroxyphenyl)-6-iminopyridazin-1 (6H)-yl) butanoate, 30

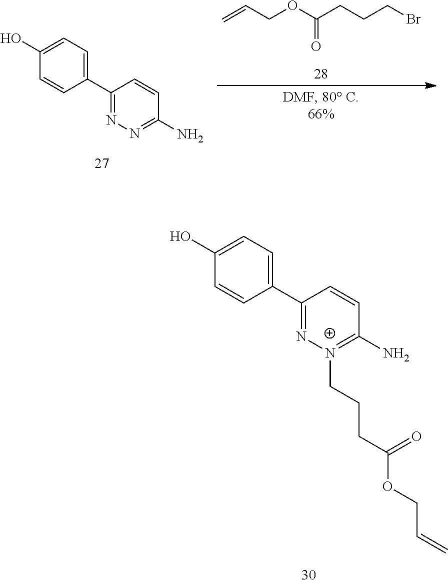

To 4-(6-aminopyridazin-3-yl) phenol, 27 (1 g, 5.34 mmol) was added allyl 4-bromobutanoate, 28 (1.14 g, 5.51 mmol) and DMF (26.7 mL) and the reaction was heated at 80° C. for 7 h after which it was concentrated to remove DMF. The resulting thick, black oil was then triturated from acetonitrile give allyl 4-(3-(4-hydroxyphenyl)-6-iminopyridazin-1 (6H)-yl) butanoate, 30 (1.1 g, 3.51 mmol, 65.7% yield) as a brown solid. Since there are three nitrogens and a phenolic oxygen which may all react with the alkyl bromide, the assignment and confirmation of this product was described in the synthesis of Gabazine.7^{DART.2} (R9). ¹H NMR (500 MHZ, Methanol-d₄) δ 8.23 (d, J=9.5 Hz, 1H), 7.84 (d, J=8.6 Hz, 2H), 7.55 (d, J=9.5 Hz, 1H), 6.91 (dd, J=9.1, 2.7 Hz, 2H), 5.86 (ddt, J=16.5, 11.0, 5.7 Hz, 1H), 5.33-5.13 (m, 2H), 4.50 (d, J=5.7 Hz, 2H), 4.43 (t, J=6.9 Hz, 2H), 2.63 (t, J=6.8 Hz, 2H), 2.27 (p, J=6.8 Hz, 2H). ¹³C NMR (125 MHZ, CD₃OD) δ 172.65, 161.38, 152.44, 150.54, 132.00, 130.83, 127.91, 125.25, 123.22, 117.22, 115.99, 65.08, 55.19, 29.94, 21.12. (ES-LCMS) m/z 314.3 (M+H)⁺.

### Step 3: Synthesis of allyl 4-(3-(4-(4, 7, 10, 13, 16, 19,22-heptaoxaoctacos-1-yn-28-yloxy)phenyl)-6-iminopyridazin-1 (6H)-yl) butanoate, 32

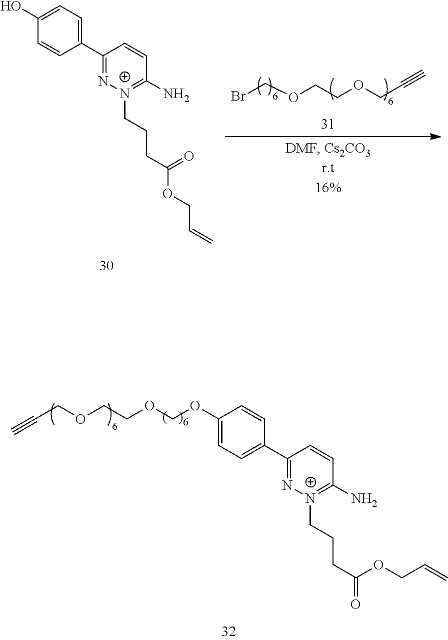

To allyl 4-(3-(4-hydroxyphenyl)-6-iminopyridazin-1 (6H)-yl) butanoate, 30 (1.1 g, 3.51 mmol) was added anhydrous DMF (35.1 mL) after which 28-bromo-4, 7, 10, 13, 16, 19,22-heptaoxaoctacos-1-yne, 31 (2 g, 4.13 mmol) was added. The solution was cooled to 0° C. and anhydrous cesium carbonate (3.43 g, 10.53 mmol) was added and the reaction was stirred for 3 h as the ice bath expired after which water (20 mL) and ethyl acetate (60 mL) were added. The organic layer was washed with water (10 mL), brine (2×10 mL), dried (sodium sulfate), filtered and concentrated to give crude material as a yellow oil which was purified by reverse phase prep HPLC in several injections eluting with 5 to 50% acetonitrile in water (0.1% formic acid conditions and other conditions as described in the general chemical synthesis section) to give allyl 4-(3-(4-(4, 7, 10, 13, 16, 19,22-heptaoxaoctacos-1-yn-28-yloxy)phenyl)-6-iminopyridazin-1 (6H)-yl) butanoate, 32 (412 mg, 0.576 mmol, 16.39% yield) as a red/yellow oil. ¹H NMR (500 MHz, CD₃OD) δ 8.39-8.24 (m, 3H), 8.03-7.85 (m, 2H), 7.61 (d, J=9.6 Hz, 1H), 7.18-6.99 (m, 2H), 5.86 (ddt, J=17.3, 10.5, 5.7 Hz, 1H), 5.34-5.11 (m, 2H), 4.57-4.38 (m, 4H), 4.25-4.02 (m, 4H), 3.75-3.44 (m, 24H), 2.87 (t, J=2.4 Hz, 1H), 2.64 (t, J=6.7 Hz, 2H), 2.29 (p, J=6.8 Hz, 2H), 1.92-1.78 (m, 2H), 1.71-1.42 (m, 6H). (ES-LCMS) m/z 716.5 (M+H)⁺.

### Step 4: Synthesis of allyl 4-(3-(4-((1-(1-(1-(4-((2-(4-(3-chloropropyl)-2-methoxyphenethoxy)ethyl) carbamoyl)phenyl)-3-oxo-6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111-hexatriacontaoxa-2-azatridecahectan-113-yl)-1H-1,2,3-triazol-4-yl)-2, 5, 8, 11, 14, 17,20-heptaoxahexacosan-26-yl)oxy)phenyl)-6-iminopyridazin-1 (6H)-yl) butanoate, 33

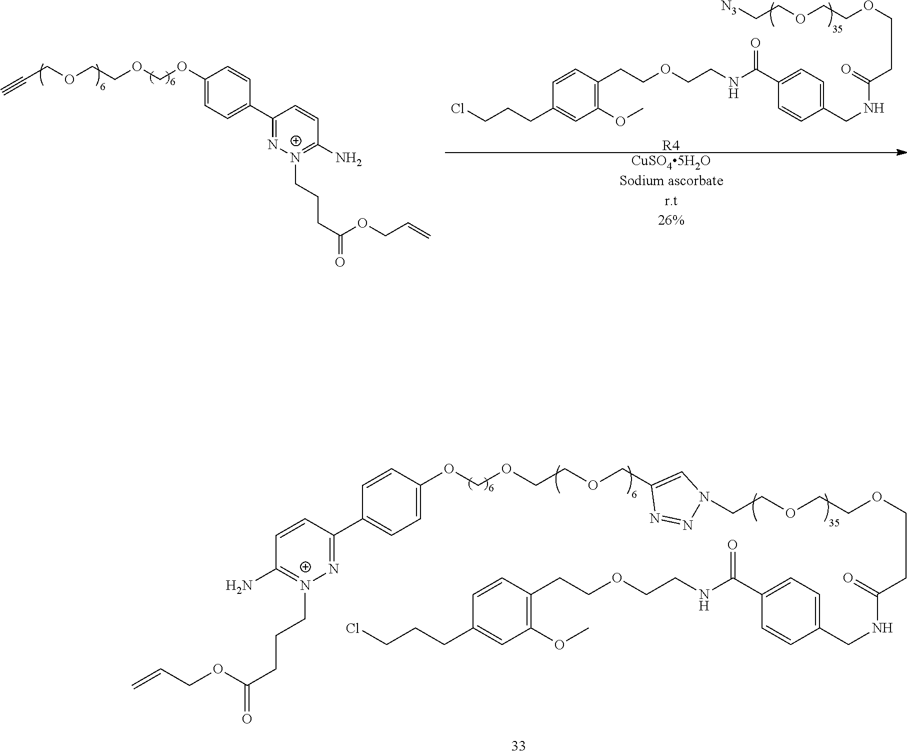

To a vial was added allyl 4-(3-(4-((4, 7, 10, 13, 16, 19,22-heptaoxaoctacos-1-yn-28-yl)oxy)phenyl)-6-iminopyridazin-1 (6H)-yl) butanoate, 32 (40 mg, 56 μmol) and EtOH/t-BuOH/water (2:1:1, 1.6 mL) after which azido-PEG36-HTL2.0, R4 (0.12 g, 56 μmol) was added. Finally, Cu(II) sulfate pentahydrate (0.7 mg, 2.8 μmol) and sodium ascorbate (0.98 mg, 5.6 μmol) were added and the reaction mixture was stirred at room temperature for 3 h after which it was purified by reverse phase prep HPLC eluting with 50 to 80% acetonitrile in water (0.1% formic acid, 220 nm wavelength for collection, and other conditions reported in the general chemical synthesis section) to give allyl 4-(3-(4-((1-(1-(1-(4-((2-(4-(3-chloropropyl)-2-methoxyphenethoxy)ethyl) carbamoyl)phenyl)-3-oxo-6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111-hexatriacontaoxa-2-azatridecahectan-113-yl)-1H-1,2,3-triazol-4-yl)-2, 5, 8, 11, 14, 17,20-heptaoxahexacosan-26-yl)oxy)phenyl)-6-iminopyridazin-1 (6H)-yl) butanoate, 33 (45 mg, 26%) as a yellow oil. ¹H NMR (500 MHZ, CDCl₃) δ 8.44 (s, 2H), 8.17 (d, J=9.5 Hz, 1H), 7.83 (d, J=9.5 Hz, 1H), 7.76-7.65 (m, 5H), 7.33 (d, J=7.9 Hz, 2H), 7.05 (d, J=7.8 Hz, 1H), 7.02-6.94 (m, 2H), 6.66 (d, J=6.5 Hz, 2H), 6.53 (s, 1H), 5.91-5.77 (m, 1H), 5.42-5.11 (m, 2H), 4.65 (s, 2H), 4.58 (d, J=5.9 Hz, 2H), 4.51 (t, J=5.2 Hz, 2H), 4.48 (d, J=6.0 Hz, 2H), 4.42 (t, J=7.8 Hz, 2H), 3.99 (d, J=6.4 Hz, 1H), 3.85 (t, J=5.1 Hz, 2H), 3.78 (s, 3H), 3.75 (t, J=5.6 Hz, 2H), 3.69-3.42 (m, 172H), 2.86 (t, J=7.0 Hz, 2H), 2.71 (t, J=7.4 Hz, 2H), 2.54 (dt, J=15.9, 6.0 Hz, 4H), 2.23 (p, J=6.8 Hz, 2H), 2.03 (dq, J=8.3, 6.5 Hz, 3H), 1.79 (q, J=6.8 Hz, 2H), 1.60 (p, J=6.9 Hz, 2H), 1.53-1.30 (m, 3H). (ES-LCMS) m/z 571.2 [(M+5H)]⁵⁺.

### Step 5: Synthesis of Gabazine.1^{DART.2} (R7)

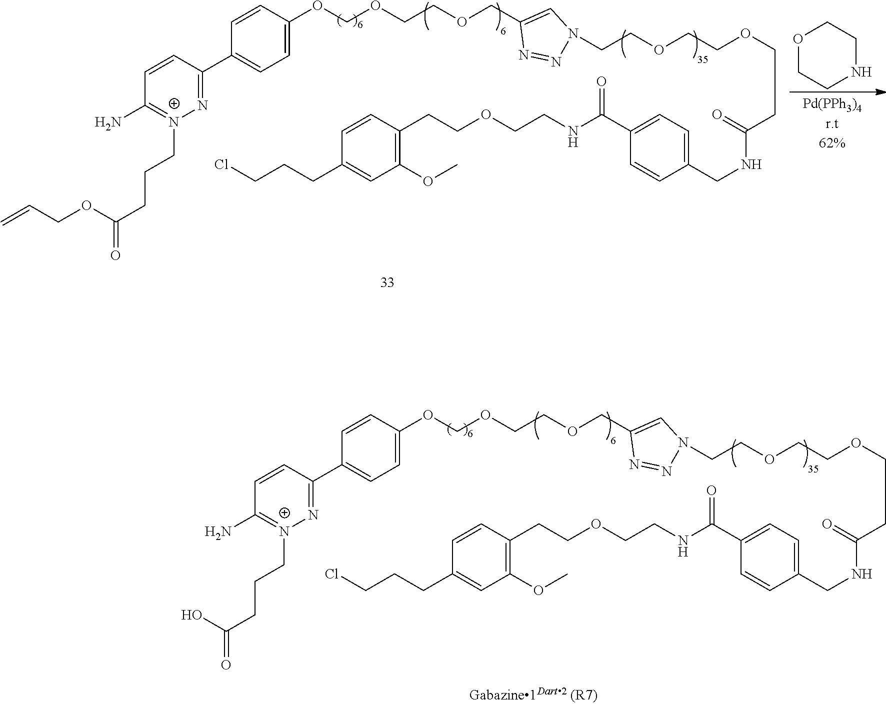

To a solution of allyl 4-(3-(4-((1-(1-(1-(4-((2-(4-(3-chloropropyl)-2-methoxyphenethoxy)ethyl) carbamoyl)phenyl)-3-oxo-6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111-hexatriacontaoxa-2-azatridecahectan-113-yl)-1H-1,2,3-triazol-4-yl)-2, 5, 8, 11, 14, 17,20-heptaoxahexacosan-26-yl)oxy)phenyl)-6-iminopyridazin-1 (6H)-yl) butanoate, 33 (52 mg, 19 μmol) in THF (0.8 mL) was added morpholine (8.1 mg, 8.1 μL, 93 μmol) and Pd(PPh₃)₄ (3 mg, 11.1 μmol). The reaction was stirred under nitrogen for 1 h after which it was purified by reverse phase prep HPLC eluting with 5 to 80% acetonitrile in water (0.1% formic acid, 220 nm wavelength for collection, and conditions reported in the general chemical synthesis section) to give 4-(3-(4-((1-(1-(1-(4-((2-(4-(3-chloropropyl)-2-methoxyphenethoxy)ethyl) carbamoyl)phenyl)-3-oxo-6, 9, 12, 15, 18,21,24,27, 30, 33, 36, 39, 42, 45, 48, 51, 54, 57, 60, 63, 66, 69, 72, 75, 78, 81, 84, 87, 90, 93, 96, 99, 102, 105, 108, 111-hexatriacontaoxa-2-azatridecahectan-113-yl)-1H-1,2,3-triazol-4-yl)-2, 5, 8, 11, 14, 17,20-heptaoxahexacosan-26-yl)oxy)phenyl)-6-iminopyridazin-1 (6H)-yl) butanoic acid Gabazine.1^{DART.2}, R7 (32 mg, 62%). ¹H NMR (500 MHZ, CDCl₃) δ10.97 (br.s, 1H), 8.55 (d, J=9.7 Hz, 1H), 7.84 (d, J=9.6 Hz, 1H), 7.77-7.72 (m, 2H), 7.71 (s, 1H), 7.69-7.65 (m, 2H), 7.33 (d, J=8.0 Hz, 2H), 7.09 (t, J=5.9 Hz, 1H), 7.05 (d, J=7.9 Hz, 1H), 6.99-6.95 (m, 2H), 6.66-6.62 (m, 2H), 6.51 (s, 1H), 4.65 (s, 2H), 4.51 (t, J=5.5 Hz, 2H), 4.48 (d, J=6.0 Hz, 2H), 4.40 (t, J=7.5 Hz, 2H), 3.99 (t, J=6.5 Hz, 2H), 3.85 (t, J=5.2 Hz, 2H), 3.77 (s, 3H), 3.75 (t, J=5.6 Hz, 2H), 3.70-3.37 (m, 175H), 3.52-3.44 (m, 9H), 2.85 (t, J=7.0 Hz, 2H), 2.71 (t, J=7.4 Hz, 2H), 2.52 (t, J=5.6 Hz, 2H), 2.50-2.41 (m, 2H), 2.12-2.07 (m, 2H), 2.06-2.00 (m, 2H), 1.80 (p, J=6.8 Hz, 2H), 1.65-1.54 (m, 2H), 1.51-1.37 (m, 4H), HRMS (ESI+): m/z calcd for C₁₃₂H₂₂₉ClN₈Na₂O₅₀: 1404.7582 [M+2Na]²⁺; found: 1404.7709 [M+2Na]²⁺

### Example 8

### Experimental for Gabazine.5^{DART.2} (R8)

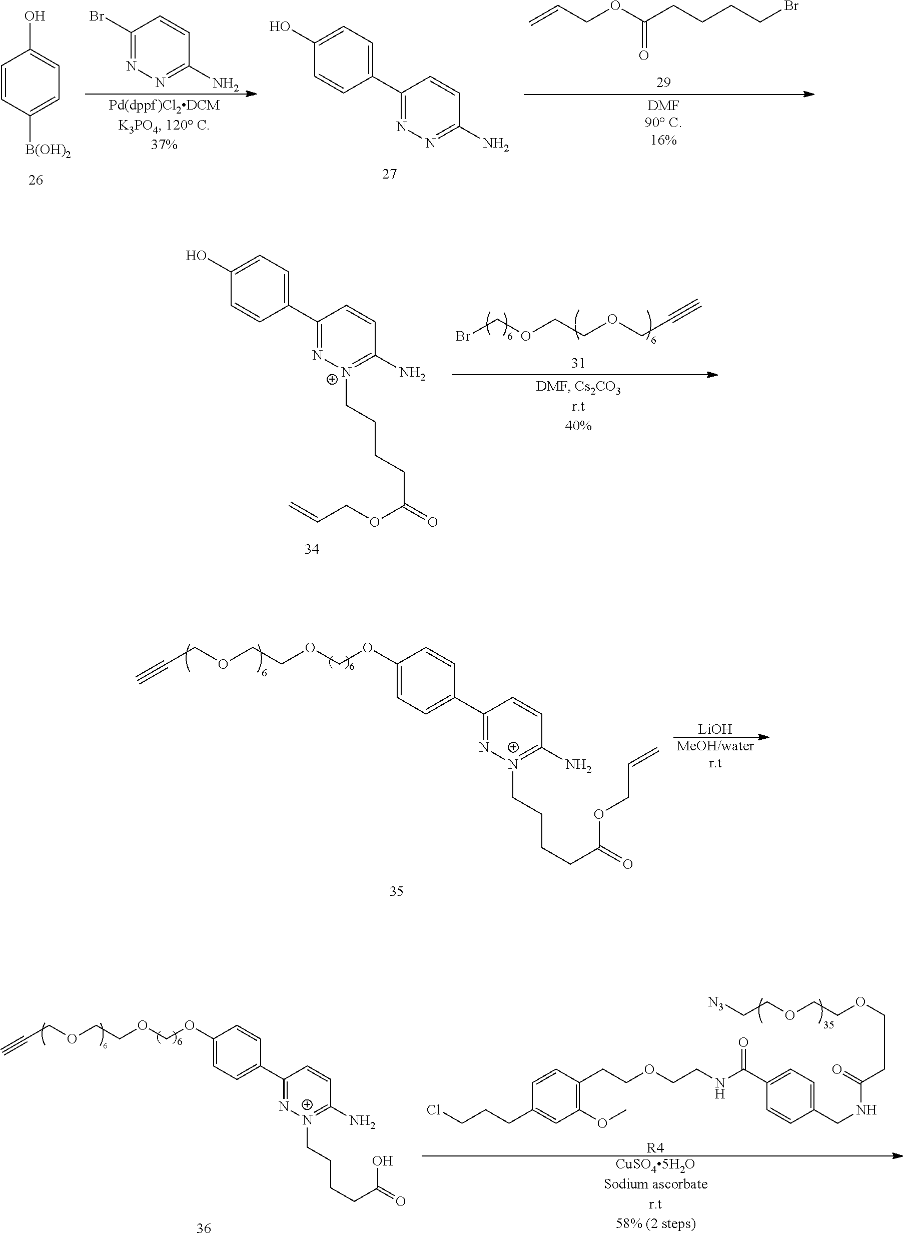

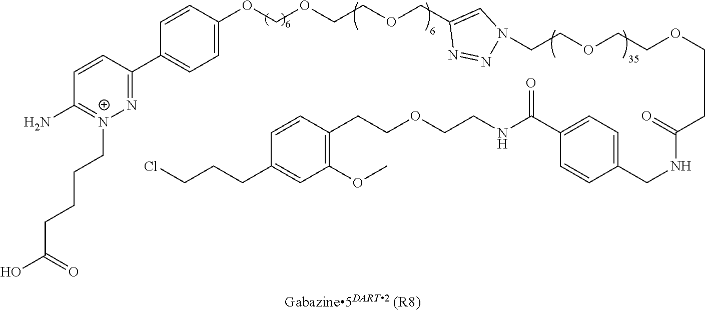

### Step 2: Synthesis of allyl 5-(3-(4-hydroxyphenyl)-6-iminopyridazin-1 (6H)-yl) pentanoate, 34

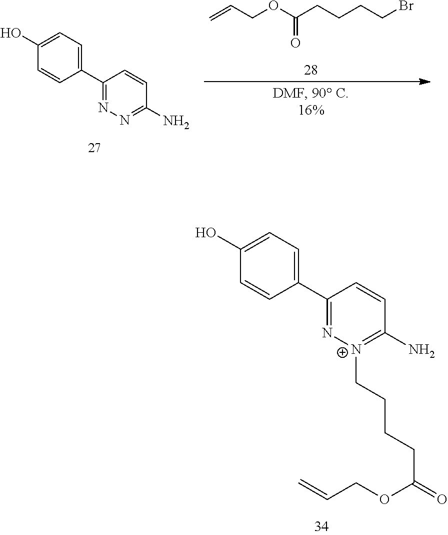 [SMILES: Nc1ccc(-c2ccc(O)cc2)nn1 ; C=CCOC(=O)CCCCBr ; C=CCOC(=O)CCCC[n+]1nc(-c2ccc(O)cc2)ccc1N]

To 4-(6-aminopyridazin-3-yl) phenol, 27 (250 mg, 1.34 mmol) was added allyl 5-bromopentanoate, 29 (295 mg, 1.34 mmol) and DMF (2 mL) and the reaction was heated at 90° C. for 2 h. The mixture was directly purified by prep HPLC eluting with 10 to 90% ACN in water (0.1% FA, 220 nm wavelength for collection) in three 750 uL injections. The main peak(s) were isolated, and fractions were concentrated to give the desired product, 34 (71 mg, 16%). Before this main peak, one byproduct with the same mass was isolated. Since three nitrogens in the product as well as the phenolic oxygen all may react with the alkyl bromide, the assignment and confirmation of product was described in the Gabazine.7^{DART.2} (R9) section. ¹H NMR (500 MHz, CD₃OD) δ 8.47 (br.s, 1H), 8.27 (d, J=9.3 Hz, 1H), 7.87 (d, J=8.4 Hz, 2H), 7.58 (d, J=9.4 Hz, 1H), 6.94 (d, J=8.4 Hz, 2H), 5.97-5.85 (m, 1H), 5.31 (dd, J=17.3, 1.9 Hz, 1H), 5.21 (d, J=10.5 Hz, 1H), 4.59 (d, J=5.6 Hz, 2H), 4.40 (t, J=7.2 Hz, 2H), 2.51 (t, J=7.3 Hz, 2H), 2.05-1.99 (m, 2H), 1.83-1.77 (m, 2H). ¹³C NMR (126 MHZ, CD₃OD) δ173.05, 160.45, 152.26, 150.88, 132.27, 131.05, 128.00, 125.02, 123.82, 116.95, 115.69, 64.77, 55.54, 32.75, 25.86, 21.21. (ES-LCMS) m/z 328.3 (M+H)⁺.

### Step 3: Synthesis of allyl 5-(3-(4-((4, 7, 10, 13, 16, 19,22-heptaoxaoctacos-1-yn-28-yl)oxy)phenyl)-6-iminopyridazin-1 (6H)-yl) pentanoate, 35

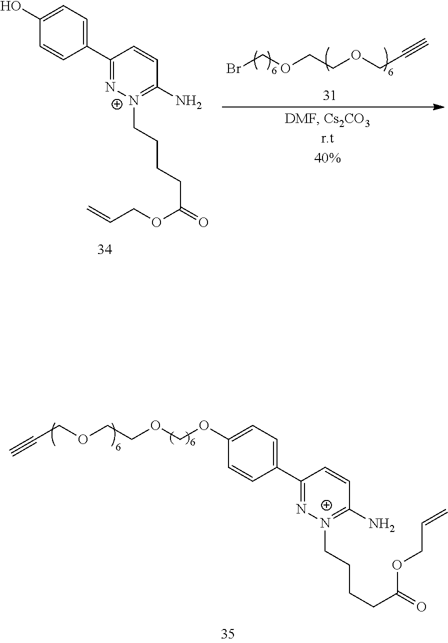

To allyl 5-(3-(4-hydroxyphenyl)-6-iminopyridazin-1 (6H)-yl) pentanoate, 34 (40 mg, 0.12 mmol) was added anhydrous DMF (1 mL) after which 28-bromo-4, 7, 10, 13, 16, 19,22-heptaoxaoctacos-1-yne, 31 (89 mg, 0.18 mmol) and anhydrous cesium carbonate (80 mg, 0.24 mmol) were added and the reaction was stirred for 3 h. DMF was removed under a stream of nitrogen, and the crude was purified by reverse phase prep HPLC eluting with 5 to 50% acetonitrile in water (0.1% formic acid conditions and other conditions as described in the general chemical synthesis section) to give allyl 5-(3-(4-((4,7,10,13,16,19,22-heptaoxaoctacos-1-yn-28-yl)oxy)phenyl)-6-iminopyridazin-1 (6H)-yl) pentanoate, 35 (36 mg, 49 μmol, 40% yield) as a red/yellow oil. ¹H NMR (500 MHZ, CD₃OD) δ 8.53 (s, 1H), 8.30 (d, J=9.6 Hz, 1H), 8.02-7.90 (m, 2H), 7.60 (d, J=9.5 Hz, 1H), 7.16-7.03 (m, 2H), 5.98-5.90 (m, 1H), 5.31 (dq, J=17.2, 1.6 Hz, 1H), 5.21 (dq, J=10.5, 1.4 Hz, 1H), 4.59 (dt, J=5.7, 1.5 Hz, 2H), 4.41 (t, J=7.2 Hz, 2H), 4.20 (d, J=2.4 Hz, 2H), 4.09 (t, J=6.4 Hz, 2H), 3.69-3.63 (m, 21H), 3.61-3.59 (m, 2H), 3.52 (t, J=6.5 Hz, 2H), 2.86 (t, J=2.4 Hz, 1H), 2.51 (t, J=7.3 Hz, 2H), 2.06-2.00 (m, 2H), 1.87-1.77 (m, 4H), 1.67-1.61 (m, 2H), 1.58-1.52 (m, 2H), 1.51-1.45 (m, 2H). (ES-LCMS) m/z 730.6 (M+H)⁺.

### Steps 4/5: Synthesis of Gabazine.5^{DART.2} (R8)

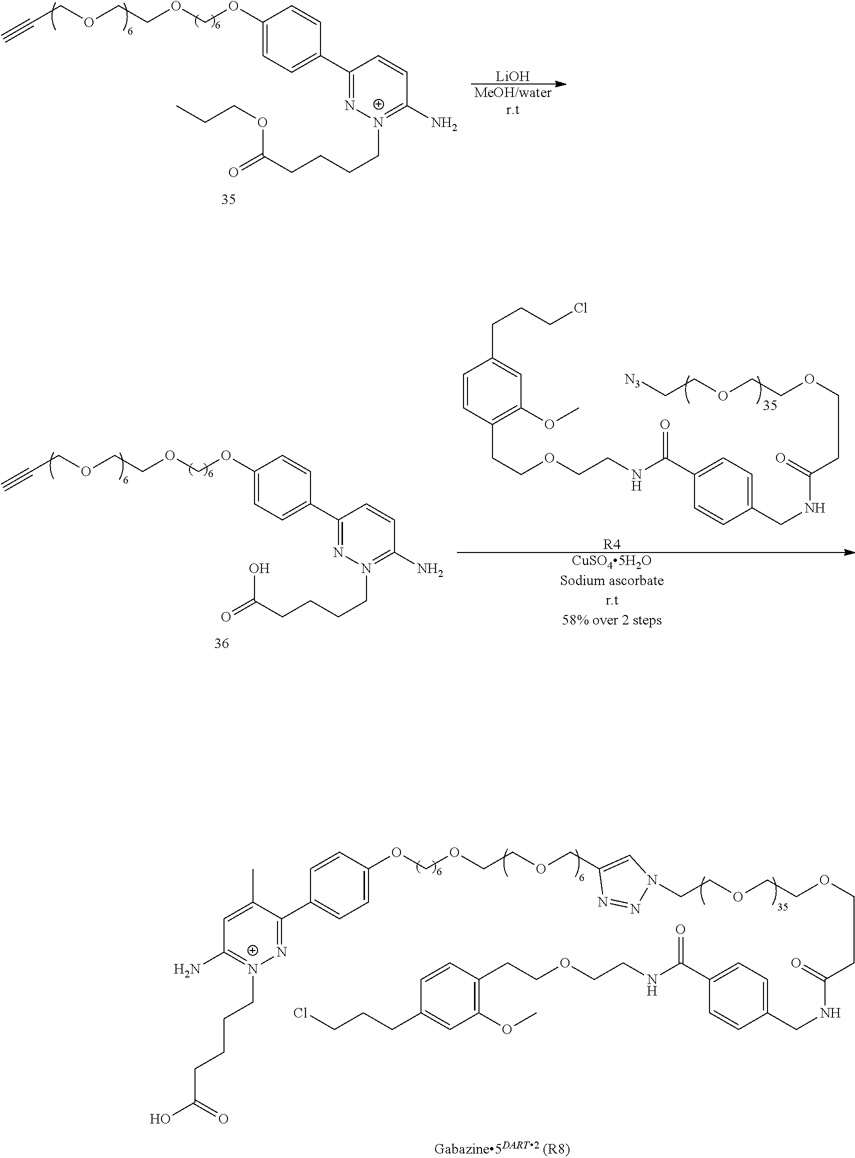

To allyl 5-(3-(4-((4, 7, 10, 13, 16, 19,22-heptaoxaoctacos-1-yn-28-yl)oxy)phenyl)-6-iminopyridazin-1 (6H)-yl) pentanoate, 35 (15 mg, 20 μmol) in MeOH/H2O (1/1, 2 ml) was added lithium hydroxide hydrate (5.1 mg, 120 μmol) at r.t, and the reaction was stirred for 2 h. LCMS indicated formation of 36 and the pH was adjusted to 8 by addition of 1N HCl (110 ul). The mixture was concentrated under a stream of nitrogen. To the crude mixture was added EtOH/t-BuOH/water (2:1:1, 1 mL) after which azido-PEG36-HTL2.0, R4 (42 mg, 20 μmol) was added. Finally, Cu(II) sulfate pentahydrate (0.45 mg, 1.8 μmol) and sodium ascorbate (0.32 mg, 1.8 μmol) were added and the reaction mixture was stirred at room temperature overnight after which it was purified by reverse phase prep HPLC eluting with 20 to 90% acetonitrile in water (0.1% formic acid, 200 nm wavelength for collection, and other conditions reported in the general chemical synthesis section) to give 5-(3-(4-((1-(1-(1-(4-((2-(4-(3-chloropropyl)-2-methoxyphenethoxy)ethyl) carbamoyl)phenyl)-3-oxo 6, 9, 12, 15, 18,21,24,27, 30, 33, 36, 39, 42, 45, 48, 51, 54, 57, 60, 63, 66, 69, 72, 75, 78, 81, 84, 87, 90, 93, 96, 99, 102, 105, 108, 111-hexatriacontaoxa-2-azatridecahectan-113-yl)-1H-1,2,3-triazol-4-yl)-2, 5, 8, 11, 14, 17,20-heptaoxahexacosan-26-yl)oxy)phenyl)-6-iminopyridazin-1 (6H)-yl) pentanoic acid, Gabazine.5^{DART.2}, R8 (32.0 mg, 2 steps 58%) ¹H NMR (500 MHZ, D₂O, water suppression used—the peaks close to the water peak between 4.4 and 4.6 ppm were partially suppressed) δ 7.89 (s, 1H), 7.87 (d, J=9.6 Hz, 1H), 7.62 (d, J=8.4 Hz, 2H), 7.47 (d, J=8.0 Hz, 2H), 7.33 (d, J=9.4 Hz, 1H), 7.17 (d, J=8.1 Hz, 2H), 6.79 (d, J=8.5 Hz, 2H), 6.75 (d, J=7.4 Hz, 1H), 6.46 (s, 1H), 6.31 (d, J=7.6 Hz, 1H), 4.47 (s, 2H), 4.42 (t, J=5.0 Hz, 2H), 4.23 (s, 2H), 4.07 (t, J=7.4 Hz, 2H), 3.76 (t, J=5.1 Hz, 4H), 3.70-3.34 (m, 170H), 3.29-3.23 (m, 5H), 2.55 (t, J=6.6 Hz, 2H), 2.40 (t, J=6.0 Hz, 2H), 2.34 (t, J=7.5 Hz, 2H), 2.12 (t, J=7.3 Hz, 2H), 1.72-1.65 (m, 4H), 1.53-1.43 (m, 4H), 1.38-1.32 (m, 2H), 1.22-1.10 (m, 4H). HRMS (ESI+): m/z calcd for C₁₃₃H₂₃₂ClN₈NaO₅₀: 1400.2733 [M+H+Na]²⁺; found: 1400.2734 [M+H+Na]²⁺.

### Example 9

### Experimental for Gabazine.7^{DART.2} (R9)

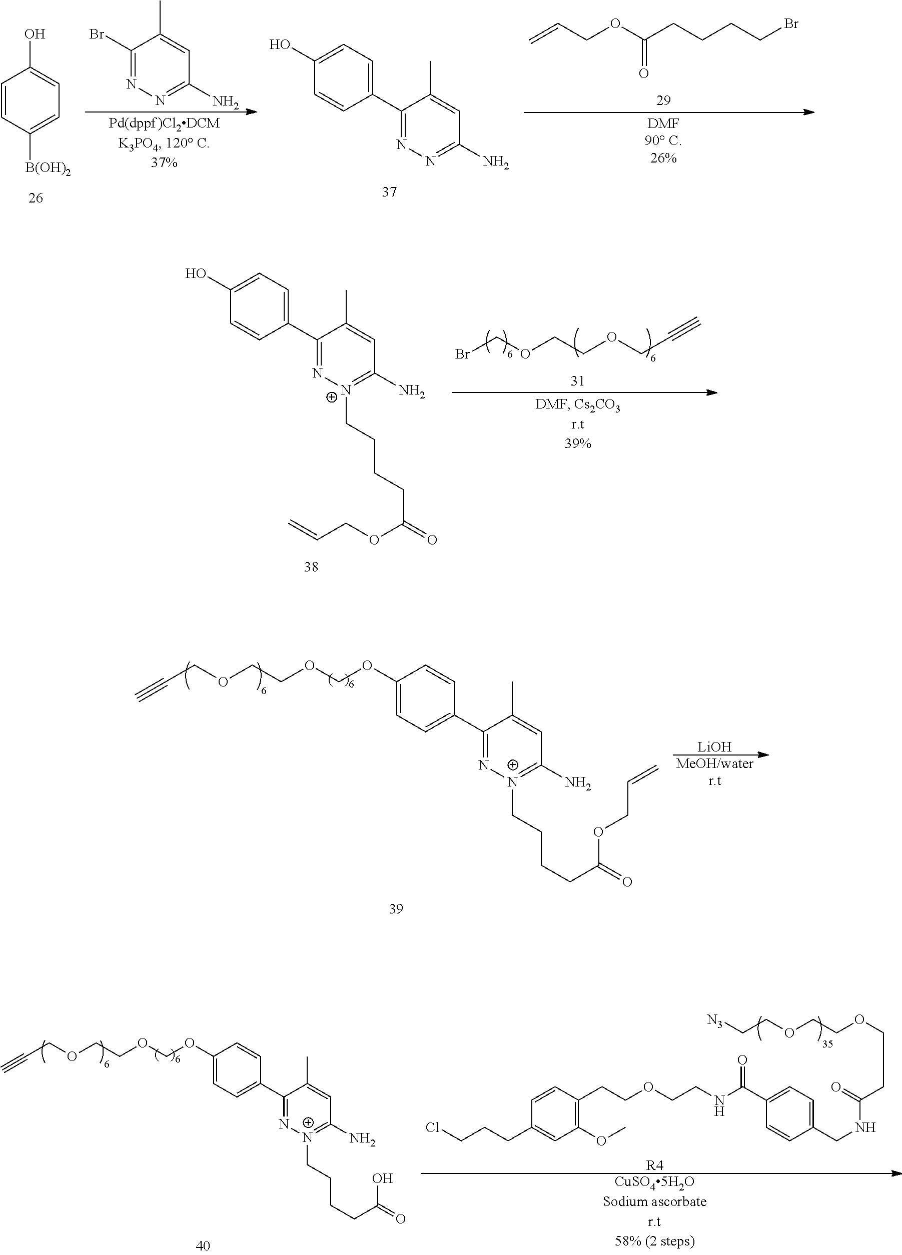

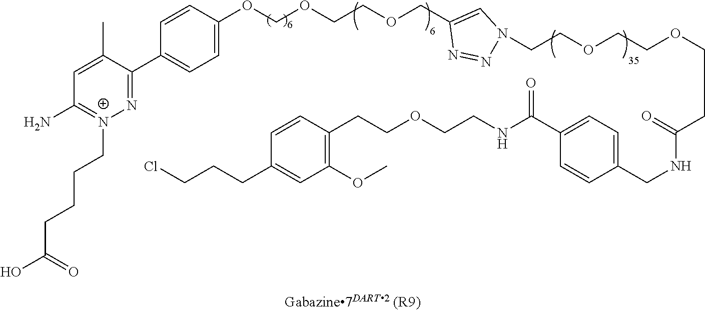

### Step 1: Synthesis of 4-(6-amino-4-methylpyridazin-3-yl) phenol, 37

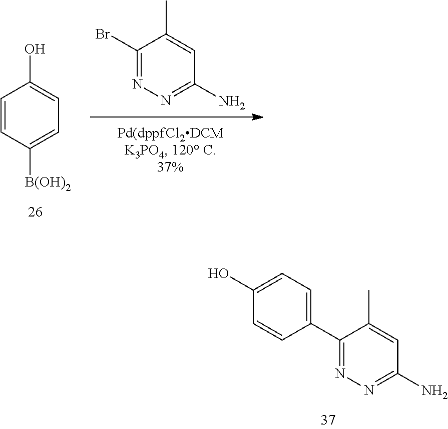

To (4-hydroxyphenyl) boronic acid, 26 (1 g, 7.25 mmol) was added K₃PO₄ (3.08 g, 14.5 mmol), Pd(dppf)Cl₂·DCM (0.355 g, 0.435 mmol), 6-bromo-5-methylpyridazin-3-amine (0.937 g, 6.53 mmol) and dioxane (7 mL)/water (4 mL). The suspension was stirred and degassed for 5 min after which it was heated at 120 degrees for 17 min, then concentrated. The material was then taken up in water and a slight amount of DCM, stirred thoroughly, then filtered. The mixture was filtered to give 4-(6-amino-4-methylpyridazin-3-yl) phenol, 37 (1.132 g, 5.62 mmol, 77% yield) as a dark, brown solid, containing some aryl halide. Used directly without further purification. ¹H NMR (500 MHZ, CD₃OD) δ 7.30 (d, J=8.6 Hz, 2H), 6.89 (d, J=8.6 Hz, 2H), 6.86 (d, J=1.1 Hz, 1H), 2.23 (d, J=1.0 Hz, 3H). (ES-LCMS) m/z 202.2 (M+H)⁺

### Step 2: Synthesis of allyl 5-(3-(4-hydroxyphenyl)-6-imino-4-methylpyridazin-1 (6H)-yl) pentanoate, 38

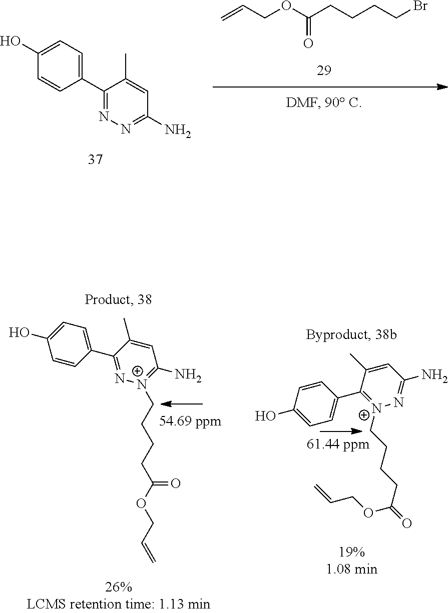 [SMILES: Cc1cc(N)nnc1-c1ccc(O)cc1 ; C=CCOC(=O)CCCCBr ; C=CCOC(=O)CCCC[n+]1nc(-c2ccc(O)cc2)c(C)cc1N ; C=CCOC(=O)CCCC[n+]1nc(N)cc(C)c1-c1ccc(O)cc1]

To 4-(6-amino-4-methylpyridazin-3-yl) phenol, 37 (250 mg, 1.24 mmol) was added allyl allyl 5-bromopentanoate, 29 (275 mg, 1.24 mmol) and DMF (2 mL) and the reaction was heated at 90° C. for 2 h. The mixture was directly purified by prep HPLC eluting with 10 to 90% ACN in water (0.1% FA, 220 nm wavelength for collection, and the rest of the conditions as described in the general chemistry synthesis section) in three 750 uL injections. Relevant fractions were combined and concentrated. The undesired byproduct eluted first and the desired product eluted later. Since three nitrogens and the phenolic oxygen may all react with the alkyl bromide, the structures were fully assigned and confirmed by H/C NMR, HSQC, and HMBC in DMSO-d6. The most distinguishing peak for the product and byproduct is the CH₂ that is directly connected to the nitrogen which reacted. The ¹³C NMR of the CH₂ immediately adjacent to the nucleophilic nitrogen has a shift of 54.69 ppm in the desired product vs 61.44 ppm in the undesired byproduct. The ¹³C NMR shift of this CH₂ in the desired compound is the same as the shift of a CH₂ adjacent to the same nucleophilic nitrogen in a similar compound reported in reference 4 in which the structure was confirmed by X-ray crystallography. The regioselectivity of the alkylation of pyridazine has been documented previously. The reactivities of the structurally related analogs, Gabazine 1, Gabazine 5, and Gabazine 7, are very similar: in all cases, the undesired byproduct eluted first on RP HPLC. Additionally, the shifts of the CH₂ directly connected to the same nitrogen are very similar for all 3 analogs. It worthwhile to mention that the products and byproducts exist as tautomers under acidic and basic conditions. Furthermore, the undesired byproduct does not react in the following alkylation step (the reaction with the alkyl bromide in DMF with cesium carbonate) while the desired product does react, further confirming structural assignments. Desired product 38 yield: 108 mg (26%). Byproduct 38b yield: 80 mg (19%). The NMR shifts of each compound in both CD₃OD and DMSO-d₆ are reported below.

Desired Product: ¹H NMR (500 MHZ, CD₃OD) δ 8.35 (br.s, 1H), 7.43-7.39 (m, 3H), 6.93 (d, J=8.6 Hz, 2H), 5.97-5.89 (m, 1H), 5.30 (dq, J=17.2, 1.7 Hz, 1H), 5.21 (dt, J=10.5, 1.5 Hz, 1H), 4.58 (dt, J=5.6, 1.5 Hz, 2H), 4.36 (t, J=7.2 Hz, 2H), 2.48 (t, J=7.3 Hz, 2H), 2.42 (s, 3H), 2.01-1.95 (m, 2H), 1.79-1.73 (m, 2H). ¹³C NMR (126 MHZ, CD₃OD) δ 173.06, 159.15, 154.36, 152.42, 145.60, 132.27, 130.21, 124.56, 123.28, 116.95, 115.04, 64.77, 55.03, 32.73, 25.92, 21.18, 18.99. ¹H NMR (500 MHZ, DMSO-d₆) δ 8.48 (s, 1H), 7.50 (s, 1H), 7.39 (d, J=8.3 Hz, 2H), 6.91 (d, J=8.4 Hz, 2H), 5.95-5.87 (m, 1H), 5.29 (dt, J=17.2, 1.7 Hz, 1H), 5.21 (dd, J=10.5, 1.6 Hz, 1H), 4.55 (dd, J=4.4, 2.8 Hz, 2H), 4.29 (t, J=7.2 Hz, 2H), 2.43 (t, J=7.4 Hz, 2H), 2.32 (s, 3H), 1.85-1.79 (m, 2H), 1.67-1.60 (m, 2H). ¹³C NMR (126 MHZ, DMSO-d₆)· 172.67, 159.57, 152.94, 152.48, 144.20, 133.10, 130.75, 124.41, 124.34, 118.06, 115.70, 64.67, 54.69, 33.18, 26.49, 21.52, 20.13. (ES-LCMS) m/z 342.3 (M+H)⁺.

Byproduct: ¹H NMR (500 MHZ, CD₃OD) δ 8.32 (br.s, 1H), 7.41 (s, 1H), 7.36-7.25 (m, 2H), 7.12-6.98 (m, 2H), 5.98-5.90 (m, 1H), 5.31 (dt, J=17.3, 1.7 Hz, 1H), 5.23 (dd, J=10.4, 1.6 Hz, 1H), 4.57 (dd, J=5.7, 1.6 Hz, 2H), 4.27 (t, J=7.4 Hz, 2H), 2.31 (t, J=7.2 Hz, 2H), 2.13 (s, 3H), 1.99-1.92 (m, 2H), 1.59-1.53 (m, 2H). ¹³C NMR (126 MHZ, CD₃OD) δ 172.80, 160.60, 160.04, 151.83, 146.60, 132.27, 129.52, 122.20, 119.60, 116.97, 116.25, 64.77, 61.52, 32.48, 28.54, 21.11, 18.70. ¹H NMR (500 MHZ, DMSO-d₆) δ 8.53 (br.s, 1H), 7.85 (s, 2H), 7.49 (s, 1H), 7.38 (d, J=8.4 Hz, 2H), 7.06 (d, J=8.3 Hz, 2H), 5.99-5.92 (m, 1H), 5.34 (dq, J=17.2, 1.7 Hz, 1H), 5.27 (dt, J=10.4, 1.5 Hz, 1H), 4.58 (dt, J=5.5, 1.5 Hz, 2H), 4.19 (t, J=7.4 Hz, 2H), 2.31 (t, J=7.3 Hz, 2H), 2.08 (s, 3H), 1.90-1.84 (m, 2H), 1.52-1.46 (m, 2H). ¹³C NMR (126 MHz, DMSO-d₆) δ 172.41, 160.41, 160.35, 151.86, 146.48, 133.06, 130.10, 122.68, 119.39, 118.17, 116.68, 64.73, 61.44, 32.91, 28.64, 21.46, 19.97. (ES-LCMS) m/z 342.3 (M+H)⁺.

### Example 10

### Intermediates for Gabazine.1 (30), Gabazine.5 (34), and Gabazine.7 (38)

### Step 3: allyl 5-(3-(4-((4, 7, 10, 13, 16, 19,22-heptaoxaoctacos-1-yn-28-yl)oxy)phenyl)-6-imino-4-methylpyridazin-1 (6H)-yl) pentanoate, 39

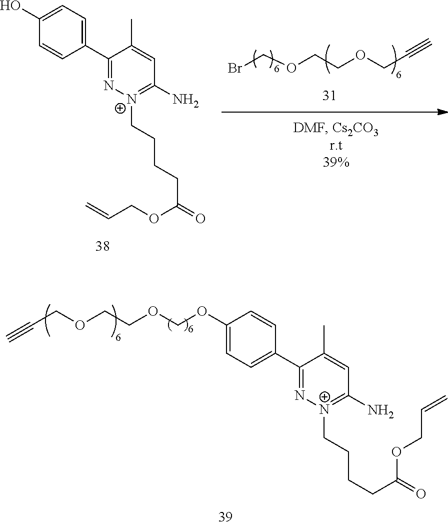

To allyl 5-(3-(4-hydroxyphenyl)-6-imino-4-methylpyridazin-1 (6H)-yl) pentanoate, 38 (40 mg, 0.12 mmol) was added anhydrous DMF (1 mL) after which 28-bromo-4, 7, 10, 13, 16,19,22-heptaoxaoctacos-1-yne, 31 (85 mg, 0.18 mmol) and anhydrous cesium carbonate (76 mg, 0.23 mmol) were added and the reaction was stirred for 3 h. DMF was removed under a stream of nitrogen, and the crude was purified by reverse phase prep HPLC eluting with 5 to 50% acetonitrile in water (0.1% formic acid conditions and other conditions as described in the general chemical synthesis section) to give allyl 5-(3-(4-((4, 7, 10, 13, 16, 19,22-heptaoxaoctacos-1-yn-28-yl)oxy)phenyl)-6-imino-4-methylpyridazin-1 (6H)-yl) pentanoate, 39 (34 mg, 46 μmol, 39% yield) as a red/yellow oil. ¹H NMR (500 MHZ, CD₃OD) δ 8.49 (s, 1H), 7.57-7.45 (m, 2H), 7.39 (d, J=1.3 Hz, 1H), 7.09-7.06 (m, 2H), 5.97-5.89 (m, 1H), 5.30 (dq, J=17.2, 1.6 Hz, 1H), 5.21 (dq, J=10.4, 1.4 Hz, 1H), 4.59 (dt, J=5.7, 1.5 Hz, 2H), 4.36 (t, J=7.2 Hz, 2H), 4.20 (d, J=2.4 Hz, 2H), 4.08 (t, J=6.4 Hz, 2H), 3.70-3.64 (m, 21H), 3.61-3.59 (m, 2H), 3.52 (t, J=6.5 Hz, 2H), 2.86 (t, J=2.4 Hz, 1H), 2.48 (t, J=7.3 Hz, 2H), 2.42 (s, 3H), 2.01-1.95 (m, 2H), 1.88-1.82 (m, 2H), 1.79-1.73 (m, 2H), 1.67-1.62 (m, 2H), 1.59-1.53 (m, 2H), 1.51-1.45 (m, 2H). ES-LCMS) m/z 744.6 (M+H)⁺.

### Steps 4/5: Synthesis of Gabazine.7^{DART.2} (R9)

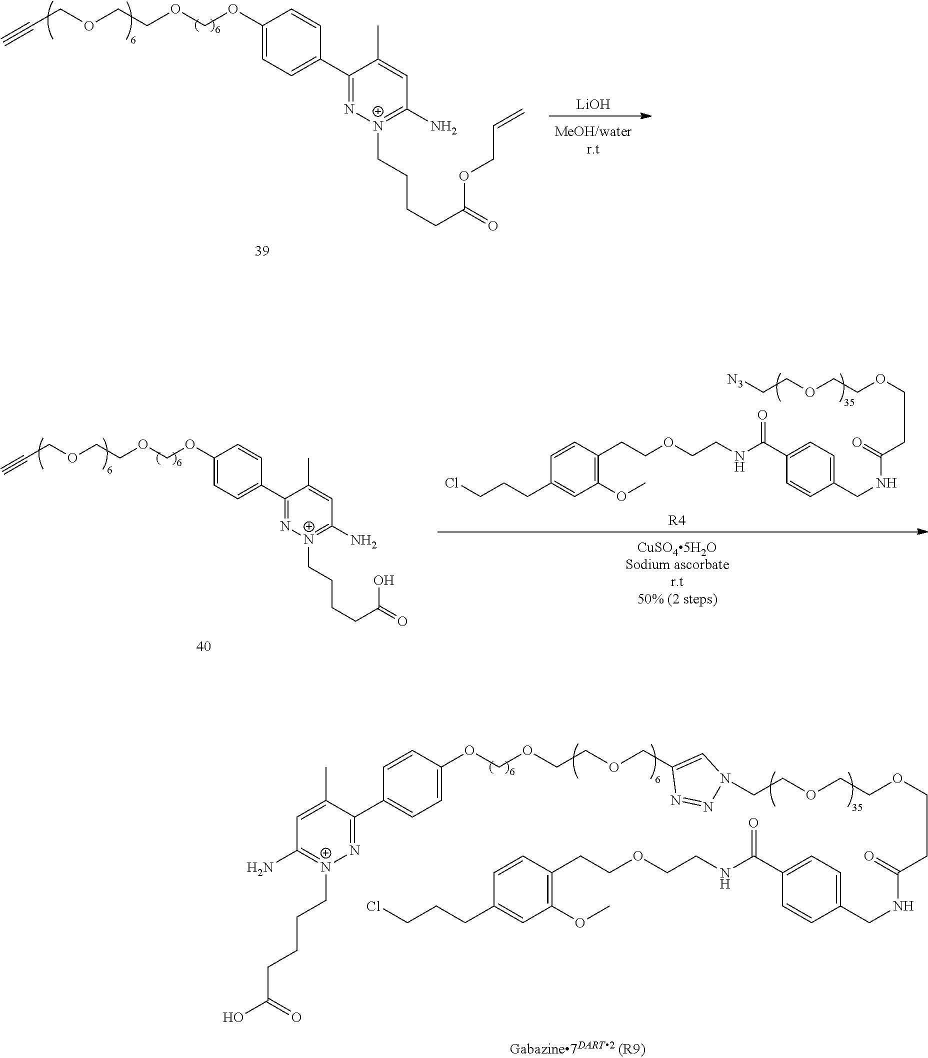

To allyl 5-(3-(4-((4, 7, 10, 13, 16, 19,22-heptaoxaoctacos-1-yn-28-yl)oxy)phenyl)-6-imino-4-methylpyridazin-1 (6H)-yl) pentanoate, 39 (15 mg, 20.0 μmol) in MeOH/water (1:1, 2 ml) was added lithium hydroxide hydrate (5.1 mg, 120 μmol) at room temperature and the reaction was stirred for 2 h after which the pH was adjusted to 8 by addition of 1N HCl (110 μL) and solvent was removed under a stream of nitrogen. The crude material 40 was taken up in EtOH/t-BuOH/water (2:1:1, 1 mL) after which azido-PEG36-HTL2.0, R4 (38 mg, 18 μmol) was added. Finally, Cu(II) sulfate pentahydrate (0.45 mg, 1.8 μmol) and sodium ascorbate (0.32 mg, 1.8 μmol) were added and the reaction mixture was stirred at room temperature overnight after which it was purified by reverse phase prep HPLC eluting with 20 to 90% acetonitrile in water (0.1% formic acid, 200 nm wavelength for collection, and other conditions reported in the general chemical synthesis section) to give 5-(3-(4-((1-(1-(1-(4-((2-(4-(3-chloropropyl)-2-methoxyphenethoxy)ethyl) carbamoyl)phenyl)-3-oxo-6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111-hexatriacontaoxa-2-azatridecahectan-113-yl)-1H-1,2,3-triazol-4-yl)-2, 5, 8, 11, 14, 17,20-heptaoxahexacosan-26-yl)oxy)phenyl)-6-imino-4-methylpyridazin-1 (6H)-yl) pentanoic acid, Gabazine.7^{DART.2}, R9 (25.0 mg, 50% over 2 steps) ¹H NMR (500 MHZ, D20, water suppression used—the peaks close to the water peak between 4.4 and 4.6 ppm were partially suppressed) δ 7.91 (s, 1H), 7.49 (d, J=7.9 Hz, 2H), 7.30 (d, J=8.2 Hz, 2H), 7.22 (d, J=8.0 Hz, 2H), 7.19 (s, 1H), 6.89 (d, J=8.3 Hz, 2H), 6.83 (d, J=7.6 Hz, 1H), 6.56 (s, 1H), 6.36 (d, J=7.5 Hz, 1H), 4.49 (s, 2H), 4.44 (t, J=5.2 Hz, 2H), 4.28 (s, 2H), 4.09 (t, J=7.4 Hz, 2H), 3.87 (t, J=6.5 Hz, 2H), 3.78 (t, J=5.1 Hz, 3H), 3.68-3.62 (m, 5H), 3.53-3.43 (m, 183H), 3.38-3.28 (m, 6H), 2.60 (t, J=6.6 Hz, 2H), 2.43-2.37 (m, 4H), 2.12 (s, 3H), 2.07 (t, J=7.5 Hz, 2H), 1.75-1.66 (m, 4H), 1.61-1.56 (m 2H), 1.49-1.38 (dp, J=28.1, 7.4 Hz, 4H), 1.30-1.24 (m, 2H), 1.23-1.17 (m, 2H). HRMS (ESI+): m/z calcd for C₁₃₄H₂₃₄ClN₈NaO₅₀: 1407.2812 [M+H+Na]²⁺; found: 1407.2808 [M+H+Na]²⁺.

### Example 11

### Experimental for Alexa488.1^{DART.2} (R10)

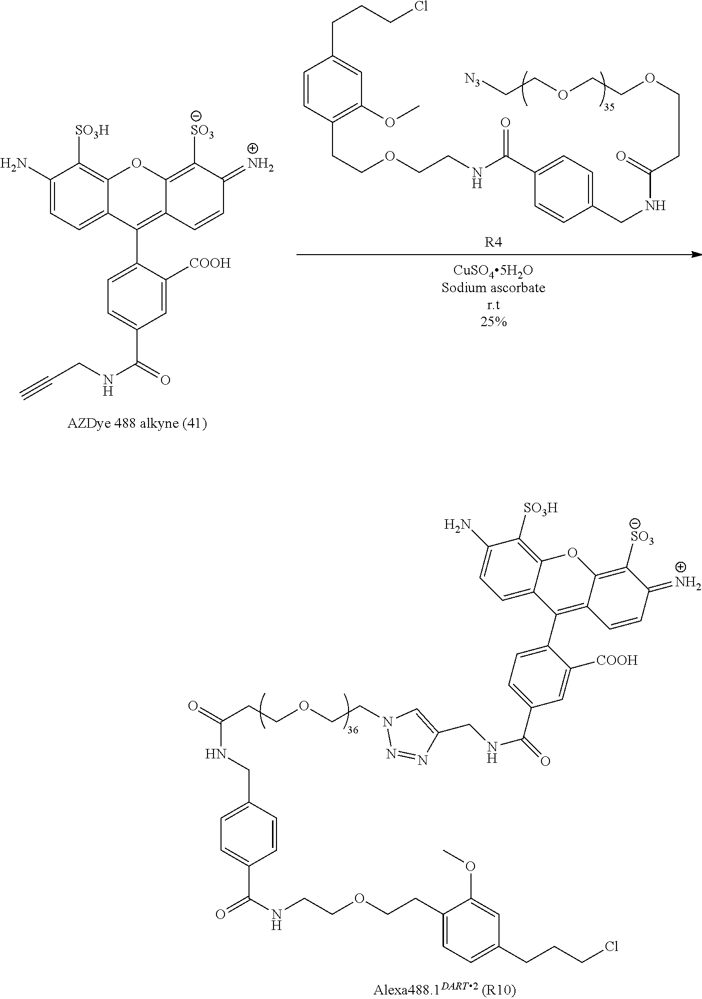

Note: the attachment point of the alkyne in AF488 alkyne, 41 (Click Chemistry Tools; Catalog #1277-25, MW 773.91 g/mol reported, 571.53 g/mol protonated) or the linker length is not directly disclosed by the vendor, however, based on LCMS as well as ¹H NMR shown below, it can reasonably be said that the structure is as drawn (presumably existing as a di-triethylamine salt).

### ¹H NMR of AZDye 488 Alkyne (41) in CD₃OD

To azido-PEG36-HTL2.0, R4 (13 mg, 6.5 μmol) was added AF488 alkyne, 41 (5 mg, 6.5 μmol) followed by EtOH/t-BuOH/water (2:1:1, 0.8 mL). Then, copper (II) sulfate pentahydrate (0.1 mg, 0.325 μmol) and sodium ascorbate (0.1 mg, 0.65 μmol) were added and the reaction was stirred overnight after which it was concentrated under a stream of nitrogen, taken up in acetonitrile/DMSO and purified by 15 to 80% acetonitrile in water (0.1% FA) with a Phenomenex 50×30 mm column, a 12 min gradient, 50 mL/min flow rate, and collection wavelength of 230 nm. Relevant fractions were concentrated to give 6-amino-9-(2-carboxy-4-(((1-(1-(4-((2-(4-(3-chloropropyl)-2-methoxyphenethoxy)ethyl) carbamoyl)phenyl)-3-oxo-6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111-hexatriacontaoxa-2-azatridecahectan-113-yl)-1H-1,2,3-triazol-4-yl)methyl) carbamoyl)phenyl)-3-iminio-5-sulfo-3H-xanthene-4-sulfonate, Alexa488.1^{DART.2} (R10) (5 mg, 29%). ¹H NMR (500 MHZ, CD₃OD) δ 8.47-8.23 (m, 4H), 8.07 (s, 1H), 7.86 (s, 1H), 7.79 (d, J=7.8 Hz, 2H), 7.42 (d, J=8.0 Hz, 2H), 7.15-6.86 (m, 4H), 6.78 (s, 1H), 6.67 (d, J=7.5 Hz, 1H), 4.62 (d, J=61.7 Hz, 3H), 4.48 (d, J=5.4 Hz, 2H), 3.80 (d, J=11.4 Hz, 7H), 3.71-3.49 (m, 149H), 2.86 (t, J=7.0 Hz, 2H), 2.73 (t, J=7.4 Hz, 2H), 2.54 (t, J=6.0 Hz, 2H), 2.05 (p, J=6.9 Hz, 2H). (ES-LCMS) m/z 665.9 (M+4H) 4+; Compound decomposed on HRMS.

### Example 12

### Experimental for Alexa647.1^{DART.2} (R11)

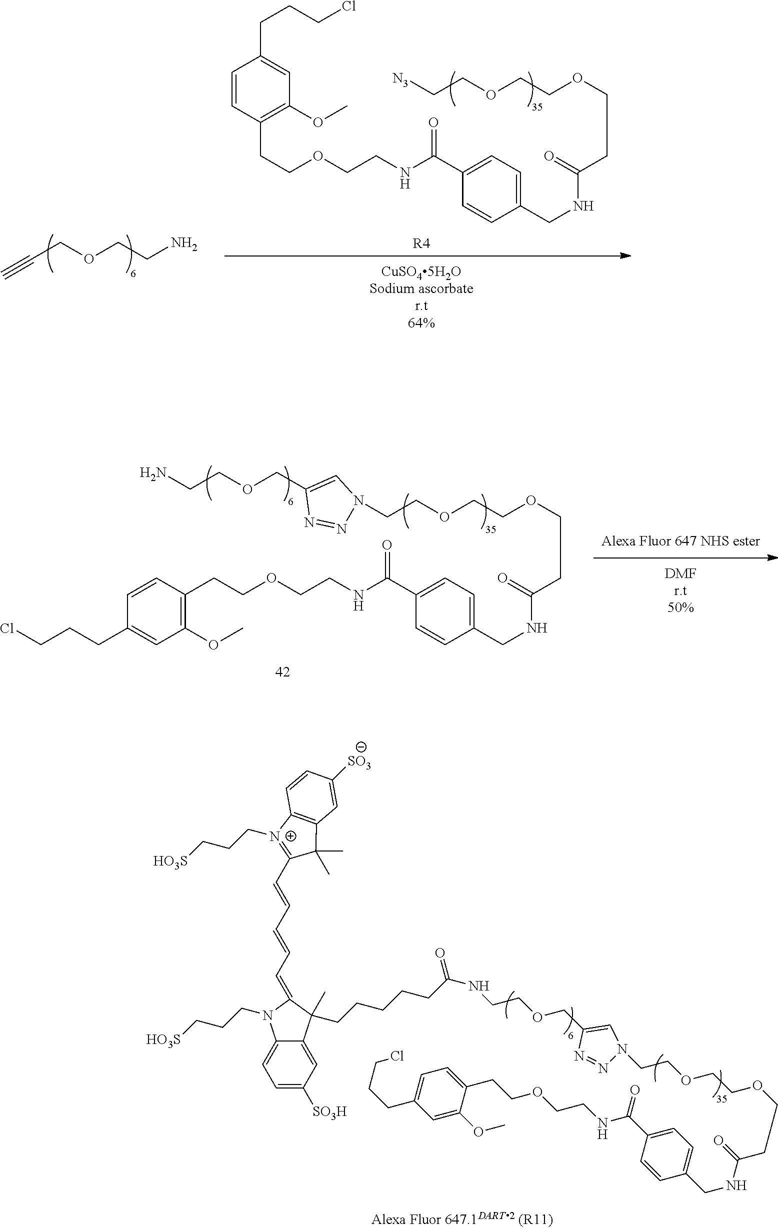

### Step 1: Synthesis of 1-(4-(19-amino-2, 5, 8, 11, 14, 17-hexaoxanonadecyl)-1H-1,2,3-triazol-1-yl)-N-(4-((2-(4-(3-chloropropyl)-2-methoxyphenethoxy)ethyl) carbamoyl)benzyl)-3,6,9,12, 15, 18,21,24,27, 30, 33, 36, 39, 42, 45, 48, 51, 54, 57, 60, 63, 66, 69, 72, 75, 78, 81, 84, 87, 90, 93, 96, 99, 102, 105, 108-hexatriacontaoxaundecahectan-111-amide, 42

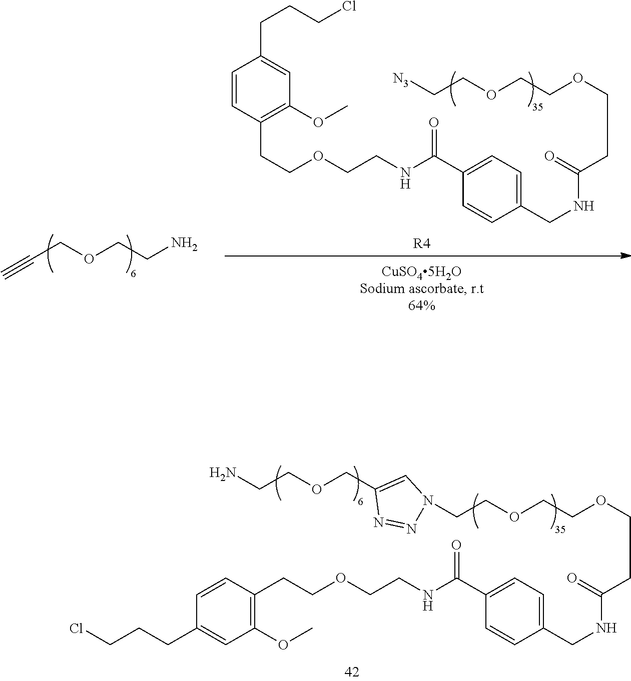

To 3, 6, 9, 12, 15, 18-hexaoxahenicos-20-yn-1-amine (7.6 mg, 24 μmol) was added azido-PEG36-HTL2.0, R4 (50 mg, 24 μmol) after which EtOH (0.4 mL)/t-BuOH (0.2 mL)/H2O (0.2 mL) was added. Then, copper (II) sulfate pentahydrate (0.3 mg, 1.2 μmol) and sodium ascorbate (0.42 mg, 2.4 μmol) were added and the reaction mixture was stirred at room temperature for 2 h after which it was directly purified by reverse phase HPLC eluting with 20 to 90% acetonitrile in water (0.1% formic acid, 200 nm wavelength for collection and conditions as described in the general chemical synthesis section) to give 1-(4-(19-amino-2, 5, 8, 11, 14, 17-hexaoxanonadecyl)-1H-1,2,3-triazol-1-yl)-N-(4-((2-(4-(3-chloropropyl)-2-methoxyphenethoxy)ethyl) carbamoyl)benzyl)-3,6,9,12, 15, 18,21,24,27, 30, 33, 36, 39, 42, 45, 48, 51, 54, 57, 60, 63, 66, 69, 72, 75, 78, 81, 84, 87, 90, 93, 96, 99, 102, 105, 108-hexatriacontaoxaundecahectan-111-amide, 42 (37 mg, 64%) as a light brown oil. ¹H NMR (500 MHZ, D₂O, water suppression used—the peaks close to the water peak between 4.4 and 4.6 ppm were partially suppressed) δ 7.94 (s, 1H), 7.57-7.39 (m, 2H), 7.37-7.18 (m, 2H), 6.94 (d, J=7.6 Hz, 1H), 6.65 (d, J=1.6 Hz, 1H), 6.43 (dd, J=7.6, 1.6 Hz, 1H), 4.53 (s, 2H), 4.51-4.42 (m, 2H), 4.35 (d, J=4.7 Hz, 2H), 3.82 (dd, J=5.6, 4.5 Hz, 2H), 3.72-3.28 (m, 174H), 3.05 (t, J=5.0 Hz, 2H), 2.68 (t, J=6.2 Hz, 2H), 2.45 (td, J=7.6, 6.7, 3.3 Hz, 4H), 1.79 (dq, J=8.5, 6.6 Hz, 2H). (ES-LCMS) m/z 602.9 (M+4H)⁴⁺.

### Step 2: Synthesis of Alexa Fluor-647.1^{DART.2} (R11)

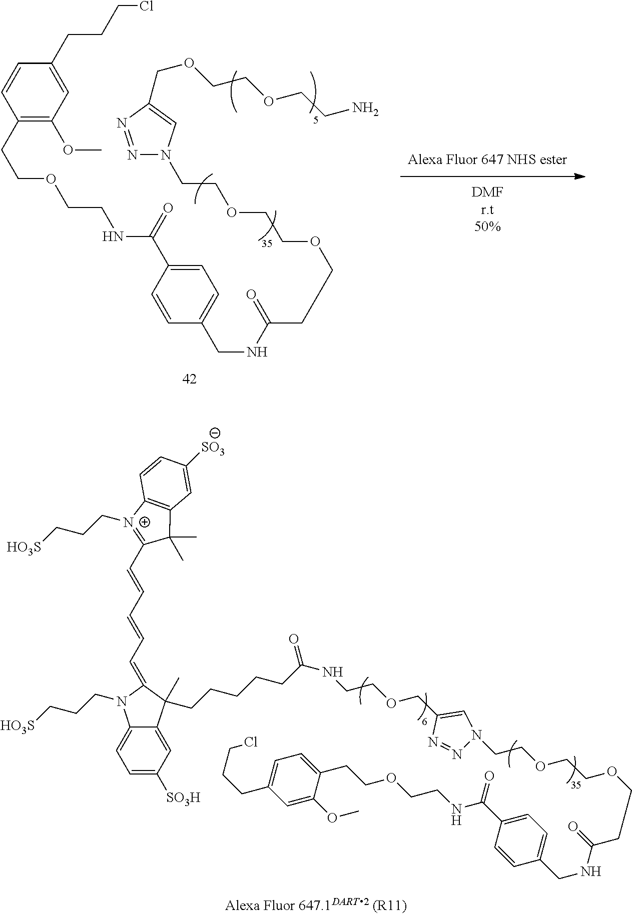

To 1-(4-(19-amino-2, 5, 8, 11, 14, 17-hexaoxanonadecyl)-1H-1,2,3-triazol-1-yl)-N-(4-((2-(4-(3-chloropropyl)-2-methoxyphenethoxy)ethyl) carbamoyl)benzyl)-3,6,9,12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108-hexatriacontaoxaundecahectan-111-amide, 42 (25 mg, 10.4 μmol) was added DMF (1 mL) followed by AlexaFluor 647 NHS ester (20 mg, 20.8 μmol, ThermoFisher Scientific Catalog No. A37566). The mixture was purged with nitrogen, covered in aluminum foil and stirred overnight after which the reaction mixture was purified by reverse phase prep HPLC eluting with 30 to 60% acetonitrile in water (0.1% TFA, 647 nm wavelength for collection and other conditions as described in the general chemical synthesis method) in several injections and concentrated under a stream of nitrogen in the dark to give 2-((1E,3E)-5-(€-3-(1-(1-(1-(4-((2-(4-(3-chloropropyl)-2-methoxyphenethoxy)ethyl) carbamoyl)phenyl)-3-oxo-6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108, 111-hexatriacontaoxa-2-azatridecahectan-113-yl)-1H-1,2,3-triazol-4-yl)-21-oxo-2, 5, 8, 11, 14, 17-hexaoxa-20-azahexacosan-26-yl)-3-methyl-5-sulfo-1-(3-sulfopropyl) indolin-2-ylidene) penta-1,3-dien-1-yl)-3,3-dimethyl-1-(3-sulfopropyl)-3H-114-indole-5-sulfonic acid, Alexa Fluor-647.1^{DART.2} (R11) (17 mg, 50%) as a deep blue oil which was stored in a vial covered with aluminum foil. ¹H NMR (500 MHz, D₂O, water suppression used—the peaks close to the water peak between 4.25 and 4.5 ppm were partially suppressed) δ 8.00 (td, J=13.2, 5.4 Hz, 2H), 7.92 (s, 1H), 7.73-7.66 (m, 4H), 7.49-7.45 (m, 2H), 7.24 (dt, J=8.5, 2.1 Hz, 4H), 6.89 (d, J=7.6 Hz, 1H), 6.61 (d, J=1.6 Hz, 1H), 6.55 (t, J=12.5 Hz, 1H), 6.38 (dd, J=7.6, 1.6 Hz, 1H), 6.30 (d, J=13.5 Hz, 1H), 6.36 (d, J=13.5 Hz, 1H), 4.53 (s, 2H), 4.44 (t, J=5.0 Hz, 2H), 4.31 (s, 2H), 4.09 (q, J=8.1 Hz, 4H), 3.77 (dd, J=5.5, 4.4 Hz, 2H), 3.65 (d, J=5.9 Hz, 2H), 3.59-3.39 (m, 162H), 3.34 (ddt, J=10.4, 6.9, 3.2 Hz, 6H), 3.10 (t, J=5.3 Hz, 2H), 2.87 (dt, J=10.9, 7.1 Hz, 4H), 2.63 (t, J=6.3 Hz, 2H), 2.47-2.38 (m, 4H), 2.18 (t, J=12.3 Hz, 1H), 2.07 (ddt, J=14.2, 10.5, 6.0 Hz, 5H), 1.89 (t, J=7.3 Hz, 2H), 1.75 (dq, J=8.5, 6.5 Hz, 2H), 1.54-1.49 (m, 9H), 1.22 (q, J=7.4 Hz, 2H), 0.95 (p, J=7.5 Hz, 2H), 0.67 (d, J=11.6 Hz, 1H), 0.41 (dd, J=12.3, 6.9 Hz, 1H). Note: the structure of AlexaFluor 647 NHS ester was assumed to be consistent with that reported in reference 7. (ES-LCMS) m/z 813.5 (M+4H) 4+; Compound decomposed on HRMS.

### Example 13

### Experimental for Blank.1^{DART.2} (R12)

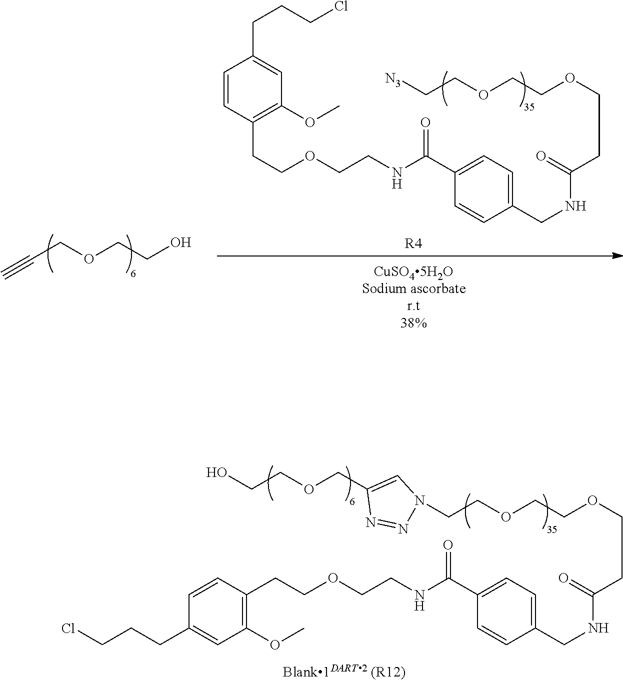

To 3, 6, 9, 12, 15, 18-hexaoxahenicos-20-yn-1-ol (4.0 mg, 12.5 μmol) was added EtOH:t-BuOH:water (2:1:1, 0.8 mL) followed by azido-PEG36-HTL2.0, R4 (26.1 mg, 12.5 μmol). Then, copper (II) sulfate pentahydrate (0.16 mg, 0.62 μmol) and sodium ascorbate (0.2 mg, 1.25 μmol) were added and the reaction was stirred overnight at room temperature after which it was purified by reverse phase prep HPLC eluting with 25 to 90% acetonitrile in water (0.1% formic acid, 220 nm wavelength for collection and the parameters as described in the general chemical synthesis section). The fraction was lyophilized to give Blank.1^{DART.2}, R12 (11.4 mg, 38%). ¹H NMR (500 MHZ, CDCl₃) δ 7.73 (br.s, 1H), 7.69-7.66 (m, 2H), 7.34 (d, J=7.9 Hz, 2H), 7.09 (br.s, 1H), 7.03 (d, J=7.8 Hz, 1H), 6.71-6.61 (m, 2H), 6.51 (br, 1H), 4.66 (s, 2H), 4.52 (t, J=5.0 Hz, 2H), 4.48 (dd, J=5.0 Hz, 2H), 3.86 (t, J=4.9 Hz, 2H), 3.79-3.47 (m, 170H), 2.88 (t, J=7.0 Hz, 2H), 2.71 (t, J=7.4 Hz, 2H), 2.53 (t, J=5.6 Hz, 2H), 2.03 (dq, J=8.3, 6.6 Hz, 2H). HRMS (ESI+): m/z calcd for C₁₁₂H₂₀₅ClN₅NaO₄₇: 1215.6707 [M+Na+H]²⁺; found: 1215.6749, [M+Na+H]²⁺.

### Example 14

### Experimental for Diazepam.1^{DART.2} (R13)

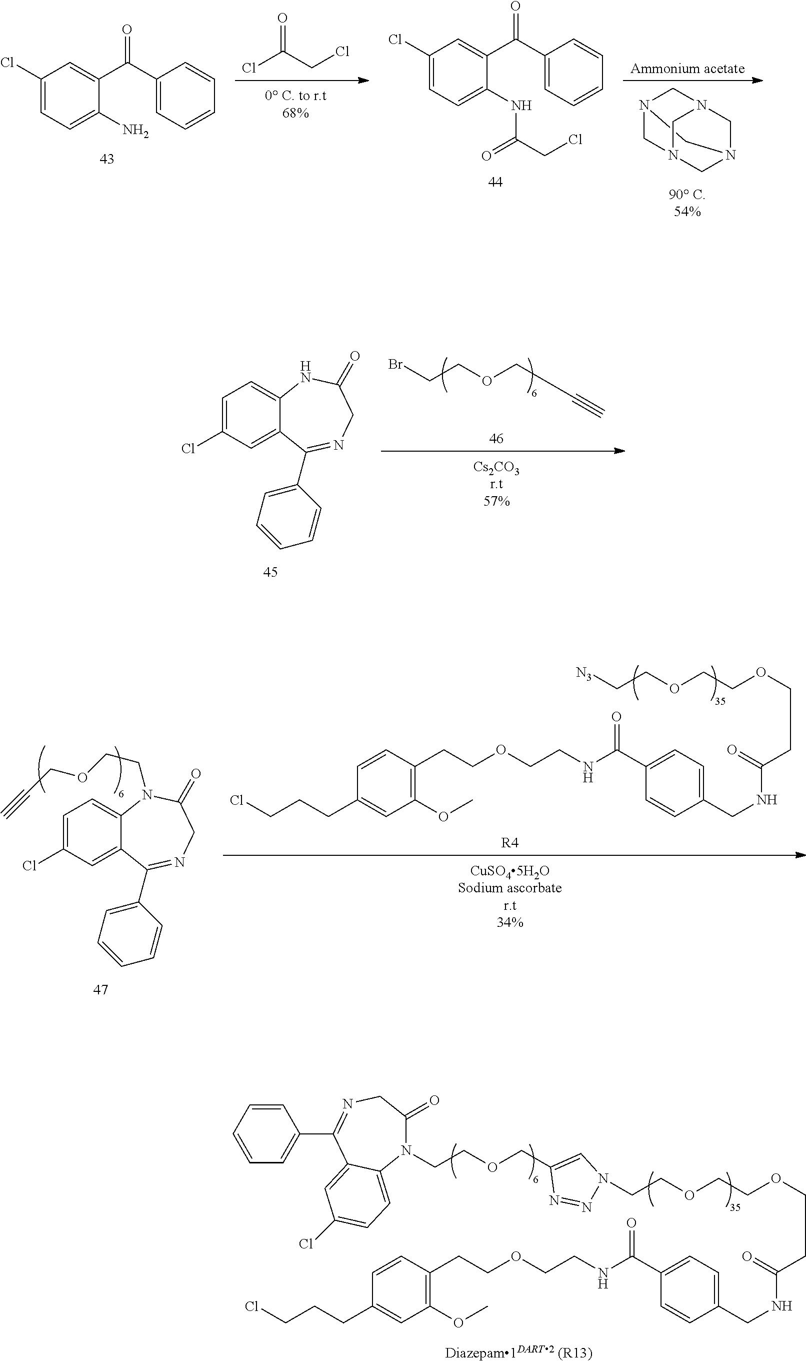

### Synthesis of 1-bromo-3, 6, 9, 12, 15, 18-hexaoxahenicos-20-yne, 46

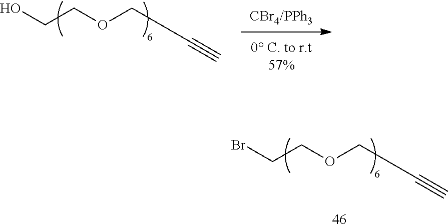

To 3, 6, 9, 12, 15, 18-hexaoxahenicos-20-yn-1-ol (1.00 g, 1 equiv., 3.12 mmol) in THF (20 mL) in a cooled ice/water bath was added CBr₄ (1.55 g, 1.50 equiv., 4.68 mmol) and triphenylphosphine (1.23 g, 1.50 equiv., 4.68 mmol) and the reaction mixture was stirred as the ice bath expired for 4 h after which hexanes (15 mL) were added. The mixture was filtered and the solids were discarded. The filtrate was concentrated, triturated in a mixture of hexanes/THF (˜ 1:1), filtered and concentrated again to remove more triphenylphosphine oxide. The yellow oil was purified by silica gel chromatography eluting with 0 to 100% ethyl acetate in hexanes to give 1-bromo-3,6,9,12, 15, 18-hexaoxahenicos-20-yne, 46 (688 mg, 1.80 mmol, 57.5%) as a yellow oil. ¹H NMR (500 MHZ, CDCl₃, contains a small amount of PPh₃═O) δ 4.19 (d, J=2.4 Hz, 2H), 3.79 (t, J=6.4 Hz, 2H), 3.70-3.59 (m, 20H), 3.45 (t, J=6.4 Hz, 2H), 2.41 (t, J=2.4 Hz, 1H). (ES-LCMS) m/z 385.3 (M+H)⁺.

### Step 1: Synthesis of N-(2-benzoyl-4-chlorophenyl)-2-chloroacetamide, 44

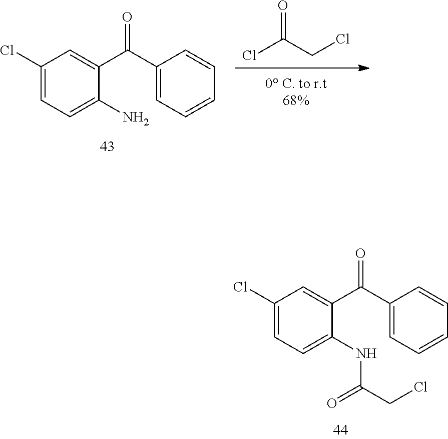

To a cooled (ice/water bath) solution of (2-amino-5-chlorophenyl) (phenyl) methanone, 43 (1 g, 4.32 mmol) in dichloromethane (14.39 mL) under nitrogen flow was added triethylamine (0.902 ml, 6.47 mmol) and DMAP (5.27 mg, 0.043 mmol) after which chloroacetyl chloride (0.380 ml, 4.75 mmol) was added dropwise. Additional chloroacetyl chloride (0.2 ml, 2.497 mmol) was added and the ice bath was removed. The reaction was stirred for another 2 h at room temperature after which the reaction was filtered to remove solids, which were washed with DCM. The organic layer was washed with saturated NaHCO₃ solution (2×5 mL), water (5 mL), brine (5 mL), and filtered through an isolute phase separator. The filtrate was concentrated to a brown solid which was crystallized from EtOH to give N-(2-benzoyl-4-chlorophenyl)-2-chloroacetamide, 44 (0.9 g, 2.92 mmol, 67.7% yield) as a light brown/light yellow solid. ¹H NMR (500 MHZ, CDCl₃) δ 11.44 (s, 1H), 8.61-8.55 (m, 1H), 7.76-7.67 (m, 2H), 7.66-7.58 (m, 1H), 7.58-7.46 (m, 4H), 4.17 (s, 2H). (ES-LCMS) m/z 310.1 (M+H)⁺.

### Step 2: Synthesis of 7-chloro-5-phenyl-1H-benzo[e][1,4]diazepin-2 (3H)-one, 45

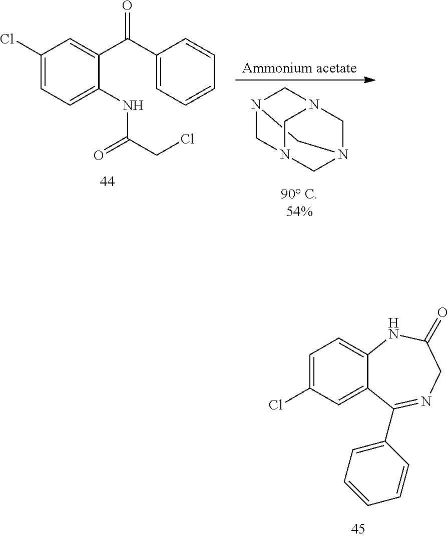

To N-(2-benzoyl-4-chlorophenyl)-2-chloroacetamide, 44 (0.9 g, 2.92 mmol) in ethanol (58.4 mL) was added ammonium acetate (0.45 g, 5.84 mmol) and hexamethylenetetramine (0.82 g, 5.84 mmol) after which the reaction was heated to 90° C. for 1 h. Additional ammonium acetate (120 mg, 1.56 mmol) and hexamethylenetetramine (240 mg, 1.7 mmol) were added and the reaction was heated at 75° C. overnight after which the reaction was diluted with water (240 mL). The suspension was extracted with ethyl acetate (2×100 mL), brine (30 mL), and filtered through an isolute phase separator after which the filtrate was concentrated. The residue was triturated in hexanes/acetone and filtered to give 7-chloro-5-phenyl-1H-benzo[e][1,4]diazepin-2 (3H)-one, 45 (0.43 g, 1.588 mmol, 54.4% yield) as a tan solid. ¹H NMR (500 MHz, Chloroform-d) δ 9.05 (s, 1H), 7.54-7.49 (m, 2H), 7.49-7.35 (m, 4H), 7.29 (d, J=2.4 Hz, 1H), 7.10 (d, J=8.7 Hz, 1H), 4.57-4.02 (m, 2H). (ES-LCMS) m/z 271.2 (M+H)⁺.

### Step 3: Synthesis of 7-chloro-1-(3, 6, 9, 12, 15, 18-hexaoxahenicos-20-yn-1-yl)-5-phenyl-1H-benzo[e][1,4]diazepin-2 (3H)-one

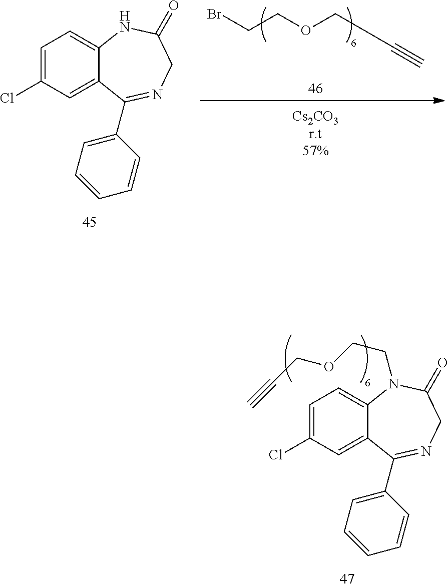

To a vial was added 7-chloro-5-phenyl-1,3-dihydro-2H-benzo[e][1,4]diazepin-2-one, 45 (100 mg, 1 equiv., 369 μmol) and DMF (3 mL) after which 1-bromo-3, 6, 9, 12, 15,18-hexaoxahenicos-20-yne, 46 (142 mg, 1 equiv., 369 μmol) was added followed by cesium carbonate (181 mg, 1.5 Eq, 554 μmol) and the reaction mixture was allowed to stir overnight. The reaction mixture was diluted with water (5 mL) and ethyl acetate (20 mL). The aqueous layer was extracted once with ethyl acetate (20 mL). Combined organic layers were washed with brine, filtered through an isolute phase separator, concentrated and purified by reverse phase prep HPLC eluting with 20 to 80% acetonitrile in water (0.1% formic acid and other conditions as reported in the general chemical synthesis section) to give 7-chloro-1-(3, 6, 9, 12, 15, 18-hexaoxahenicos-20-yn-1-yl)-5-phenyl-1,3-dihydro-2H-benzo[e][1,4]diazepin-2-one, 47 (121 mg, 211 μmol, 57.2%) as a yellow oil. ¹H NMR (500 MHZ, CDCl₃) δ 7.63 (d, J=8.8 Hz, 1H), 7.61-7.57 (m, 2H), 7.49-7.43 (m, 2H), 7.42-7.36 (m, 2H), 7.22 (d, J=2.5 Hz, 1H), 4.77 (d, J=10.4 Hz, 1H), 4.17 (d, J=2.4 Hz, 2H), 4.16-4.09 (m, 1H), 3.89 (ddd, J=14.3, 5.4, 4.0 Hz, 1H), 3.79-3.71 (m, 2H), 3.70-3.38 (m, 21H), 2.40 (t, J=2.4 Hz, 1H). (ES-LCMS) m/z 573.4 (M+H)⁺.

### Step 4: Synthesis of Diazepam.1^{DART.2} (R13)

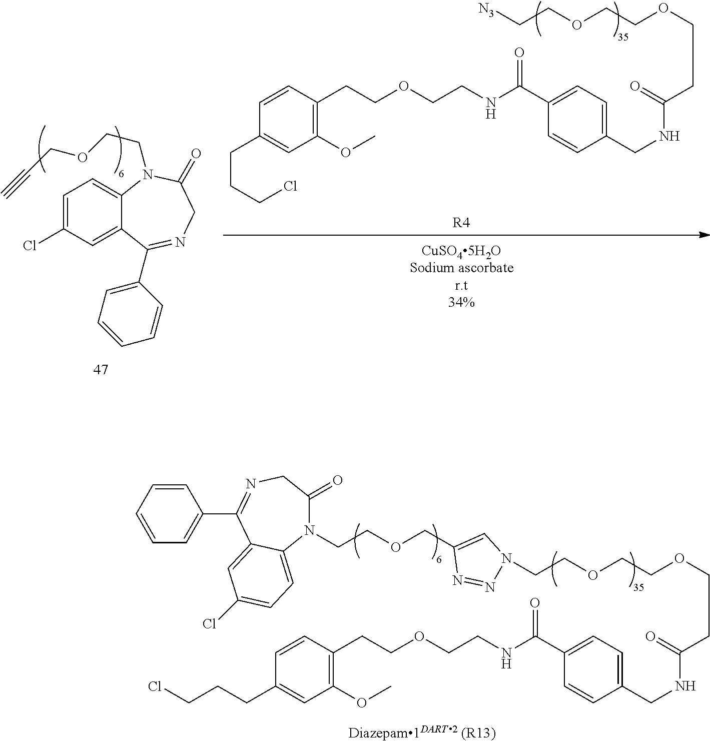

To -chloro-1-(3, 6, 9, 12, 15, 18-hexaoxahenicos-20-yn-1-yl)-5-phenyl-1,3-dihydro-2H-benzo[e][1,4]diazepin-2-one (5 mg, 8.7 μmol), 47 was added a solution azido-PEG36-HTL2.0, R4 (18 mg, 8.7 μmol) in EtOH (0.4 mL)/t-BuOH (0.2 mL)/Water (0.2 mL). Then, copper (II) sulfate pentahydrate (0.1 mg, 0.44 μmol) and sodium ascorbate (0.15 mg, 0.87 μmol) were added and the reaction mixture was stirred overnight after which it was purified by reverse phase prep HPLC eluting with 5 to 95% acetonitrile in water (0.1% formic acid, 220 nm wavelength for collection and other conditions as reported in the general chemical synthesis methods section) to give 1-(4-(19-(7-chloro-2-oxo-5-phenyl-2,3-dihydro-1H-benzo[e][1,4]diazepin-1-yl)-2, 5, 8, 11, 14, 17-hexaoxanonadecyl)-1H-1,2,3-triazol-1-yl)-N-(4-((2-(4-(3-chloropropyl)-2-methoxyphenethoxy)ethyl) carbamoyl)benzyl)-3,6,9,12, 15, 18,21,24,27, 30, 33, 36, 39, 42, 45, 48, 51, 54, 57, 60, 63, 66, 69, 72, 75, 78, 81, 84, 87, 90, 93, 96, 99, 102, 105, 108-hexatriacontaoxaundecahectan-111-amide, Diazepam.1^{DART.2}, R13 (8 mg, 34%). ¹H NMR (500 MHZ, CDCl₃) δ 7.71 (s, 1H), 7.69-7.66 (m, 2H), 7.63 (d, J=8.9 Hz, 1H), 7.60-7.57 (m, 2H), 7.48-7.44 (m, 2H), 7.41-7.37 (m, 2H), 7.33 (d, J=8.2 Hz, 2H), 7.22 (d, J=2.5 Hz, 1H), 7.10 (t, J=6.4 Hz, 1H), 7.05 (d, J=7.8 Hz, 1H), 6.66 (d, J=6.4 Hz, 2H), 6.52 (br.s, 1H), 4.77 (d, J=10.5 Hz, 1H), 4.65 (s, 2H), 4.51 (t, J=5.1 Hz, 2H), 4.48 (d, J=6.0 Hz, 2H), 4.16-4.12 (m, 1H), 3.91-3.42 (m, 184H), 2.85 (t, J=7.0 Hz, 2H), 2.71 (t, J=7.4 Hz, 2H), 2.53 (t, J=5.6 Hz, 2H), 2.03 (dq, J=8.2, 6.6 Hz, 2H). HRMS (ESI+): m/z calcd for C₁₂₇H₂₁₄Cl₂N₇NaO₄₇: 1341.6934 [M+Na+H]²⁺; found: 1341.6982 [M+Na+H]²⁺.

### Example 15

### Experimental for Flumazenil.1^{DART.2} (R14)

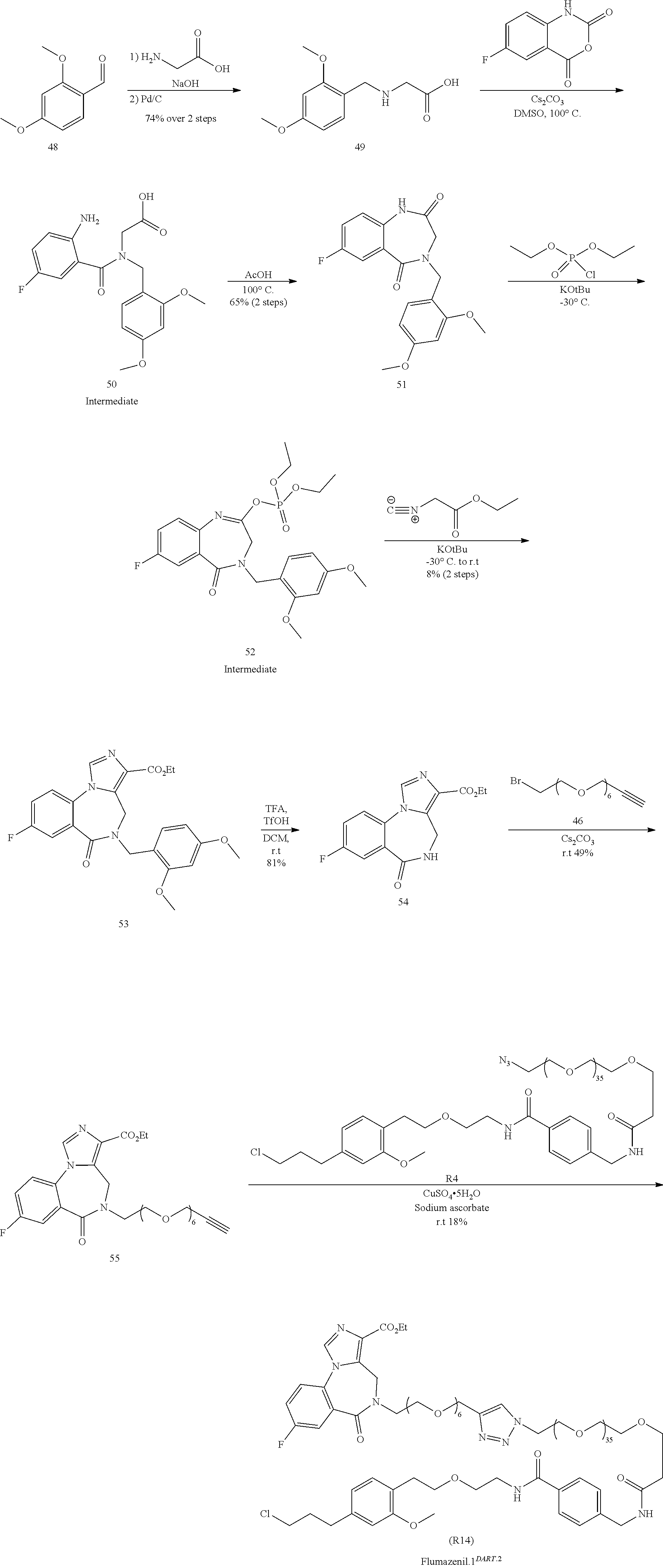

### Step 1: Synthesis of 2-((2,4-dimethoxybenzyl)amino) acetic acid, 49

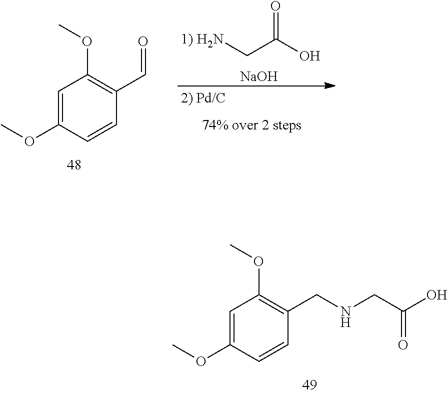

To a round bottom flask was added glycine (2 g, 26.6 mmol) to which 1M NaOH (32 mL) was added. In a separate vial was added 2,4-dimethoxybenzaldehyde, 48 (3.98 g, 23.98 mmol) in methanol (16 mL) and this stirred suspension was added portionwise to the round bottom flask containing glycine/NaOH. A white solid precipitated and the suspension was stirred for 10 min at room temperature after which Pd/C (0.8 g, 0.752 mmol) was added under a stream of nitrogen. The reaction was stirred overnight under an atmosphere of hydrogen after which it was filtered through a pad of celite, washing with hot methanol. The filtrate was concentrated on the rotary evaporator to give a yellow aqueous solution which was cooled in an ice bath. The mixture was acidified (3 M HCl) until pH=4. The mixture was concentrated to remove water, taken up in methanol and filtered. The filtrate was concentrated then triturated from acetone to give 2-((2,4-dimethoxybenzyl)amino) acetic acid, 49 (4.45 g, 19.76 mmol, 74.2% yield) as a white solid. ¹H NMR (500 MHZ, DMSO-d₆) δ 13.71 (br.s, 1H), 9.26 (br.s, 2H), 7.36 (d, J=8.4 Hz, 1H), 6.62 (d, J=2.4 Hz, 1H), 6.57 (dd, J=8.4, 2.4 Hz, 1H), 4.08 (s, 2H), 3.82 (s, 3H), 3.79 (s, 3H), 3.72 (s, 2H). (ES-LCMS) m/z 226.1 (M+H)⁺.

### Step 2/3: Synthesis of 4-(2,4-dimethoxybenzyl)-7-fluoro-3,4-dihydro-1H-benzo[e][1,4]diazepine-2,5-dione, 51

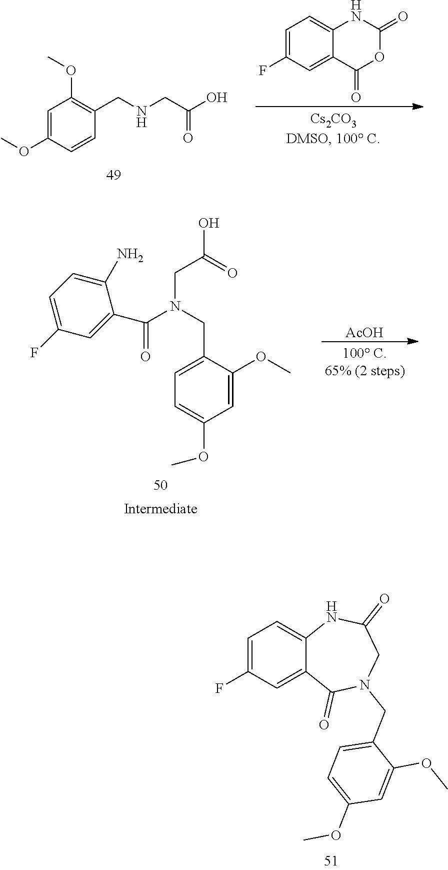 [SMILES: COc1ccc(CNCC(=O)O)c(OC)c1 ; "AcOH" ; O=c1[nH]c2ccc(F)cc2c(=O)o1 ; COc1ccc(CN(CC(=O)O)C(=O)c2cc(F)ccc2N)c(OC)c1 ; COc1ccc(CN2CC(=O)Nc3ccc(F)cc3C2=O)c(OC)c1]

To a vial was added 2-((2,4-dimethoxybenzyl)amino) acetic acid, 49 (2 g, 8.88 mmol) to which DMSO (11 mL) was added. Then, 6-fluoro-1H-benzo[d][1,3]oxazine-2,4-dione (1.59 g, 8.78 mmol) was added and the mixture was stirred until all solids dissolved. Cesium carbonate (2.89 g, 8.88 mmol) was added and the reaction mixture was heated at 100° C. for 10 min after the intermediate 50 was observed on LCMS. Pressure buildup was observed during this reaction. Acetic acid (2.033 ml, 35.5 mmol) was added and the reaction mixture was heated at 100° C. overnight. To this mixture was added water and the suspension was thoroughly stirred, then filtered. The collected tan solid was washed generously with water and dried overnight to give 4-(2,4-dimethoxybenzyl)-7-fluoro-3,4-dihydro-1H-benzo[e][1,4]diazepine-2,5-dione, 51 (2 g, 5.81 mmol, 65.4% yield) as a tan solid. ¹H NMR (500 MHZ, Chloroform-d) δ 8.38 (s, 1H), 7.67 (dd, J=9.0, 3.1 Hz, 1H), 7.27 (d, J=8.1 Hz, 1H), 7.15 (ddd, J=8.8, 7.3, 3.1 Hz, 1H), 6.90 (dd, J=8.8, 4.6 Hz, 1H), 6.44 (d, J=7.6 Hz, 2H), 4.77 (s, 2H), 3.88 (s, 2H), 3.80 (s, 3H), 3.77 (s, 3H). (ES-LCMS) m/z 345.0 (M+H)⁺.

### Step 4/5: Synthesis of ethyl 5-(2,4-dimethoxybenzyl)-8-fluoro-6-oxo-5,6-dihydro-4H-benzo[f]imidazo[1,5-a][1,4]diazepine-3-carboxylate, 53

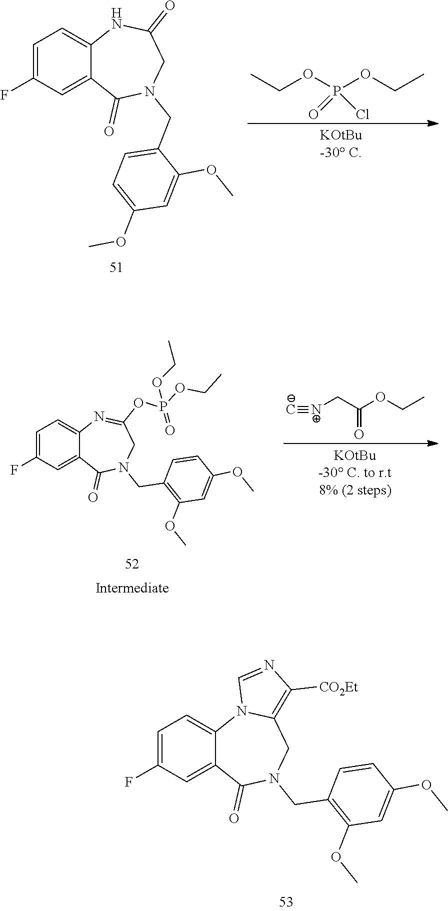

4-(2,4-dimethoxybenzyl)-7-fluoro-3,4-dihydro-1H-benzo[e][1,4]diazepine-2,5-dione, 51 (2 g, 5.81 mmol) was added to a vial to which dry DMF (8 mL) was added followed by potassium tert-butoxide (0.782 g, 6.97 mmol). After stirring for 15 min under nitrogen, the reaction was placed in an acetonitrile/dry ice bath at −30° C. and diethyl chlorophosphate (0.919 ml, 6.39 mmol) was added dropwise over 2 minutes. Upon addition, the reaction was stirred for 20 min and maintained in the cold bath during this time.

In a separate vial, potassium tert-butoxide (0.782 g, 6.97 mmol) was dissolved in anhydrous DMF (3 mL) and after stirring for 5 min at room temperature, this solution was cooled in an acetonitrile/dry ice bath at −50° C. and placed under nitrogen after which ethyl 2-isocyanoacetate (0.698 ml, 6.39 mmol) in DMF (1 mL) was added dropwise. This dark brown solution was immediately added dropwise over 5 min to the solution (which at this point has warmed to −20° C.) containing the intermediate described in the previous paragraph. The resulting mixture was stirred at room temperature for 2 hours after which it was quenched with acetic acid (0.499 mL, 8.71 mmol). Saturated aqueous NH₄Cl solution (5 mL) was added. The reaction mixture was diluted with ethyl acetate (40 mL) and the aqueous layer was extracted with EtOAc (2×30 mL). Combined organic extracts were washed with brine and dried (sodium sulfate). The organic layer was decanted off and concentrated to give a black oil (˜ 3 g) with a stench odor.

The mixture was purified by silica gel chromatography eluting with 5 to 50% ethylacetate in dichloromethane to give 600 mg of a slightly unclean product. This solid was recrystallized from ethyl acetate to give desired product, 53 as a white powder (200 mg, 8%). ¹H NMR (500 MHZ, Chloroform-d) δ 7.86-7.75 (m, 2H), 7.38 (dd, J=8.8, 4.5 Hz, 1H), 7.31 (ddd, J=8.8, 7.1, 3.0 Hz, 1H), 7.19 (d, J=8.2 Hz, 1H), 6.52-6.28 (m, 2H), 5.36 (d, J=15.9 Hz, 1H), 5.00-4.55 (m, 2H), 4.31 (q, J=7.1 Hz, 2H), 4.15 (d, J=15.9 Hz, 1H), 3.77 (d, J=17.1 Hz, 6H), 1.37 (t, J=7.1 Hz, 3H). (ES-LCMS) m/z 440.3 (M+H)⁺.

### Step 6: Synthesis of ethyl 8-fluoro-6-oxo-5,6-dihydro-4H-benzo[f]imidazo[1,5-a][1,4]diazepine-3-carboxylate, 54

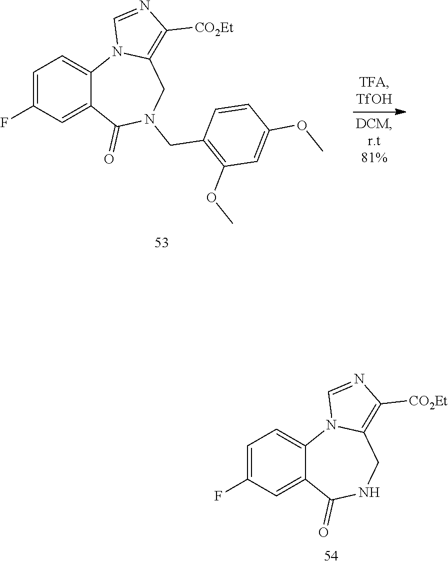 [SMILES: CCOC(=O)c1ncn2c1CN(Cc1ccc(OC)cc1OC)C(=O)c1cc(F)ccc1-2 ; CCOC(=O)c1ncn2c1CNC(=O)c1cc(F)ccc1-2]

To a vial containing ethyl 5-(2,4-dimethoxybenzyl)-8-fluoro-6-oxo-5,6-dihydro-4H-benzo[f]imidazo[1,5-a][1,4]diazepine-3-carboxylate, 53 (0.15 g, 0.341 mmol) was added DCM (1 mL) and the mixture was placed in an ice/water bath after which TFA (1 mL, 12.98 mmol) and trifluoromethanesulfonic acid (69 uL, 0.777 mmol) were added. The reaction mixture turned from light pink to dark purple over the course of 90 minutes. The reaction mixture was concentrated and diluted with DCM/saturated aqueous sodium bicarbonate solution. The solution was stirred and the color changed from purple to a very light pink, and the aqueous layer was slightly basic (pH=8). The aqueous layer was extracted with DCM and all combined organic extracts were washed with brine, filtered through an isolute phase separator, and concentrated to give a white solid which was triturated from hot ethyl

acetate and diethyl ether to give ethyl 8-fluoro-6-oxo-5,6-dihydro-4H-benzo[f]imidazo[1,5-a][1,4]diazepine-3-carboxylate, 54 (80 mg, 0.277 mmol, 81% yield) as a white solid.

¹H NMR (500 MHZ, CDCl₃) δ 7.85-7.77 (m, 2H), 7.44 (dd, J=8.8, 4.5 Hz, 1H), 7.37 (ddd, J=8.9, 7.0, 2.9 Hz, 1H), 6.52 (t, J=6.3 Hz, 1H), 4.41 (q, J=7.1 Hz, 2H), 3.84-3.63 (m, 1H), 1.42 (t, J=7.1 Hz, 3H). (ES-LCMS) m/z 290.1 (M+H)⁺.

### Step 7: Synthesis of ethyl 8-fluoro-5-(3, 6, 9, 12, 15, 18-hexaoxahenicos-20-yn-1-yl)-6-oxo-5,6-dihydro-4H-benzo[f]imidazo[1,5-a][1,4]diazepine-3-carboxylate, 55

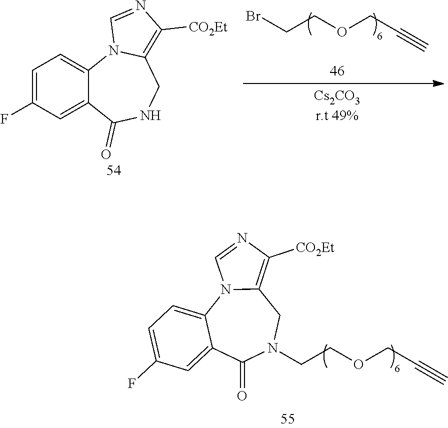

In a vial was added ethyl 8-fluoro-6-oxo-5,6-dihydro-4H-benzo[f]imidazo[1,5-a][1,4]diazepine-3-carboxylate, 54 (10 mg, 0.035 mmol) followed by anhydrous DMF (700 uL) and 1-bromo-3, 6, 9, 12, 15, 18-hexaoxahenicos-20-yne, 46 (13.25 mg, 0.035 mmol) followed by anhydrous cesium carbonate (22.53 mg, 0.069 mmol) and the reaction was stirred at room temperature overnight after which additional alkyl bromide (5 mg) was added. After stirring for several hours, no change was detected by LCMS (still some SM remaining).

The reaction was diluted with ethyl acetate (20 mL) and water (5 mL). The organic layer was washed with brine (5 mL), filtered through an isolute phase separator, concentrated and purified by RP HPLC eluting with 5 to 70% acetonitrile in water (0.1% FA and the rest of conditions as described in the general chemistry synthesis section) to give ethyl 8-fluoro-5-(3, 6, 9, 12, 15, 18-hexaoxahenicos-20-yn-1-yl)-6-oxo-5,6-dihydro-4H-benzo[f]imidazo[1,5-a][1,4]diazepine-3-carboxylate, 55 (10 mg, 0.017 mmol, 48.9% yield) as a brown oil.

¹H NMR (500 MHZ, CDCl₃) δ 7.85 (s, 1H), 7.77 (d, J=8.8 Hz, 1H), 7.42 (dd, J=9.2, 4.4 Hz, 1H), 7.33 (t, J=8.2 Hz, 1H), 5.39 (d, J=16.1 Hz, 1H), 4.48-4.23 (m, 4H), 4.17 (d, J=5.7 Hz, 4H), 3.98 (d, J=13.6 Hz, 1H), 3.84-3.37 (m, 45H), 2.40 (s, 2H), 1.42 (t, J=7.2 Hz, 3H). (ES-LCMS) m/z 592.1 (M+H)⁺.

### Step 6: Synthesis of Flumazenil.1^{DART.2} (R14)

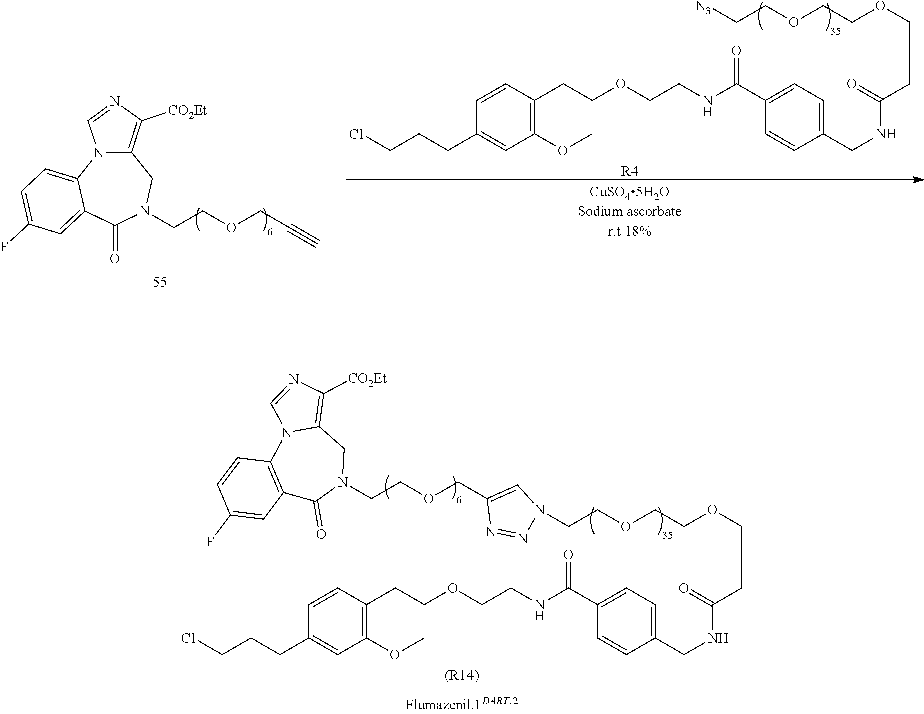

To a vial containing ethyl 8-fluoro-5-(3, 6, 9, 12, 15, 18-hexaoxahenicos-20-yn-1-yl)-6-oxo-5,6-dihydro-4H-benzo[f]imidazo[1,5-a][1,4]diazepine-3-carboxylate, 55 (5 mg, 8.5 μmol) was added a solution of azido-PEG36 HTL2.0, R4 (18 mg, 8.5 μmol) in EtOH:t-BuOH:water (2:1:1, 0.8 mL). Then, copper (II) sulfate pentahydrate (0.1 mg, 0.42 μmol) and sodium ascorbate (0.15 mg, 0.85 μmol) were added and the reaction mixture was stirred overnight after which it was purified by RP HPLC eluting with 10 to 80% acetonitrile in water (0.1% FA, 220 nm wavelength) and other conditions as described in the general chemistry synthesis section to give Flumazenil.1^{DART.2}, R14 (4 mg, 18%).

¹H NMR (500 MHZ, CDCl₃) δ 7.86 (s, 1H), 7.77 (dd, J=8.8, 2.9 Hz, 1H), 7.72-7.63 (m, 3H), 7.43 (dt, J=8.8, 4.5 Hz, 1H), 7.36-7.30 (m, 3H), 7.14-7.01 (m, 2H), 6.68-6.60 (m, 2H), 6.50 (s, 1H), 5.39 (d, J=16.0 Hz, 1H), 4.65 (d, J=5.5 Hz, 2H), 4.53-4.46 (m, 4H), 4.44-4.28 (m, 2H), 3.98 (d, J=13.7 Hz, 1H), 3.85 (t, J=5.2 Hz, 2H), 3.79-3.72 (m, 6H), 3.70-3.43 (m, 170H), 2.89-2.78 (m, 3H), 2.71 (t, J=7.4 Hz, 2H), 2.53 (t, J=5.6 Hz, 2H), 2.03 (dq, J=8.3, 6.6 Hz, 2H), 1.42 (t, J=7.1 Hz, 3H). HRMS (ESI+): m/z calcd for C₁₂₆H₂₁₄ClFN₈Na₂O₄₉: 1362.1995 [M+2Na]²⁺; found: 1362.1700 [M+2Na]²⁺.

### Example 16

### Experimental for CMPDA.1^{DART.1}

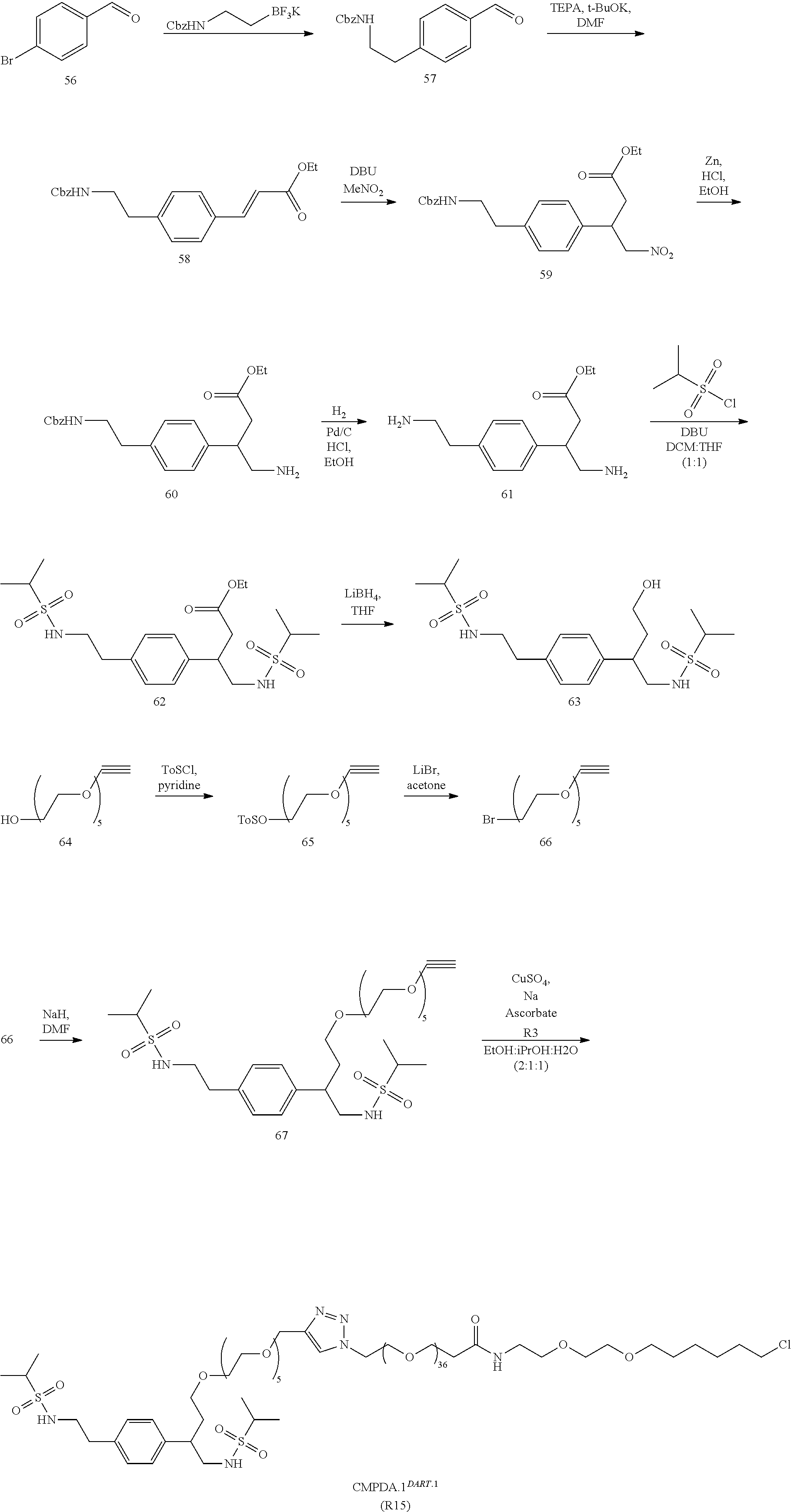

### Step 1: Synthesis of benzyl 4-formylphenethylcarbamate, 57

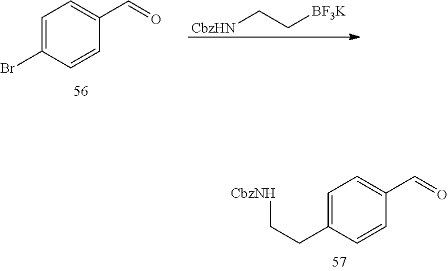

To solution of 4-bromobenzaldehyde (2.36 g, 12.7 mmol) in toluene (75 mL) in a sealed tube were added potassium potassium benzyl N-[2-(trifluoroboranuidyl)ethyl]carbamate (4.0 g, 14.0 mmol), Cs₂CO₃ (12.4 g, 38.2 mmol), PdCl2 (dppf)·CH₂Cl₂ (0.52 g, 0.64 mmol) and water (25 mL). The reaction mixture was purged with nitrogen. The tube was sealed and heated to 80° C. for 18 h. The reaction was allowed to cool to room temperature and saturated solution of NH₄Cl was added. The suspension was extracted with CH₂Cl₂ (30 mL×3 times). The organic extracts were combined, dried (Na₂SO₄), and concentrated under reduced pressure to provide crude compound. Purification by flash chromatography (24 g silica cartridge, EtOAc/Hexane) gave the title compound (2.0 g, 56% yield).

### Step 2: Synthesis of benzyl-4-((E/Z)-2-(ethoxycarbonyl) vinyl) phenethylcarbamate, 58

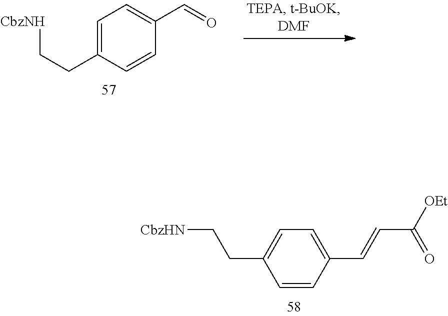

Triethylphosphonoacetate (9.6 g, 2.4 mmol) and potassium t-butoxide (1.1 g, 27.1 mmol) were dissolved in DMF (10 mL) at 0° C. and stirred 30 minutes. Benzyl-4-formylphenethylcarbamate (3.3 g, 13.5 mmol) dissolved in DMF (15 mL) was then added over 5 minutes. Reaction stirred overnight. The reaction was diluted with water and then extracted thrice (EtOAc). The organic extracts were combined, dried (Na₂SO₄), and concentrated under reduced pressure to provide crude compound. Purification by flash chromatography (24 g silica cartridge, EtOAc/Hexane) gave the title compound (1.96 g, 78% yield). (ESI) m/z 354 (M+1)⁺.

### Step 3: Synthesis of benzyl 4-(1-(ethoxycarbonyl)-3-nitropropan-2-yl) phenethylcarbamate, 59

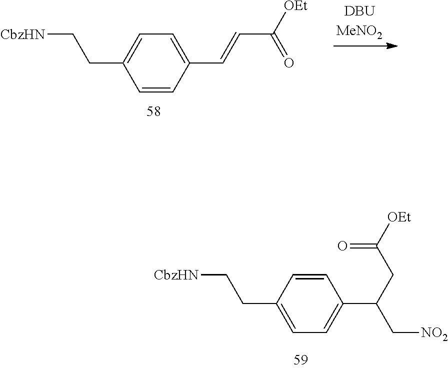 [SMILES: CCOC(=O)/C=C/c1ccc(CCNC(=O)OCc2ccccc2)cc1 ; CCOC(=O)CC(C[N+](=O)[O-])c1ccc(CCNC(=O)OCc2ccccc2)cc1]

The starting material benzyl-4-((E/Z)-2-(ethoxycarbonyl) vinyl) phenethylcarbamate (1.96 g, 5.55 mmol) was dissolved in nitromethane (20 mL) and 1,8-diazabicyclo 5.4.0 undec-7-ene (1.5 g, 10 mmol) was then added. The reaction mixture was stirred at room temperature for 48 h. The reaction mixture was diluted with ethyl acetate and washed twice with aqueous 1N HCl. The organic layer was dried (Na₂SO₄), and concentrated under reduced pressure to provide crude product. Purification by flash chromatography (EtOAc/Hexane) gave the title compound (2.34 g, quant. yield). ¹H NMR (300 MHZ, CHLOROFORM-d) δ=7.36 (s, 5H), 7.16 (s, 4H), 5.10 (s, 2H), 4.82-4.54 (m, 3H), 4.17-3.91 (m, 4H), 3.51-3.37 (m, 2H), 2.83-2.69 (m, 4H), 2.06 (s, 1H), 1.42-1.07 (m, 3H). (ESI) m/z 415 (M+1)⁺.

### Step 4: Synthesis of benzyl 4-(1-(ethoxycarbonyl)-3-aminopropan-2-yl) phenethylcarbamate, 60

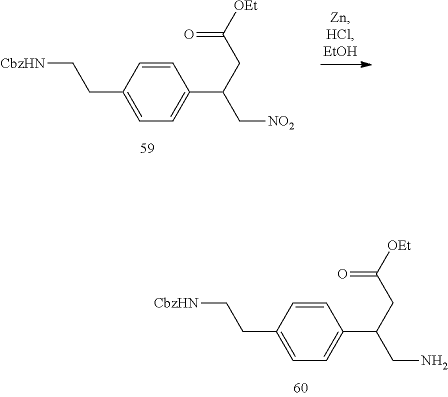 [SMILES: CCOC(=O)CC(C[N+](=O)[O-])c1ccc(CCNC(=O)OCc2ccccc2)cc1 ; CCOC(=O)CC(CN)c1ccc(CCNC(=O)OCc2ccccc2)cc1]

To benzyl 4-(1-(ethoxycarbonyl)-3-nitropropan-2-yl) phenethylcarbamate (3.35 g, 8.1 mmol) in ethanol (100 mL) was added zinc (5.25 g, 81 mmol) and 4N HCl in dioxane (24 mL, 97 mmol). After 1 hour, the reaction was filtered through celite and the filtrate concentrated. The residue was dissolved in ethyl acetate and washed with aqueous sodium bicarbonate. The aqueous layer was extracted twice (EtOAC). The organic extracts were combined, dried (Na₂SO₄), and concentrated under reduced pressure to provide crude compound. Purification by flash chromatography (24 g silica cartridge, EtOAc/Hexane) gave the title compound (3.11 g, quant. yield). (ESI) m/z 385 (M+1)⁺.

### Step 5: Synthesis of ethyl 4-amino-3-(4-(2-aminoethyl)phenyl) butanoate, 61

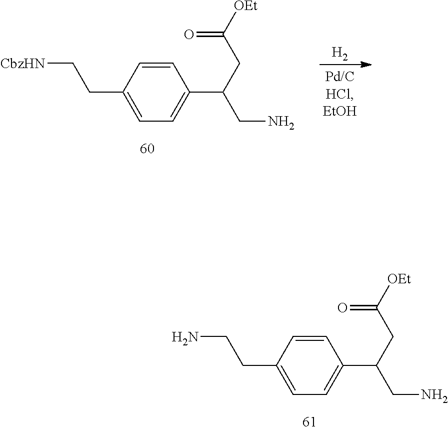 [SMILES: CCOC(=O)CC(CN)c1ccc(CCNC(=O)OCc2ccccc2)cc1 ; "H2" ; CCOC(=O)CC(CN)c1ccc(CCN)cc1]

To benzyl-4-(1-(ethoxycarbonyl)-3-aminopropan-2-yl) phenethylcarbamate (3.11 g, 8.1 mmol) in ethanol (100 mL) in a Parr bottle was added 10% Pd/C (350 mg) and 4N HCl in dioxane (6.1 mL). The sample was hydrogenated (50 psi) for 20 hours. Methanol was added to solubilize the product and the reaction was then filtered through celite and concentrated to crude product used without further purification. ¹H NMR (300 MHZ, CHLOROFORM-d) δ=7.19 (br s, 4H), 4.08 (br s, 3H), 3.40 (br s, 3H), 3.10 (br d, J=5.9 Hz, 2H), 2.86 (br s, 1H), 2.81-2.71 (m, 1H), 2.70-2.59 (m, 1H), 1.39-1.25 (m, 5H), 1.20 (br s, 2H). (ESI) m/z 251 (M+1)⁺.

### Step 6: Synthesis of ethyl 4-(propane-2-sulfonamido)-3-{4-[2-(propane-2-sulfonamido)ethyl]phenyl}butanoate, 62

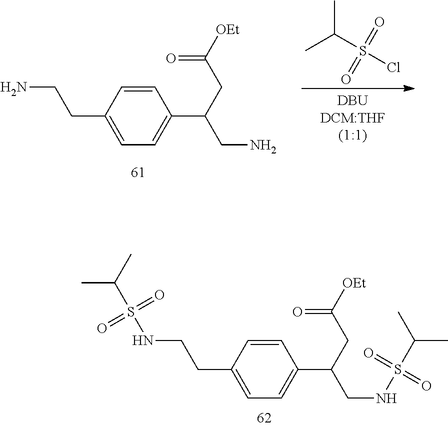

Ethyl 4-amino-3-(4-(2-aminoethyl)phenyl) butanoate (2.07 g, 6.4 mmol) in CH₂Cl₂:THF (60 mL, 1:1) was cooled to 0° C. 1,8-Diazabicyclo 5.4.0 undec-7-ene (5.7 mL, 38.4 mmol), and isopropyl sulfonyl chloride (2.73 g, 19.2 mmol) were then added. The reaction was stirred at room temperature for 22 h. The reaction was poured into 1N HCl and the solid collected by filtration to yield title compound (1.73 g, 58%).

### Step 7: Synthesis of N-(2-{4-[4-hydroxy-1-(propane-2-sulfonamido) butan-2-yl]phenyl}ethyl)propane-2-sulfonamide, 63

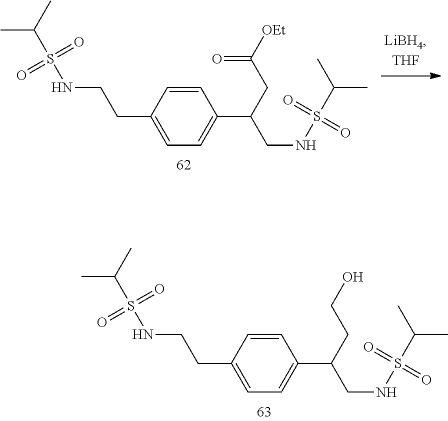

To ethyl 4-(propane-2-sulfonamido)-3-{4-[2-(propane-2-sulfonamido)ethyl]phenyl}butanoate (200 mg, 0.45 mmol) in THF (15 mL) was added lithium borohydride (4M in THF, 110 mL, 0.45 mmol) and the reaction heated to reflux for 2 hours. After cooling, the reaction was poured into aqueous buffer (pH=7) and extracted thrice with ethyl acetate. The organic extracts were combined, dried (Na₂SO₄), and concentrated under reduced pressure. Purification by flash chromatography (EtOAc/Hexane) afforded the title compound (0.16 g, 83 yield). (ESI) m/z 421 (M+1)⁺.

### Step 8: Synthesis of 2-(2-(2-(2-(2-(prop-2-ynyloxy) ethoxy) ethoxy) ethoxy) ethoxy)ethyl 4-methylbenzenesulfonate, 65

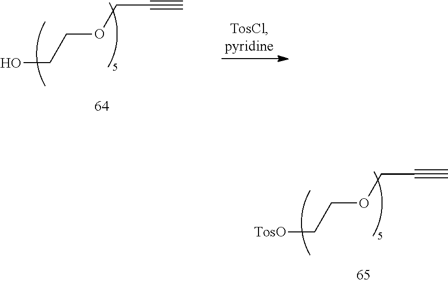

To 2-(2-(2-(2-(2-(prop-2-ynyloxy) ethoxy) ethoxy) ethoxy) ethoxy) ethanol (250 mg, 0.91 mmol) in pyridine (3 mL) at 0° C. was added tosyl chloride (345 mg, 1.81 mmol). The reaction warmed to room temperature while stirring overnight. The reaction was then concentrated and purified by flash chromatography (EtOAc/Hexane) to yield the title compound (57 mg, 14% yield). ¹H NMR (300 MHZ, CHLOROFORM-d) δ=7.72 (br d, J=6.7 Hz, 2H), 7.27-6.99 (m, 2H), 4.24-3.99 (m, 4H), 3.64-3.50 (m, 18H), 2.38 (br s, 4H).

### Step 9: Synthesis of 3-(2-(2-(2-(2-(2-bromoethoxy) ethoxy) ethoxy) ethoxy) ethoxy) prop-1-yne, 66

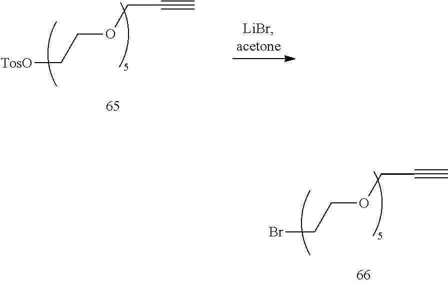

2-(2-(2-(2-(2-(Prop-2-ynyloxy) ethoxy) ethoxy) ethoxy) ethoxy)ethyl 4-methylbenzenesulfonate (57 mg, 0.132 mmol), and lithium bromide (35 mg, 0.397 mmol) were refluxed in acetone (5 mL) for 3 hours. The reaction was concentrated and residue purified by flash chromatography (EtOAc/Hexane) to yield the title compound (45 mg, quant. yield). ¹H NMR (300 MHZ, CHLOROFORM-d) δ=4.21 (br s, 2H), 3.86-3.76 (m, 2H), 3.67 (br s, 16H), 3.59-3.38 (m, 2H), 2.44 (br s, 1H).

### Step 10: Synthesis of N-(2-{4-[23-(propane-2-sulfonamido)-4, 7, 10, 13, 16, 19-hexaoxatricos-1-yn-22-yl]phenyl}ethyl)propane-2-sulfonamide, 67

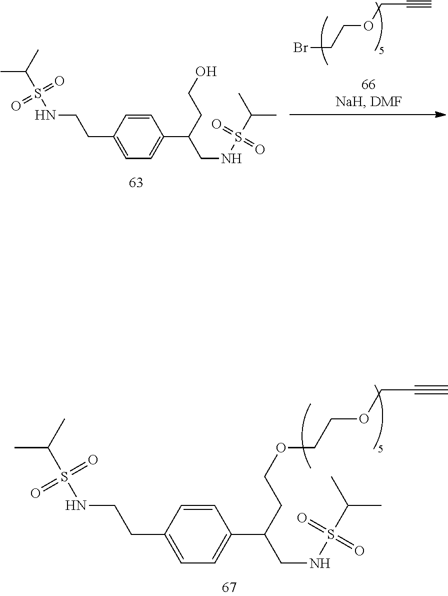

To N-(2-{4-[4-hydroxy-1-(propane-2-sulfonamido) butan-2-yl]phenyl}ethyl)propane-2-sulfonamide (63, 51 mg, 0.12 mmol) in DMF (1 mL) was added 60% sodium hydride in mineral oil (15 mg, 0.37 mmol) and the reaction stirred at 0° C. for 30 minutes. 3-(2-(2-(2-(2-(2-Bromoethoxy) ethoxy) ethoxy) ethoxy) ethoxy) prop-1-yne (66, 45 mg, 0.133 mmol) was added and the reaction stirred at ambient temperature overnight. The reaction was purified via reverse phase chromatography to yield the title compound (19 mg, 23%). (ESI) m/z 679 (M+1)⁺.

### Step 11: Synthesis of_N-(2-{2-[(6-chlorohexyl)oxy]ethoxy}ethyl)-1-{4-[21-(propane-2-sulfonamido)-20-{4-[2-(propane-2-sulfonamido)ethyl]phenyl}-2, 5, 8, 11, 14, 17-hexaoxahenicosan-1-yl]-1H-1,2,3-triazol-1-yl}-3, 6, 9, 12, 15, 18, 21, 24, 27, 30, 33, 36, 39, 42, 45, 48, 51, 54, 57, 60, 63, 66, 69, 72, 75, 78, 81, 84, 87, 90, 93, 96, 99, 102, 105, 108-hexatriacontaoxa111n-111-amide, CMPDA.1^{DART.1}

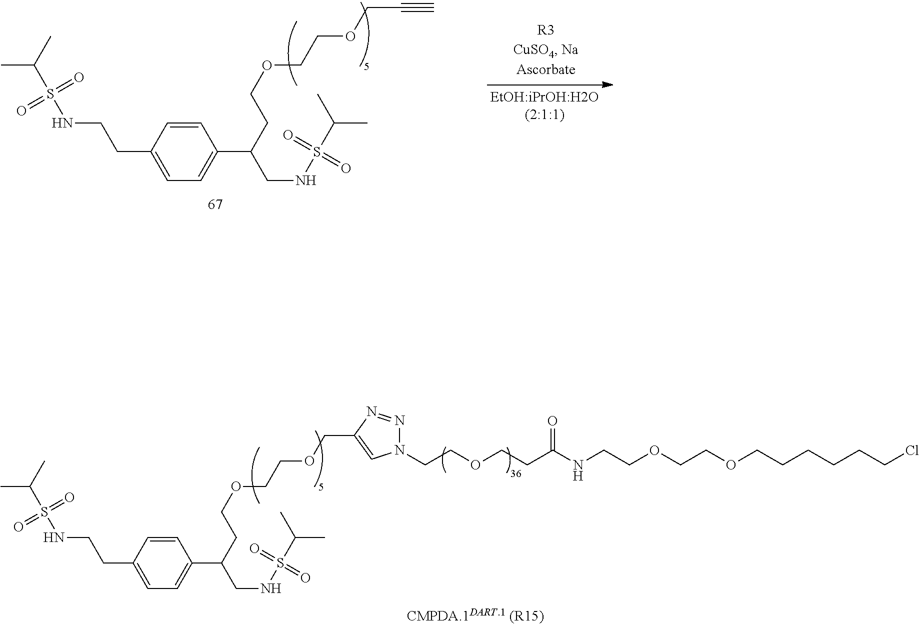

To a vial containing azido-PEG₃₆-HTL.1 (azido DART.1, R3) (37 mg, 0.019 mmol), N-(2-{4-[23-(propane-2-sulfonamido)-4, 7, 10, 13, 16, 19-hexaoxatricos-1-yn-22-yl]phenyl}ethyl)propane-2-sulfonamide (67, 13 mg, 0.19 mmol), and the solvents EtOH:i-PrOH:H₂O (2:1:1) (1 mL) was added CuSO₄·5H₂O (0.3 mg, 0.001 mmol) and (+)-sodium L-ascorbate (0.4 mg, 0.002 mmol) pre-dissolved in solutions of EtOH:i-PrOH:H₂O (2:1:1) (1 mL). The reaction was stirred for 20 h at room temperature. The reaction mixture was then filtered, concentrated, and purified by reverse phase HPLC to afford the title compound, R15 (36 mg, 72% yield). (ESI) m/z 1293 (^{1/2}M+1)⁺.

### Example 17

### Synthesis of reagent CMPDA.2^{DART.2}

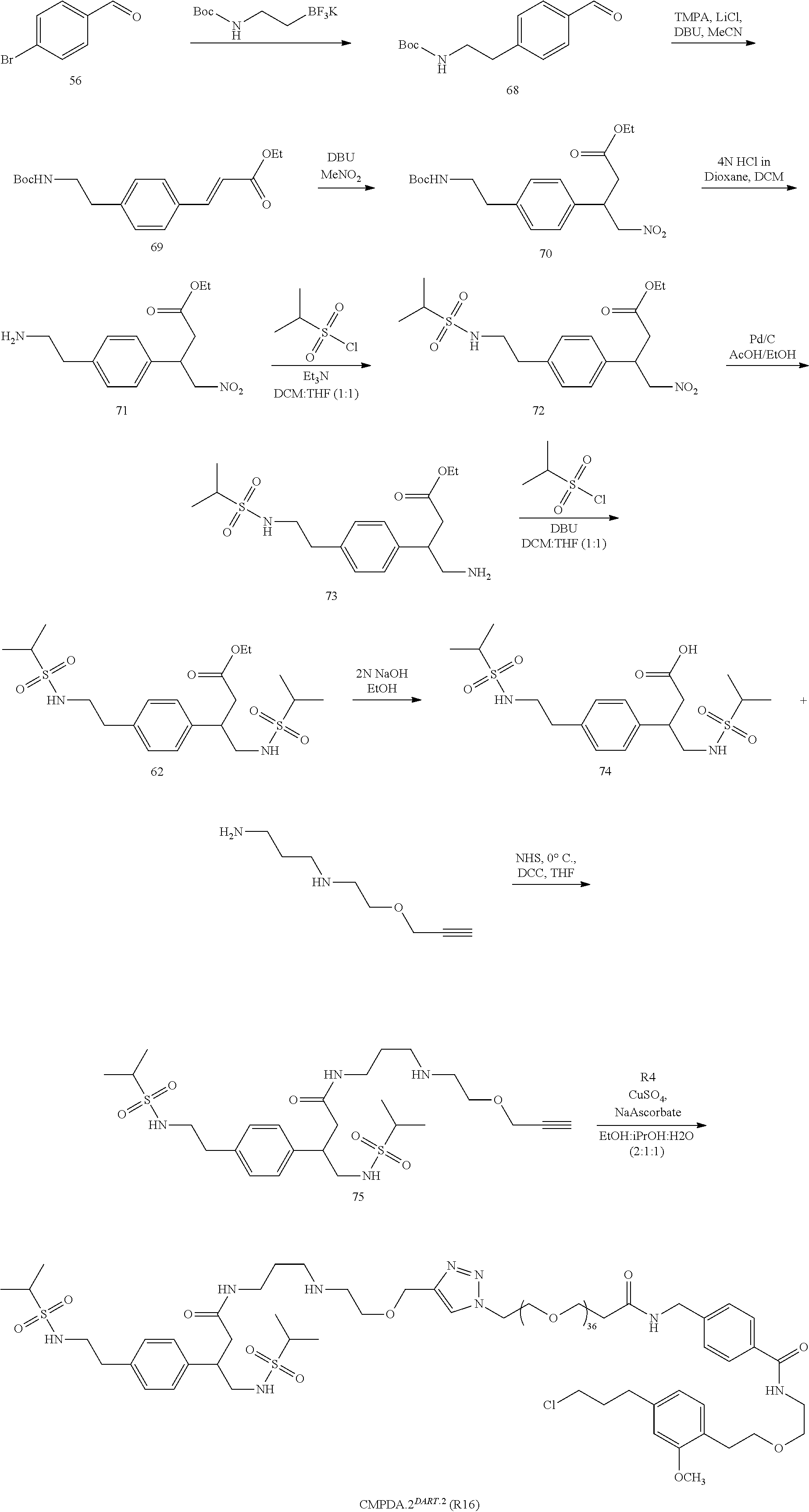

### Step 1: tert-butyl (4-formylphenethyl)carbamate, 68

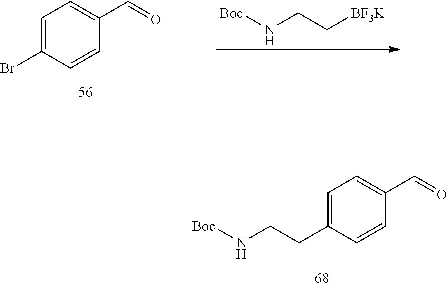

To solution of 4-bromobenzaldehyde (3.0 g, 16.3 mmol) in toluene (25 mL) in a sealed tube were added potassium tert-butyl N-[2-(trifluoroboranuidyl)ethyl]carbamate (4.9 g, 19.6 mmol), Cs₂CO₃ (15.9 g, 48.9 mmol), PdCl2 (dppf)·CH₂Cl₂ (2.6 g, 3.2 mmol) and water (5 mL). The reaction mixture was degassed under vacuum and filled with nitrogen gas, the process repeated three times. The tube with reaction mixture was sealed and heated to 80° C. for 18 h. The reaction was allowed to cool to room temperature, saturated solution of NH₄Cl (20 mL) was added, and the suspension was extracted with CH₂Cl₂ (30 mL×3 times). The organic extracts were combined, dried (Na₂SO₄), and concentrated under reduced pressure to provide crude compound. Purification by flash chromatography (24 g silica cartridge, EtOAc/Hexane) gave the title compound (3.3 g, 73% yield). ¹H NMR (CHLOROFORM-d, 300 MHz): δ=9.93-10.04 (m, 1H), 7.74-7.90 (m, 2H), 7.32-7.42 (m, 2H), 4.39-4.63 (m, 1H), 3.23-3.54 (m, 2H), 2.73-3.02 (m, 2H), 1.52-1.60 (m, 1H), 1.43 ppm (s, 9H). (ESI) m/z 273 (M+23)⁺.

### Step 2: Ethyl-3-(4-(2-((tert-butoxycarbonyl)amino)ethyl)phenyl)-(E/Z)-acrylate, 69

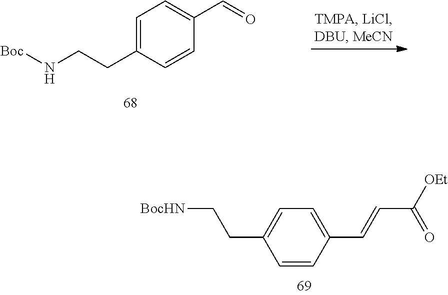 [SMILES: CC(C)(C)OC(=O)NCCc1ccc(C=O)cc1 ; CCOC(=O)/C=C/c1ccc(CCNC(=O)OC(C)(C)C)cc1]

Tert-butyl (4-formylphenethyl) carbamate (3.3 g, 13.5 mmol) was dissolved in acetonitrile (15 mL) and added ethyl 2-(diethoxyphosphoryl)acetate (9.6 g, 2.4 mmol), lithium chloride (1.1 g, 27.1 mmol), 1,8-diazabicyclo 5.4.0 undec-7-ene (1.3 g, 3.9 mmol) added, the reaction mixture was maintained at room temperature for 18 h. After completion of reaction, the reaction mixture was concentrated and added CH₂Cl₂ (30 mL) and H₂O (30 mL) the suspension was extracted with CH₂Cl₂ (3×30 mL). The organic extracts were combined, washed with brine (20 mL), dried (Na₂SO₄), and concentrated under reduced pressure to provide crude compound. Purification by flash chromatography (40 g silica cartridge, EtOAc/Hexane) gave the title compound (2.6 g, 60% yield). ¹H NMR (CHLOROFORM-d, 300 MHz): δ=7.66 (d, J=15.8 Hz, 4H), 7.46 (d, J=8.2 Hz, 7H), 7.17-7.28 (m, J=11.1 Hz, 9H), 6.40 (d, J=15.8 Hz, 4H), 4.26 (q, J=7.0 Hz, 7H), 3.38 (q, J=6.2 Hz, 7H), 2.81 (t, J=7.0 Hz, 7H), 1.43 (s, 32H), 1.33 ppm (t, J=7.0 Hz, 12H). (ESI) m/z 343 (M+23)⁺.

### Step 3: Synthesis of ethyl 3-(4-(2-((tert-butoxycarbonyl)amino)ethyl)phenyl)-4-nitrobutanoate, 70

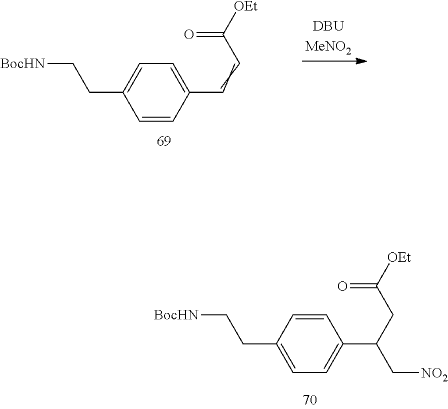 [SMILES: CCOC(=O)C=Cc1ccc(CCNC(=O)OC(C)(C)C)cc1 ; CCOC(=O)CC(C[N+](=O)[O-])c1ccc(CCNC(=O)OC(C)(C)C)cc1]

The starting material ethyl-3-(4-(2-((tert-butoxycarbonyl)amino)ethyl)phenyl)-(E/Z)-acrylate (1.3 g, 3.9 mmol) was dissolved in nitromethane (6.5 mL) and added 1,8-diazabicyclo 5.4.0 undec-7-ene (1.6 g, 3.9 mmol), the reaction mixture was maintained at room temperature for 18 h. After completion of reaction, the reaction mixture was diluted with ethyl acetate (20 mL) and washed with 1N HCl (15 mL×2 times) the combined aqueous layers were reextracted with ethyl acetate (15 mL). The organic extracts were combined again washed with 1N HCl (10 mL), dried (Na₂SO₄), and concentrated under reduced pressure to provide crude. Purification by flash chromatography (40 g silica cartridge, 30% EtOAc in Hexane) gave the title compound (0.8 g, 49% yield). (ESI) m/z 382 (M+1)⁺.

### Step 4: Synthesis of ethyl 3-(4-(2-aminoethyl)phenyl)-4-nitrobutanoate hydrochloride, 71

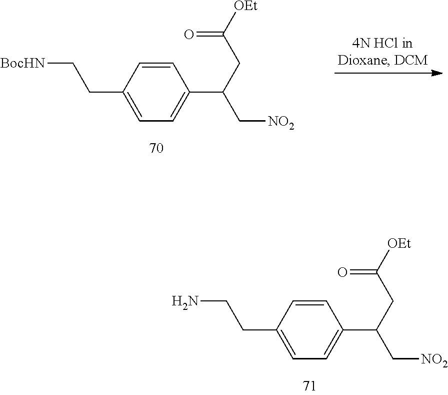 [SMILES: CCOC(=O)CC(C[N+](=O)[O-])c1ccc(CCNC(=O)OC(C)(C)C)cc1 ; CCOC(=O)CC(C[N+](=O)[O-])c1ccc(CCN)cc1]

The ethyl 3-(4-(2-((tert-butoxycarbonyl)amino)ethyl)phenyl)-4-nitrobutanoate (1.3 g, 3.4 mmol) was dissolved in dichloromethane (10 mL) and added 4N HCl (3 mL). The reaction was maintained at room temperature overnight. Reaction was monitored by LC-MS. After completion of reaction, the reaction mixture was concentrated in vacuo and resultant solids were washed with ether to get desired compound in quantitative yield (0.85 g, 88.7% yield). ¹H NMR (METHANOL-d₄, 300 MHz): δ=7.16-7.42 (m, 4H), 4.82 (s, 2H), 3.83-4.13 (m, 2H), 3.43-3.54 (m, 1H), 3.09-3.23 (m, 2H), 2.88-2.99 (m, 2H), 2.65-2.87 (m, 2H), 1.15 ppm (s, 3H). (ESI) m/z 282 (M+1)⁺.

### Step 5: Synthesis of ethyl 3-(4-(2-((1-methylethyl) sulfonamido)ethyl)phenyl)-4-nitrobutanoate, 72

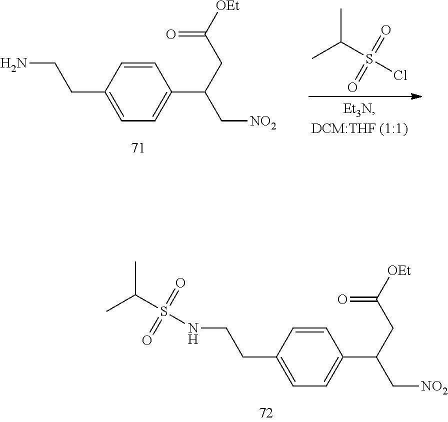

The starting material ethyl 3-(4-(2-aminoethyl)phenyl)-4-nitrobutanoate HCl (397 mg, 1.42 mmol) in CH₂Cl₂:THF (8 mL, 1:1) was cooled to 0° C. To reaction mixture added triethylamine (418 uL, 3.0 mmol) and then isopropyl sulfonyl chloride (221 uL, 1.5 mmol) drop wise. The reaction mixture progress was monitored by LC-MS. The reaction was maintained at room temperature for 18 h. To reaction mixture added EtOAc and the suspension was extracted with EtOAc (30 mL×3 times). The organic extracts were combined, dried (Na₂SO₄), and concentrated under reduced pressure to provide crude compound. Purification by flash chromatography (24 g silica cartridge, 10% MeOH in CH₂Cl₂) gave the title compound (207 mg, 45%). (ESI) m/z 388 (M+1)⁺.

### Step 6: Synthesis of ethyl 4-amino-3-(4-(2-((1-methylethyl) sulfonamido)ethyl)phenyl) butanoate, 73

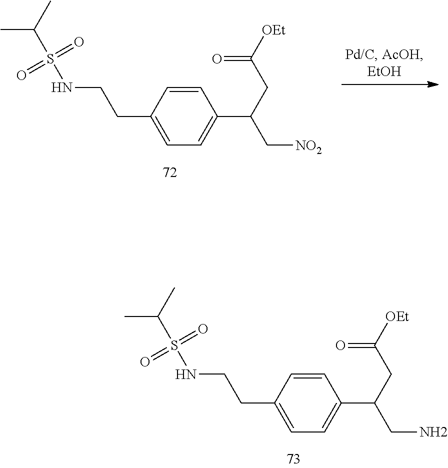 [SMILES: CCOC(=O)CC(C[N+](=O)[O-])c1ccc(CCNS(=O)(=O)C(C)C)cc1 ; CCOC(=O)CC(CN)c1ccc(CCNS(=O)(=O)C(C)C)cc1]

To a solution of ethyl 3-(4-(2-((1-methylethyl) sulfonamido)ethyl)phenyl)-4-nitrobutanoate (200 mg, 0.51 mmol) in ethanol, under nitrogen atmosphere added 10% Pd/C (110.6 mg, 0.15 mmol) and setup was placed on par hydrogenator and maintained under hydrogen atmosphere 50 psi for 18 hours. The reaction was checked by LC-MS and after complete conversion, the reaction mixture was filtered through celite and concentrated in vacuo to get crude compound (82 mg, 44%). The crude product was used as is without further purification. (ESI) m/z 358 (M+1)⁺.

### Step 7: Synthesis of ethyl 4-((1-methylethyl) sulfonamido)-3-(4-(2-((1-methylethyl) sulfonamido)ethyl)phenyl) butanoate, 62

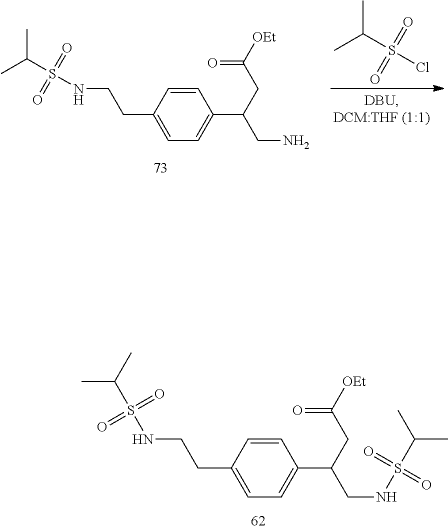

The starting material ethyl 4-amino-3-(4-(2-((1-methylethyl) sulfonamido)ethyl)phenyl) butanoate (67 mg, 0.189 mmol) in CH₂Cl₂:THF (1 mL, 1:1) was cooled to 0° C. 1,8-Diazabicyclo 5.4.0 undec-7-ene (68 mg, 0.45 mmol), and isopropyl sulfonyl chloride (59 mg, 0.41 mmol) were then added. The reaction was maintained at room temperature for 18 h with stirring. To the reaction mixture was added HCl (5 mL) and EtOAc (10 mL) and the suspension was extracted with EtOAc (3×10 mL). The organic extracts were combined, dried (Na₂SO₄), and concentrated under reduced pressure to provide crude compound. Purification by flash chromatography (12 g silica cartridge, 10% MeOH in CH₂Cl₂) to give the title compound (35 mg, 40%). ¹H NMR (CHLOROFORM-d, 300 MHz): δ=7.17 (s, 4H), 4.19-4.33 (m, 2H), 4.00-4.13 (m, 2H), 3.27-3.43 (m, 5H), 2.99-3.18 (m, 2H), 2.56-2.88 (m, 4H), 1.30 (d, J=7.0 Hz, 12H), 1.14-1.21 ppm (m, 3H). (ESI) m/z 464 (M+1)⁺.

### Step 8: Synthesis of 4-((1-methylethyl) sulfonamido)-3-(4-(2-((1-methylethyl) sulfonamido)ethyl)phenyl) butanoic acid, 74

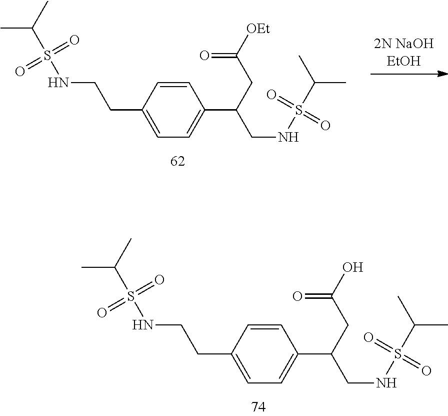

The starting material ethyl 4-((1-methylethyl) sulfonamido)-3-(4-(2-((1-methylethyl) sulfonamido)ethyl)phenyl) butanoate (33 mg, 0.09 mmol) was dissolved in ethanol (0.5 mL). To this solution, sodium hydroxide (36.8 mg, 0.87 mmol) was added and reaction stirred at room temperature for 3 h. After completion of reaction, the reaction mixture was concentrated and CH₂Cl₂ and 1N HCl (3 mL) were added. The suspension was extracted with CH₂Cl₂ (3×5 mL). The organic extracts were combined dried (Na₂SO₄), and concentrated under reduced pressure to provide crude compound, which is used as is for next step. ¹H NMR (METHANOL-d₄, 300 MHz): δ=7.17-7.31 (m, 4H), 3.35 (s, 1H), 3.30 (br. s., 4H), 3.07 (dq, J=13.5, 6.8 Hz, 2H), 2.78-2.94 (m, J=7.0 Hz, 3H), 2.52-2.69 (m, J=4.7 Hz, 1H), 1.18-1.34 ppm (m, 12H). (ESI) m/z 436 (M+1)⁺.

### Step 9: Synthesis of 4-((1-methylethyl) sulfonamido)-3-(4-(2-((1-methylethyl) sulfonamido)ethyl)phenyl)-N-(3-((2-(prop-2-yn-1-yloxy)ethyl)amino) propyl) butanamide, 75

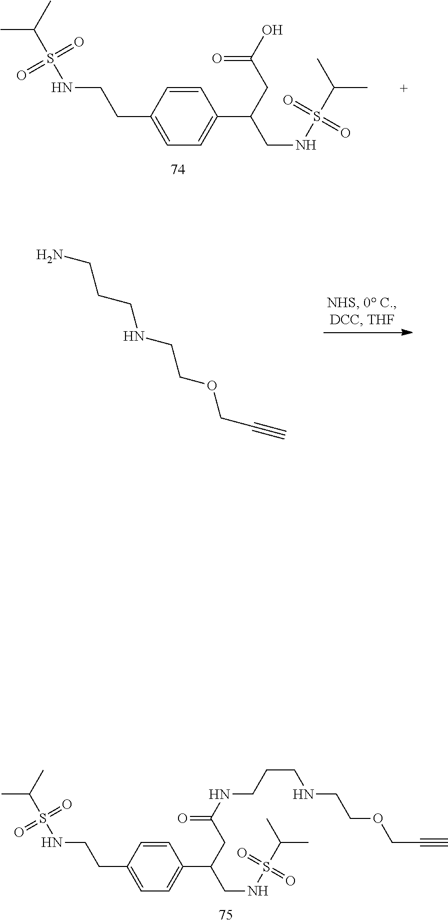

A solution of 4-((1-methylethyl) sulfonamido)-3-(4-(2-((1-methylethyl) sulfonamido)ethyl)phenyl) butanoic acid (30.2 mg, 0.07 mmol) and N-hydroxysuccinimide (8 mg, 0.07 mmol) in dry THF (0.3 mL) was cooled to 0° C. in an ice bath. Dicyclohexylcarbodiimide (17.9 mg, 0.09 mmol) dissolved in dry THF (0.5 mL) was added drop wise to the mixture and the reaction stirred at room temperature for 24 h. The reaction mixture was filtered to remove dicyclohexylurea by-product formed. The filtrate was concentrated under reduced pressure. The residue was dissolved in CHCl₃ and washed consecutively with water, a saturated solution of NaHCO₃ and water. The organic layer was dried (Na₂SO₄), and concentrated under reduced pressure to obtain the N-hydroxysuccinimide active ester intermediate. This intermediate (37.0 mg, 0.07 mmol) and N1-(2-(prop-2-yn-1-yloxy)ethyl) propane-1,3-diamine (20.0 mg, 0.07 mmol) were dissolved in CHCl₃ (0.3 mL) with addition of small amount of DMF (35 uL). The reaction stirred at room temperature for 48 h. After completion of reaction, the reaction mixture was washed with H₂O, sat. NaHCO₃, and H₂O. filtered off the desiccant, dried over Na₂SO₄, concentrated and purified by reverse phase preparative HPLC (mobile phase: 0.1% TFA in acetonitrile and 0.1% TFA in water) to give pure compound 9.0 mg, 23% yield. ¹H NMR (METHANOL-d₄, 300 MHZ): δ=7.23 (s, 4H), 4.22-4.30 (m, 2H), 3.75 (t, J=5.0 Hz, 2H), 3.24 (br. s., 4H), 2.93-3.17 (m, 6H), 2.71-2.87 (m, J=7.0, 7.0 Hz, 4H), 2.35-2.58 (m, J=14.4, 9.1 Hz, 2H), 1.57-1.80 (m, 2H), 1.16-1.37 ppm (m, 12H). (ESI) m/z 574 (M+1)⁺.

### Example 18

### Synthesis of reagent CMPDA.2^{DART.2} (R16)

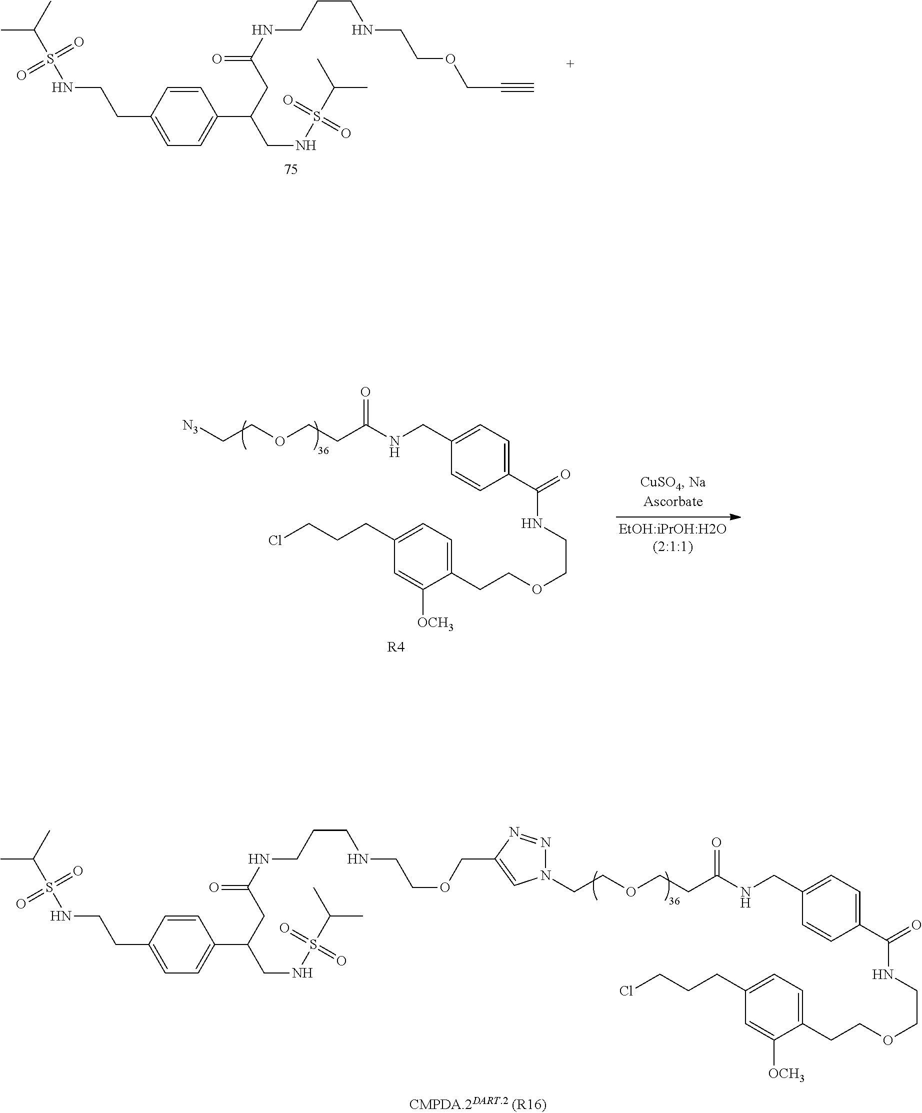

To a vial containing the azido-PEG36-HTL.2 (^{azido}DART.2, R4) (28.4 mg, 0.013 mmol) and 4-((1-methylethyl) sulfonamido)-3-(4-(2-((1-methylethyl) sulfonamido)ethyl)phenyl)-N-(3-((2-(prop-2-yn-1-yloxy)ethyl)amino) propyl) butanamide (7.8 mg, 0.013 mmol), the solvent mixture EtOH:iPrOH:H₂O (2:1:1) (200 uL) was added followed by CuSO₄·5H₂O (2.39 mg, 0.015 mmol) and (+)-sodium L-ascorbate (3.0 mg, 0.015 mmol) was pre-dissolved in solutions of EtOH:iPrOH:H₂O (2:1:1) (400 uL). The reaction was stirred for 18 h at room temperature. After completion of reaction, reaction mixture was filtered, concentrated and purified by reverse phase preparative HPLC (mobile phase: 0.1% TFA in acetonitrile and 0.1% TFA in water) to get pure compound R16 17.0 mg, 47% yield. ¹H NMR (300 MHZ, METHANOL-d₄) δ ppm 8.19-8.25 (m, 1H) 7.84-7.97 (m, 2H) 7.51 (d, J=7.03 Hz, 2H) 7.33 (s, 4H) 7.17 (d, J=7.03 Hz, 1H) 6.87 (br. s., 1H) 6.76 (d, J=7.62 Hz, 1H) 4.86 (s, 1H) 4.80 (br. s., 1H) 4.71 (br. s., 1H) 4.57 (br. s., 2H) 3.99 (m, J=3.51 Hz, 3H) 3.83-3.94 (m, 8H) 3.57-3.83 (m, 158H) 3.20 (m, J=11.72 Hz, 4H) 2.88 (m, J=7.62 Hz, 8H) 2.50-2.70 (m, 4H) 2.14 (t, J=6.44 Hz, 2H) 1.81 (br. s., 2H) 1.37 (m, J=2.34 Hz, 12H); (ESI) m/z 1332 (M/2+1)⁺; HRMS (ESI): m/z calcd for C₁₂₃H₂₂₁ClN₉O₄₆S₂: 2659.4341 [M+H]⁺; found: 2659.43551, [M+H]⁺.

### Example 19

### Improvement Over HTL.1

Various compounds were assessed by measuring their EC50 (nM); the concentration needed to achieve half-maximal capture on +HTP neurons following 15 minutes of incubation with the given ligand.

| Structure | EC 50 (nM) | |
|---|---|---|
| 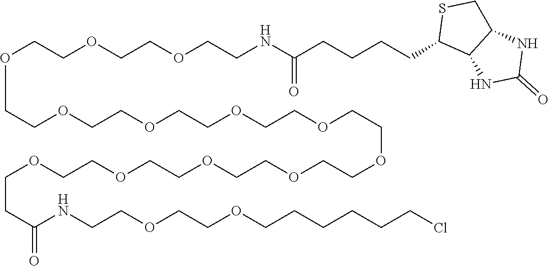 [SMILES: O=C(CCCC[C@@H]1SC[C@@H]2NC(=O)N[C@@H]21)NCCOCCOCCOCCOCCOCCOCCOCCOCCOCCOCCOCCOCCC(=O)NCCOCCOCCCCCCCl] | 1,000 | nM |
| 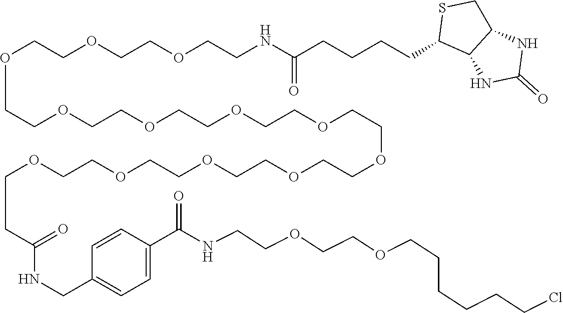 [SMILES: O=C(CCCC[C@@H]1SC[C@@H]2NC(=O)N[C@@H]21)NCCOCCOCCOCCOCCOCCOCCOCCOCCOCCOCCOCCOCCC(=O)NCc1ccc(C(=O)NCCOCCOCCCCCCCl)cc1] | 150 | nM |
| 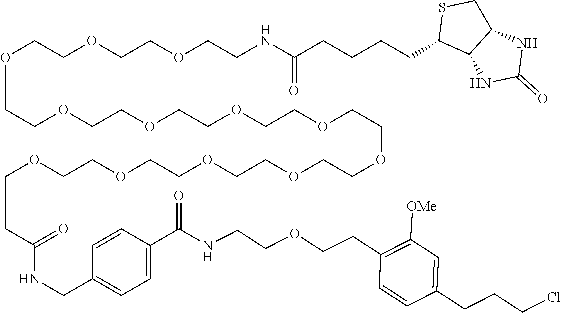 [SMILES: COc1cc(CCCCl)ccc1CCOCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 10 | nM |
| 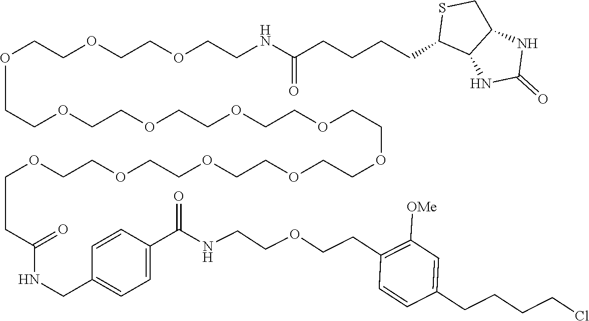 [SMILES: COc1cc(CCCCCl)ccc1CCOCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 30 | nM |
| 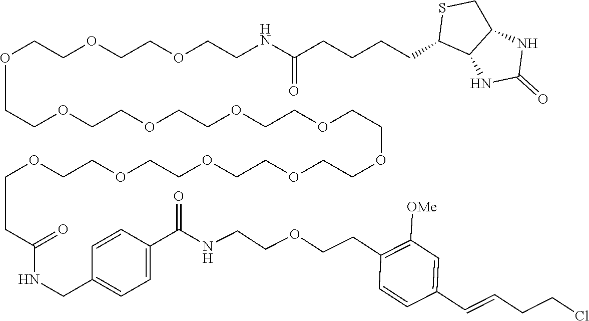 [SMILES: COc1cc(/C=C/CCCl)ccc1CCOCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 20 | nM |
| 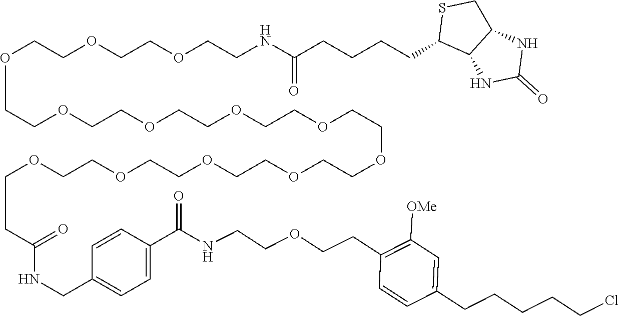 [SMILES: COc1cc(CCCCCCl)ccc1CCOCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 4,000 | nM |
| 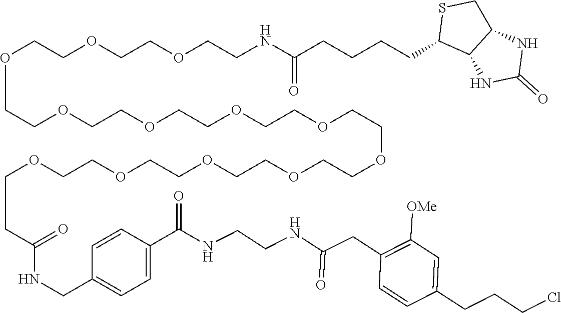 [SMILES: COc1cc(CCCCl)ccc1CC(=O)NCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 20,000 | nM |
| 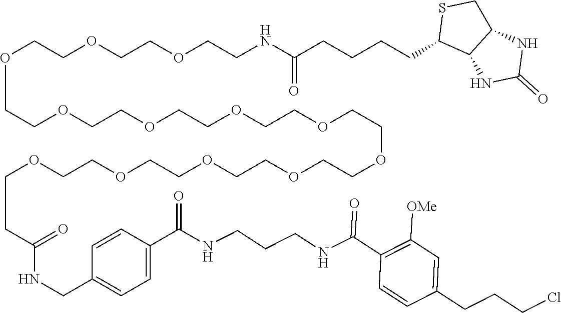 [SMILES: COc1cc(CCCCl)ccc1C(=O)NCCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 200 | nM |
| 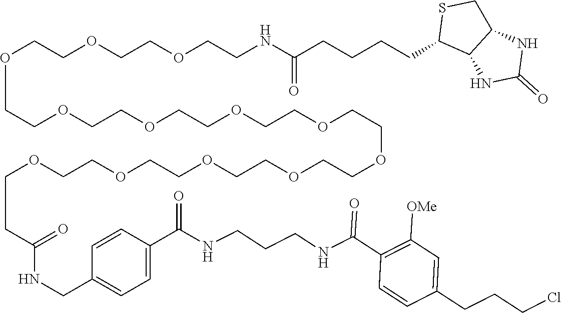 [SMILES: COc1cc(CCCCl)ccc1C(=O)NCCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 280 | nM |
| 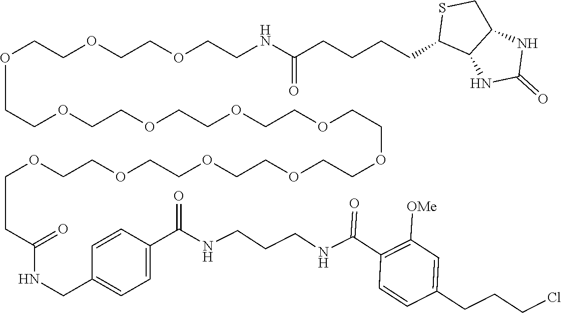 [SMILES: COc1cc(CCCCl)ccc1C(=O)NCCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 200 | nM |
| 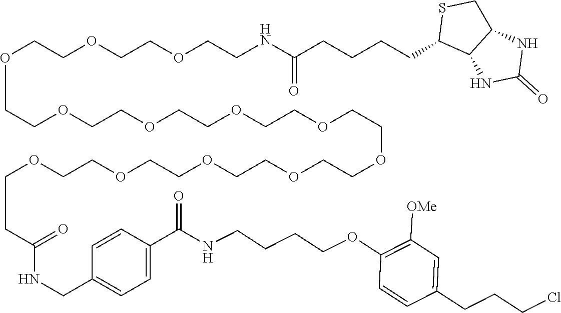 [SMILES: COc1cc(CCCCl)ccc1OCCCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 50 | nM |
| 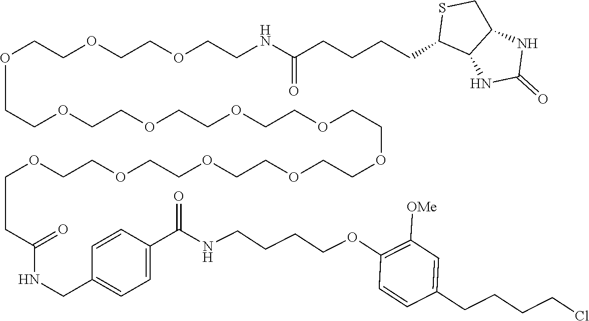 [SMILES: COc1cc(CCCCCl)ccc1OCCCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 30 | nM |
| 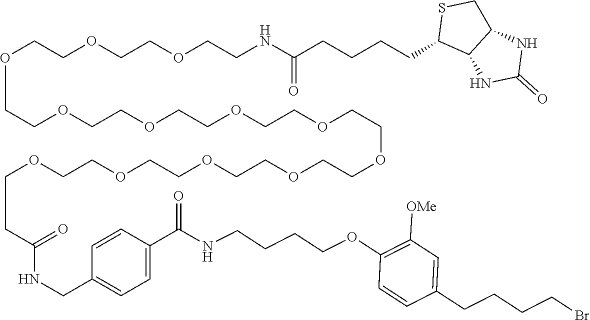 [SMILES: COc1cc(CCCCBr)ccc1OCCCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 30 | nM |
| 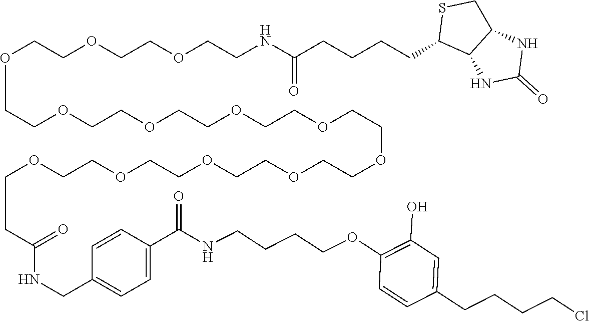 [SMILES: O=C(CCCC[C@@H]1SC[C@@H]2NC(=O)N[C@@H]21)NCCOCCOCCOCCOCCOCCOCCOCCOCCOCCOCCOCCOCCC(=O)NCc1ccc(C(=O)NCCCCOc2ccc(CCCCCl)cc2O)cc1] | 30 | nM |
| 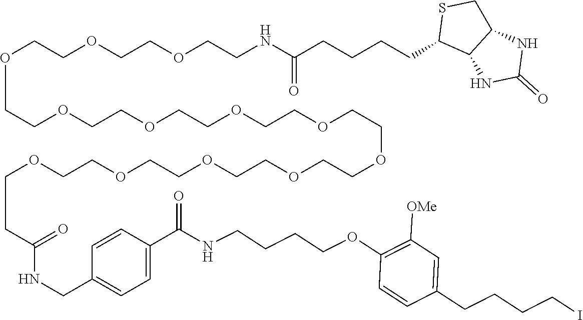 [SMILES: COc1cc(CCCCI)ccc1OCCCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 30 | nM |
| 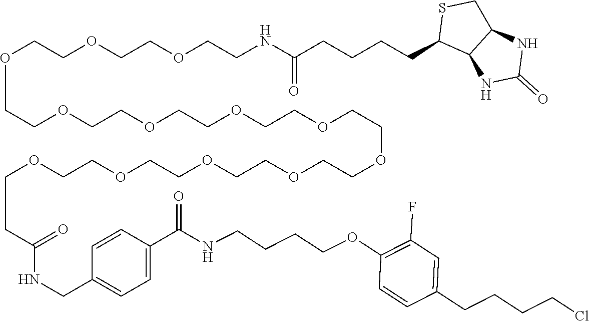 [SMILES: O=C(CCCC[C@H]1SC[C@H]2NC(=O)N[C@H]21)NCCOCCOCCOCCOCCOCCOCCOCCOCCOCCOCCOCCOCCC(=O)NCc1ccc(C(=O)NCCCCOc2ccc(CCCCCl)cc2F)cc1] | 45 | nM |
| 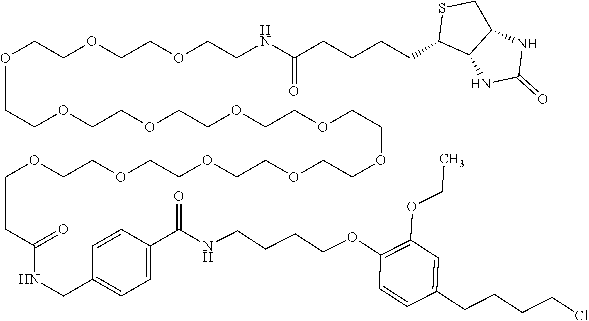 [SMILES: CCOc1cc(CCCCCl)ccc1OCCCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 70 | nM |
| 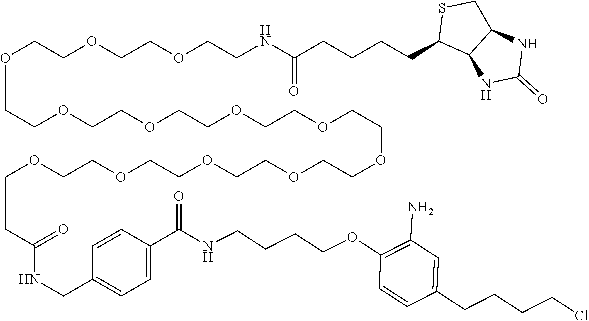 [SMILES: Nc1cc(CCCCCl)ccc1OCCCCNC(=O)c1ccc(CNC(=O)CCOCCOCCOCCOCCOCCOCCOCCOCCOCCOCCOCCOCCNC(=O)CCCC[C@H]2SC[C@H]3NC(=O)N[C@H]32)cc1] | 70 | nM |
| 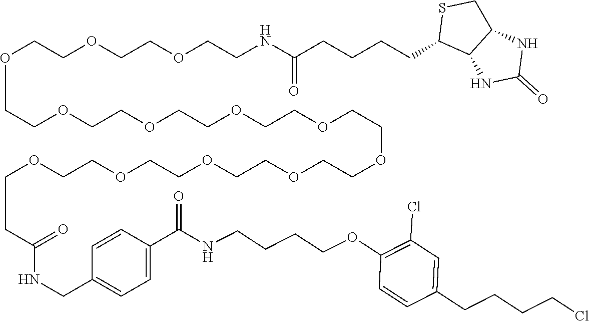 [SMILES: O=C(CCCC[C@@H]1SC[C@@H]2NC(=O)N[C@@H]21)NCCOCCOCCOCCOCCOCCOCCOCCOCCOCCOCCOCCOCCC(=O)NCc1ccc(C(=O)NCCCCOc2ccc(CCCCCl)cc2Cl)cc1] | 100 | nM |
| 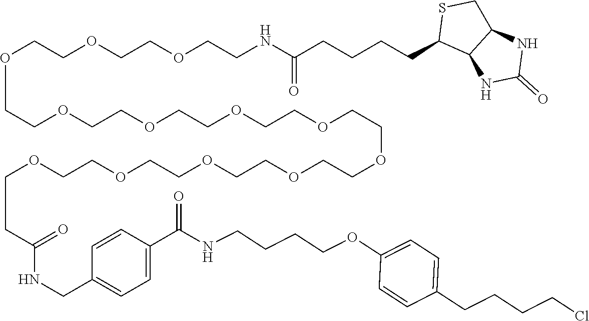 [SMILES: O=C(CCCC[C@H]1SC[C@H]2NC(=O)N[C@H]21)NCCOCCOCCOCCOCCOCCOCCOCCOCCOCCOCCOCCOCCC(=O)NCc1ccc(C(=O)NCCCCOc2ccc(CCCCCl)cc2)cc1] | 110 | nM |
| 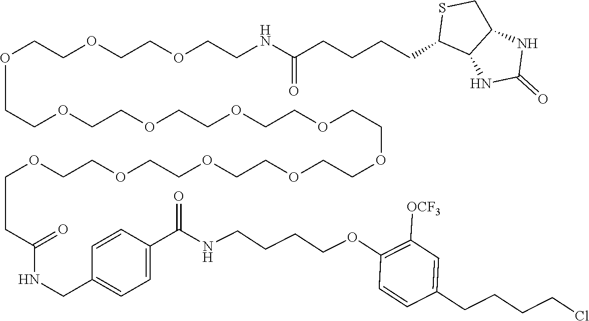 [SMILES: O=C(CCCC[C@@H]1SC[C@@H]2NC(=O)N[C@@H]21)NCCOCCOCCOCCOCCOCCOCCOCCOCCOCCOCCOCCOCCC(=O)NCc1ccc(C(=O)NCCCCOc2ccc(CCCCCl)cc2OC(F)(F)F)cc1] | 1,500 | nM |
| 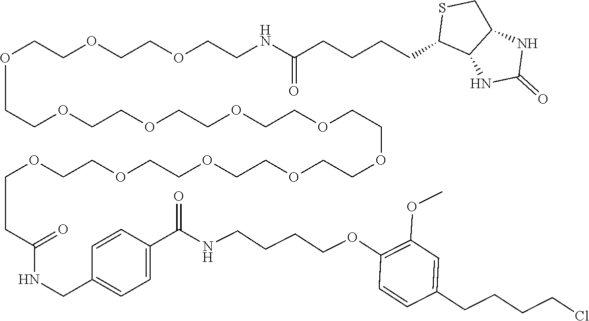 [SMILES: COc1cc(CCCCCl)ccc1OCCCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 3,000 | nM |
| 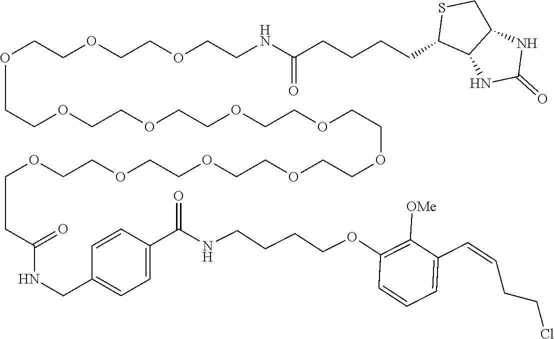 [SMILES: COc1c(/C=C\CCCl)cccc1OCCCCNC(=O)c1ccc(CNC(=O)CCOCCOCCOCCOCCOCCOCCOCCOCCOCCOCCOCCOCCNC(=O)CCCC[C@@H]2SC[C@@H]3NC(=O)N[C@@H]32)cc1] | 55 | nM |

### Example 20

### Principles of DART Dosing Window

DART exploits differences in Rx vs HTL characteristics to impact ⁺HTP (genetically programmed, on-target) cells while sparing ⁻HTP (non-programmed, off-target) cells. A key difference is that HTP cells can only interact with ambient Rx via low-affinity reversible binding to native receptors. In contrast, ⁺HTP cells covalently bind the HTL moiety to irreversibly accumulate tethered Rx as time proceeds. This difference is manifested by the units of dosing, with concentration (nM) used for ⁻HTP cells, and integrated concentration (nM×min) for ⁺HTP cells. The metrics of cell-specificity thus become:
Rx₅₀ (nM)—50% off-target effect, ⁻HTP cells (concentration)
HTL₅₀ (nM×min)—50% on-target effect, ⁺HTP cells (integrated concentration)
TV=Rx₅₀/HTL₅₀ (fold/min)—Therapeutic Velocity
TI₍ₜ₎=TV×t (fold)—Therapeutic Index

For example, given a reagent in which off-target effects occur at Rx₅₀=10,000 nM, and on-target covalent tethering requires HTL₅₀=5,000 nM×min, the ratio of these terms specifies the Therapeutic Velocity, TV=2-fold/min, a constant that describes the rate at which cellular specificity is achieved. The textbook quantity, Therapeutic Index (TI), thus grows with time: at 15 min, TI₁₅ₘ=30-fold; and at 60 min, TI₆₀ₘ=120-fold.

A point that merits clarification is that TI can, in principle, become arbitrarily large with t. This is because experiments that permit a longer, pre-allocated incubation time, would permit tethering at a proportionally lower concentration. The technical limit to trading time for concentration is degradation of HTP after it has captured an Rx^{DART}; this limits linear regime to ˜tens of hours (Shields et al., 2017, which is incorporated by reference herein in its entirety). However, faster manipulations are often needed for other reasons. In particular, electrophysiology is difficult beyond 1 hr, and behavioral causality is more interpretable if immediate effects (minutes) can be disambiguated from slower changes (hours to days).

### Example 21

### Development of DART.2 Platform

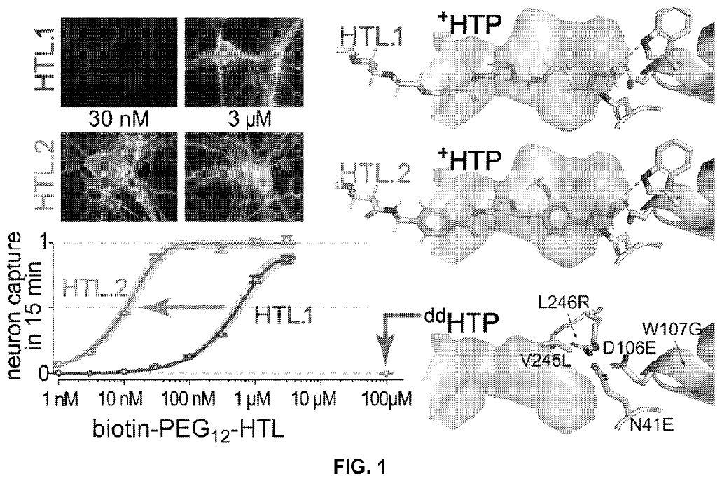

To enable direct quantification of capture efficiency, commercially available HTL (i.e., HTL.1) were conjugated to biotin via a flexible polyethylene glycol linker of moderate length (PEG12), to mimic the longer (PEG36) linker typically used in an RxDART. Capture kinetics were characterized on cultured neurons expressing HTP on their surface [HTPNLG(432-648), as in, to model the cellular microenvironment of interest. Dose-response curves were performed by incubating neuronal coverslips with a given ligand for 15 min, followed by washing, fluorescent labeling of biotin, and quantification of capture. It was found that capture efficiency was relatively poor, with little if any capture in the low-nanomolar range (FIG. 1A). Thus, it was reasoned that improving HTL50 would widen the DW for any Rx cargo.

To devise an improved strategy, the atomic structure of the HTP in complex with HTL.1 was examined. A few stabilizing contacts were noted between the straight-chain portion of HTL.1 and the inner surface of the HTP tunnel, and hypothesized, without being bound by a particular theory, that a transiently docked HTL.1 (FIG. 1A) may tend to exit rather than react, requiring numerous attempts for covalent capture. To test this hypothesis, the HTL was modified to improve shape/charge complementarity to the HTP tunnel. The strategy was conceptually similar to a modular “synthon-based” approach, wherein a list of candidate minimal moieties were assembled (FIG. 1A), and focused the synthetic effort towards improvement of short interconnecting linkers. The best variant, named HTL.2, contains non-trivial modifications to the original design (FIG. 1A). In side-by-side capture experiments on cultured neurons, HTL.2 features an HTL50 (t=15 m)=10 nM, which outperformed HTL.1 by 40-fold (FIG. 1A).

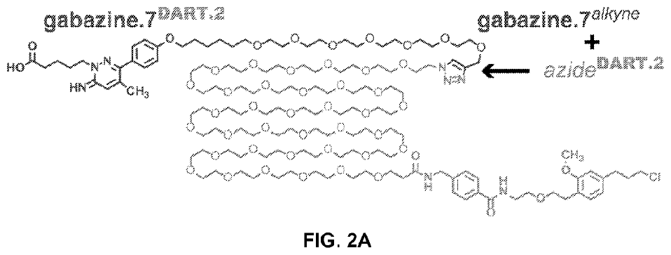

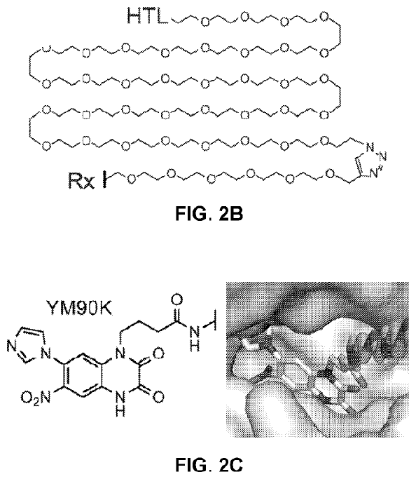

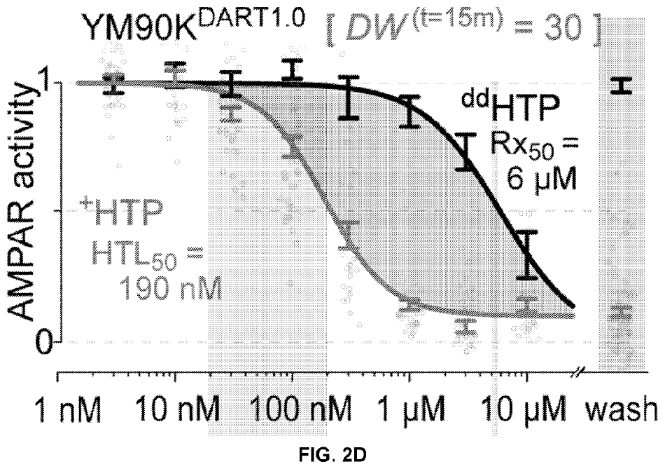

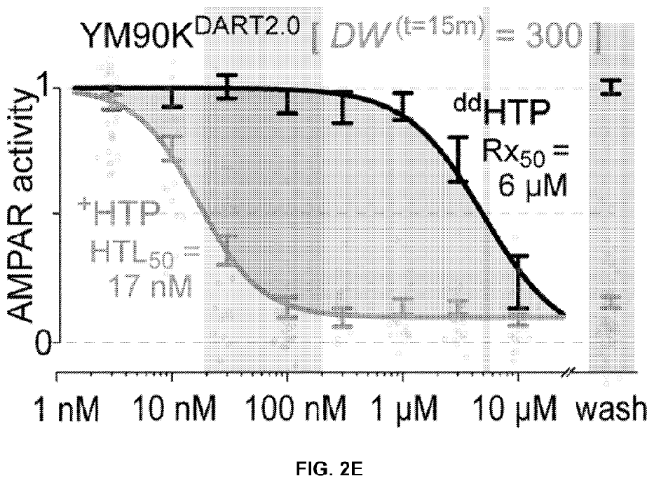

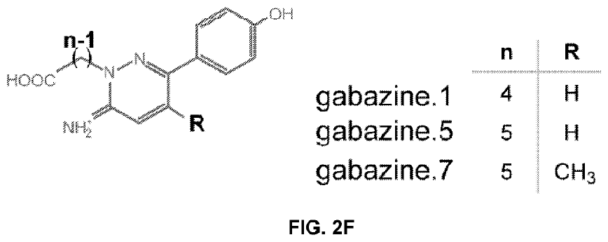

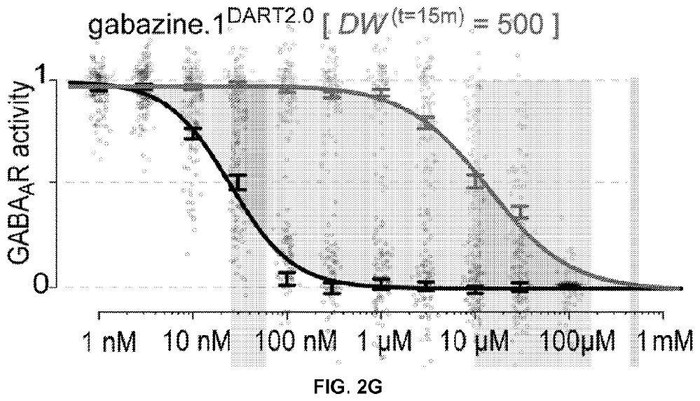

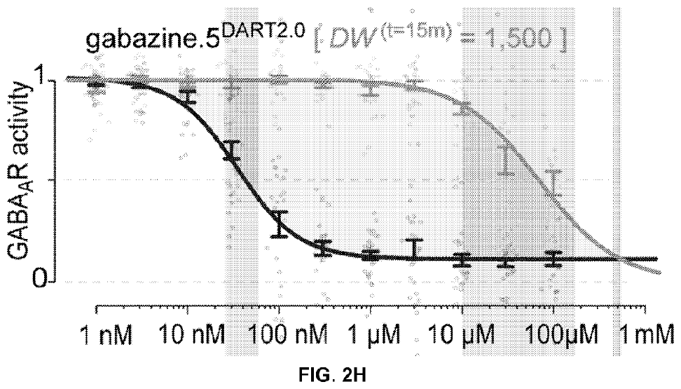

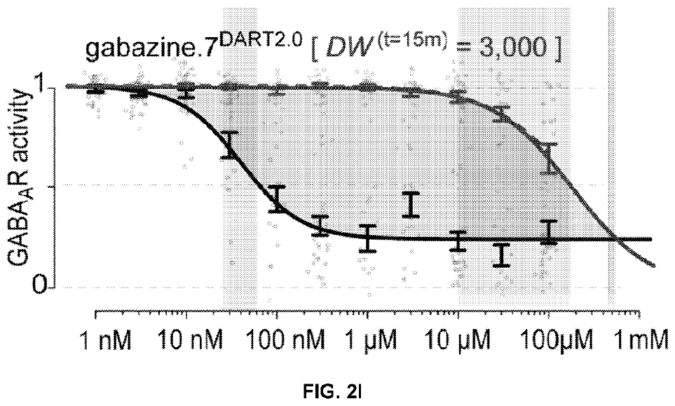

A core advance of DART.2 is widening of the dosing window, which was achieved via two parallel approaches: lowering HTL50 and raising Rx50. Although intuitive, certain aspects of the data differed in quantitative detail from the expectations, and thus merit further discussion. For example, it was expected that an HTL.1 to HTL.2 upgrade would yield an equivalent improvement in HTL50 regardless of the payload. However, in contrast to the 40-fold HTL50 (t=15 m) improvement with a biotin payload (FIG. 1), only a 10-fold HTL50 (t=15 m) change was observed when delivering a pharmaceutical YM90K payload (FIG. 2C, FIG. 2D and FIG. 2E). This discrepancy could not be simply explained, given identical Rx50 for the two variants. It was confirmed that a fresh synthesis of biotin HTL.1 exhibited similar performance, mitigating concerns that its poor capture efficiency could have been an artifact. Thus, a definitive explanation has yet to be determined. One possibility is that the HTL.1 moiety may be so inefficient that the YM90K moiety may offer some assistance, either via transient interactions with the AMPAR, or perhaps via nonspecific interactions with the HTP surface. Indeed, similar assistance has been reported with aromatic dye payloads, which have been reported to interact with micromolar affinity to the surface of the HTP. Such assistance would become negligible with HTL.2, which drives robust capture at substantially lower doses.

### Example 22

Development of ddHTP

To account for any potential impact of HTP expression on cellular physiology, a control protein was developed that does not bind HTL.2, while retaining all other characteristics of its active counterpart. Mutation of the HTP at its D106 residue was first explored, to which HTL forms a covalent bond. However, it was found that any single mutation at this site either retained residual catalysis (e.g., D106E) or disrupted surface trafficking in cultured neurons (e.g., D106A). Next, the distributed mutations of the catalytic triad was explored, combining the subtle D106E mutation with W107G and N41E mutations. A second strategy was also explored, in which the tunnel entrance was blocked by installing bulky side-chains, with V245L and L246R mutations. Combining the two strategies yielded a “double dead” protein (ddHTP), which exhibited zero measurable binding to HTL.2, while maintaining proper folding and trafficking (FIG. 1).

### Example 23

### Modular Chemistry and Naming Convention

To facilitate expansion of the DART toolset, a standardized click-chemistry approach was used wherein “drug modules” are appended to an alkyne (C≡C) moiety, and “capture modules” are attached to an azido (N=N+=N−) moiety (FIG. 2A and FIG. 2B). Click chemistry allows these modules to be assembled in a single step, without the need for harsh conditions or chemical protecting groups, which can complicate traditional coupling strategies. Thus, the approach facilitates parallel development of components by independent chemistry teams.

To standardize naming and provide clarity as reagents evolve, a version number was assigned to each module. Thus, first drug module was named YM90K.1alkyne, designating it as the first attempt, wherein the drug was attached to an alkyne via a short spacer. If a second attempt were needed, it would be named YM90K.2alkyne, and so on. The use of a period separating the drug name from the version number was used because many reagents have names that end in a number (e.g., Alexa647.1alkyne).

The capture modules were next developed, keeping a similar convention for version numbers. It was decided that capture modules would contain the HTL moiety along with a PEG36 linker. This linker was found to be long enough to span the distance from the HTP to native receptors on the cell surface, yet short enough to prevent impact of a tethered drug on neighboring cells. Thus, an azido PEG36 HTL.1 capture module was synthesized, concisely named DART.1, and an azido-PEG36 HTL.2 module, named DART.2. Click-chemistry conjugation of YM90K.1alkyne to these capture modules yielded YM90K.1DART.1 and YM90K.1DART.2. Thus, following assembly, the “alkyne” superscript is replaced with the name of the capture module, indicating conversion from a small drug module to a full-length DART reagent.

Identification of a specific reagent requires two numbers (YM90K.1^{DART.2}, spoken “YM90K point 1 DART point 2”). For a class of reagents, one could omit the number for the varied element. Thus, a set of reagents with variable capture module would be named YM90K.1DART (variable element: DART.1, DART.2, DART.3). Likewise, a drug-variant set of reagents would be named YM90KDART.2 (variable element: YM90K.1, YM90K.2, YM90K.3).

### Example 24

### Improving the Dosing Window

Homologous YM90K.1DART.1 and YM90K.1^{DART.2} reagents were assembled, and their impact was assayed on endogenous AMPARs with an all-optical neuronal assay. Primary rat hippocampal neurons were dissociated, split into groups, electroporated separately with different constructs, and co-cultured to enable optical read/write operations. The “presynaptic” group of neurons expressed ChR2, while the intermingled “postsynaptic” group of neurons expressed GCaMP6s along with either the active +HTP or control ddHTP. Assays were performed under conditions that isolate AMPAR signaling, with other synaptic receptors blocked. Thus, GCaMP6s signals reflect the activation of endogenous AMPARs via synaptic neurotransmission. It was confirmed that +HTP vs ddHTP cells exhibited no difference in baseline GCaMP signals, or in dose-response characterizations of a traditional AMPAR antagonist. These parameters were also similar in −HTP neurons, consistent with unperturbed endogenous AMPARs. Similar to prior results, YM90K.1DART.1 reproduced a DW (t=15 m)=30, with tethered-drug effects on +HTP cells occurring at a 30-fold lower dose than needed to produce ambient-drug effects on ddHTP cells. In parallel coverslips from the same neural prep, YM90K.1DART.2 provided a DW (t=15 m)=300, which outperformed YM90K.1DART.1 by an order of magnitude (FIG. 2D and FIG. 2E). The performance boost could be attributed to improvement in HTL50, with no impact on Rx50, given identical YM90K.1 moieties.

It was next focused on blocking the GABAAR, a ubiquitous transducer of synaptic inhibition. Given the risk of transient seizure induction, improvement of Rx50 would likely be needed, in concert with HTL50 improvement. The structure-activity literature of gabazine, a selective GABAAR antagonist, was explored. The core gabazine pharmacophore includes of a 3-amino, 6-phenoxy-pyridazine ring, with GABA embedded in the 2-position (2 butyric acid). Based on historical, low-potency predecessors of gabazine, a longer GABA moiety (2 pentanoic acid), and methyl substituents (e.g., 5-CH3) were explored (FIG. 2F). For each variant, the 6-phenoxy moiety was modified to make a gabazinealkyne module, which was click-conjugated to DART.2. Several variants were made, including gabazine.1^{DART.2} (2 butyric acid, 5-H), gabazine.5DART.2 (2 pentanoic acid, 5-H), and gabazine.7DART.2 (2 pentanoic acid, 5-CH3). An all-optical assay of endogenous GABAARs in primary neuronal culture was then developed. Several assays were tested and converged upon a configuration in which ChR2 and GCaMP6s were co-expressed in the same neuron. Excitatory synapses were blocked, such that opto-stimulation with ChR2 became the only source of neural excitation. Under these cell autonomous conditions, the GCaMP signal was attenuated by bath application of 15 uM GABA, indicating that endogenous GABAARs can overpower ChR2. Subsequent addition of gabazine restored the ChR2-driven GCaMP response, affording a means to perform dose-response characterizations of GABAAR antagonism. ⁺HTP and ^{dd}HTP cells were equally affected by the traditional (non-DART) version of gabazine. In contrast, the DART reagents exhibited pronounced cellular specificity. The first, most potent gabazine.1^{DART.2} yielded a DW (t=15 m)=500. The attenuated variants provided even greater cellular specificity, with gabazine.5^{DART.2} yielding DW (t=15 m)=1,500, and gabazine.7^{DART.2} achieving a DW (t=15 m)=3,000. Results for different gabazine. DARTS can be seen in FIG. 2G, FIG. 2H, and FIG. 2I.

A surprise challenged the intuition that attenuating the pharmacophore would not only reduce impact of ambient compound (raise Rx50), but also that of tethered drugs (raise HTL50 to a similar extent). Thus, in developing the attenuated gabazine variants, it would have been unsurprising if the DW shifted to higher doses with no impact on its width. In contrast to this intuition, converting gabazine.1DART.2 to gabazine.7DART.2 had a larger impact on Rx50 (increased ˜20-fold) than on HTL50 (increased only ˜3-fold), yielding a substantial ˜6-fold increase in the DW. To explain this phenomenon, models were explored that relate how changes in Rx50 would impact HTL50 given a range of possible expression levels of the HTP and GABAAR. It was found that the data could be explained by a model in which an average of 4 HTP proteins are within proximity to each GABAAR site. In this “stoichiometry-limited” regime, HTL50 is specified predominantly by the number of tethered drugs, rather than their affinity for the GABAAR.

### Example 25

### Reagents for Rigor and Reproducibility in Behaving Animals

Next, AAV viral-vector modifications were explored in parallel to the chemistry described above. In particular, although the original HTPNLG (432-648) expressed well via plasmid transfection in culture, its virally packaged length (5.2 kb) was ˜10% longer than AAV capacity (4.7 kb). Shorter single-pass transmembrane anchors were tested, but none matched the trafficking performance of the original design. In contrast, fusion of HTP, via a (GGSGG)₈ (SEQ ID NO:3) flexible linker, to a Thy1 GPI anchor afforded a short design (4.6 kb), while matching the trafficking performance and pharmacological efficacy of HTPTM-NLG in cultured neurons. Thus, active AAV-DIO-+HTPGPI and matched control AAV-DIO-ddHTPGPI viruses were generated, and a functional titer calibration procedure was established to ensure equipotent expression from different viral lots. In the dorsal striatum, it was found that substantially fewer viral particles of the new HTPGPI design (˜1e12 GC/mL×1 μL) achieved comparable expression to the original HTPNLG (432-648) design (˜5e12 GC/mL×1 μL), indicative of a higher fraction of properly packaged capsids. The newer design thus afforded a means of viral transduction that minim/zed nonspecific toxicity.

Next, it was sought to address the issue of pharmaceutical target engagement in live mice. This feature was lacking in the original DART method, making it difficult to detect an HTP trafficking deficit in any given cell type, or a failed infusion in any given mouse. To address this, it was reasoned that a 10:1 mixture of an RxDART.2 with a fluorescent tracerDART.2 would maintain this ratio during biodistribution and tethering, such that each captured tracerDART.2 molecule would serve as a visible proxy for ˜10 RxDART.2 molecules in the same vicinity. These assumptions rely upon equivalent capture kinetics of the HTL.2 moiety, and similar mass and electrostatic properties of the two reagents; a reasonable assumption given that PEG36 dominates the physical properties. Thus, Alexa488.1DART.2 and Alexa647.1DART.2 reagents were developed for easy mixing and matching of either tracerDART.2 with any RxDART.2 reagent. As an additional control, a blank.1DART.2 was developed, with neither drug nor dye, as an inert control. For example, a 10:1 mixture of blank.1DART.2+Alexa647.1DART.2 would enable a control experiment to rule out pharmaceutical properties of the tracer dye fraction, or theoretical impact of PEG accumulation on the surface of a neuron.

### Example 26

### AMPAR Antagonism in Live Mice

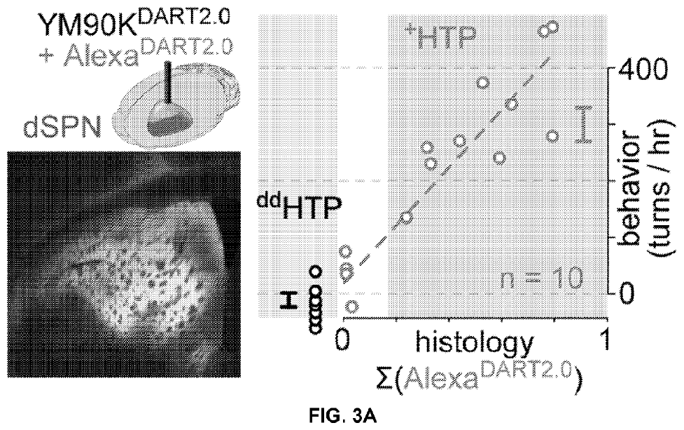

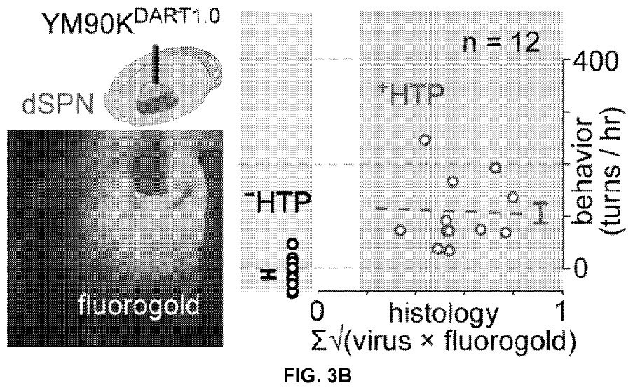

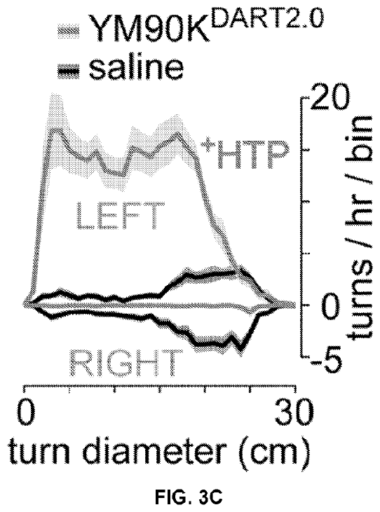

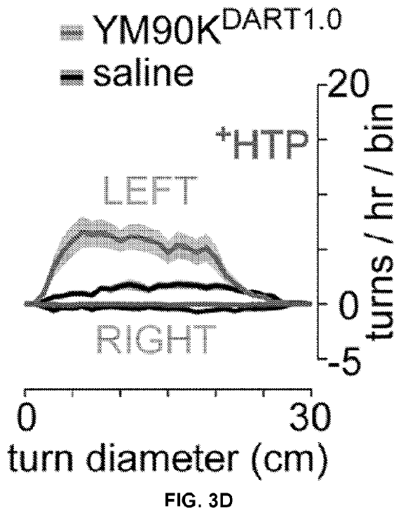

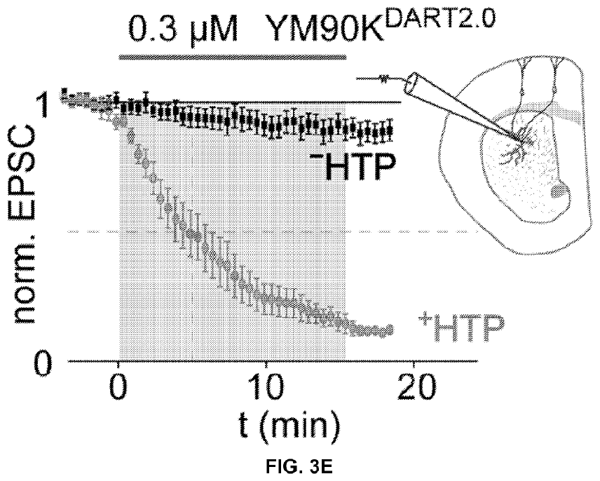

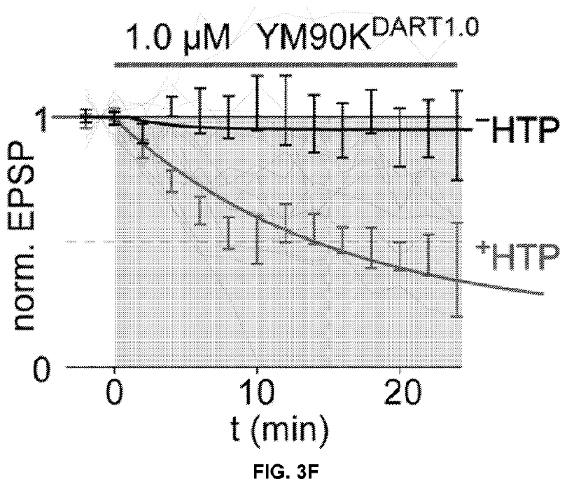

These advances were combined in a mouse locomotor assay. AAV-DIO-⁺HTPGPI or AAV-DIO-ddHTPGPI were expressed in the left dorsal striatum of D1-Cre mice, and implanted a unilateral cannula. Three weeks later, a baseline open-field behavior (saline infusion) was obtained; followed one week later by infusion of a 10:1 mixture of YM90K.1DART.2/Alexa488.1DART.2 (FIG. 3A). Mice were perfused for histology 24 hrs later, and quantitative measurement of the integrated Alexa capture over the dorsal striatum was performed. The magnitude of open-field turning vs dye capture (FIG. 3C) was then plotted A control was also performed as above using DART.1 and non-DART fluorescent dye (fluorogold) (FIG. 3B and FIG. 3D). Three points merit emphasis. First, a turning bias in +HTP mice was seen, with no turning bias in ddHTP animals, confirming that behavioral effects are mediated by tethered drug, not ambient drug. Second, the data yielded a tight correlation between behavioral effects and histology; in particular the four animals with the weakest behavioral impact corresponded to those with little dye capture, providing an unbiased parameter with which to exclude these animals due to technical failure. Such a correlation was not seen with the original technology, despite efforts to estimate cannula patency with a non-DART fluorescent tracer. Third, in these behaving mice, YM90K.1DART.2 was substantially more efficacious than its predecessor, driving a 3-fold larger impact in a locomotor assay following unilateral delivery to dSPNs of the dorsal striatum (FIG. 3E and FIG. 3F). Experiments in acute striatal brain slice confirmed the performance boost afforded by DART.2. Low-dose, YM90K.1DART.2 produced rapid AMPAR antagonism on +HTP neurons, achieving half-block after only 5 min of a 0.3 UM dose. This represents a ˜10-fold acceleration in comparison to its predecessor, which required 15 min of a 1 UM dose to achieve a similar block. Neither reagent produced any measurable impact on −HTP or ddHTP neurons at these doses. However, only YM90K.1DART.2 offered kinetics fast enough for routine slice physiology experiments.

### Example 27

### GABAAR Antagonism in Live Mice

Next, the feasibility of using DART.2 for GABAAR antagonism in live mice was explored. The analysis began with cerebellar granule cells, which exhibit an unusual form of sparse coding-being the most abundant, yet least active type of neuron in the brain. Whether this sparsity results from low excitatory drive, or high inhibitory GABAAR tone, has been difficult to address with prior tools. For example, one may attempt to reduce inhibition onto granule cells by silencing local GABA-producing neurons [golgi cells]. However, this would disinhibit local excitatory neurons [unipolar brush cells]. Thus the approach would inadvertently elevate excitatory drive onto granule cells, concomitant with the desired goal of reducing their inhibition, confounding interpretation. DART offers a new opportunity to address this question, by making it possible to manipulate the GABAAR on a sparse subset of granule cells, without disrupting other aspects of cerebellar circuit dynamics. AAV DIO ⁺HTPGPI or AAV DIO ^{dd}HTPGPI were expressed along with GCaMPxx in xxx-Cre mice; 2p. Experiments in acute slice revealed a pronounced tonic GABAAR current in granule cells, which was antagonized by gabazine.1DART.2 with an HTL50 (t=15 m)˜50 nM for ⁺HTP cells, and an Rx50˜1,000 nM for ddHTP cells. Next, in vivo experiments, using two-photon imaging of granule cells were performed. Given potency of the ambient reagent, an improved procedure was used for local infusion of gabazine.1DART.2. This dosing procedure produced no impact on GCaMP signals in ddHTP mice, indicating no measurable impact of the ambient compound. In contrast, a pronounced increase in +HTP granule cell activity was seen. These data demonstrate a direct, causal role for inhibitory GABAAR tone on granule cells in maintaining their firing stringency, and set the stage for future studies examining behavioral roles of sparse granule cell coding.

In contrast to granule cells, midbrain dopamine (mDA) neurons are few in number, and exhibit high levels of intrinsically generated pacemaker activity, akin to a heartbeat. A practical issue for intrinsically active neurons is that one can no longer leverage the concept of a floor-effect (i.e., silencing an already quiet neuron), making it difficult to apply optogenetic principles of necessity and sufficiency. Thus, it was sought to examine the technical feasibility of using DART to block neurotransmitter signaling onto these cells; in essence, preventing extrinsic neurotransmitter modulation of dopamine neurons, without disrupting their intrinsic rhythm. Moreover, it was sought to use behavior as the readout, which would likely require manipulation of more cells over a larger brain volume than in the prior experiment. Given this goal, a wide dosing window is key, and thus effort was focused on gabazine.7DART.2, with expression of AAV DIO +HTPGPI or AAV DIO ddHTPGPI in DAT-Cre mice. Acute slice confirmed a pronounced block of evoked GABAAR current onto +HTP mDA neurons, with no measurable effect on ddHTP cells. The same gabazine.7DART.2 manipulation on +HTP cells had no impact on NMDA or AMPA-mediated transmission, nor impact on other measurable electrophysiological parameters, indicating a selective manipulation of the GABAAR. Finally, it was confirmed that a 10:1 ratio of blank.1DART.2+Alexa647.1DART.2 had no impact on any physiological parameter, indicating that the tethered tracer dye is inert. Next, locomotor effects of the manipulations were assessed using the same viral parameters along with bilateral indwelling cannula. Infusion of a 10:1 mixture of gabazine.7DART.2 and Alexa647.1DART.2 produced a significant increase in running speed in ⁺HTP mice, whereas the same dose had no impact in ^{dd}HTP animals. Subsequent histology confirmed uniform xxx (histo vs behavior). This is consistent with prior studies showing that increased dopaminergic tone increases locomotion. In contrast to these pronounced locomotor effects, YM90K.1DART.2 had no impact on locomotion in these animals, despite producing potent antagonism of the AMPAR in slice experiments. The data establish the safety and efficacy of a set of gabazineDART.2 reagents in behaving mice. Moreover, the findings indicate that the GABAAR may play a more pronounced role than the AMPAR in modulating locomotor aspects of dopamine neuron activity.

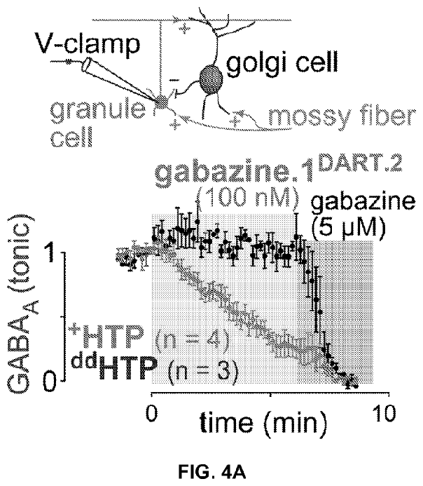

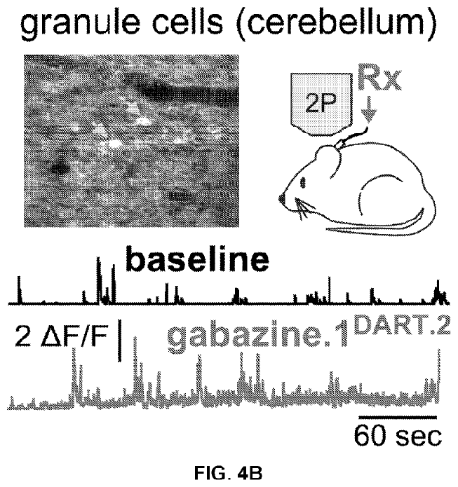

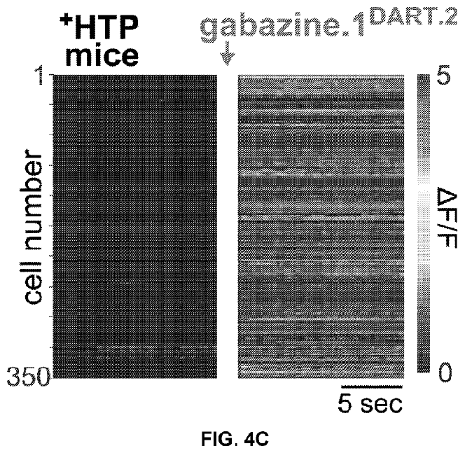

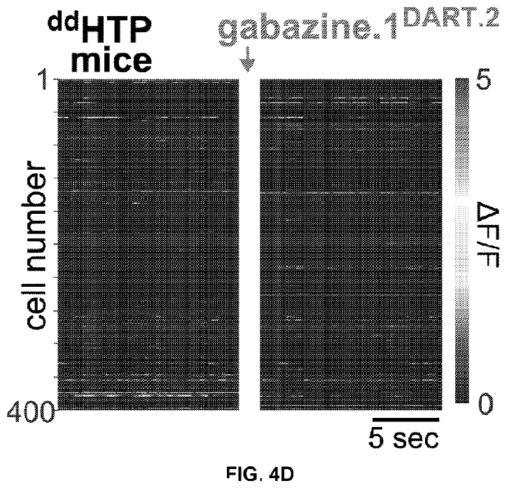

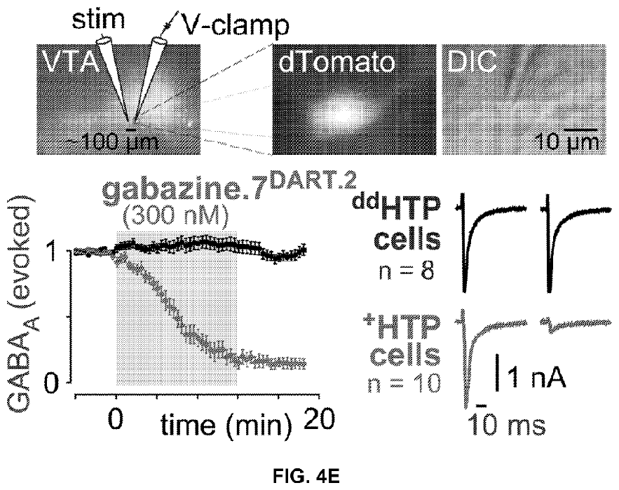

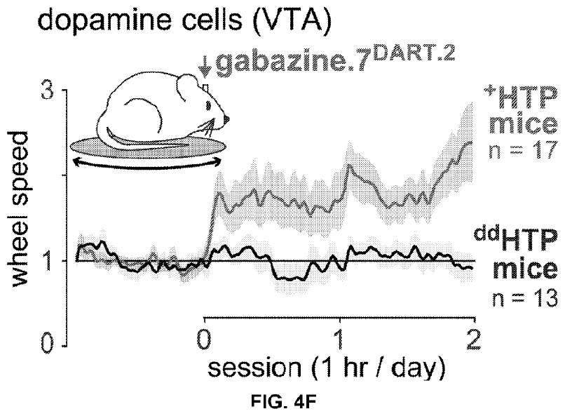

Results for this Example can be seen in FIG. 4A, FIG. 4B, FIG. 4C, FIG. 4D, FIG. 4E, and FIG. 4F.

### Example 28

### Extension to PAMS

Next it was sought to extend DART to allosteric modulators. The allosteric binding site of the GABAAR, known to bind several drugs, with effects ranging from negative to positive allosteric modulation was first analyzed. Diazepam represents the prototypic PAM at this site, and is the most clinically significant of the class. Thus, a diazepam analog was synthesized with a short alkyne attached to the amide of the diazepam core; an attachment site known to tolerate substituents and thought from structural inspection of the GABAAR-bound diazepam pose to allow linker access to free solution. Click chemistry was used to assemble the full-length diazepam.1DART.2 and leveraged an all-optical assay of endogenous GABAARs in primary neuronal culture, with ChR2 and GCaMP6s in the same cell. Here, a sub-effective 5 UM dose of GABA was used, such that allosteric modulation of the endogenous GABAAR was required to overpower ChR2. +HTP and ddHTP cells were equally affected by the traditional (non-DART) version of diazepam. In contrast, diazepam.1DART.2 exhibited pronounced cellular specificity, with Rx50=30 μM on ddHTP neurons and HTL50 (t=15 m)=40 nM on +HTP neurons, altogether yielding a DW (t=15 m)˜750. Thus, the impact of the tether attenuated the binding affinity of diazepam, without affecting its allosteric impact once bound; a pattern reminiscent of the experience with antagonists.

The ease with which diazepam was developed into a DART was fortuitous, albeit unexpected. Thus, it was wondered whether full-strength positive allosteric modulators, like diazepam, might be special. In particular, it was envisioned that receptor modulation by a PAM could reach an allosteric ceiling effect, wherein any additional binding-site perturbation caused by the tether would produce little added impact on receptor function. To test this idea, flumazenil was explored, which has neither positive nor negative allosteric impact on the GABAAR, despite binding the same allosteric site as diazepam. It was hypothesized, without being bound by a particular theory, that flumazenil would not push the GABAAR into a conformational extreme, and thus might be more sensitive to subtle steric impacts of a tether. Thus, a flumazenil.1DART.2 reagent was developed via conjugation to an analogous amide of the flumazenil core, and click-chemistry assembly. Whereas traditional flumazenil showed no receptor modulation in the assay, it was found that flumazenil.1DART.2 took on features of a PAM, similar to that of diazepam.1DART.2. Thus, greater conformational sensitivity with flumazenil was overserved, in comparison to diazepam, consistent with the concept of an allosteric ceiling effect. Though tentative, these examples reveal a putative design principle, wherein drugs that impart maximal conformational changes may be more likely to retain their original function upon tether attachment than those with partial or neutral conformational impact.

To complete the bidirectional ionotropic toolkit, allosteric modulators of the AMPAR were explored. For many pharmaceuticals of this class, including cyclothiazide, two drug molecules are known to bind in close proximity, one on each side of the interface between adjacent ligand-binding domains. Thus, newer pharmaceuticals of this class were focused on, which can span the two binding sites simultaneously, reasoning that it would be easier to accommodate a single tether into the binding interface. In particular, CMPDA (phenyl-1,4-bisalkylsulfonamide) was focused on, owing to its structural simplicity and availability of an experimental atomic structure. Moreover, CMPDA has been shown to produce potent allosteric effects, stronger than cyclothiazide, and thus it was reasoned that the drug may operate near the allosteric ceiling, making it less sensitive to conformational perturbation by the tether. A synthetic scheme to resynthesize CMPDA while installing a short PEG spacer domain at a point of attachment was devised to allow tether access from the allosteric binding site to free solution. The resulting CMPDA.1alkyne retained its ability to positively modulate the AMPAR, however, its function was lost upon conjugation to a longer PEG36 moiety. Further occlusion experiments indicated that the failure mode was a lack of binding, rather than a change in allosteric efficacy once bound. Taken together, these data suggested that the local impact of alkylation by a short PEG could be tolerated by CMPDA, but that a longer PEG36 linker could not span the distance from the binding pocket out to free solution. Upon further inspection of the atomic structure, it was noted that this path had an overall negative surface potential, which was speculated could be repulsive to the negative electrostatic properties of PEG. Altering the electrostatic properties of the spacer domain was explored. In silico docking and experimental iteration to redesign the electronegative spacer (CCOCCOCC) into a polar neutral spacer (CBNCCCNC) was used, yielding CMPDA.2alkyne, which was converted into the full-length CMPDA.2DART.2. This modification restored the ability of the reagent to bind and produce positive allosterically modulation of the AMPAR.

### Example 29

### Whole Brain Delivery

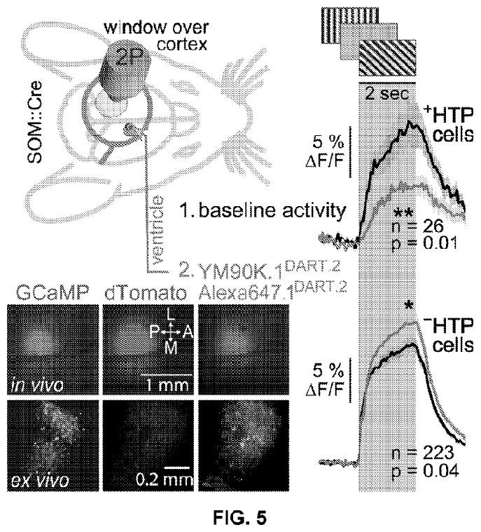

To maintain control of the ambient drug concentration in vivo, thus far a cannula was implanted in close proximity to the desired brain target. (FIG. 5) However, optical and electrical brain interfaces often occupy this precious real estate. For example, cranial window technologies, in which a portion of the skull is replaced with a glass window, have become instrumental in studying the neocortex. Application of drugs through a glass window remains an unsolved problem, as access ports will generally close due to scarring, and temporary removal of the window can compromise the preparation. Thus, it was asked whether DART.2 could enable dosing from a distance via the cerebrospinal fluid (CSF). Delivery via the cisterna magna was first explored, given reports that dextran tracers delivered at this site achieve rapid biodistribution throughout the neocortex parenchyma. Although there was some preliminary success with this approach, it was found difficult to establish mechanically stable cannulation of the cisterna magna, owing to its position below the base of the skull and to the mobility of the neck muscles. Delivery via the lateral ventricle was explored, which is amenable to stable cannulation in mice. Given that CSF flows from the lateral ventricle to the cisterna magna, there was concern that there could be a long delay in reaching the cortex. Thus, the biodistribution kinetics were first characterized. Mice received a surgery to implant a cannula in one lateral ventricle, along with a glass window over the contralateral visual cortex, with no virus (i.e., −HTP mice). Following surgical recovery, a high dose of Alexa647.1DART.2 was fused into the LV (dose, volume) and monitored wide-field fluorescence intensity through the cranial window. Fluorescence intensity peaked 2 hr later, and largely cleared by 24 hours (i2055,i2056). Thus, in principle, this delivery strategy can enable DART reagents to reach the cortex.

Next, it was examined whether this strategy could deliver a pharmacologically active DART to genetically defined cells. A surgery was performed in which AAV DIO +HTPGPI was injected into the V1 visual cortex of SOM-Cre mice for cell-specific expression in somatostatin interneurons, along with a co-injected AAV-GCaMP8s virus for pan-neuronal neural activity monitoring. A cannula was implanted in the contralateral ventricle, and glass window over V1. Following surgical recovery, a mixture of YM90K.1DART.2+Alexa647.1DART.2 was infused. Comparison of widefield fluorescence of +HTP (somatostatin interneurons) and −HTP (all other neurons) showed that DART capture reached a steady state within 4-8 hours, suggesting that +HTP cells in the field of view were fully tethered by then. To test for pharmacological effects, 2P calcium imaging of +HTP and −HTP was performed while mice were presented with oriented gratings. A reduction in +HTP visual response was apparent 4 hours post DART infusion. This reduction persisted even after 24 hours post infusion. 24 hours post infusion of YM90K.1DART.2, +HTP cells showed a decrease in visual response (i2044, i2051, i2052, i2053, 50% contrast) relative to pre-DART response while −HTP cells showed no change or increase in visual response. The increase in visual response in some −HTP cells suggest that these cells may be more dependent on inhibition from SOM cells than neighboring −HTP cells, which were less affected by the manipulation.

Achieving consistent dosing across experimental contexts is a major challenge in behavioral neuropharmacology, particularly in local-dosing strategies. For example, infusion of a drug into a given brain region will generally have a physiological effect at a high-enough dose. However, one can never be certain whether effects are mediated by the brain region of interest, or if effects are mediated by spillover into one or more adjacent brain structures. The fundamental unknown is the ambient concentration profile in the intact brain, which can differ depending on brain region, brain state, drug clearance, and other complex mechanisms. DART solves this problem by making the ambient drug concentration essentially irrelevant. So long as HTP saturation is achieved, the tethered Rx concentration becomes specified entirely by HTP expression level. The practical implications are substantial. For example, the approach makes it possible to achieve in vivo dosing identical to that in a slice electrophysiology experiment. The tethered Rx concentration can be measured quantitatively on every cell via measurement of the fluorescent tracer. In other scenarios, one could imagine co-delivering two RxDART reagents to the same cell (e.g., gabazineDART.2+YM90K.1DART.2) in a dual-treatment group, and comparing this to single-treatment groups in which blank.1DART.2 serves as an inert substitute for either reagent.

### Example 30

### Bidirectional Manipulation of Chemical Neurotransmission

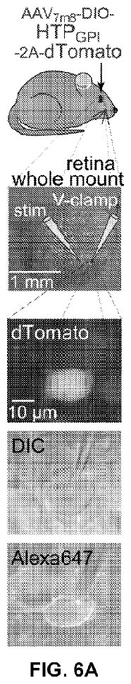

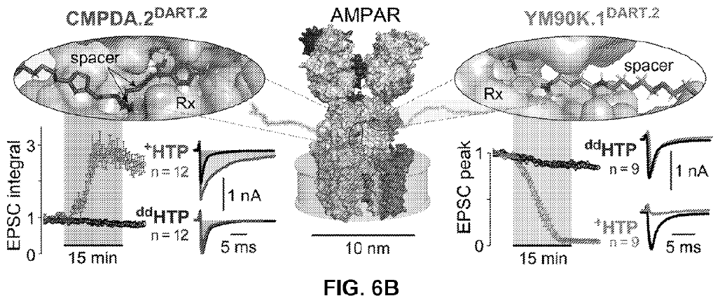

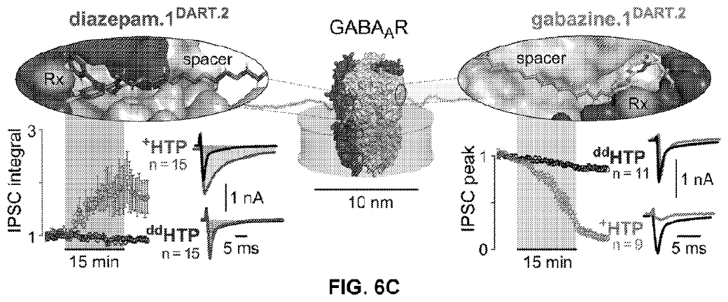

Next it was asked whether bidirectional modulation of the AMPAR and GABAAR could be achieved in the same cell type. A preparation was focused on in which electrophysiology is the primary readout, enabling resolution of the amplitude and millisecond time-constant of synaptic transmission. The retina was selected as the tissue of interest, given potential opportunities afforded by the whole-mount preparation, wherein the intact circuit can be studied under conditions amenable to electrophysiology (Masland and Raviola, 2000, which is incorporated by reference herein in its entirety). A subset of retinal ganglion cells (RGCs) were focused on and a combination of viral serotype and mouse strain were used to genetically instruct parvalbumin-positive RGCs to express ⁺HTPGPI or ^{dd}HTPGPI (FIG. 6A). A whole-mount retina was later prepared and evoked synaptic currents were measured under conditions that isolate the AMPAR or GABAAR. For each cell, a stable baseline was established, then delivered a given RxDART.2 (300 nM)+Alexa647.1DART.2 (30 nM) for 15 min, followed by washout. At the end of each experiment the Alexa647 channel was imaged and a robust capture on the surface of ⁺HTP cells was observed, with no detectible capture on ^{dd}HTP or −HTP cells (FIG. 6A).

Starting with PAM reagents, CMPDA.2DART.2 strongly potentiated AMPAR-mediated EPSC (excitatory post-synaptic current) on +HTP RGCs, both by augmenting the peak amplitude and by prolonging the decay time, altogether yielding a three-fold increase in the integrated EPSC charge transfer (FIG. 6B). No impact of CMPDA.2DART.2 was seen in analogous experiments in ddHTP RGCs (FIG. 6B). The impact of diazepam.1DART.2 on the GABAAR-mediated IPSC (inhibitory post-synaptic current) was evaluated. It was observed that this reagent significantly potentiated IPSCs, with an analogous increase in peak amplitude and prolongation of decay time. Again, the effects were only seen on +HTP RGCs, with no effect on ddHTP RGCs (FIG. 6C). Finally, it was confirmed that gabazine.1DART.2 blocked the GABAAR-mediated IPSC (FIG. 6C), and that YM90K.1DART.2 blocked the AMPAR-mediated EPSC (FIG. 6B). With all reagents, maximal effects took hold within 15 min for ⁺HTP RGCs, and no effects were seen on ^{dd}HTP RGCs.

It is understood that the foregoing detailed description and accompanying examples are merely illustrative and are not to be taken as limitations upon the scope of the invention.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. A compound of formula (I)

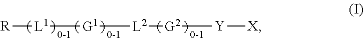

or a salt thereof, wherein: R is a functional group selected from the group consisting of a binding group, a detection label, a biologically active agent, a biorthogonal functional group, and a substrate; L¹, at each occurrence, is a linker; L² is a linker; G¹, at each occurrence, is a C₆₋₁₂arylene, a 5- to 12-membered heteroarylene, C₃₋₆cycloalkylene, or a 4- to 6-membered heterocyclylene, wherein G¹ is optionally substituted with 1-4 R^{1x}, wherein, at each occurrence, R^{1x} is independently selected from the group consisting of halogen, oxo, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, and —C₁₋₃alkylene-Q¹; R^{1a}, R^{1b}, and R^{1c}, at each occurrence, are each independently hydrogen, C₁₋₆alkyl,
C₁₋₆haloalkyl, G^{1a}, or —C₁₋₃alkylene-G^{1a}; R^{1d}, at each occurrence, is independently C₁₋₆alkyl, C₁₋₆haloalkyl, G^{1a}, or —C₁₋₃alkylene-G^{1a}; G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂; Q¹, at each occurrence, is independently-OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂; G², at each occurrence, is a C₆₋₁₂arylene, a 5- to 12-membered heteroarylene, C₃₋₆cycloalkylene, or a 4- to 6-membered heterocyclylene, wherein G² is optionally substituted with 1-4 R^{2x}, wherein, at each occurrence, R^{2x} is independently selected from the group consisting of halogen, oxo, C₁₋₆alkyl, C₁₋₆haloalkyl, C₂₋₆alkenyl, —OR^{2a}, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, and —C₁₋₃alkylene-Q²; R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a}; R^{2d}, at each occurrence, is independently C₁₋₆alkyl, C₁₋₆haloalkyl, G^{2a}, or —C₁₋₃alkylene-G^{2a}; G^{2a}, at each occurrence, is independently a C₃₋₈cycloalkyl, a 4- to 12-membered heterocyclyl, a 6- to 12-membered aryl, or a 5- to 12-membered heteroaryl, wherein G^{2a} is optionally substituted with 1-5 substituents independently selected from the group consisting of halogen, oxo, C₁₋₄alkyl, —OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O) NH₂, —C(O)NHC₁₋₄alkyl, and —C(O)N(C₁₋₄alkyl)₂; Q², at each occurrence, is independently-OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O) NH₂, —C(O)NHC₁₋₄alkyl, or —C(O)N(C₁₋₄alkyl)₂; Y is alkylene, alkenylene, or alkynylene; and X is halogen, wherein at least one of G¹ and G² is present.

Clause 2. The compound of clause 1, or a salt thereof, wherein: L¹ is alkylene, heteroalkylene, heteroalkylene-C(O)NR^{a}-heteroalkylene, heteroalkylene-C(O)NR^{a}-alkylene, heteroalkylene-C(O)O-heteroalkylene, heteroalkylene-OC(O)NR^{a}-heteroalkylene, heteroalkylene-C(O)O-heteroalkylene-C(O)-alkylene, heteroalkylene-C(O)NR^{a}-heteroalkylene-C(O)-alkylene, or heteroalkylene-OC(O)NR^{a}-heteroalkylene-C(O)-alkylene; L² is —C(O)NR^{a}-heteroalkylene, —C(O)NR^{a}-alkylene-NR^{a}C(O)—, or —C(O)NR^{a}-alkylene-C(O)NR^{a}-alkylene; and R^{a}, at each occurrence, is independently hydrogen or alkyl.

Clause 3. The compound of clause 1 or 2, or a salt thereof, wherein: L¹ is

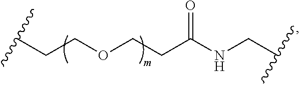

wherein m is 1 to 6,000; L² is —C(O)NR^{a}—C₂₋₁₀heteroalkylene, —C(O)NR^{a}—C₁₋₁₀alkylene-NR^{a}C(O)—, or —C(O)NR^{a}—C₁₋₁₀alkylene-C(O)NR^{a}—C₁₋₆alkylene; and R^{a} is hydrogen or C₁₋₃alkyl.

Clause 4. The compound of any one of clauses 1-3, or a salt thereof, wherein: G¹ is optionally substituted with 1-2 R^{1x}, wherein, at each occurrence, R^{1x} is selected from the group consisting of halogen, C₁₋₆haloalkyl, —OR^{1a}, —NR^{1a}R^{2b}, —SR^{1a}, —NR^{1a}C(O)R^{1c}, —C(O)OR^{1a}, —C(O)NR^{1a}R^{1b}, and —C(O)R^{1c}; and R^{1a}, R^{1b}, and R^{1c}, at each occurrence, are each independently hydrogen, C₁₋₆alkyl, or C₁₋₆haloalkyl.

Clause 5. The compound of any one of clauses 1-4, or a salt thereof, wherein: G² is substituted with 1-2 R^{2x}, wherein, at each occurrence, R^{2x} is selected from the group consisting of halogen, C₁₋₆haloalkyl, —OR^{2a}, —NR^{2a}R^{2b}, —SR^{2a}, —NR^{2a}C(O)R^{2c}, —C(O)OR^{2a}, —C(O)NR^{2a}R^{2b}, —C(O)R^{2c}, —SO₂R^{2d}, and —SO₂NR^{2a}R^{2b}; and R^{2a}, R^{2b}, R^{2c}, and R^{2d}, at each occurrence, are each independently hydrogen, C₁₋₆alkyl, or C₁₋₆haloalkyl.

Clause 6. The compound of any one of clauses 1-5, or a salt thereof, wherein: G¹ is C₆₋₈arylene, C₄₋₆cycloalkylene, the 4- to 6-membered heterocyclylene, or the 5- to 12-membered heteroarylene, where the ring system of the 5- to 12-membered heteroarylene at G¹ is a 5- to 8-membered heteroarylene; G² is C₆₋₈arylene, C₄₋₆cycloalkylene, the 4- to 6-membered heterocyclylene, or the 5- to 12-membered heteroarylene, where the ring system of the 5- to 12-membered heteroarylene at G² is a 5- to 8-membered heteroarylene, wherein G² is substituted with 1-2 R^{2x}, wherein, at each occurrence, R^{2x} is independently selected from the group consisting of halogen, C₁₋₆haloalkyl, —OR^{2a}, —NR^{2a}R^{2b}, —SR^{2a}, —NR^{2a}C(O)R^{2c}, —C(O)OR^{2a}, —C(O)NR^{2a}R^{2b}, —C(O)R^{2c}, —SO₂R^{2d}, and —SO₂NR^{2a}R^{2b}; and R^{2a}, R^{2b}, R^{2c}, and R^{2d}, at each occurrence, are each independently hydrogen, C₁₋₆alkyl, or C₁₋₆haloalkyl.

Clause 7. The compound of any one of clauses 1-6, or a salt thereof, wherein: G¹ is C₆₋₈arylene, the 5- to 8-membered heteroarylene, C₄₋₆cycloalkylene, or the 4- to 6-membered heterocyclylene; G² is C₆₋₈arylene, the 5- to 8-membered heteroarylene, C₄₋₆cycloalkylene, or the 4- to 6-membered heterocyclylene, wherein G² is substituted with 1 R^{2x}, wherein R^{2x} is selected from the group consisting of halogen and —OR^{2a}; and R^{2a} is hydrogen or C₁₋₄alkyl.

Clause 8. The compound of any one of clauses 1-7, or a salt thereof, wherein: Y is C₁₋₁₀alkylene, C₁₋₁₀alkenylene, or C₁₋₁₀alkynylene.

Clause 9. The compound of any one of clauses 1-8, or a salt thereof, wherein: X is Cl, Br, or I.

Clause 10. The compound of clause 1, or a salt thereof, having formula (I-b):

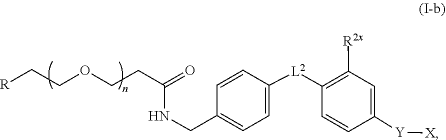

wherein: R is a functional group selected from the group consisting of a binding group, a detection label, a biologically active agent, a biorthogonal functional group, and a substrate; L²

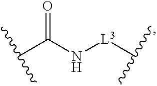

is wherein: L³ is heteroalkylene, alkylene-NR^{a}C(O)—, or alkylene-C(O)NR^{a}-alkylene; R^{a}, at each occurrence, is hydrogen or C₁₋₄alkyl; Y is alkylene, alkenylene, or alkynylene; X is halogen; R^{2x} is C₁₋₆haloalkyl, —OR^{2a}, —NR^{2a}R^{2b}, —SR^{2a}, —NR^{2a}C(O)R^{2c}, —C(O) OR^{2a}, —C(O)NR^{2a}R^{2b}, —C(O)R^{2c}, —SO₂R^{2d}, or —SO₂NR^{2a}R^{2b}; R^{2a}, R^{2b}, R^{2c}, and R^{2d}, at each occurrence, are each independently hydrogen, C₁₋₆alkyl, or C₁₋₆haloalkyl; and n is 1 to 6,000.

Clause 11. The compound of clause 1 or 10, or a salt thereof, wherein: L³ is C₂-10heteroalkylene, C₁₋₁₀alkylene-NR^{a}C(O)—, or C₁₋₁₀alkylene-C(O)NR^{a}—C₁₋₆alkylene; Y is C₁₋₁₀alkylene, C₁₋₁₀alkenylene, or C₁₋₁₀alkynylene; R^{2x} is halogen or —OR^{2a}; R^{2a} is hydrogen or C₁₋₄alkyl; and n is 1 to 1,000.

Clause 12. The compound of any one of clauses 1-11, or a salt thereof, wherein the binding group comprises a peptide, a protein, a carbohydrate, a lipid, a small molecule, a nucleic acid, or a combination thereof.

Clause 13. The compound of any one of clauses 1-12, or a salt thereof, wherein the detection label comprises a chromophore, a fluorophore, a radiolabel, a polynucleotide, a small molecule, an enzyme, a nanoparticle, a microparticle, a quantum dot, or an upconverter.

Clause 14. The compound of any one of clauses 1-13, or a salt thereof, wherein the detection label comprises Alexa 647, Alexa 488, TMR, or Oregon Green.

Clause 15. The compound of any one of clauses 1-14, or a salt thereof, wherein the biologically active agent comprises an agonist, an antagonist, an allosteric modulator, an inverse agonist, a partial agonist, or a biased agonist.

Clause 16. The compound of any one of clauses 1-15, or a salt thereof, wherein the biologically active agent comprises gabazine, diazepam, flumazenil, CMPDA, YM90K, atropine, naloxone, oxymorphone, THC rimonabant, PPHT, NAPS, or haloperidol, or a pharmaceutically acceptable salt thereof.

Clause 17. The compound of any one of clauses 1-16, or a salt thereof, wherein the biorthogonal functional group comprises an azide, an alkyne, a maleimide, an iodoacetamide, a thiol, a disulfide, a NHS ester, a tetrazine, a trans-cyclooctene, a ketone/aldehyde, a hydrazine, a hydrazide, or a thioacid.

Clause 18. The compound of any one of clauses 1-17, or a salt thereof, wherein the substrate comprises a particle, an electrical conducting support, or a magnetic bead.

Clause 19. A pharmaceutical composition comprising the compound of any one of clauses 1-18, or a pharmaceutically acceptable salt thereof, and a pharmaceutically acceptable excipient.

Clause 20. A composition comprising a protein linked to a functional group by a linker of formula (II)

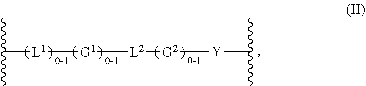

wherein: the functional group is selected from the group consisting of a binding group, a detection label, a biologically active agent, a biorthogonal functional group, and a substrate; L¹, G¹, L², G², and Y are defined as in clause 1; the protein comprises a mutant dehalogenase, and wherein at least one of G¹ and G² is present.

Clause 21. A recombinant protein comprising a mutant dehalogenase having at least 85% sequence identity to SEQ ID NO: 1, wherein the recombinant protein has three amino acid substitutions within a catalytic triad of the mutant dehalogenase and two amino acid substitutions within a tunnel entrance of the mutant dehalogenase.

Clause 22. The recombinant protein of clause 21, wherein the recombinant protein has amino acid substitutions at residues 41, 106, 107, 245, and 246 of a mutant dehalogenase having SEQ ID NO:1.

Clause 23. The recombinant protein of clause 21 or 22, wherein the amino acid substitutions are N41E, D106E, W107G, V245L, and L246R.

Clause 24. The recombinant protein of any one of clauses 21-23, wherein the mutant dehalogenase has at least 95% sequence identity to SEQ ID NO:1.

Clause 25. The recombinant protein of any one of clauses 21-24, wherein the mutant dehalogenase is Rhodococcus dehalogenase having the amino acid sequence of SEQ ID NO:2.

Clause 26. A method of modulating a cell, the method comprising: contacting a cell comprising a mutant dehalogenase on a surface of the cell with the compound of any one of clauses 1-18, wherein the mutant dehalogenase forms a bond with the compound, and wherein R binds to a receptor on a surface of the cell.

Clause 27. The method of clause 26, wherein the receptor is an ionotropic receptor, a metabotropic receptor, a voltage-gated ion channel, or a tyrosine kinase receptor.

Clause 28. The method of clause 26 or 27, wherein the receptor is a GAB_{A}A receptor, an AMPA receptor, a GPCR, a NaV voltage-gated channel, a KV voltage-gated channel, a CaV voltage-gated channel, or a TrkB.

Clause 29. A method of labeling a cell, the method comprising contacting a cell comprising a mutant dehalogenase located at a surface of the cell with the compound of any one of clauses 1-18, wherein the mutant dehalogenase forms a bond with the compound, thereby labeling the cell with R.

Clause 30. A method of detecting or determining a presence or an amount of a mutant dehalogenase, the method comprising: contacting a mutant dehalogenase with the compound of any one of clauses 1-18, wherein the mutant dehalogenase forms a bond with the compound; and detecting or determining the presence or the amount of R, thereby detecting or determining the presence or the amount of the mutant dehalogenase.

Clause 31. The method of clause 30, wherein the mutant dehalogenase is located at a surface of a cell.

Clause 32. The method of any one of clauses 26-31, wherein the cell is a neuron, a muscle cell, an endocrine cell, a keratinocyte, a glial cell, a cell line expressing voltage-gated ion channels, an immune cell, or a CAR-T cell.

Clause 33. The method of any one of clauses 26-32, wherein the cell is present in a subject.

Clause 34. A method of modulating neurotransmission in a subject in need thereof, the method comprising administering to the subject an effective amount of the compound of any one of clauses 1-18, or a pharmaceutically acceptable salt thereof, optionally in combination with a pharmaceutically acceptable excipient.

Clause 35. The method of clause 34, wherein R is a biologically active agent.

Clause 36. The method of clause 34 or 35, wherein modulating neurotransmission in the subject affects locomotion, mood, anxiety, addiction, attention, psychosis, inflammation, or a combination thereof of the subject.

Clause 37. The method of any one of clauses 34-36, wherein the compound, or a pharmaceutically acceptable salt thereof, is localized to a specific region of the subject's brain.

Clause 38. The method of any one of clauses 34-37, wherein the compound, or a pharmaceutically acceptable salt thereof, is administered to the subject through the cerebrospinal fluid of the subject.

SEQUENCES
SEQ ID NO: 1
GSEIGTGFPFDPHYVEVLGERMHYVDVGPRDGTPVLFLHGNPTSS
YVWRNIIPHVAPTHRCIAPDLIGMGKSDKPDLGYFFDDHVRFMDA
FIEALGLEEVVLVIHDWGSALGFHWAKRNPERVKGIAFMEFIRPI
PTWDEWPEFARETFQAFRTTDVGRKLIIDQNVFIEGTLPMGVVRP
LTEVEMDHYREPFLNPVDREPLWRFPNELPIAGEPANIVALVEEY
MDWLHQSPVPKLLFWGTPGVLIPPAEAARLAKSLPNCKAVDIGPG
LNLLQEDNPDLIGSEIARWLSTLEISG
SEQ ID NO: 2
GSEIGTGFPFDPHYVEVLGERMHYVDVGPRDGTPVLFLHGEPTSS
YVWRNIIPHVAPTHRCIAPDLIGMGKSDKPDLGYFFDDHVRFMDA
FIEALGLEEVVLVIHEGGSALGFHWAKRNPERVKGIAFMEFIRPI
PTWDEWPEFARETFQAFRTTDVGRKLIIDQNVFIEGTLPMGVVRP
LTEVEMDHYREPFLNPVDREPLWRFPNELPIAGEPANIVALVEEY
MDWLHQSPVPKLLFWGTPGLRIPPAEAARLAKSLPNCKAVDIGPG
LNLLQEDNPDLIGSEIARWLSTLEISG
SEQ ID NO: 3
(GGSGG)₈

What is claimed is:

1. A compound of formula (I) 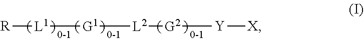
or a salt thereof, wherein:
R is a functional group selected from the group consisting of a binding group, a detection label, a biologically active agent, a biorthogonal functional group, and a substrate;
L¹, at each occurrence, is a linker;
L² is a linker;
G¹, at each occurrence, is a C₆₋₁₂arylene, a 5- to 12-membered heteroarylene, C₃₋₆cycloalkylene, or a 4- to 6-membered heterocyclylene, wherein G¹ is optionally substituted with 1-4 R^{1x}, wherein, at each occurrence, R^{1x} is independently selected from the group consisting of halogen, oxo, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, and —C₁₋₃alkylene-Q¹;
R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q¹, at each occurrence, is independently-OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
G², at each occurrence, is a C₆₋₁₂arylene, a 5- to 12-membered heteroarylene, C₃₋₆cycloalkylene, or a 4- to 6-membered heterocyclylene, wherein G² is optionally substituted with 1-4 R^{2x}, wherein, at each occurrence, R^{2x} is independently selected from the group consisting of halogen, oxo, C₁₋₆alkyl, C₁₋₆haloalkyl, C₂₋₆alkenyl, —OR^{2a}, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G^{2a}, —C₁₋₃alkylene-G^{2a}, and —C₁₋₃alkylene-Q²;
R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a};
R^{2d}, at each occurrence, is independently C₁₋₆alkyl, C₁₋₆haloalkyl, G^{2a}, or —C₁₋₃alkylene-G^{2a};
G^{2a}, at each occurrence, is independently a C₃₋₈cycloalkyl, a 4- to 12-membered heterocyclyl, a 6- to 12-membered aryl, or a 5- to 12-membered heteroaryl, wherein G^{2a} is optionally substituted with 1-5 substituents independently selected from the group consisting of halogen, oxo, C₁₋₄alkyl, —OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q², at each occurrence, is independently-OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂,
Y is alkylene, alkenylene, or alkynylene; and
X is halogen,
wherein at least one of G¹ and G² is present.

2. The compound of claim 1, or a salt thereof, wherein:
L¹ is alkylene, heteroalkylene, heteroalkylene-C(O)NR^{a}-heteroalkylene, heteroalkylene-C(O)NR^{a}-alkylene, heteroalkylene-C(O)O-heteroalkylene, heteroalkylene-OC(O)NR^{a}-heteroalkylene, heteroalkylene-C(O)O-heteroalkylene-C(O)-alkylene, heteroalkylene-C(O)NR^{a}-heteroalkylene-C(O)-alkylene, or heteroalkylene-OC(O)NR^{a}-heteroalkylene-C(O)-alkylene;
L² is —C(O)NR^{a}-heteroalkylene, —C(O)NR^{a}-alkylene-NR^{a}C(O)—, or —C(O)NR^{a}-alkylene-C(O)NR^{a}-alkylene; and
R^{a}, at each occurrence, is independently hydrogen or alkyl.

3. The compound of claim 1, or a salt thereof, wherein:
L¹ is 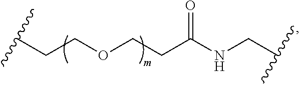
wherein m is 1 to 6,000;
L² is —C(O)NR^{a}—C₂₋₁₀heteroalkylene, —C(O)NR^{a}—C₁₋₁₀alkylene-NR^{a}C(O)—, or —C(O)NR^{a}—C₁₋₁₀alkylene-C(O)NR^{a}—C₁₋₆alkylene; and
R^{a} is hydrogen or C₁₋₃alkyl.

4. The compound of claim 1, or a salt thereof, wherein:
G¹ is optionally substituted with 1-2 R^{1x}, wherein, at each occurrence, R^{1x} is selected from the group consisting of halogen, C₁₋₆haloalkyl, —OR^{1a}, —NR^{1a}R^{2b}, —SR^{1a}, —NR^{1a}C(O)R^{1c}, —C(O)OR^{1a}, —C(O)NR^{1a}R^{1b}, and —C(O)R^{1c}; and
R^{1a}, R^{1b}, and R^{1c}, at each occurrence, are each independently hydrogen, C₁₋₆alkyl, or C₁₋₆haloalkyl.

5. The compound of claim 1, or a salt thereof, wherein:
G² is substituted with 1-2 R^{2x}, wherein, at each occurrence, R^{2x} is selected from the group consisting of halogen, C₁₋₆haloalkyl, —OR^{2a}, —NR^{2a}R^{2b}, —SR^{2a}, —NR^{2a}C(O)R^{2c}, —C(O)OR^{2a}, —C(O)NR^{2a}R^{2b}, —C(O)R^{2c}, —SO₂R^{2d}, and —SO₂NR^{2a}R^{2b}; and
R^{2a}, R^{2b}, R^{2c}, and R^{2d}, at each occurrence, are each independently hydrogen, C₁₋₆alkyl, or C₁₋₆haloalkyl.

6. The compound of claim 1, or a salt thereof, wherein:
G¹ is C₆₋₈arylene, C₄₋₆cycloalkylene, the 4- to 6-membered heterocyclylene, or the 5- to 12-membered heteroarylene, where the ring system of the 5- to 12-membered heteroarylene at G¹ is a 5- to 8-membered heteroarylene;
G² is C₆₋₈arylene, C₄₋₆cycloalkylene, the 4- to 6-membered heterocyclylene, or the 5- to 12-membered heteroarylene, where the ring system of the 5- to 12-membered heteroarylene at G² is a 5- to 8-membered heteroarylene, wherein G² is substituted with 1-2 R^{2x}, wherein, at each occurrence, R^{2x} is independently selected from the group consisting of halogen, C₁₋₆haloalkyl, —OR^{2a}, —NR^{2a}R^{2b}, —SR^{2a}, —NR^{2a}C(O)R^{2c}, —C(O)OR^{2a}, —C(O)NR^{2a}R^{2b}, —C(O)R^{2c}, —SO₂R^{2d}, and —SO₂NR^{2a}R^{2b}; and
R^{2a}, R^{2b}, R^{2c}, and R^{2d}, at each occurrence, are each independently hydrogen, C₁₋₆alkyl, or C₁₋₆haloalkyl.

7. The compound of claim 6, or a salt thereof, wherein:
G¹ is C₆₋₈arylene, the 5- to 8-membered heteroarylene, C₄₋₆cycloalkylene, or the 4- to 6-membered heterocyclylene;
G² is C₆₋₈arylene, the 5- to 8-membered heteroarylene, C₄₋₆cycloalkylene, or the 4- to 6-membered heterocyclylene, wherein G² is substituted with 1 R^{2x}, wherein R^{2x} is selected from the group consisting of halogen and —OR^{2a}; and
R^{2a} is hydrogen or C₁₋₄alkyl.

8. The compound of claim 1, or a salt thereof, wherein:
Y is C₁₋₁₀alkylene, C₁₋₁₀alkenylene, or C₁₋₁₀alkynylene.

9. The compound of claim 1, or a salt thereof, wherein:
X is Cl, Br, or I.

10. The compound of claim 1, or a salt thereof, having formula (I-b): 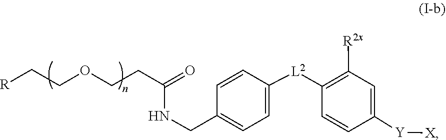
wherein:
R is a functional group selected from the group consisting of a binding group, a detection label, a biologically active agent, a biorthogonal functional group, and a substrate;
L² is 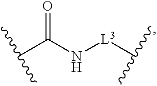
wherein:
L³ is heteroalkylene, alkylene-NR^{a}C(O)—, or alkylene-C(O)NR^{a}-alkylene;
R^{a}, at each occurrence, is hydrogen or C₁₋₄alkyl;
Y is alkylene, alkenylene, or alkynylene;
X is halogen;
R^{2x} is C₁₋₆haloalkyl, —OR^{2a}, —NR^{2a}R^{2b}, —SR^{2a}, —NR^{2a}C(O)R^{2c}, —C(O)OR^{2a}, —C(O)NR^{2a}R^{2b}, —C(O)R^{2c}, —SO₂R^{2d}, or —SO₂NR^{2a}R^{2b};
R^{2a}, R^{2b}, R^{2c}, and R^{2d}, at each occurrence, are each independently hydrogen, C₁₋₆alkyl, or C₁₋₆haloalkyl; and
n is 1 to 6,000.

11. The compound of claim 10, or a salt thereof, wherein:
L³ is C₂₋₁₀heteroalkylene, C₁₋₁₀alkylene-NR^{a}C(O)—, or C₁₋₁₀alkylene-C(O)NR^{a}—C₁₋₆alkylene;
Y is C₁₋₁₀alkylene, C₁₋₁₀alkenylene, or C₁₋₁₀alkynylene;
R^{2x} is halogen or —OR^{2a};
R^{2a} is hydrogen or C₁₋₄alkyl; and
n is 1 to 1,000.

12. The compound of claim 1, or a salt thereof, wherein the binding group comprises a peptide, a protein, a carbohydrate, a lipid, a small molecule, a nucleic acid, or a combination thereof.

13. The compound of claim 1, or a salt thereof, wherein the detection label comprises a chromophore, a fluorophore, a radiolabel, a polynucleotide, a small molecule, an enzyme, a nanoparticle, a microparticle, a quantum dot, or an upconverter.

14. The compound of claim 1, or a salt thereof, wherein the detection label comprises Alexa 647, Alexa 488, TMR, or Oregon Green.

15. The compound of claim 1, or a salt thereof, wherein the biologically active agent comprises an agonist, an antagonist, an allosteric modulator, an inverse agonist, a partial agonist, or a biased agonist.

16. The compound of claim 15, or a salt thereof, wherein the biologically active agent comprises gabazine, diazepam, flumazenil, CMPDA, YM90K, atropine, naloxone, oxymorphone, THC rimonabant, PPHT, NAPS, or haloperidol, or a pharmaceutically acceptable salt thereof.

17. The compound of claim 1, or a salt thereof, wherein the biorthogonal functional group comprises an azide, an alkyne, a maleimide, an iodoacetamide, a thiol, a disulfide, a NHS ester, a tetrazine, a trans-cyclooctene, a ketone/aldehyde, a hydrazine, a hydrazide, or a thioacid.

18. The compound of claim 1, or a salt thereof, wherein the substrate comprises a particle, an electrical conducting support, or a magnetic bead.

19. A pharmaceutical composition comprising the compound of claim 1, or a pharmaceutically acceptable salt thereof, and a pharmaceutically acceptable excipient.

20. A composition comprising a protein linked to a functional group by a linker of formula (II) 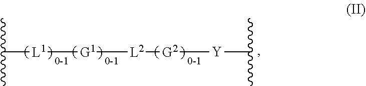
wherein:
the functional group is selected from the group consisting of a binding group, a detection label, a biologically active agent, a biorthogonal functional group, and a substrate;
L¹, at each occurrence, is a linker;
L² is a linker;
G¹, at each occurrence, is a C₆₋₁₂arylene, a 5- to 12-membered heteroarylene, C₃₋₆cycloalkylene, or a 4- to 6-membered heterocyclylene, wherein G¹ is optionally substituted with 1-4 R^{1x}, wherein, at each occurrence, R^{1x} is independently selected from the group consisting of halogen, oxo, C₁₋₆alkyl, C₁₋₆haloalkyl, C₂₋₆alkenyl, —OR^{1a}, —NR^{1a}R^{1b}, —SR^{1a}, —NR^{1a}C(O)R^{1c}, cyano, —C(O)OR^{1a}, —C(O)NR^{1a}R^{1b}, —C(O)R^{1c}, —SO₂R^{1d}, —SO₂NR^{1a}R^{1b}, G^{1a}, —C₁₋₃alkylene-G^{1a}, and —C₁₋₃alkylene-Q¹;
R^{1a}, R^{1b}, and R^{1c}, at each occurrence, are each independently hydrogen, C₁₋₆alkyl, C₁₋₆haloalkyl, G^{1a}, or —C₁₋₃alkylene-G^{1a};
R^{1d}, at each occurrence, is independently C₁₋₆alkyl, C₁₋₆haloalkyl, G^{1a}, or —C₁₋₃alkylene-G^{1a};
G^{1a}, at each occurrence, is independently a C₃₋₈cycloalkyl, a 4- to 12-membered heterocyclyl, a 6- to 12-membered aryl, or a 5- to 12-membered heteroaryl, wherein G^{1a} is optionally substituted with 1-5 substituents independently selected from the group consisting of halogen, oxo, C₁₋₄alkyl, —OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂;
Q¹, at each occurrence, is independently-OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
G², at each occurrence, is C₆₋₁₂arylene, a 5- to 12-membered heteroarylene, C₃₋₆cycloalkylene, or a 4- to 6-membered heterocyclylene, wherein G² is optionally substituted with 1-4 R^{2x}, wherein, at each occurrence, R^{2x} is independently selected from the group consisting of halogen, oxo, C₁₋₆alkyl, C₁₋₆haloalkyl, C₂₋₆alkenyl, —OR^{2a}, —NR^{2a}R^{2b}, —SR^{2a}, —NR^{2a}C(O)R^{2c}, cyano, —C(O)OR^{2a}, —C(O)NR^{2a}R^{2b}, —C(O)R^{2c}, —SO₂R^{2d}, —SO₂NR^{2a}R^{2b}, G²a, —C₁₋₃alkylene-G^{2a}, and —C₁₋₃alkylene-Q²;
R^{2a}, R^{2b}, and R^{2c}, at each occurrence, are each independently hydrogen, C₁₋₆alkyl, C₁₋₆haloalkyl, G^{2a}, or —C₁₋₃alkylene-G^{2a};
R^{2d}, at each occurrence, is independently C₁₋₆alkyl, C₁₋₆haloalkyl, G^{2a}, or —C₁₋₃alkylene-G^{2a};
G^{2a}, at each occurrence, is independently a C₃₋₈cycloalkyl, a 4- to 12-membered heterocyclyl, a 6- to 12-membered aryl, or a 5- to 12-membered heteroaryl, wherein G^{2a} is optionally substituted with 1-5 substituents independently selected from the group consisting of halogen, oxo, C₁₋₄alkyl, —OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, and —C(O)N(C₁₋₄alkyl)₂,
Q², at each occurrence, is independently-OC₁₋₄alkyl, —OC₁₋₄haloalkyl, OH, NH₂, —NHC₁₋₄alkyl, —N(C₁₋₄alkyl)₂, cyano, —C(O)OC₁₋₄alkyl, —C(O)NH₂, —C(O)NHC₁₋₄alkyl, or —C(O)N(C₁₋₄alkyl)₂;
Y is alkylene, alkenylene, or alkynylene; and
the protein comprises a mutant dehalogenase,
wherein at least one of G¹ and G² is present.

21. A recombinant protein comprising a mutant dehalogenase having at least 85% sequence identity to SEQ ID NO: 1, wherein the recombinant protein has three amino acid substitutions within a catalytic triad of the mutant dehalogenase and two amino acid substitutions within a tunnel entrance of the mutant dehalogenase.

22. The recombinant protein of claim 21, wherein the recombinant protein has amino acid substitutions at residues 41, 106, 107, 245, and 246 of a mutant dehalogenase having SEQ ID NO: 1.

23. The recombinant protein of claim 22, wherein the amino acid substitutions are N41E, D106E, W107G, V245L, and L246R.

24. The recombinant protein of claim 21, wherein the mutant dehalogenase has at least 95% sequence identity to SEQ ID NO: 1.

25. The recombinant protein of claim 21, wherein the mutant dehalogenase is Rhodococcus dehalogenase having the amino acid sequence of SEQ ID NO:2.

26. A method of modulating a cell, the method comprising:
contacting a cell comprising a mutant dehalogenase on a surface of the cell with the compound of claim 1, wherein the mutant dehalogenase forms a bond with the compound, and wherein R binds to a receptor on a surface of the cell.

27. The method of claim 26, wherein the receptor is an ionotropic receptor, a metabotropic receptor, a voltage-gated ion channel, or a tyrosine kinase receptor.

28. The method of claim 27, wherein the receptor is a GAB_{A}A receptor, an AMPA receptor, a GPCR, a NaV voltage-gated channel, a KV voltage-gated channel, a CaV voltage-gated channel, or a TrkB.

29. A method of labeling a cell, the method comprising:
contacting a cell comprising a mutant dehalogenase located at a surface of the cell with the compound of claim 1, wherein the mutant dehalogenase forms a bond with the compound, thereby labeling the cell with R.

30. A method of detecting or determining a presence or an amount of a mutant dehalogenase, the method comprising:
contacting a mutant dehalogenase with the compound of claim 1, wherein the mutant dehalogenase forms a bond with the compound; and
detecting or determining the presence or the amount of R, thereby detecting or determining the presence or the amount of the mutant dehalogenase.

31. The method of claim 30, wherein the mutant dehalogenase is located at a surface of a cell.

32. The method of claim 26, wherein the cell is a neuron, a muscle cell, an endocrine cell, a keratinocyte, a glial cell, a cell line expressing voltage-gated ion channels, an immune cell, or a CAR-T cell.

33. The method of claim 26, wherein the cell is present in a subject.

34. A method of modulating neurotransmission in a subject in need thereof, the method comprising administering to the subject an effective amount of the compound of claim 1, or a pharmaceutically acceptable salt thereof, optionally in combination with a pharmaceutically acceptable excipient.

35. The method of claim 34, wherein R is a biologically active agent.

36. The method of claim 34, wherein modulating neurotransmission in the subject affects locomotion, mood, anxiety, addiction, attention, psychosis, inflammation, or a combination thereof of the subject.

37. The method of claim 34, wherein the compound, or a pharmaceutically acceptable salt thereof, is localized to a specific region of the subject's brain.

38. The method of claim 34, wherein the compound, or a pharmaceutically acceptable salt thereof, is administered to the subject through the cerebrospinal fluid of the subject.
